(19) United States
(12) Patent Application Publication
(10) Pub. No.: US 2022/0388980 A1
(43) Pub. Date: Dec. 8, 2022

(54) QUINOLINE INHIBITORS OF RAD52 AND METHODS OF USE

(71) Applicant: Drexel University, Philadelphia, PA (US)

(72) Inventors: Alexander V. Mazin, Philadelphia, PA (US); Patrick Y. S. Lam, Chadds Ford, PA (US); Kritika Hanamshet, Philadelphia, PA (US); Mikir Sunil Patel, Philadelphia, PA (US); Yanming Du, Cheshire, CT (US); Nicki Hwang, Doylestown, PA (US)

(21) Appl. No.: 17/762,257

(22) PCT Filed: Oct. 1, 2020
(86) PCT No.: PCT/US20/53813

§ 371 Date: Mar. 21, 2022

Related U.S. Application Data
(60) Provisional application No. 62/909,017, filed on Oct. 1, 2019.

(51) Int. Cl. C07D 401/12; C07D 215/38; C07D 401/14

(52) U.S. Cl. CPC C07D 401/12; C07D 215/38; C07D 401/14

(57) ABSTRACT

The present disclosure related to compounds of Formula I and Formula I and to their pharmaceutically acceptable salts, pharmaceutical compositions, methods of use, and methods for their preparation. The compounds disclosed herein are useful for modulating RAD52 activity and may be used in the treatment of disorders in which RAD52 activity is implication, such as a cancer.

### CROSS REFERENCE TO RELATED APPLICATIONS

This application claims priority under 35 U.S.C. § 119(e) to U.S. Provisional Application No. 62/909,017, filed Oct. 1, 2019, the content of which is incorporated by reference herein in its entirety.

### BACKGROUND

The present disclosure relates to small molecule modulators of RAD52, designed for the treatment of cancer and other disorders associated with RAD52.

DNA repair is essential for maintenance of genome integrity in all organisms. Numerous DNA repair systems evolved to eliminate a broad variety of DNA lesions caused by exogenous agents or genotoxic products of metabolism. In normal cells, the specificities of different DNA repair mechanisms overlap to assure efficient genome protection. However, cancer cells often lose some DNA repair pathways due to intrinsic genome instability. In this case, cancer cell viability depends on the remaining alternative DNA repair mechanisms.

Poly (ADP-ribose) polymerase 1 (PARP1), a protein involved in DNA damage signaling and repair of DNA single-stranded breaks (SSB), is essential for viability of cancer cells that are deficient in the homologous recombination (HR) pathway. Furthermore, hereditary breast cancer and ovarian cancer cells, which often carry mutations in HR proteins BRCA1 or BRCA2, can be eliminated using PARP1 inhibitors with a minimal harm to normal cells with at least one copy of functional BRCA1/2 genes.

BRCA1/2-deficient cancer cells are not viable when RAD52 protein is inactivated. In addition, RAD52 knockdown also causes lethality to human cells deficient in PALB2 (partner and localizer of BRCA2) and five RAD51 paralogs, including RAD51C. Mutations in PALB2 and RAD51C also contribute to hereditary breast and ovarian cancer. Previously, inviability of double mutations in RAD52 and RAD51C genes was reported in chicken DT-40 cells. Inactivation of PARP1 and RAD52 causes lethality of BRCA1/2-deficient and PALB2-deficient cells through different mechanisms. Inactivation of PARP1 causes disruption of repair of DNA SSBs. During DNA replication, unrepaired SSBs may cause formation of DNA double-stranded breaks (DSBs) or stalled replication forks, which are repaired by the HR pathway. BRCA1/2/PALB2 constitute the major sub-pathway of HR; mutations in these proteins incapacitates HR making hereditary breast and ovarian cancer cells vulnerable to PARP1 inhibitors. However, recent data have demonstrated that, in addition to the BRCA1/2/PALB2 sub-pathway, the secondary HR sub-pathway operates in mammalian cell that depends on RAD52 protein. In normal mammalian cells, this pathway plays a minor role, as RAD52−/− mice are viable and fertile and do not display DNA damage sensitivity, abnormalities, or significant cancer predisposition. However, this sub-pathway becomes essential for viability in cells that lack the BRCA1/2/PALB2 sub-pathway.

Thus, there is a need in the art to develop compounds with improved RAD52 modulation (e.g., inhibition of RAD52) that are useful for the treatment of cancers, as well as diseases and disorders which are modulated by RAD52. The present disclosure addresses this unmet need.

### SUMMARY

In some aspects, the present disclosure provides compounds of Formula I′:

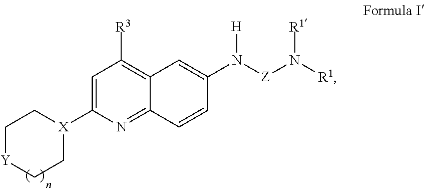

and pharmaceutically acceptable salts and solvates thereof, wherein:

X is CH or N;

Y is CH₂ or N—R²;

Z is

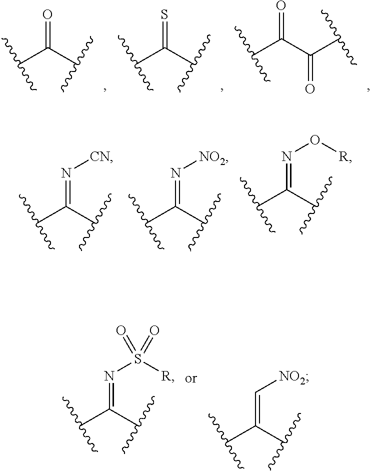

R¹ is H, C₁₋₆ alkyl optionally substituted with one or more N(R⁴)(R^{4′}), —C₀₋₆ alkyl-(4- to 8-membered heterocyclyl), or -(4- to 8-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl;

R^{1′} is H or C₁₋₆ alkyl, or

R¹ and R^{1′}, together with the nitrogen atom to which R¹ and R^{1′} are attached, form a 5- to 6-membered heterocyclyl optionally substituted with one or more R⁵;

R² is H, C₁₋₆ alkyl, —C₃₋₆ cycloalkyl-C₀₋₆ alkyl, —C₀₋₆ alkyl-C₃₋₆ cycloalkyl, -(3- to 7-membered heterocyclyl)-C₀₋₆ alkyl, —C₀₋₅ alkyl-(3- to 7-membered heterocyclyl), —(C₆₋₁₀ aryl)-C₀₋₆ alkyl, —C₀₋₅ alkyl-(C₆₋₁₀ aryl), -(3- to 7-membered heteroaryl)-C₀₋₆ alkyl, —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl, cycloalkyl, heterocyclyl, aryl, heteroaryl represented by R² is optionally substituted with one or more —OH, —NH₂, —NH—C(═O)—O—(C₁₋₆ alkyl), halogen, C₁₋₆ alkyl, or phenyl;

R³ is H, C₁₋₆ alkyl, C₂₋₆ alkenyl, C₂₋₆ alkynyl, C₁₋₆ haloalkyl, halogen, —CN, —NO₂, —OR, —SR, —S(═O)₂R, —C(═O)R, —OC(═O)R, —NR₂, or —CO₂R;

each R⁴ and R^{4′} is independently H, —C(═O)—O—(C₁₋₆ alkyl), C₁₋₆ alkyl, C₂₋₆ alkenyl, C₂₋₆ alkynyl, or C₃₋₆ cycloalkyl;

R⁵ is -(5- to 7-membered heterocyclyl)-C₀₋₆-alkyl, —C₀₋₆-alkyl-(5- to 7-membered heterocyclyl), —(C₃₋₆ cycloalkyl)-C₀₋₆-alkyl, or —C₀₋₆-alkyl-(C₃₋₆ cycloalkyl), wherein the alkyl, heterocyclyl, or cycloalkyl represented by R⁵ is optionally substituted by one or more C₁₋₆ alkyl;

each R is independently H, C₁₋₆ alkyl, C₂₋₆ alkenyl, C₂₋₆ alkynyl, or C₃₋₆ cycloalkyl; and

n is 0 or 1,

provided that R² and R³ are not simultaneously CH₃.

In some aspects, the present disclosure provides a compound obtainable by, or obtained by, a method for preparing a compound as described herein (e.g., a method comprising one or more steps described in Schemes 1-23).

In some aspects, the present disclosure provides a pharmaceutical composition comprising a compound of the present disclosure, or a pharmaceutically acceptable salt thereof, and a pharmaceutically acceptable diluent or carrier.

In some aspects, the present disclosure provides an intermediate as described herein, being suitable for use in a method for preparing a compound as described herein (e.g., the intermediate is selected from the intermediates described in Example 1).

In some aspects, the present disclosure provides a method of modulating RAD52 activity (e.g., in vitro or in vivo), comprising contacting a cell with an effective amount of a compound of the present disclosure or a pharmaceutically acceptable salt thereof.

In some aspects, the present disclosure provides a method of treating or preventing a disease or disorder disclosed herein, comprising administering to a subject in need thereof, a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein, comprising administering to a subject in need thereof, a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in modulating RAD52 activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides use of a compound of the present disclosure or a pharmaceutically acceptable salt thereof in the manufacture of a medicament for modulating RAD52 activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides a method of preparing a compound of the present disclosure.

In some aspects, the present disclosure provides a method of preparing a compound, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE FIGURES

The drawings illustrate generally, by way of example, but not by way of limitation, some embodiments of the present disclosure.

FIGS. 1A-1C depicts identification and characterization of RAD52 small molecule inhibitors. FIG. 1A depicts the experimental scheme of fluorescence-quenching assay for the RAD52 ssDNA annealing activity (FLU stands for fluorescein; BHQ 1 stands for black hole quencher 1; and DNA substrates contain a mismatch to block spontaneous reaction). FIG. 1B depicts the kinetics of ssDNA annealing measured on a FluoroMax3 fluorimeter. FIG. 1C depicts the scheme of the D-loop assay, wherein RAD52 forms a complex with ssDNA and promotes its homologous pairing with pUC19 plasmid DNA (the asterisk denotes ³²P label on ssDNA).

FIG. 2A depicts the results of a CellTiterGlo Luminescence assay for DLD1 BRCA2+/+ and BRCA2−/− cells with compound 0047. FIG. 2B depicts the results of a CellTiterGlo Luminescence assay for DLD1 BRCA2+/+ and BRCA2−/− cells with compound 0056.

FIG. 3A depicts viability of BRCA proficient (black) and deficient (grey) cells from the pancreatic adenocarcinoma cancer cell line CAPAN-1 following treatment with 10 μM 6-hydroxydopamine (6-OH-dopa). FIG. 3B depicts viability of BRCA proficient (black) and deficient (grey) cells from the BRCA1 deficient triple negative breast cancer cell line HCC1937 following treatment with 5 μM 6-OH-dopa. FIG. 3C depicts clonogenic survival of acute myeloid leukemia (AML) cells from patients with low expression of BRCA1/2 following treatments with 6-OH-dopa. FIG. 3D depicts clonogenic survival of chronic myelogenous leukemia (CML) cells from patients with low expression of BRCA1 following treatments with 6-OH-dopa. FIG. 3E depicts clonogenic survival of BRCA1-deficient breast cancer cells from the cell line MDA-MB-436 following treatments with 6-OH-dopa. FIGS. 3A, 3B, 3C, 3D, and 3E are adapted from Chandramouly, Gurushankar et al. “Small-Molecule Disruption of RAD52 Rings as a Mechanism for Precision Medicine in BRCA-Deficient Cancers.” Chemistry & Biology vol. 22, 11 (2015): 1491-1504.

FIG. 4 depicts growth of BRCA1-null HCC1937 cells (grey dots) and their BRCA1-reconstitued counterparts (black dots) in the presence of indicated concentrations of 5-aminoimidazole-4-carboxamide ribonucleotide. FIG. 4 is adapted from Sullivan, Katherine et al. “Identification of a Small Molecule Inhibitor of RAD52 by Structure-Based Selection.” PloS one vol. 11, 1 e0147230. 19 Jan. 2016.

### DETAILED DESCRIPTION OF THE INVENTION

The present disclosure relates to quinoline derivatives, prodrugs, and pharmaceutically acceptable salts thereof, which may modulate RAD52 activity and are accordingly useful in methods of treatment of the human or animal body. The present disclosure also relates to processes for the preparation of these compounds, to pharmaceutical compositions comprising them and to their use in the treatment of disorders wherein RAD52 is implicated, such as cancer.

While the disclosed subject matter will be described in conjunction with the enumerated claims, it will be understood that the exemplified subject matter is not intended to limit the claims to the disclosed subject matter.

### Definitions

Unless otherwise stated, the following terms used in the specification and claims have the following meanings set out below.

Throughout this document, values expressed in a range format should be interpreted in a flexible manner to include not only the numerical values recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. In some embodiments, examples of organic groups include, but are not limited to, OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include, but are not limited to, F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. In some embodiments, examples of straight chain alkyl groups include, but are not limited to, those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any one of the groups listed herein, for example, but not limited to, amino, hydroxy, cyano, carboxy, nitro, thio, alkoxy, and halogen groups.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkenyl” as used herein refers to straight and branched chain and cyclic alkyl groups as defined herein, but that contain at least one double bond between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to, vinyl, —CH═C═CH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

The term “alkynyl” as used herein refers to straight and branched chain alkyl groups, but that contain at least one triple bond between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, −CH₂C≡C(CH₃), and —CH₂C≡C(CH₂CH₃) among others.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of “aryl” and “heteroaryl” groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

As used herein, the term “hydroxy” or “hydroxyl” includes groups with an —OH or —O—.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like. Any number of hydrogen atoms in the haloalkyl group can be substituted with halogen atoms.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

A substituent may comprise more than one functional group in sequence (e.g. C₀₋₆ alkyl-C₄₋₈ heterocyclyl). Whenever a variable is defined in this way, the substituent may be connected to the rest of the molecule at either end. For example, the term “C₄₋₈ heterocyclyl-C₀₋₆ alkyl” should be understood to encompass at least both of the following substituents [symbol] (where represents the point of attachment to the rest of the molecule):

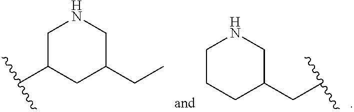

When a substituent comprising more than one functional group is indicated to be “optionally substituted,” one, both, or neither functional group may be substituted as indicated, unless the context indicates otherwise. For example, the term “optionally substituted C₃₋₆ heterocyclyl-C₁₋₆ alkyl, wherein the optional substitution is chlorine” should be understood to encompass at least the following substituents:

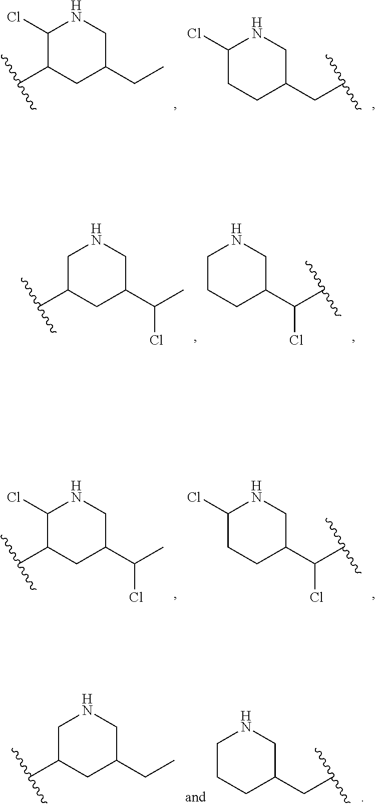

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise.

Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

The term “room temperature” as used herein refers to a temperature of about 15° C. to 28° C.

The term “standard temperature and pressure” as used herein refers to 20° C. and 101 kPa.

It is to be understood that the present disclosure provides methods for the synthesis of the compounds of any of the Formulae described herein. The present disclosure also provides detailed methods for the synthesis of various disclosed compounds of the present disclosure according to the following scheme as well as those shown in the Examples.

It is to be understood that, throughout the description, where compositions are described as having, including, or comprising specific components, it is contemplated that compositions may optionally consist of the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field.

Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art

One of ordinary skill in the art will note that, during the reaction sequences and synthetic scheme described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject in need thereof can be one who has been previously diagnosed or identified as having a disease or disorder disclosed herein. A subject in need thereof can also be one who is suffering from a disease or disorder disclosed herein.

Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “composition” or “pharmaceutical composition” refers to a formulation of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system.

An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to those compounds, anions, cations, materials, compositions, carriers, and/or dosage forms, which do not abrogate the biological activity or properties of the compound, and are relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment,” “treating” or “treat” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

As used herein, the term “preventing,” “prevent,” or “protecting against” describes reducing or eliminating the onset of the symptoms or complications of such disease, condition or disorder.

Techniques for formulation and administration of the disclosed compounds of the disclosure can be found in Remington: the Science and Practice of Pharmacy, 191^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic scheme described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Compounds of the Present Disclosure

Compounds of Formula I′ and Formula I, and pharmaceutically acceptable salts or solvates thereof, or otherwise described herein can be prepared by the general schemes described herein, using the synthetic method known by those skilled in the art. The following examples illustrate non-limiting embodiments of the compound(s) described herein and their preparation.

In some aspects, the present disclosure provides compounds of Formula I′:

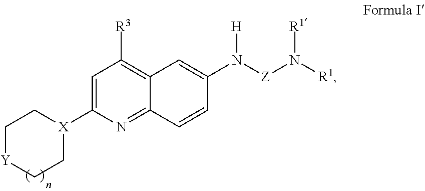

and pharmaceutically acceptable salts and solvates thereof, wherein:

X is CH or N;

Y is CH₂ or N—R²;

Z is

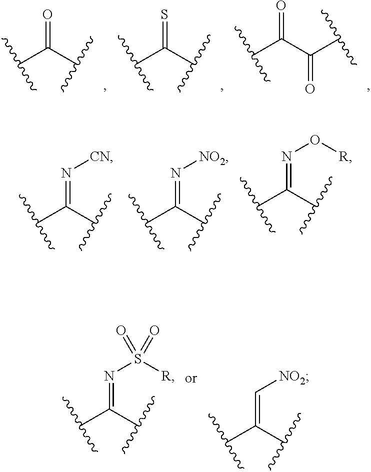

R¹ is H, C₁₋₆ alkyl optionally substituted with one or more N(R⁴)(R^{4′}), —C₀₋₆ alkyl-(4- to 8-membered heterocyclyl), or -(4- to 8-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl;

R^{1′} is H or C₁₋₆ alkyl, or

R¹ and R^{1′}, together with the nitrogen atom to which R¹ and R^{1′} are attached, form a 5- to 6-membered heterocyclyl optionally substituted with one or more R⁵;

R² is H, C₁₋₆ alkyl, —C₃₋₆ cycloalkyl-C₀₋₆ alkyl, —C₀₋₆ alkyl-C₃₋₆ cycloalkyl, -(3- to 7-membered heterocyclyl)-C₀₋₆ alkyl, —C₀₋₆ alkyl-(3- to 7-membered heterocyclyl), —(C₆₋₁₀ aryl)-C₀₋₆ alkyl, —C₀₋₆ alkyl-(C₆₋₁₀ aryl), -(3- to 7-membered heteroaryl)-C₀₋₆ alkyl, —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl, cycloalkyl, heterocyclyl, aryl, heteroaryl represented by R² is optionally substituted with one or more —OH, —NH₂, —NH—C(═O)—O—(C₁₋₆ alkyl), halogen, C₁₋₆ alkyl, or phenyl;

R³ is H, C₁₋₆ alkyl, C₂₋₆ alkenyl, C₂₋₆ alkynyl, C₁₋₆ haloalkyl, halogen, —CN, —NO₂, —OR, —SR, —S(═O)₂R, —C(═O)R, —OC(═O)R, —NR₂, or —CO₂R;

each R⁴ and R^{4′} is independently H, —C(═O)—O—(C₁₋₆ alkyl), C₁₋₆ alkyl, C₂₋₆ alkenyl, C₂₋₆ alkynyl, or C₃₋₆ cycloalkyl;

R⁵ is -(5- to 7-membered heterocyclyl)-C₀₋₆-alkyl, —C₀₋₆-alkyl-(5- to 7-membered heterocyclyl), —(C₃₋₆ cycloalkyl)-C₀₋₆-alkyl, or —C₀₋₆-alkyl-(C₃₋₆ cycloalkyl), wherein the alkyl, heterocyclyl, or cycloalkyl represented by R⁵ is optionally substituted by one or more C₁₋₆ alkyl;

each R is independently H, C₁₋₆ alkyl, C₂₋₆ alkenyl, C₂₋₆ alkynyl, or C₃₋₆ cycloalkyl; and

n is 0 or 1,

provided that R² and R³ are not both CH₃.

In some embodiments, the present disclosure provides compounds of Formula I:

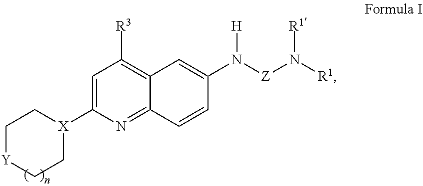

and pharmaceutically acceptable salts and solvates thereof, wherein:

X is CH or N;

Y is CH₂ or N—R²;

Z is selected from the group consisting of

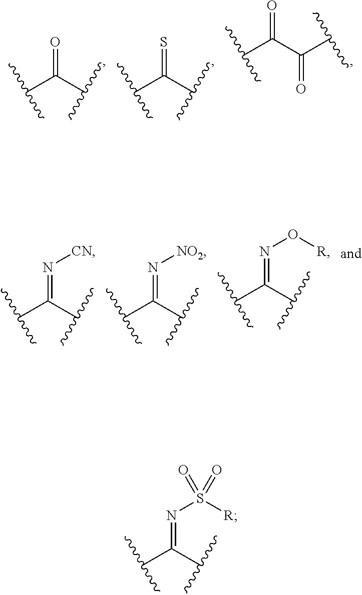

R¹ is selected from the group consisting of H, C₁₋₆ alkyl substituted with one or more N(R⁴)(R^{4′}), and C₄₋₈ heterocyclyl-C₀₋₆ alkyl optionally substituted on the heterocyclyl with ═O, heterocyclyl-C₀₋₂ alkyl, cycloalkyl-C₀₋₆ alkyl, CH₃, and CH₂CH₃;

R^{1′} is H or CH₃, or

R¹ and R^{1′}, together with the nitrogen atom to which R¹ and R^{1′} are attached, form a 5- to 6-membered heterocyclyl optionally substituted with one or more R⁵;

R² is selected from the group consisting of H, Boc, optionally substituted C₁₋₅ alkyl, optionally substituted C₃₋₆ cycloalkyl-C₁₋₅ alkyl, optionally substituted C₃₋₇ heterocyclyl-C₀₋₅ alkyl, optionally substituted aryl-C₁₋₅ alkyl, optionally substituted heteroaryl-C₁₋₅ alkyl, optionally substituted C(═O)—C₁₋₅ alkyl, optionally substituted C(═O)—C₅₋₇ heterocyclyl, optionally substituted C(═O)—O—C₁₋₅ alkyl, optionally substituted SO₂-aryl, optionally substituted C(═O)—NH-aryl, and optionally substituted C(═O)—NH—C₁₋₅ alkyl, wherein the optional substitution is from 1 to 4 substituents independently selected from the group consisting of OH, NH₂, NHBoc, halogen, C₁₋₃ alkyl, and phenyl;

R³ is selected from the group consisting of hydrogen, C₁₋₆ alkyl, C₁₋₆ haloalkyl, C₁₋₆ heteroalkyl, F, Cl, Br, I, CN, NO₂, OR, SR, S(═O)₂R, C(═O)R, OC(═O)R, NR₂, and CO₂R;

R⁴ and R^{4′} are each independently selected from the group consisting of H, Boc, and C₁₋₆ hydrocarbyl, or R⁴ and R^{4′}, together with the nitrogen to which R⁴ and R^{4′} are connected, form a C₅₋₇ heterocyclyl ring;

each occurrence of R is independently selected from the group consisting of C₁₋₁₀ hydrocarbyl and hydrogen; and

n is 0 or 1,

provided that R² and R³ are not simultaneously CH₃, and

provided that the compound is not 1-(2-(diethylamino)ethyl)-3-(4-methyl-2-(4-ethylpiperazin-1-yl)quinolin-6-yl)thiourea, 1-isopropyl-3-(4-methyl-2-(pyrrolidin-1-yl)quinolin-6-yl)thiourea, 1-(4-Ethyl-phenyl)-3-[2-(4-ethyl-piperazin-1-yl)-4-methyl-quinolin-6-yl]-1-propyl-thiourea, 1-Benzyl-3-[2-(4-ethyl-piperazin-1-yl)-4-methyl-quinolin-6-yl]-1-methyl-thiourea, 1-(4-Ethoxy-phenyl)-1-ethyl-3-[2-(4-ethyl-piperazin-1-yl)-4-methyl-quinolin-6-yl]-thiourea, 1-[2-(4-Ethyl-piperazin-1-yl)-4-methyl-quinolin-6-yl]-3-thiophen-2-ylmethyl-thiourea, 1-[2-(4-Ethyl-piperazin-1-yl)-4-methyl-quinolin-6-yl]-3-(2-methoxy-benzyl)-thiourea, 1-[2-(4-Ethyl-piperazin-1-yl)-4-methyl-quinolin-6-yl]-3-(4-fluoro-benzyl)-thiourea, 1-[2-(4-Ethyl-piperazin-1-yl)-4-methyl-quinolin-6-yl]-3-furan-2-ylmethyl-thiourea, 1-Ethyl-3-[2-(4-ethyl-piperazin-1-yl)-4-methyl-quinolin-6-yl]-1-(4-fluoro-phenyl)-thiourea, 1-(2-Ethyl-phenyl)-3-[2-(4-ethyl-piperazin-1-yl)-4-methyl-quinolin-6-yl]-1-methyl-thiourea, 1-Benzo[1,3]dioxol-5-ylmethyl-3-[2-(4-ethyl-piperazin-1-yl)-4-methyl-quinolin-6-yl]-thiourea, 1-(2-(dimethylamino)ethyl)-3-(4-methyl-2-(pyrrolidin-1-yl)quinolin-6-yl)thiourea, 1-(3-(3,5-dimethylpiperidin-1-yl)propyl)-3-(2-(4-ethylpiperazin-1-yl)-4-methylquinolin-6-yl)thiourea, N-(2-(4-ethylpiperazin-1-yl)-4-methylquinolin-6-yl)-[1,4′-bipiperidine]-1′-carbothioamide, 1-(2-(4-ethylpiperazin-1-yl)-4-methylquinolin-6-yl)-3-(3-(2-ethylpiperidin-1-yl)propyl)thiourea, or 1-((1-benzylpiperidin-4-yl)methyl)-3-(2-(piperazin-1-yl)quinolin-6-yl)thiourea.

In some aspects, the present disclosure provides compounds of Formula I′, and pharmaceutically acceptable salts and solvates thereof, wherein:

X is N;

Y is CH₂ or N—R²;

Z is

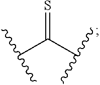 [SMILES: *C(*)=S]

R¹ is H, C₁₋₆ alkyl optionally substituted with one or more N(R⁴)(R^{4′}), or —C₀₋₆ alkyl-(4- to 8-membered heterocyclyl), wherein the heterocyclyl is optionally substituted with one or more oxo, —C₀₋₆ alkyl-(4- to 8-membered heterocyclyl), —C₀₋₆ alkyl-(C₃₋₇ cycloalkyl), or C₁₋₆ alkyl;

R^{1′} is H or —CH₃, or

R¹ and R^{1′}, together with the nitrogen atom to which R¹ and R^{1′} are attached, form a 5- to 6-membered heterocyclyl optionally substituted with one or more R⁵;

R² is H, C₁₋₆ alkyl, —C₀₋₅ alkyl-C₃₋₆ cycloalkyl, —C₀₋₆ alkyl-(3- to 7-membered heterocyclyl), —C₀₋₆ alkyl-(C₆₋₁₀ aryl), —C₀₋₅ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl, cycloalkyl, heterocyclyl, aryl, heteroaryl represented by R² is optionally substituted with one or more —OH, —NH₂, —NH—C(═O)—O—(C₁₋₅ alkyl), halogen, C₁₋₆ alkyl, or phenyl;

R³ is H or C₁₋₆ alkyl;

each R⁴ and R^{4′} is independently H, —C(═O)—O—(C₁₋₆ alkyl), or C₁₋₆ alkyl;

R⁵ is —C₀₋₆-alkyl-(5- to 7-membered heterocyclyl) or —C₀₋₆-alkyl-(C₃₋₆ cycloalkyl), wherein the alkyl, heterocyclyl, or cycloalkyl represented by R⁵ is optionally substituted by one or more C₁₋₆ alkyl;

each R is independently H or C₁₋₆ alkyl; and

n is 0 or 1,

provided that R² and R³ are not simultaneously CH₃.

In some embodiments, R² and R³ are not simultaneously CH₃.

In some embodiments, R² is H, C₂₋₅ alkyl, —C₃₋₆ cycloalkyl-C₀₋₅ alkyl, —C₀₋₅ alkyl-C₃₋₆ cycloalkyl, -(3- to 7-membered heterocyclyl)-C₀₋₅ alkyl, —C₀₋₅ alkyl-(3- to 7-membered heterocyclyl), —(C₆₋₁₀ aryl)-C₀₋₅ alkyl, —C₀₋₅ alkyl-(C₆₋₁₀ aryl), -(3- to 7-membered heteroaryl)-C₀₋₅ alkyl, —C₀₋₅ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl, cycloalkyl, heterocyclyl, aryl, heteroaryl represented by R² is optionally substituted with one or more —OH, —NH₂, —NH—C(═O)—O—(C₁₋₆ alkyl), halogen, C₁₋₆ alkyl, or phenyl and R³ is CH₃.

In some embodiments, R² is CH₃ and R³ is H, C₂₋₆ alkyl, C₂₋₆ alkenyl, C₂₋₆ alkynyl, C₁₋₆ haloalkyl, halogen, —CN, —NO₂, —OR, —SR, —S(═O)₂R, —C(═O)R, —OC(═O)R, —NR₂, and —CO₂R.

In some embodiments, the compound of Formula I or Formula I′ is not 1-(2-(diethylamino)ethyl)-3-(4-methyl-2-(4-ethylpiperazin-1-yl)quinolin-6-yl)thiourea, 1-isopropyl-3-(4-methyl-2-(pyrrolidin-1-yl)quinolin-6-yl)thiourea, 1-(4-Ethyl-phenyl)-3-[2-(4-ethyl-piperazin-1-yl)-4-methyl-quinolin-6-yl]-1-propyl-thiourea, 1-Benzyl-3-[2-(4-ethyl-piperazin-1-yl)-4-methyl-quinolin-6-yl]-1-methyl-thiourea, 1-(4-Ethoxy-phenyl)-1-ethyl-3-[2-(4-ethyl-piperazin-1-yl)-4-methyl-quinolin-6-yl]-thiourea, 1-[2-(4-Ethyl-piperazin-1-yl)-4-methyl-quinolin-6-yl]-3-thiophen-2-ylmethyl-thiourea, 1-[2-(4-Ethyl-piperazin-1-yl)-4-methyl-quinolin-6-yl]-3-(2-methoxy-benzyl)-thiourea, 1-[2-(4-Ethyl-piperazin-1-yl)-4-methyl-quinolin-6-yl]-3-(4-fluoro-benzyl)-thiourea, 1-[2-(4-Ethyl-piperazin-1-yl)-4-methyl-quinolin-6-yl]-3-furan-2-ylmethyl-thiourea, 1-Ethyl-3-[2-(4-ethyl-piperazin-1-yl)-4-methyl-quinolin-6-yl]-1-(4-fluoro-phenyl)-thiourea, 1-(2-Ethyl-phenyl)-3-[2-(4-ethyl-piperazin-1-yl)-4-methyl-quinolin-6-yl]-1-methyl-thiourea, 1-Benzo[1,3]dioxol-5-ylmethyl-3-[2-(4-ethyl-piperazin-1-yl)-4-methyl-quinolin-6-yl]-thiourea, 1-(2-(dimethylamino)ethyl)-3-(4-methyl-2-(pyrrolidin-1-yl)quinolin-6-yl)thiourea, 1-(3-(3,5-dimethylpiperidin-1-yl)propyl)-3-(2-(4-ethylpiperazin-1-yl)-4-methylquinolin-6-yl)thiourea, N-(2-(4-ethylpiperazin-1-yl)-4-methylquinolin-6-yl)-[1,4′-bipiperidine]-1′-carbothioamide, 1-(2-(4-ethylpiperazin-1-yl)-4-methylquinolin-6-yl)-3-(3-(2-ethylpiperidin-1-yl)propyl)thiourea, or 1-((1-benzylpiperidin-4-yl)methyl)-3-(2-(piperazin-1-yl)quinolin-6-yl)thiourea.

In some embodiments, X is CH or N. In some embodiments, X is CH. In some embodiments, X is N.

In some embodiments, n is 0 or 1. In some embodiments, n is 1. In some embodiments, n is 0.

In some embodiments, Z is

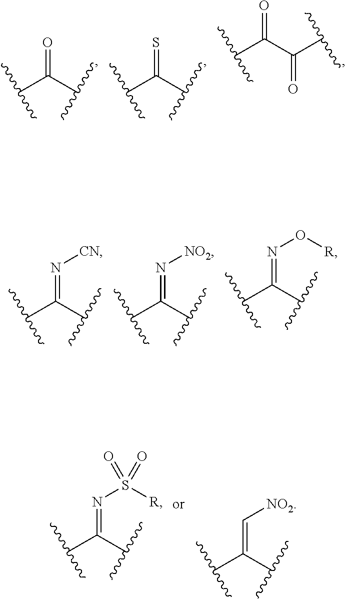

In some embodiments, Z is

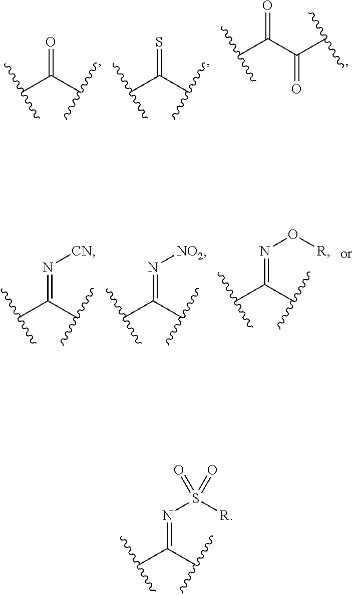

In some embodiments, Z is

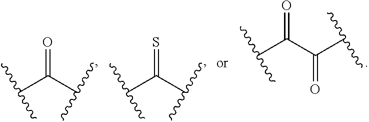

In some embodiments, Z is

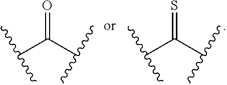

In some embodiments, Z is

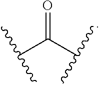 [SMILES: *C(*)=O]

In some embodiments, Z is

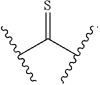 [SMILES: *C(*)=S]

In some embodiments, Z is

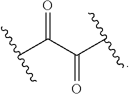 [SMILES: *C(=O)C(*)=O]

In some embodiments, Z is

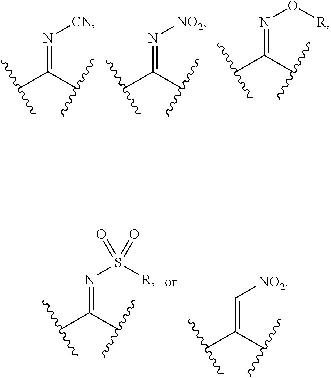

In some embodiments, Z is

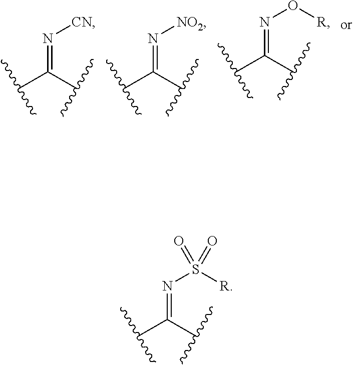

In some embodiments, Z is

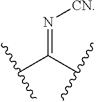 [SMILES: *C(*)=NC#N]

In some embodiments, Z is

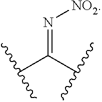 [SMILES: *C(*)=N[N+](=O)[O-]]

In some embodiments, Z is

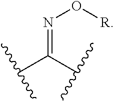

In some embodiments, Z is

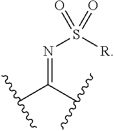

In some embodiments, Z is

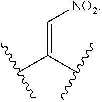 [SMILES: *C(*)=C[N+](=O)[O-]]

In some embodiments, Y is CH₂ or N—R². In some embodiments, Y is CH₂. In some embodiments, Y is N—R².

In some embodiments, R¹ is H, C₁₋₆ alkyl optionally substituted with one or more N(R⁴)(R^{4′}), —C₀₋₆ alkyl-(4- to 8-membered heterocyclyl), or -(4- to 8-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

In some embodiments, R¹ is H.

In some embodiments, R¹ is C₁₋₆ alkyl optionally substituted with one or more N(R⁴)(R^{4′}), —C₀₋₆ alkyl-(4- to 8-membered heterocyclyl), or -(4- to 8-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

In some embodiments, R¹ is C₁₋₆ alkyl optionally substituted with one or more N(R⁴)(R^{4′}), —C₀₋₆ alkyl-(4- to 8-membered heterocyclyl), or -(4- to 8-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

In some embodiments, R¹ is C₁₋₆ alkyl.

In some embodiments, R¹ is C₁₋₆ alkyl optionally substituted with one or more N(R⁴)(R^{4′}).

In some embodiments, R¹ is methyl. In some embodiments, R¹ is ethyl. In some embodiments, R¹ is propyl. In some embodiments, R¹ is isopropyl. In some embodiments, R¹ is butyl. In some embodiments, R¹ is isobutyl. In some embodiments, R¹ is sec-butyl. In some embodiments, R¹ is tert-butyl. In some embodiments, R¹ is pentyl. In some embodiments, R¹ is hexyl.

In some embodiments, R¹ is C₁ alkyl optionally substituted with one or more N(R⁴)(R^{4′}). In some embodiments, R¹ is C₂ alkyl optionally substituted with one or more N(R⁴)(R^{4′}). In some embodiments, R¹ is C₃ alkyl optionally substituted with one or more N(R⁴)(R^{4′}). In some embodiments, R¹ is C₄ alkyl optionally substituted with one or more N(R⁴)(R^{4′}). In some embodiments, R¹ is C₅ alkyl optionally substituted with one or more N(R⁴)(R^{4′}). In some embodiments, R¹ is C₆ alkyl optionally substituted with one or more N(R⁴)(R^{4′}).

In some embodiments, R¹ is C₁₋₆ alkyl substituted with one or more N(R⁴)(R^{4′}).

In some embodiments, R¹ is C₁ alkyl substituted with one or more N(R⁴)(R^{4′}). In some embodiments, R¹ is C₂ alkyl substituted with one or more N(R⁴)(R^{4′}). In some embodiments, R¹ is C₃ alkyl substituted with one or more N(R⁴)(R^{4′}). In some embodiments, R¹ is C₄ alkyl substituted with one or more N(R⁴)(R^{4′}). In some embodiments, R¹ is C₅ alkyl substituted with one or more N(R⁴)(R^{4′}). In some embodiments, R¹ is C₆ alkyl substituted with one or more N(R⁴)(R^{4′}).

In some embodiments, R¹ is C₁₋₆ alkyl substituted with one N(R⁴)(R^{4′}).

In some embodiments, R¹ is C₁ alkyl substituted with one N(R⁴)(R^{4′}). In some embodiments, R¹ is C₂ alkyl substituted with one N(R⁴)(R^{4′}). In some embodiments, R¹ is C₃ alkyl substituted with one N(R⁴)(R^{4′}). In some embodiments, R¹ is C₄ alkyl substituted with one N(R⁴)(R^{4′}). In some embodiments, R¹ is C₅ alkyl substituted with one N(R⁴)(R^{4′}). In some embodiments, R¹ is C₆ alkyl substituted with one N(R⁴)(R^{4′}).

In some embodiments, R¹ is C₁₋₆ alkyl substituted with two N(R⁴)(R^{4′}).

In some embodiments, R¹ is C₁ alkyl substituted with two N(R⁴)(R^{4′}). In some embodiments, R¹ is C₂ alkyl substituted with two N(R⁴)(R^{4′}). In some embodiments, R¹ is C₃ alkyl substituted with two N(R⁴)(R^{4′}). In some embodiments, R¹ is C₄ alkyl substituted with two N(R⁴)(R^{4′}). In some embodiments, R¹ is C₅ alkyl substituted with two N(R⁴)(R^{4′}). In some embodiments, R¹ is C₆ alkyl substituted with two N(R⁴)(R^{4′}).

In some embodiments, R¹ is C₁₋₆ alkyl substituted with three N(R⁴)(R^{4′}).

In some embodiments, R¹ is C₁ alkyl substituted with three N(R⁴)(R^{4′}). In some embodiments, R¹ is C₂ alkyl substituted with three N(R⁴)(R^{4′}). In some embodiments, R¹ is C₃ alkyl substituted with three N(R⁴)(R^{4′}). In some embodiments, R¹ is C₄ alkyl substituted with three N(R⁴)(R^{4′}). In some embodiments, R¹ is C₅ alkyl substituted with three N(R⁴)(R^{4′}).

In some embodiments, R¹ is C₆ alkyl substituted with three N(R⁴)(R^{4′}).

In some embodiments, R¹ is C₁₋₆ alkyl substituted with four N(R⁴)(R^{4′}).

In some embodiments, R¹ is C₁ alkyl substituted with four N(R⁴)(R^{4′}). In some embodiments, R¹ is C₂ alkyl substituted with four N(R⁴)(R^{4′}). In some embodiments, R¹ is C₃ alkyl substituted with four N(R⁴)(R^{4′}). In some embodiments, R¹ is C₄ alkyl substituted with four N(R⁴)(R^{4′}). In some embodiments, R¹ is C₅ alkyl substituted with four N(R⁴)(R^{4′}). In some embodiments, R¹ is C₆ alkyl substituted with four N(R⁴)(R^{4′}).

In some embodiments, R¹ is —C₀₋₆ alkyl-(4- to 8-membered heterocyclyl) or -(4- to 8-membered heterocyclyl)-C₀₋₆ alkyl.

In some embodiments, R¹ is —C₀₋₆ alkyl-(4- to 8-membered heterocyclyl) or -(4- to 8-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

In some embodiments, R¹ is —C₀₋₆ alkyl-(4- to 8-membered heterocyclyl).

In some embodiments, R¹ is —C₀₋₆ alkyl-(4-membered heterocyclyl). In some embodiments, R¹ is —C₀₋₆ alkyl-(5-membered heterocyclyl). In some embodiments, R¹ is —C₀₋₆ alkyl-(6-membered heterocyclyl). In some embodiments, R¹ is —C₀₋₆ alkyl-(7-membered heterocyclyl). In some embodiments, R¹ is —C₀₋₆ alkyl-(8-membered heterocyclyl).

In some embodiments, R¹ is —C₀₋₆ alkyl-(4- to 8-membered heterocyclyl), wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

In some embodiments, R¹ is —C₀₋₆ alkyl-(4-membered heterocyclyl), wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl. In some embodiments, R¹ is —C₀₋₆ alkyl-(5-membered heterocyclyl), wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl. In some embodiments, R¹ is —C₀₋₆ alkyl-(6-membered heterocyclyl), wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl. In some embodiments, R¹ is —C₀₋₆ alkyl-(7-membered heterocyclyl), wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl. In some embodiments, R¹ is —C₀₋₆ alkyl-(8-membered heterocyclyl), wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

In some embodiments, R¹ is —C₀₋₆ alkyl-(4- to 8-membered heterocyclyl), wherein the heterocyclyl is substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

In some embodiments, R¹ is —C₀₋₆ alkyl-(4-membered heterocyclyl), wherein the heterocyclyl is substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl. In some embodiments, R¹ is —C₀₋₆ alkyl-(5-membered heterocyclyl), wherein the heterocyclyl is substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl. In some embodiments, R¹ is —C₀₋₆ alkyl-(6-membered heterocyclyl), wherein the heterocyclyl is substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl. In some embodiments, R¹ is —C₀₋₆ alkyl-(7-membered heterocyclyl), wherein the heterocyclyl is substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl. In some embodiments, R¹ is —C₀₋₆ alkyl-(8-membered heterocyclyl), wherein the heterocyclyl is substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

In some embodiments, R¹ is —C₀₋₆ alkyl-(4- to 8-membered heterocyclyl), wherein the heterocyclyl is substituted with one or more oxo, —C₀₋₆ alkyl-(4- to 8-membered heterocyclyl), —C₀₋₆ alkyl-(C₃₋₇ cycloalkyl), or C₁₋₆ alkyl.

In some embodiments, R¹ is —C₀₋₆ alkyl-(4-membered heterocyclyl), wherein the heterocyclyl is substituted with one or more oxo, —C₀₋₆ alkyl-(4- to 8-membered heterocyclyl), —C₀₋₆ alkyl-(C₃₋₇ cycloalkyl), or C₁₋₆ alkyl. In some embodiments, R¹ is —C₀₋₆ alkyl-(5-membered heterocyclyl), wherein the heterocyclyl is substituted with one or more oxo, —C₀₋₆ alkyl-(4- to 8-membered heterocyclyl), —C₀₋₆ alkyl-(C₃₋₇ cycloalkyl), or C₁₋₆ alkyl. In some embodiments, R¹ is —C₀₋₆ alkyl-(6-membered heterocyclyl), wherein the heterocyclyl is substituted with one or more oxo, —C₀₋₆ alkyl-(4- to 8-membered heterocyclyl), —C₀₋₆ alkyl-(C₃₋₇ cycloalkyl), or C₁₋₆ alkyl. In some embodiments, R¹ is —C₀₋₆ alkyl-(7-membered heterocyclyl), wherein the heterocyclyl is substituted with one or more oxo, —C₀₋₆ alkyl-(4- to 8-membered heterocyclyl), —C₀₋₆ alkyl-(C₃₋₇ cycloalkyl), or C₁₋₆ alkyl. In some embodiments, R¹ is —C₀₋₆ alkyl-(8-membered heterocyclyl), wherein the heterocyclyl is substituted with one or more oxo, —C₀₋₆ alkyl-(4- to 8-membered heterocyclyl), —C₀₋₆ alkyl-(C₃₋₇ cycloalkyl), or C₁₋₆ alkyl.

In some embodiments, R¹ is -(4- to 8-membered heterocyclyl)-C₀₋₆ alkyl.

In some embodiments, R¹ is -(4-membered heterocyclyl)-C₀₋₆ alkyl. In some embodiments, R¹ is -(5-membered heterocyclyl)-C₀₋₆ alkyl. In some embodiments, R¹ is -(6-membered heterocyclyl)-C₀₋₆ alkyl. In some embodiments, R¹ is -(7-membered heterocyclyl)-C₀₋₆ alkyl. In some embodiments, R¹ is -(8-membered heterocyclyl)-C₀₋₆ alkyl.

In some embodiments, R¹ is -(4- to 8-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

In some embodiments, R¹ is -(4-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl. In some embodiments, R¹ is -(5-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl. In some embodiments, R¹ is -(6-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl. In some embodiments, R¹ is -(7-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl. In some embodiments, R¹ is -(8-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

In some embodiments, R^{1′} is H or C₁₋₆ alkyl. In some embodiments, R^{1′} is H or —CH₃.

In some embodiments, R^{1′} is H. In some embodiments, R^{1′} is C₁₋₆ alkyl.

In some embodiments, R^{1′} is methyl. In some embodiments, R^{1′} is methyl. In some embodiments, R^{1′} is ethyl. In some embodiments, R^{1′} is propyl. In some embodiments, R^{1′} is isopropyl. In some embodiments, R^{1′} is butyl. In some embodiments, R^{1′} is sec-butyl. In some embodiments, R^{1′} is tert-butyl. In some embodiments, R^{1′} is pentyl. In some embodiments, R^{1′} is hexyl.

In some embodiments, R¹ is H,

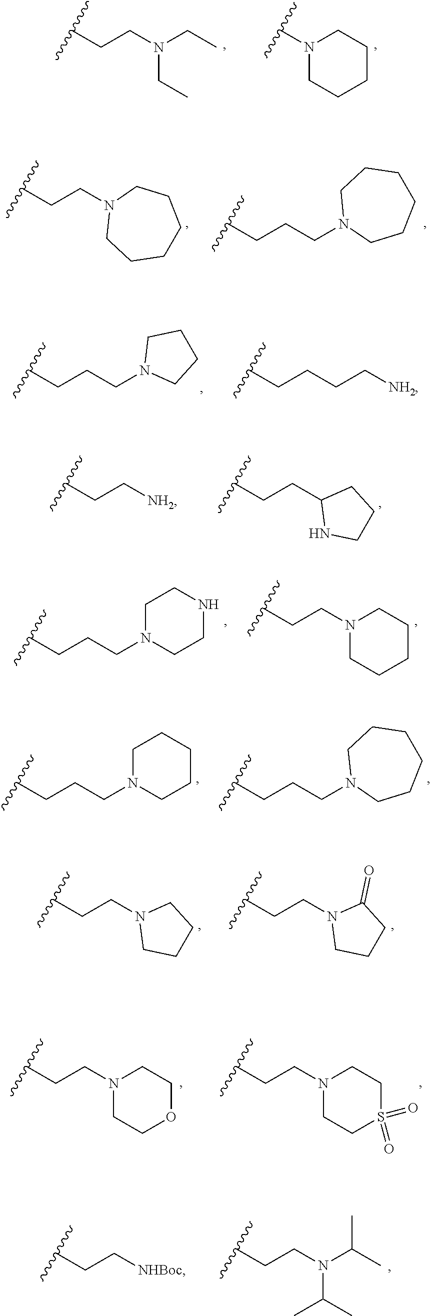

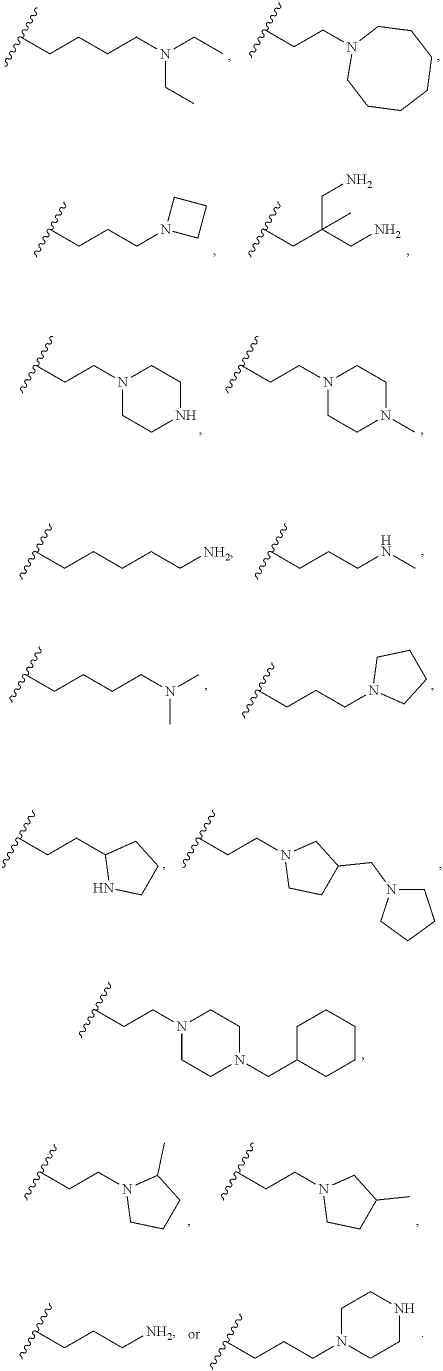

In some embodiments, R¹ and R^{1′}, together with the nitrogen atom to which R¹ and R^{1′} are attached, form a 5- to 6-membered heterocyclyl.

In some embodiments, R¹ and R^{1′}, together with the nitrogen atom to which R¹ and R^{1′} are attached, form a 5-membered heterocyclyl. In some embodiments, R¹ and R^{1′}, together with the nitrogen atom to which R¹ and R^{1′} are attached, form a 6-membered heterocyclyl.

In some embodiments, R¹ and R^{1′}, together with the nitrogen atom to which R¹ and R^{1′} are attached, form a 5- to 6-membered heterocyclyl optionally substituted with one or more R⁵.

In some embodiments, R¹ and R^{1′}, together with the nitrogen atom to which R¹ and R^{1′} are attached, form a 5-membered heterocyclyl optionally substituted with one or more R⁵.

In some embodiments, R¹ and R^{1′}, together with the nitrogen atom to which R¹ and R^{1′} are attached, form a 6-membered heterocyclyl optionally substituted with one or more R⁵.

In some embodiments, R¹ and R^{1′}, together with the nitrogen atom to which R¹ and R^{1′} are attached, form a 5- to 6-membered heterocyclyl substituted with one or more R⁵.

In some embodiments, R¹ and R^{1′}, together with the nitrogen atom to which R¹ and R^{1′} are attached, form a 5-membered heterocyclyl substituted with one or more R⁵. In some embodiments, R¹ and R^{1′}, together with the nitrogen atom to which R¹ and R^{1′} are attached, form a 6-membered heterocyclyl substituted with one or more R⁵.

In some embodiments, R¹ and R^{1′}, together with the nitrogen atom to which R¹ and R^{1′} are attached, form a 5- to 6-membered heterocyclyl substituted with one R⁵.

In some embodiments, R¹ and R^{1′}, together with the nitrogen atom to which R¹ and R^{1′} are attached, form a 5-membered heterocyclyl substituted with one R⁵. In some embodiments, R¹ and R^{1′}, together with the nitrogen atom to which R¹ and R^{1′} are attached, form a 6-membered heterocyclyl substituted with one R⁵.

In some embodiments, R¹ and R^{1′}, together with the nitrogen atom to which R¹ and R^{1′} are attached, form a heterocycle selected from the group consisting of consisting of

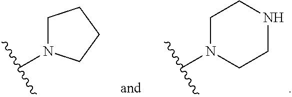 [SMILES: *N1CCCC1 ; *N1CCNCC1]

In some embodiments, R¹ and R^{1′}, together with the nitrogen atom to which R¹ and R^{1′} are attached, form a heterocycle selected from

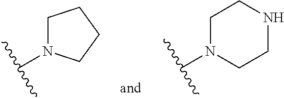 [SMILES: *N1CCCC1 ; *N1CCNCC1]

and wherein the heterocycle is optionally substituted with one or more R⁵.

In some embodiments, R¹ and R^{1′}, together with the nitrogen atom to which R¹ and R^{1′} are attached, form a substituted heterocycle selected from

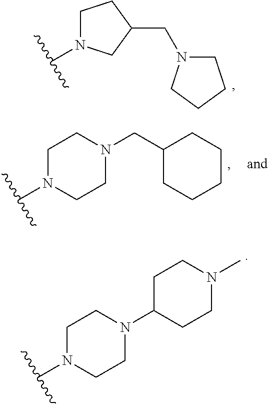

In some embodiments, R² is H, C₁₋₆ alkyl, —C₃₋₆ cycloalkyl-C₀₋₆ alkyl, —C₀₋₅ alkyl-C₃₋₆ cycloalkyl, -(3- to 7-membered heterocyclyl)-C₀₋₆ alkyl, —C₀₋₆ alkyl-(3- to 7-membered heterocyclyl), —(C₆₋₁₀ aryl)-C₀₋₆ alkyl, —C₀₋₆ alkyl-(C₆₋₁₀ aryl), -(3- to 7-membered heteroaryl)-C₀₋₆ alkyl, —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl).

In some embodiments, R² is H, C₁₋₆ alkyl, —C₃₋₆ cycloalkyl-C₀₋₆ alkyl, —C₀₋₆ alkyl-C₃₋₆ cycloalkyl, -(3- to 7-membered heterocyclyl)-C₀₋₆ alkyl, —C₀₋₆ alkyl-(3- to 7-membered heterocyclyl), —(C₆₋₁₀ aryl)-C₀₋₆ alkyl, —C₀₋₆ alkyl-(C₆₋₁₀ aryl), -(3- to 7-membered heteroaryl)-C₀₋₆ alkyl, —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl, cycloalkyl, heterocyclyl, aryl, heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is H.

In some embodiments, R² is C₁₋₆ alkyl, —C₃₋₆ cycloalkyl-C₀₋₆ alkyl, —C₀₋₆ alkyl-C₃₋₆ cycloalkyl, -(3- to 7-membered heterocyclyl)-C₀₋₆ alkyl, —C₀₋₆ alkyl-(3- to 7-membered heterocyclyl), —(C₆₋₁₀ aryl)-C₀₋₆ alkyl, —C₀₋₆ alkyl-(C₆₋₁₀ aryl), -(3- to 7-membered heteroaryl)-C₀₋₆ alkyl, —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl).

In some embodiments, R² is C₁₋₆ alkyl, —C₃₋₆ cycloalkyl-C₀₋₆ alkyl, —C₀₋₆ alkyl-C₃₋₆ cycloalkyl, -(3- to 7-membered heterocyclyl)-C₀₋₆ alkyl, —C₀₋₆ alkyl-(3- to 7-membered heterocyclyl), —(C₆₋₁₀ aryl)-C₀₋₆ alkyl, —C₀₋₆ alkyl-(C₆₋₁₀ aryl), -(3- to 7-membered heteroaryl)-C₀₋₆ alkyl, —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl, cycloalkyl, heterocyclyl, aryl, heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is C₁₋₆ alkyl.

In some embodiments, R² is methyl. In some embodiments, R² is ethyl. In some embodiments, R² is propyl. In some embodiments, R² is isopropyl. In some embodiments, R² is butyl. In some embodiments, R² is isobutyl. In some embodiments, R² is sec-butyl. In some embodiments, R² is tert-butyl. In some embodiments, R² is pentyl. In some embodiments, R² is hexyl.

In some embodiments, R² is C₁₋₆ alkyl optionally substituted with one or more —OH, —NH₂, —NH—C(═O)—O—(C₁₋₆ alkyl), halogen, C₁₋₆ alkyl, or phenyl.

In some embodiments, R² is methyl optionally substituted with one or more —OH, —NH₂, —NH—C(═O)—O—(C₁₋₆ alkyl), halogen, C₁₋₆ alkyl, or phenyl. In some embodiments, R² is ethyl optionally substituted with one or more —OH, —NH₂, —NH—C(═O)—O—(C₁₋₆ alkyl), halogen, C₁₋₆ alkyl, or phenyl. In some embodiments, R² is propyl optionally substituted with one or more —OH, —NH₂, —NH—C(═O)—O—(C₁₋₆ alkyl), halogen, C₁₋₆ alkyl, or phenyl. In some embodiments, R² is isopropyl optionally substituted with one or more —OH, —NH₂, —NH—C(═O)—O—(C₁₋₆ alkyl), halogen, C₁₋₆ alkyl, or phenyl. In some embodiments, R² is butyl optionally substituted with one or more —OH, —NH₂, —NH—C(═O)—O—(C₁₋₆ alkyl), halogen, C₁₋₆ alkyl, or phenyl. In some embodiments, R² is isobutyl optionally substituted with one or more —OH, —NH₂, —NH—C(═O)—O—(C₁₋₆ alkyl), halogen, C₁₋₆ alkyl, or phenyl. In some embodiments, R² is sec-butyl optionally substituted with one or more —OH, —NH₂, —NH—C(═O)—O—(C₁₋₆ alkyl), halogen, C₁₋₆ alkyl, or phenyl. In some embodiments, R² is tert-butyl optionally substituted with one or more —OH, —NH₂, —NH—C(═O)—O—(C₁₋₆ alkyl), halogen, C₁₋₆ alkyl, or phenyl. In some embodiments, R² is pentyl optionally substituted with one or more —OH, —NH₂, —NH—C(═O)—O—(C₁₋₆ alkyl), halogen, C₁₋₆ alkyl, or phenyl.

In some embodiments, R² is C₁₋₆ alkyl substituted with one or more —OH, —NH₂, —NH—C(═O)—O—(C₁₋₆ alkyl), halogen, C₁₋₆ alkyl, or phenyl.

In some embodiments, R² is methyl substituted with one or more —OH, —NH₂, —NH—C(═O)—O—(C₁₋₆ alkyl), halogen, C₁₋₆ alkyl, or phenyl. In some embodiments, R² is ethyl substituted with one or more —OH, —NH₂, —NH—C(═O)—O—(C₁₋₆ alkyl), halogen, C₁₋₆ alkyl, or phenyl. In some embodiments, R² is propyl substituted with one or more —OH, —NH₂, —NH—C(═O)—O—(C₁₋₆ alkyl), halogen, C₁₋₆ alkyl, or phenyl. In some embodiments, R² is isopropyl substituted with one or more —OH, —NH₂, —NH—C(═O)—O—(C₁₋₆ alkyl), halogen, C₁₋₆ alkyl, or phenyl. In some embodiments, R² is butyl substituted with one or more —OH, —NH₂, —NH—C(═O)—O—(C₁₋₆ alkyl), halogen, C₁₋₆ alkyl, or phenyl. In some embodiments, R² is isobutyl substituted with one or more —OH, —NH₂, —NH—C(═O)—O—(C₁₋₆ alkyl), halogen, C₁₋₆ alkyl, or phenyl. In some embodiments, R² is sec-butyl substituted with one or more —OH, —NH₂, —NH—C(═O)—O—(C₁₋₆ alkyl), halogen, C₁₋₆ alkyl, or phenyl. In some embodiments, R² is tert-butyl substituted with one or more —OH, —NH₂, —NH—C(═O)—O—(C₁₋₆ alkyl), halogen, C₁₋₆ alkyl, or phenyl. In some embodiments, R² is pentyl substituted with one or more —OH, —NH₂, —NH—C(═O)—O—(C₁₋₆ alkyl), halogen, C₁₋₆ alkyl, or phenyl.

In some embodiments, R² is —C₃₋₆ cycloalkyl-C₀₋₆ alkyl, —C₀₋₆ alkyl-C₃₋₆ cycloalkyl, -(3- to 7-membered heterocyclyl)-C₀₋₆ alkyl, or —C₀₋₆ alkyl-(3- to 7-membered heterocyclyl).

In some embodiments, R² is —C₃₋₆ cycloalkyl-C₀₋₆ alkyl, —C₀₋₆ alkyl-C₃₋₆ cycloalkyl, -(3- to 7-membered heterocyclyl)-C₀₋₆ alkyl, or —C₀₋₆ alkyl-(3- to 7-membered heterocyclyl), wherein the alkyl, cycloalkyl, or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₃₋₆ cycloalkyl-C₀₋₆ alkyl, —C₀₋₆ alkyl-C₃₋₆ cycloalkyl, -(3- to 7-membered heterocyclyl)-C₀₋₆ alkyl, or —C₀₋₆ alkyl-(3- to 7-membered heterocyclyl), wherein the alkyl, cycloalkyl, or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₃₋₆ cycloalkyl-C₀₋₆ alkyl or -(3- to 7-membered heterocyclyl)-C₀₋₆ alkyl.

In some embodiments, R² is —C₃₋₆ cycloalkyl-C₀₋₆ alkyl or -(3- to 7-membered heterocyclyl)-C₀₋₆ alkyl, wherein the alkyl, cycloalkyl, or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₃₋₆ cycloalkyl-C₀₋₆ alkyl or -(3- to 7-membered heterocyclyl)-C₀₋₆ alkyl, wherein the alkyl, cycloalkyl, or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₃₋₆ cycloalkyl-C₀₋₆ alkyl.

In some embodiments, R² is —C₃₋₆ cycloalkyl-C₀₋₆ alkyl, wherein the alkyl or cycloalkyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₃₋₆ cycloalkyl-C₀₋₆ alkyl, wherein the alkyl or cycloalkyl represented by R² is substituted with one or more —OH, —NH₂, —NH—C(═O)—O—(C₁₋₆ alkyl), halogen, C₁₋₆ alkyl, or phenyl.

In some embodiments, R² is -(3- to 7-membered heterocyclyl)-C₀₋₆ alkyl.

In some embodiments, R² is -(3- to 7-membered heterocyclyl)-C₀₋₆ alkyl, wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is -(3- to 7-membered heterocyclyl)-C₀₋₆ alkyl, wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-C₃₋₆ cycloalkyl or —C₀₋₆ alkyl-(3- to 7-membered heterocyclyl).

In some embodiments, R² is —C₀₋₆ alkyl-C₃₋₆ cycloalkyl or —C₀₋₆ alkyl-(3- to 7-membered heterocyclyl), wherein the alkyl, cycloalkyl, or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-C₃₋₆ cycloalkyl or —C₀₋₆ alkyl-(3- to 7-membered heterocyclyl), wherein the alkyl, cycloalkyl, or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-C₃₋₆ cycloalkyl.

In some embodiments, R² is —C₀₋₆ alkyl-C₃₋₆ cycloalkyl, wherein the alkyl or cycloalkyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-C₃₋₆ cycloalkyl, wherein the alkyl or cycloalkyl represented by R² is substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-C₃ cycloalkyl.

In some embodiments, R² is —C₀₋₆ alkyl-C₃ cycloalkyl, wherein the alkyl or cycloalkyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-C₃ cycloalkyl, wherein the alkyl or cycloalkyl represented by R² is substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-C₄ cycloalkyl.

In some embodiments, R² is —C₀₋₆ alkyl-C₄ cycloalkyl, wherein the alkyl or cycloalkyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-C₄ cycloalkyl, wherein the alkyl or cycloalkyl represented by R² is substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-C₅ cycloalkyl.

In some embodiments, R² is —C₀₋₆ alkyl-C₅ cycloalkyl, wherein the alkyl or cycloalkyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-C₅ cycloalkyl, wherein the alkyl or cycloalkyl represented by R² is substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-C₆ cycloalkyl.

In some embodiments, R² is —C₀₋₆ alkyl-C₆ cycloalkyl, wherein the alkyl or cycloalkyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-C₆ cycloalkyl, wherein the alkyl or cycloalkyl represented by R² is substituted with one or more —OH, —NH₂, —NH—C(═O)—O—(C₁₋₆ alkyl), halogen, C₁₋₆ alkyl, or phenyl.

In some embodiments, R² is —C₃₋₆ cycloalkyl.

In some embodiments, R² is —C₃₋₆ cycloalkyl, optionally substituted with one or more —OH, —NH₂, —NH—C(═O)—O—(C₁₋₆ alkyl), halogen, C₁₋₆ alkyl, or phenyl.

In some embodiments, R² is —C₃₋₆ cycloalkyl substituted with one or more —OH, —NH₂, —NH—C(═O)—O—(C₁₋₆ alkyl), halogen, C₁₋₆ alkyl, or phenyl.

In some embodiments, R² is —C₁ alkyl-C₃₋₆ cycloalkyl.

In some embodiments, R² is —C₁ alkyl-C₃₋₆ cycloalkyl, wherein the alkyl or cycloalkyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₁ alkyl-C₃₋₆ cycloalkyl, wherein the alkyl or cycloalkyl represented by R² is substituted with one or more —OH, —NH₂, —NH—C(═O)—O—(C₁₋₆ alkyl), halogen, C₁₋₆ alkyl, or phenyl.

In some embodiments, R² is —C₂ alkyl-C₃₋₆ cycloalkyl.

In some embodiments, R² is —C₂ alkyl-C₃₋₆ cycloalkyl, wherein the alkyl or cycloalkyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₂ alkyl-C₃₋₆ cycloalkyl, wherein the alkyl or cycloalkyl represented by R² is substituted with one or more —OH, —NH₂, —NH—C(═O)—O—(C₁₋₆ alkyl), halogen, C₁₋₆ alkyl, or phenyl.

In some embodiments, R² is —C₃ alkyl-C₃₋₆ cycloalkyl.

In some embodiments, R² is —C₃ alkyl-C₃₋₆ cycloalkyl, wherein the alkyl or cycloalkyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₃ alkyl-C₃₋₆ cycloalkyl, wherein the alkyl or cycloalkyl represented by R² is substituted with one or more —OH, —NH₂, —NH—C(═O)—O—(C₁₋₆ alkyl), halogen, C₁₋₆ alkyl, or phenyl.

In some embodiments, R² is —C₄ alkyl-C₃₋₆ cycloalkyl.

In some embodiments, R² is —C₄ alkyl-C₃₋₆ cycloalkyl, wherein the alkyl or cycloalkyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₄ alkyl-C₃₋₆ cycloalkyl, wherein the alkyl or cycloalkyl represented by R² is substituted with one or more —OH, —NH₂, —NH—C(═O)—O—(C₁₋₆ alkyl), halogen, C₁₋₆ alkyl, or phenyl.

In some embodiments, R² is —C₅ alkyl-C₃₋₆ cycloalkyl.

In some embodiments, R² is —C₅ alkyl-C₃₋₆ cycloalkyl, wherein the alkyl or cycloalkyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₅ alkyl-C₃₋₆ cycloalkyl, wherein the alkyl or cycloalkyl represented by R² is substituted with one or more —OH, —NH₂, —NH—C(═O)—O—(C₁₋₆ alkyl), halogen, C₁₋₆ alkyl, or phenyl.

In some embodiments, R² is —C₆ alkyl-C₃₋₆ cycloalkyl.

In some embodiments, R² is —C₆ alkyl-C₃₋₆ cycloalkyl, wherein the alkyl or cycloalkyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₆ alkyl-C₃₋₆ cycloalkyl, wherein the alkyl or cycloalkyl represented by R² is substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(3- to 7-membered heterocyclyl).

In some embodiments, R² is —C₀₋₆ alkyl-(3- to 7-membered heterocyclyl), wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(3- to 7-membered heterocyclyl), wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(3-membered heterocyclyl).

In some embodiments, R² is —C₀₋₆ alkyl-(3-membered heterocyclyl), wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(3-membered heterocyclyl), wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(4-membered heterocyclyl).

In some embodiments, R² is —C₀₋₆ alkyl-(4-membered heterocyclyl), wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(4-membered heterocyclyl), wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(5-membered heterocyclyl).

In some embodiments, R² is —C₀₋₆ alkyl-(5-membered heterocyclyl), wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(5-membered heterocyclyl), wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(6-membered heterocyclyl).

In some embodiments, R² is —C₀₋₆ alkyl-(6-membered heterocyclyl), wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(6-membered heterocyclyl), wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(7-membered heterocyclyl).

In some embodiments, R² is —C₀₋₆ alkyl-(7-membered heterocyclyl), wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(7-membered heterocyclyl), wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is 3- to 7-membered heterocyclyl.

In some embodiments, R² is 3- to 7-membered heterocyclyl, optionally substituted with one or more —OH, —NH₂, —NH—C(═O)—O—(C₁₋₆ alkyl), halogen, C₁₋₆ alkyl, or phenyl.

In some embodiments, R² is 3- to 7-membered heterocyclyl, substituted with one or more —OH, —NH₂, —NH—C(═O)—O—(C₁₋₆ alkyl), halogen, C₁₋₆ alkyl, or phenyl.

In some embodiments, R² is —C₁ alkyl-(3- to 7-membered heterocyclyl).

In some embodiments, R² is —C₁ alkyl-(3- to 7-membered heterocyclyl), wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₁ alkyl-(3- to 7-membered heterocyclyl), wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₂ alkyl-(3- to 7-membered heterocyclyl).

In some embodiments, R² is —C₂ alkyl-(3- to 7-membered heterocyclyl), wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₂ alkyl-(3- to 7-membered heterocyclyl), wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₃ alkyl-(3- to 7-membered heterocyclyl).

In some embodiments, R² is —C₃ alkyl-(3- to 7-membered heterocyclyl), wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₃ alkyl-(3- to 7-membered heterocyclyl), wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₄ alkyl-(3- to 7-membered heterocyclyl).

In some embodiments, R² is —C₄ alkyl-(3- to 7-membered heterocyclyl), wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₄ alkyl-(3- to 7-membered heterocyclyl), wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₅ alkyl-(3- to 7-membered heterocyclyl).

In some embodiments, R² is —C₅ alkyl-(3- to 7-membered heterocyclyl), wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₅ alkyl-(3- to 7-membered heterocyclyl), wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —C₆ alkyl-(3- to 7-membered heterocyclyl).

In some embodiments, R² is —C₆ alkyl-(3- to 7-membered heterocyclyl), wherein the alkyl or heterocyclyl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₆ alkyl-(3- to 7-membered heterocyclyl), wherein the alkyl or heterocyclyl represented by R² is substituted with one or more —OH, —NH₂, —NH—C(═O)—O—(C₁₋₆ alkyl), halogen, C₁₋₆ alkyl, or phenyl.

In some embodiments, R² is —(C₆₋₁₀ aryl)-C₀₋₆ alkyl, —C₀₋₆ alkyl-(C₆₋₁₀ aryl), -(3- to 7-membered heteroaryl)-C₀₋₆ alkyl, or —C₀₋₆ alkyl-(3- to 7-membered heteroaryl).

In some embodiments, R² is —(C₆₋₁₀ aryl)-C₀₋₆ alkyl, —C₀₋₆ alkyl-(C₆₋₁₀ aryl), -(3- to 7-membered heteroaryl)-C₀₋₆ alkyl, or —C₀₋₆ alkyl-(3- to 7-membered heteroaryl), wherein the alkyl, aryl, or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —(C₆₋₁₀ aryl)-C₀₋₆ alkyl, —C₀₋₆ alkyl-(C₆₋₁₀ aryl), -(3- to 7-membered heteroaryl)-C₀₋₆ alkyl, or —C₀₋₆ alkyl-(3- to 7-membered heteroaryl), wherein the alkyl, aryl, or heteroaryl represented by R² is substituted with one or more —OH, —NH₂, —NH—C(═O)—O—(C₁₋₆ alkyl), halogen, C₁₋₆ alkyl, or phenyl.

In some embodiments, R² is —(C₆₋₁₀ aryl)-C₀₋₆ alkyl or -(3- to 7-membered heteroaryl)-C₀₋₆ alkyl.

In some embodiments, R² is —(C₆₋₁₀ aryl)-C₀₋₆ alkyl or -(3- to 7-membered heteroaryl)-C₀₋₆ alkyl, wherein the alkyl, aryl, or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —(C₆₋₁₀ aryl)-C₀₋₆ alkyl or -(3- to 7-membered heteroaryl)-C₀₋₆ alkyl, wherein the alkyl, aryl, or heteroaryl represented by R² is substituted with one or more —OH, —NH₂, —NH—C(═O)—O—(C₁₋₆ alkyl), halogen, C₁₋₆ alkyl, or phenyl.

In some embodiments, R² is —(C₆₋₁₀ aryl)-C₀₋₆ alkyl.

In some embodiments, R² is —(C₆₋₁₀ aryl)-C₀₋₆ alkyl, wherein the alkyl or aryl represented by R² is optionally substituted with one or more —OH, —NH₂, —NH—C(═O)—O—(C₁₋₆ alkyl), halogen, C₁₋₆ alkyl, or phenyl.

In some embodiments, R² is —(C₆₋₁₀ aryl)-C₀₋₆ alkyl, wherein the alkyl or aryl represented by R² is substituted with one or more —OH, —NH₂, —NH—C(═O)—O—(C₁₋₆ alkyl), halogen, C₁₋₆ alkyl, or phenyl.

In some embodiments, R² is -(3- to 7-membered heteroaryl)-C₀₋₆ alkyl.

In some embodiments, R² is -(3- to 7-membered heteroaryl)-C₀₋₆ alkyl, wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is -(3- to 7-membered heteroaryl)-C₀₋₆ alkyl, wherein the alkyl or heteroaryl represented by R² is substituted with one or more —OH, —NH₂, —NH—C(═O)—O—(C₁₆ alkyl), halogen, C₁₋₆ alkyl, or phenyl.

In some embodiments, R² —C₀₋₆ alkyl-(C₆₋₁₀ aryl) or —C₀₋₆ alkyl-(3- to 7-membered heteroaryl).

In some embodiments, R² is —C₀₋₆ alkyl-(C₆₋₁₀ aryl) or —C₀₋₆ alkyl-(3- to 7-membered heteroaryl), wherein the alkyl, aryl, or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(C₆₋₁₀ aryl) or —C₀₋₆ alkyl-(3- to 7-membered heteroaryl), wherein the alkyl, aryl, or heteroaryl represented by R² is substituted with one or more —OH, —NH₂, —NH—C(═O)—O—(C₁₋₆ alkyl), halogen, C₁₋₆ alkyl, or phenyl.

In some embodiments, R² —C₀₋₆ alkyl-(C₆₋₁₀ aryl).

In some embodiments, R² is —C₀₋₆ alkyl-(C₆₋₁₀ aryl), wherein the alkyl or aryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(C₆₋₁₀ aryl), wherein the alkyl or aryl represented by R² is substituted with one or more —OH, —NH₂, —NH—C(═O)—O—(C₁₋₆ alkyl), halogen, C₁₋₆ alkyl, or phenyl.

In some embodiments, R² —C₀₋₆ alkyl-(phenyl).

In some embodiments, R² is —C₀₋₆ alkyl-(phenyl), wherein the alkyl or aryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(phenyl), wherein the alkyl or aryl represented by R² is substituted with one or more —OH, —NH₂, —NH—C(═O)—O—(C₁₋₆ alkyl), halogen, C₁₋₆ alkyl, or phenyl.

In some embodiments, R² C₆₋₁₀ aryl.

In some embodiments, R² is C₆₋₁₀ aryl, optionally substituted with one or more —OH, —NH₂, —NH—C(═O)—O—(C₁₋₆ alkyl), halogen, C₁₋₆ alkyl, or phenyl.

In some embodiments, R² is C₆₋₁₀ aryl, substituted with one or more —OH, —NH₂, —NH—C(═O)—O—(C₁₋₆ alkyl), halogen, C₁₋₆ alkyl, or phenyl.

In some embodiments, R² —C₁ alkyl-(C₆₋₁₀ aryl).

In some embodiments, R² is —C₁ alkyl-(C₆₋₁₀ aryl), wherein the alkyl or aryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₁ alkyl-(C₆₋₁₀ aryl), wherein the alkyl or aryl represented by R² is substituted with one or more —OH, —NH₂, —NH—C(═O)—O—(C₁₋₆ alkyl), halogen, C₁₋₆ alkyl, or phenyl.

In some embodiments, R² —C₂ alkyl-(C₆₋₁₀ aryl).

In some embodiments, R² is —C₂ alkyl-(C₆₋₁₀ aryl), wherein the alkyl or aryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₂ alkyl-(C₆₋₁₀ aryl), wherein the alkyl or aryl represented by R² is substituted with one or more —OH, —NH₂, —NH—C(═O)—O—(C₁₋₆ alkyl), halogen, C₁₋₆ alkyl, or phenyl.

In some embodiments, R² —C₃ alkyl-(C₆₋₁₀ aryl).

In some embodiments, R² is —C₃ alkyl-(C₆₋₁₀ aryl), wherein the alkyl or aryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₃ alkyl-(C₆₋₁₀ aryl), wherein the alkyl or aryl represented by R² is substituted with one or more —OH, —NH₂, —NH—C(═O)—O—(C₁₋₆ alkyl), halogen, C₁₋₆ alkyl, or phenyl.

In some embodiments, R² —C₄ alkyl-(C₆₋₁₀ aryl).

In some embodiments, R² is —C₄ alkyl-(C₆₋₁₀ aryl), wherein the alkyl or aryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₄ alkyl-(C₆₋₁₀ aryl), wherein the alkyl or aryl represented by R² is substituted with one or more —OH, —NH₂, —NH—C(═O)—O—(C₁₋₆ alkyl), halogen, C₁₋₆ alkyl, or phenyl.

In some embodiments, R² —C₅ alkyl-(C₆₋₁₀ aryl).

In some embodiments, R² is —C₅ alkyl-(C₆₋₁₀ aryl), wherein the alkyl or aryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₅ alkyl-(C₆₋₁₀ aryl), wherein the alkyl or aryl represented by R² is substituted with one or more —OH, —NH₂, —NH—C(═O)—O—(C₁₋₆ alkyl), halogen, C₁₋₆ alkyl, or phenyl.

In some embodiments, R² —C₆ alkyl-(C₆₋₁₀ aryl).

In some embodiments, R² is —C₆ alkyl-(C₆₋₁₀ aryl), wherein the alkyl or aryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₆ alkyl-(C₆₋₁₀ aryl), wherein the alkyl or aryl represented by R² is substituted with one or more —OH, —NH₂, —NH—C(═O)—O—(C₁₋₆ alkyl), halogen, C₁₋₆ alkyl, or phenyl.

In some embodiments, R² —C₀₋₆ alkyl-(3- to 7-membered heteroaryl).

In some embodiments, R² is —C₀₋₆ alkyl-(3- to 7-membered heteroaryl), wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(3- to 7-membered heteroaryl), wherein the alkyl or heteroaryl represented by R² is substituted with one or more —OH, —NH₂, —NH—C(═O)—O—(C₁₋₆ alkyl), halogen, C₁₋₆ alkyl, or phenyl.

In some embodiments, R² —C₀₋₆ alkyl-(3-membered heteroaryl).

In some embodiments, R² is —C₀₋₆ alkyl-(3-membered heteroaryl), wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(3-membered heteroaryl), wherein the alkyl or heteroaryl represented by R² is substituted with one or more —OH, —NH₂, —NH—C(═O)—O—(C₁₋₆ alkyl), halogen, C₁₋₆ alkyl, or phenyl.

In some embodiments, R² —C₀₋₆ alkyl-(4-membered heteroaryl).

In some embodiments, R² is —C₀₋₆ alkyl-(4-membered heteroaryl), wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(4-membered heteroaryl), wherein the alkyl or heteroaryl represented by R² is substituted with one or more —OH, —NH₂, —NH—C(═O)—O—(C₁₋₆ alkyl), halogen, C₁₋₆ alkyl, or phenyl.

In some embodiments, R² —C₀₋₆ alkyl-(5-membered heteroaryl).

In some embodiments, R² is —C₀₋₆ alkyl-(5-membered heteroaryl), wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(5-membered heteroaryl), wherein the alkyl or heteroaryl represented by R² is substituted with one or more —OH, —NH₂, —NH—C(═O)—O—(C₁₋₆ alkyl), halogen, C₁₋₆ alkyl, or phenyl.

In some embodiments, R² —C₀₋₆ alkyl-(6-membered heteroaryl).

In some embodiments, R² is —C₀₋₆ alkyl-(6-membered heteroaryl), wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(6-membered heteroaryl), wherein the alkyl or heteroaryl represented by R² is substituted with one or more —OH, —NH₂, —NH—C(═O)—O—(C₁₋₆ alkyl), halogen, C₁₋₆ alkyl, or phenyl.

In some embodiments, R² —C₀₋₆ alkyl-(7-membered heteroaryl).

In some embodiments, R² is —C₀₋₆ alkyl-(7-membered heteroaryl), wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₀₋₆ alkyl-(7-membered heteroaryl), wherein the alkyl or heteroaryl represented by R² is substituted with one or more —OH, —NH₂, —NH—C(═O)—O—(C₁₋₆ alkyl), halogen, C₁₋₆ alkyl, or phenyl.

In some embodiments, R² 3- to 7-membered heteroaryl.

In some embodiments, R² is 3- to 7-membered heteroaryl, optionally substituted with one or more —OH, —NH₂, —NH—C(═O)—O—(C₁₋₆ alkyl), halogen, C₁₋₆ alkyl, or phenyl.

In some embodiments, R² is 3- to 7-membered heteroaryl, substituted with one or more —OH, —NH₂, —NH—C(═O)—O—(C₁₋₆ alkyl), halogen, C₁₋₆ alkyl, or phenyl.

In some embodiments, R² —C₁ alkyl-(3- to 7-membered heteroaryl).

In some embodiments, R² is —C₁ alkyl-(3- to 7-membered heteroaryl), wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₁ alkyl-(3- to 7-membered heteroaryl), wherein the alkyl or heteroaryl represented by R² is substituted with one or more —OH, —NH₂, —NH—C(═O)—O—(C₁₋₆ alkyl), halogen, C₁₋₆ alkyl, or phenyl.

In some embodiments, R² —C₂ alkyl-(3- to 7-membered heteroaryl).

In some embodiments, R² is —C₂ alkyl-(3- to 7-membered heteroaryl), wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₂ alkyl-(3- to 7-membered heteroaryl), wherein the alkyl or heteroaryl represented by R² is substituted with one or more —OH, —NH₂, —NH—C(═O)—O—(C₁₋₆ alkyl), halogen, C₁₋₆ alkyl, or phenyl.

In some embodiments, R² —C₃ alkyl-(3- to 7-membered heteroaryl).

In some embodiments, R² is —C₃ alkyl-(3- to 7-membered heteroaryl), wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₃ alkyl-(3- to 7-membered heteroaryl), wherein the alkyl or heteroaryl represented by R² is substituted with one or more —OH, —NH₂, —NH—C(═O)—O—(C₁₋₆ alkyl), halogen, C₁₋₆ alkyl, or phenyl.

In some embodiments, R² —C₄ alkyl-(3- to 7-membered heteroaryl).

In some embodiments, R² is —C₄ alkyl-(3- to 7-membered heteroaryl), wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₄ alkyl-(3- to 7-membered heteroaryl), wherein the alkyl or heteroaryl represented by R² is substituted with one or more —OH, —NH₂, —NH—C(═O)—O—(C₁₋₆ alkyl), halogen, C₁₋₆ alkyl, or phenyl.

In some embodiments, R² —C₅ alkyl-(3- to 7-membered heteroaryl).

In some embodiments, R² is —C₅ alkyl-(3- to 7-membered heteroaryl), wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₅ alkyl-(3- to 7-membered heteroaryl), wherein the alkyl or heteroaryl represented by R² is substituted with one or more —OH, —NH₂, —NH—C(═O)—O—(C₁₋₆ alkyl), halogen, C₁₋₆ alkyl, or phenyl.

In some embodiments, R² —C₆ alkyl-(3- to 7-membered heteroaryl).

In some embodiments, R² is —C₆ alkyl-(3- to 7-membered heteroaryl), wherein the alkyl or heteroaryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C₆ alkyl-(3- to 7-membered heteroaryl), wherein the alkyl or heteroaryl represented by R² is substituted with one or more —OH, —NH₂, —NH—C(═O)—O—(C₁₋₆ alkyl), halogen, C₁₋₆ alkyl, or phenyl.

In some embodiments, R² is —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl).

In some embodiments, R² is —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl, heterocyclyl, or aryl represented by R² is optionally substituted with one or more —OH, —NH₂, —NH—C(═O)—O—(C₁₋₆ alkyl), halogen, C₁₋₆ alkyl, or phenyl.

In some embodiments, R² is —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl, heterocyclyl, or aryl represented by R² is substituted with one or more —OH, —NH₂, —NH—C(═O)—O—(C₁₋₆ alkyl), halogen, C₁₋₆ alkyl, or phenyl.

In some embodiments, R² is —C(═O)—C₁₋₆ alkyl.

In some embodiments, R² is —C(═O)—C₁₋₆ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—C₁₋₆ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—C₁ alkyl.

In some embodiments, R² is —C(═O)—C₁ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—C₁ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—C₂ alkyl.

In some embodiments, R² is —C(═O)—C₂ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—C₂ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—C₃ alkyl.

In some embodiments, R² is —C(═O)—C₃ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—C₃ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—C₄ alkyl.

In some embodiments, R² is —C(═O)—C₄ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—C₄ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—C₅ alkyl.

In some embodiments, R² is —C(═O)—C₅ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—C₅ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—C₆ alkyl.

In some embodiments, R² is —C(═O)—C₆ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—C₆ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—(C₆₋₁₀ aryl).

In some embodiments, R² is —C(═O)—(C₆₋₁₀ aryl) wherein the aryl is optionally substituted with one or more —OH, —NH₂, —NH—C(═O)—O—(C₁₋₆ alkyl), halogen, C₁₋₆ alkyl, or phenyl.

In some embodiments, R² is —C(═O)—(C₆₋₁₀ aryl) wherein the aryl is substituted with one or more —OH, —NH₂, —NH—C(═O)—O—(C₁₋₆ alkyl), halogen, C₁₋₆ alkyl, or phenyl.

In some embodiments, R² is —C(═O)-phenyl.

In some embodiments, R² is —C(═O)-phenyl wherein the phenyl is optionally substituted with one or more —OH, —NH₂, —NH—C(═O)—O—(C₁₋₆ alkyl), halogen, C₁₋₆ alkyl, or phenyl.

In some embodiments, R² is —C(═O)-phenyl wherein the phenyl is substituted with one or more —OH, —NH₂, —NH—C(═O)—O—(C₁₋₆ alkyl), halogen, C₁₋₆ alkyl, or phenyl.

In some embodiments, R² is —C(═O)-(5- to 7-membered heterocyclyl).

In some embodiments, R² is —C(═O)-(5- to 7-membered heterocyclyl) wherein the heterocyclyl is optionally substituted with one or more —OH, —NH₂, —NH—C(═O)—O—(C₁₋₆ alkyl), halogen, C₁₋₆ alkyl, or phenyl.

In some embodiments, R² is —C(═O)-(5- to 7-membered heterocyclyl) wherein the heterocyclyl is substituted with one or more —OH, —NH₂, —NH—C(═O)—O—(C₁₋₆ alkyl), halogen, C₁₋₆ alkyl, or phenyl.

In some embodiments, R² is —C(═O)-(5-membered heterocyclyl).

In some embodiments, R² is —C(═O)-(5-membered heterocyclyl) wherein the heterocyclyl is optionally substituted with one or more —OH, —NH₂, —NH—C(═O)—O—(C₁₋₆ alkyl), halogen, C₁₋₆ alkyl, or phenyl.

In some embodiments, R² is —C(═O)-(5-membered heterocyclyl) wherein the heterocyclyl is substituted with one or more —OH, —NH₂, —NH—C(═O)—O—(C₁₋₆ alkyl), halogen, C₁₋₆ alkyl, or phenyl.

In some embodiments, R² is —C(═O)-(6-membered heterocyclyl).

In some embodiments, R² is —C(═O)-(6-membered heterocyclyl) wherein the heterocyclyl is optionally substituted with one or more —OH, —NH₂, —NH—C(═O)—O—(C₁₋₆ alkyl), halogen, C₁₋₆ alkyl, or phenyl.

In some embodiments, R² is —C(═O)-(6-membered heterocyclyl) wherein the heterocyclyl is substituted with one or more —OH, —NH₂, —NH—C(═O)—O—(C₁₋₆ alkyl), halogen, C₁₋₆ alkyl, or phenyl.

In some embodiments, R² is —C(═O)-(7-membered heterocyclyl).

In some embodiments, R² is —C(═O)-(7-membered heterocyclyl) wherein the heterocyclyl is optionally substituted with one or more —OH, —NH₂, —NH—C(═O)—O—(C₁₋₆ alkyl), halogen, C₁₋₆ alkyl, or phenyl.

In some embodiments, R² is —C(═O)-(5- to 7-membered heterocyclyl) wherein the heterocyclyl is substituted with one or more —OH, —NH₂, —NH—C(═O)—O—(C₁₋₆ alkyl), halogen, C₁₋₆ alkyl, or phenyl.

In some embodiments, R² is —C(═O)—O—C₁₋₆ alkyl.

In some embodiments, R² is —C(═O)—O—C₁₋₆ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—O—C₁₋₆ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—O—C₁ alkyl.

In some embodiments, R² is —C(═O)—O—C₁ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—O—C₁ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—O—C₂ alkyl.

In some embodiments, R² is —C(═O)—O—C₂ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—O—C₂ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—O—C₃ alkyl.

In some embodiments, R² is —C(═O)—O—C₃ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—O—C₃ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—O—C₄ alkyl.

In some embodiments, R² is —C(═O)—O—C₄ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—O—C₄ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—O—C₅ alkyl.

In some embodiments, R² is —C(═O)—O—C₅ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—O—C₅ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—O—C₆ alkyl.

In some embodiments, R² is —C(═O)—O—C₆ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—O—C₆ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —SO₂—(C₆₋₁₀ aryl).

In some embodiments, R² is —SO₂—(C₆₋₁₀ aryl) wherein the aryl is optionally substituted with one or more —OH, —NH₂, —NH—C(═O)—O—(C₁₋₆ alkyl), halogen, C₁₋₆ alkyl, or phenyl.

In some embodiments, R² is —SO₂—(C₆₋₁₀ aryl) wherein the aryl is substituted with one or more —OH, —NH₂, —NH—C(═O)—O—(C₁₋₆ alkyl), halogen, C₁₋₆ alkyl, or phenyl.

In some embodiments, R² is —SO₂-phenyl.

In some embodiments, R² is —SO₂—(C₆₋₁₀ aryl) wherein the phenyl is optionally substituted with one or more —OH, —NH₂, —NH—C(═O)—O—(C₁₋₆ alkyl), halogen, C₁₋₆ alkyl, or phenyl.

In some embodiments, R² is —SO₂-phenyl wherein the phenyl is substituted with one or more —OH, —NH₂, —NH—C(═O)—O—(C₁₋₆ alkyl), halogen, C₁₋₆ alkyl, or phenyl.

In some embodiments, R² is —C(═O)—NH—C₁₋₆ alkyl.

In some embodiments, R² is —C(═O)—NH—C₁₋₆ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—NH—C₁₋₆ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—NH—C₁ alkyl.

In some embodiments, R² is —C(═O)—NH—C₁ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—NH—C₁ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—NH—C₂ alkyl.

In some embodiments, R² is —C(═O)—NH—C₂ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—NH—C₂ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—NH—C₃ alkyl.

In some embodiments, R² is —C(═O)—NH—C₃ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—NH—C₃ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—NH—C₄ alkyl.

In some embodiments, R² is —C(═O)—NH—C₄ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—NH—C₄ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—NH—C₅ alkyl.

In some embodiments, R² is —C(═O)—NH—C₅ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—NH—C₅ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—NH—C₆ alkyl.

In some embodiments, R² is —C(═O)—NH—C₆ alkyl wherein the alkyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—NH—C₆ alkyl wherein the alkyl is substituted with one or more —OH, —NH₂, —NH—C(═O)—O—(C₁₋₆ alkyl), halogen, C₁₋₆ alkyl, or phenyl.

In some embodiments, R² is —C(═O)—NH—(C₆₋₁₀ aryl).

In some embodiments, R² is —C(═O)—NH—(C₆₋₁₀ aryl) wherein the aryl is optionally substituted with one or more —OH, —NH₂, —NH—C(═O)—O—(C₁₋₆ alkyl), halogen, C₁₋₆ alkyl, or phenyl.

In some embodiments, R² is —C(═O)—NH—(C₆₋₁₀ aryl) wherein the aryl is substituted with one or more —OH, —NH₂, —NH—C(═O)—O—(C₁₋₆ alkyl), halogen, C₁₋₆ alkyl, or phenyl.

In some embodiments, R² is —C(═O)—NH-phenyl.

In some embodiments, R² is —C(═O)—NH-phenyl wherein the phenyl is optionally substituted with one or more —OH, —NH₂, —NH—C(═O)—O—(C₁₋₆ alkyl), halogen, C₁₋₆ alkyl, or phenyl.

In some embodiments, R² is —C(═O)—NH-phenyl wherein the phenyl is substituted with one or more —OH, —NH₂, —NH—C(═O)—O—(C₁₋₆ alkyl), halogen, C₁₋₆ alkyl, or phenyl.

In some embodiments, R² is H, CH₃,

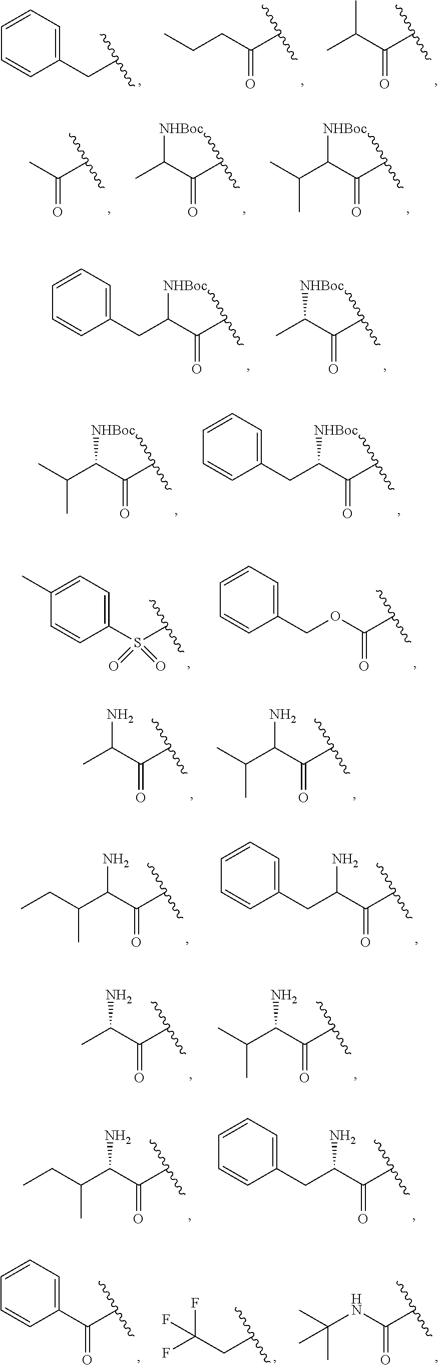

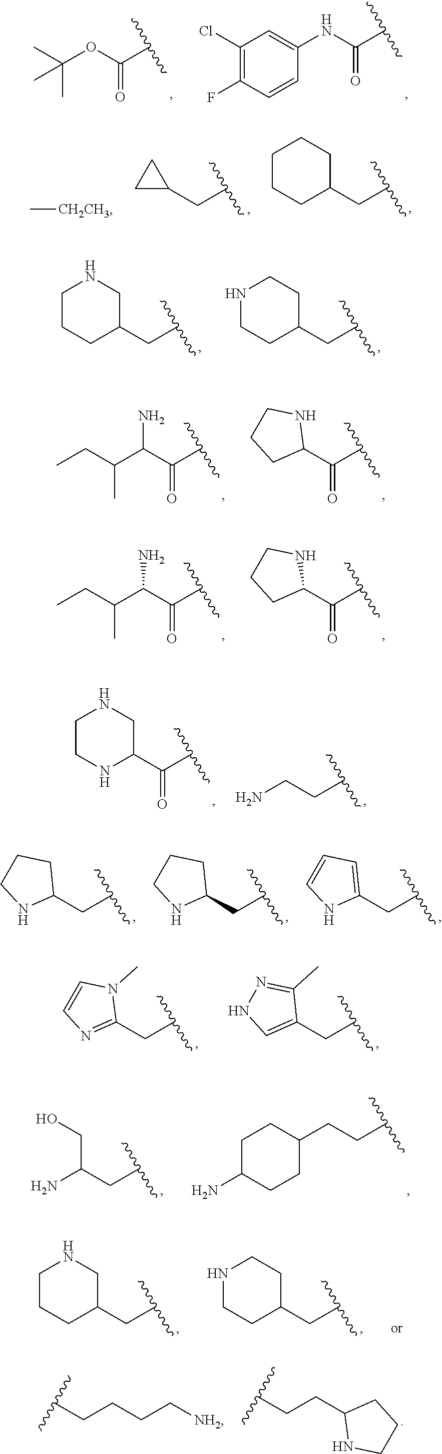

In some embodiments, R³ is H, C₁₋₆ alkyl, C₂₋₆ alkenyl, C₂₋₆ alkynyl, C₁₋₆ haloalkyl, halogen, —CN, —NO₂, —OR, —SR, —S(═O)₂R, —C(═O)R, —OC(═O)R, —NR₂, or —CO₂R.

In some embodiments, R³ is H, C₁₋₆ alkyl, C₁₋₆ haloalkyl, C₁₋₆ heteroalkyl, F, Cl, Br, I, CN, NO₂, OR, SR, S(═O)₂R, C(═O)R, OC(═O)R, NR₂, or CO₂R.

In some embodiments, R³ is H.

In some embodiments, R³ is C₁₋₆ alkyl, C₂₋₆ alkenyl, C₂₋₆ alkynyl, C₁₋₆ haloalkyl, halogen, —CN, —NO₂, —OR, —SR, —S(═O)₂R, —C(═O)R, —OC(═O)R, —NR₂, or —CO₂R.

In some embodiments, R³ is C₁₋₆ alkyl, C₂₋₆ alkenyl, C₂₋₆ alkynyl, or C₁₋₆ haloalkyl.

In some embodiments, R³ is C₁₋₆ alkyl, C₂₋₆ alkenyl, or C₂₋₆ alkynyl.

In some embodiments, R³ is C₁₋₆ alkyl.

In some embodiments, R³ is C₁ alkyl. In some embodiments, R³ is C₂ alkyl. In some embodiments, R³ is C₃ alkyl. In some embodiments, R³ is C₄ alkyl. In some embodiments, R³ is C₅ alkyl. In some embodiments, R³ is C₆ alkyl.

In some embodiments, R³ is methyl. In some embodiments, R³ is ethyl. In some embodiments, R³ is propyl. In some embodiments, R³ is isopropyl. In some embodiments, R³ is butyl. In some embodiments, R³ is isobutyl. In some embodiments, R³ is sec-butyl. In some embodiments, R³ is tert-butyl. In some embodiments, R³ is pentyl. In some embodiments, R³ is hexyl.

In some embodiments, R³ is C₂₋₆ alkenyl. In some embodiments, R³ is C₂₋₆ alkynyl.

In some embodiments, R³ is C₁₋₆ haloalkyl.

In some embodiments, R³ is C₁ haloalkyl. In some embodiments, R³ is C₂ haloalkyl.

In some embodiments, R³ is C₃ haloalkyl. In some embodiments, R³ is C₄ haloalkyl. In some embodiments, R³ is C₅ haloalkyl. In some embodiments, R³ is C₆ haloalkyl.

In some embodiments, R³ is halomethyl. In some embodiments, R³ is haloethyl. In some embodiments, R³ is halopropyl. In some embodiments, R³ is n-halopropyl. In some embodiments, R³ is haloisopropyl. In some embodiments, R³ is halobutyl. In some embodiments, R³ is n-halobutyl. In some embodiments, R³ is haloisobutyl. In some embodiments, R³ is sec-halobutyl. In some embodiments, R³ is tert-halobutyl. In some embodiments, R³ is halopentyl. In some embodiments, R³ is halohexyl.

In some embodiments, R³ is halogen, —CN, or —NO₂.

In some embodiments, R³ is halogen.

In some embodiments, R³ is F, Cl, Br, or I. In some embodiments, R³ is F, Cl, or Br.

In some embodiments, R³ is F or Cl. In some embodiments, R³ is F. In some embodiments, R³ is Cl. In some embodiments, R³ is Br. In some embodiments, R³ is I.

In some embodiments, R³ is CN. In some embodiments, R³ is NO₂.

In some embodiments, R³ is —OR, —SR, —S(═O)₂R, —C(═O)R, —OC(═O)R, —NR₂, and —CO₂R.

In some embodiments, R³ is —OR. In some embodiments, R³ is SR. In some embodiments, R³ is S(═O)₂R. In some embodiments, R³ is C(═O)R. In some embodiments, R³ is OC(═O)R. In some embodiments, R³ is NR₂. In some embodiments, R³ is CO₂R.

In some embodiments, each R⁴ and R^{4′} is independently H, —C(═O)—O—(C₁₋₆ alkyl), C₁₋₆ alkyl, C₂₋₆ alkenyl, C₂₋₆ alkynyl, or C₃₋₆ cycloalkyl.

In some embodiments, R⁴ is H, —C(═O)—O—(C₁₋₆ alkyl), C₁₋₆ alkyl, C₂₋₆ alkenyl, C₂₋₆ alkynyl, or C₃₋₆ cycloalkyl.

In some embodiments, R⁴ is H.

In some embodiments, R⁴ is —C(═O)—O—(C₁₋₆ alkyl), C₁₋₆ alkyl, C₂₋₆ alkenyl, C₂₋₆ alkynyl, or C₃₋₆ cycloalkyl.

In some embodiments, R⁴ is —C(═O)—O—(C₁₋₆ alkyl).

In some embodiments, R⁴ is —C(═O)—O—(C₁ alkyl). In some embodiments, R⁴ is —C(═O)—O—(C₂ alkyl). In some embodiments, R⁴ is —C(═O)—O—(C₃ alkyl). In some embodiments, R⁴ is —C(═O)—O—(C₄ alkyl). In some embodiments, R⁴ is —C(═O)—O—(C₅ alkyl).

In some embodiments, R⁴ is —C(═O)—O-(tert-butoxy). In some embodiments, R⁴ is —C(═O)—O—(C₆ alkyl).

In some embodiments, R⁴ is C₁₋₆ alkyl, C₂₋₆ alkenyl, or C₂₋₆ alkynyl.

In some embodiments, R⁴ is C₁₋₆ alkyl.

In some embodiments, R⁴ is C₁₋₆ alkyl, C₂₋₆ alkenyl, C₂₋₆ alkynyl, or C₃₋₆ cycloalkyl.

In some embodiments, R⁴ is C₁ alkyl. In some embodiments, R⁴ is C₂ alkyl. In some embodiments, R⁴ is C₃ alkyl. In some embodiments, R⁴ is C₄ alkyl. In some embodiments, R⁴ is C₅ alkyl. In some embodiments, R⁴ is C₆ alkyl.

In some embodiments, R⁴ is methyl. In some embodiments, R⁴ is ethyl. In some embodiments, R⁴ is propyl. In some embodiments, R⁴ is isopropyl. In some embodiments, R⁴ is butyl. In some embodiments, R⁴ is isobutyl. In some embodiments, R⁴ is sec-butyl. In some embodiments, R⁴ is tert-butyl. In some embodiments, R⁴ is pentyl. In some embodiments, R⁴ is hexyl.

In some embodiments, R⁴ is C₂₋₆ alkenyl. In some embodiments, R⁴ is C₂₋₆ alkynyl.

In some embodiments, R⁴ is C₃₋₆ cycloalkyl.

In some embodiments, R⁴ is C₃ cycloalkyl. In some embodiments, R⁴ is C₄ cycloalkyl. In some embodiments, R⁴ is C₅ cycloalkyl. In some embodiments, R⁴ is C₆ cycloalkyl.

In some embodiments, R⁴ is cyclopropyl. In some embodiments, R⁴ is cyclobutyl. In some embodiments, R⁴ is cyclopentyl. In some embodiments, R⁴ is cyclohexyl.

In some embodiments, R^{4′} is H, —C(═O)—O—(C₁₋₆ alkyl), C₁₋₆ alkyl, C₂₋₆ alkenyl, C₂₋₆ alkynyl, or C₃₋₆ cycloalkyl.

In some embodiments, R^{4′} is H.

In some embodiments, R^{4′} is —C(═O)—O—(C₁₋₆ alkyl), C₁₋₆ alkyl, C₂₋₆ alkenyl, C₂₋₆ alkynyl, or C₃₋₆ cycloalkyl.

In some embodiments, R^{4′} is —C(═O)—O—(C₁₋₆ alkyl).

In some embodiments, R^{4′} is —C(═O)—O—(C₁ alkyl). In some embodiments, R^{4′} is —C(═O)—O—(C₂ alkyl). In some embodiments, R^{4′} is —C(═O)—O—(C₃ alkyl). In some embodiments, R^{4′} is —C(═O)—O—(C₄ alkyl). In some embodiments, R^{4′} is —C(═O)—O—(C₅ alkyl).

In some embodiments, R^{4′} is —C(═O)—O-(tert-butoxy). In some embodiments, R^{4′} is —C(═O)—O—(C₆ alkyl).

In some embodiments, R^{4′} is C₁₋₆ alkyl, C₂₋₆ alkenyl, C₂₋₆ alkynyl, or C₃₋₆ cycloalkyl.

In some embodiments, R^{4′} is C₁₋₆ alkyl, C₂₋₆ alkenyl, or C₂₋₆ alkynyl.

In some embodiments, R^{4′} is C₁₋₆ alkyl.

In some embodiments, R^{4′} is C₁ alkyl. In some embodiments, R^{4′} is C₂ alkyl. In some embodiments, R^{4′} is C₃ alkyl. In some embodiments, R^{4′} is C₄ alkyl. In some embodiments, R^{4′} is C₅ alkyl. In some embodiments, R^{4′} is C₆ alkyl.

In some embodiments, R^{4′} is methyl. In some embodiments, R^{4′} is ethyl. In some embodiments, R^{4′} is propyl. In some embodiments, R^{4′} is isopropyl. In some embodiments, R^{4′} is butyl. In some embodiments, R^{4′} is isobutyl. In some embodiments, R^{4′} is sec-butyl. In some embodiments, R^{4′} is tert-butyl. In some embodiments, R^{4′} is pentyl. In some embodiments, R^{4′} is hexyl.

In some embodiments, R^{4′} is C₂₋₆ alkenyl. In some embodiments, R^{4′} is C₂₋₆ alkynyl.

In some embodiments, R^{4′} is C₃₋₆ cycloalkyl.

In some embodiments, R^{4′} is C₃ cycloalkyl. In some embodiments, R^{4′} is C₄ cycloalkyl. In some embodiments, R^{4′} is C₅ cycloalkyl. In some embodiments, R^{4′} is C₆ cycloalkyl.

In some embodiments, R^{4′} is cyclopropyl. In some embodiments, R^{4′} is cyclobutyl. In some embodiments, R^{4′} is cyclopentyl. In some embodiments, R^{4′} is cyclohexyl.

In some embodiments, R⁴ and R^{4′}, together with the nitrogen atom to which R⁴ and R_{4′} are attached, form a C₅₋₇ heterocyclyl ring.

In some embodiments, R⁴ and R^{4′}, together with the nitrogen atom to which R⁴ and R^{4′} are attached, form a C₅₋₆ heterocyclyl ring. In some embodiments, R⁴ and R^{4′}, together with the nitrogen atom to which R⁴ and R^{4′} are attached, form a C₆₋₇ heterocyclyl ring.

In some embodiments, R⁵ is -(5- to 7-membered heterocyclyl)-C₀₋₆-alkyl, —C₀₋₆-alkyl-(5- to 7-membered heterocyclyl), —(C₃₋₆ cycloalkyl)-C₀₋₆-alkyl, or —C₀₋₆-alkyl-(C₃₋₆ cycloalkyl).

In some embodiments, R⁵ is -(5- to 7-membered heterocyclyl)-C₀₋₆-alkyl, —C₀₋₆-alkyl-(5- to 7-membered heterocyclyl), —(C₃₋₆ cycloalkyl)-C₀₋₆-alkyl, or —C₀₋₆-alkyl-(C₃₋₆ cycloalkyl), wherein the alkyl, heterocyclyl, or cycloalkyl represented by R⁵ is optionally substituted by one or more C₁₋₆ alkyl.

In some embodiments, R⁵ is -(5- to 7-membered heterocyclyl)-C₀₋₆-alkyl, —C₀₋₆-alkyl-(5- to 7-membered heterocyclyl), —(C₃₋₆ cycloalkyl)-C₀₋₆-alkyl, or —C₀₋₆-alkyl-(C₃₋₆ cycloalkyl), wherein the alkyl, heterocyclyl, or cycloalkyl represented by R⁵ is substituted by one or more C₁₋₆ alkyl.

In some embodiments, R⁵ is -(5- to 7-membered heterocyclyl)-C₀₋₆-alkyl or —(C₃₋₆ cycloalkyl)-C₀₋₆-alkyl.

In some embodiments, R⁵ is -(5- to 7-membered heterocyclyl)-C₀₋₆-alkyl or —(C₃₋₆ cycloalkyl)-C₀₋₆-alkyl, wherein the alkyl, heterocyclyl, or cycloalkyl represented by R⁵ is optionally substituted by one or more C₁₋₆ alkyl.

In some embodiments, R⁵ is -(5- to 7-membered heterocyclyl)-C₀₋₆-alkyl or —(C₃₋₆ cycloalkyl)-C₀₋₆-alkyl, wherein the alkyl, heterocyclyl, or cycloalkyl represented by R⁵ is substituted by one or more C₁₋₆ alkyl.

In some embodiments, R⁵ is -(5- to 7-membered heterocyclyl)-C₀₋₆-alkyl.

In some embodiments, R⁵ is -(5- to 7-membered heterocyclyl)-C₀₋₆-alkyl, wherein the alkyl or heterocyclyl represented by R⁵ is optionally substituted by one or more C₁₋₆ alkyl.

In some embodiments, R⁵ is -(5- to 7-membered heterocyclyl)-C₀₋₆-alkyl, wherein the alkyl or heterocyclyl represented by R⁵ is substituted by one or more C₁₋₆ alkyl.

In some embodiments, R⁵ is -(5-membered heterocyclyl)-C₀₋₆-alkyl.

In some embodiments, R⁵ is -(5-membered heterocyclyl)-C₀₋₆-alkyl, wherein the alkyl or heterocyclyl represented by R⁵ is optionally substituted by one or more C₁₋₆ alkyl.

In some embodiments, R⁵ is -(5-membered heterocyclyl)-C₀₋₆-alkyl, wherein the alkyl or heterocyclyl represented by R⁵ is substituted by one or more C₁₋₆ alkyl.

In some embodiments, R⁵ is -(6-membered heterocyclyl)-C₀₋₆-alkyl.

In some embodiments, R⁵ is -(6-membered heterocyclyl)-C₀₋₆-alkyl, wherein the alkyl or heterocyclyl represented by R⁵ is optionally substituted by one or more C₁₋₆ alkyl.

In some embodiments, R⁵ is -(6-membered heterocyclyl)-C₀₋₆-alkyl, wherein the alkyl or heterocyclyl represented by R⁵ is substituted by one or more C₁₋₆ alkyl.

In some embodiments, R⁵ is -(7-membered heterocyclyl)-C₀₋₆-alkyl.

In some embodiments, R⁵ is -(7-membered heterocyclyl)-C₀₋₆-alkyl, wherein the alkyl or heterocyclyl represented by R⁵ is optionally substituted by one or more C₁₋₆ alkyl.

In some embodiments, R⁵ is -(7-membered heterocyclyl)-C₀₋₆-alkyl, wherein the alkyl or heterocyclyl represented by R⁵ is substituted by one or more C₁₋₆ alkyl.

In some embodiments, R⁵ is 5- to 7-membered heterocyclyl.

In some embodiments, R⁵ is 5- to 7-membered heterocyclyl, optionally substituted by one or more C₁₋₆ alkyl.

In some embodiments, R⁵ is 5- to 7-membered heterocyclyl, substituted by one or more C₁₋₆ alkyl.

In some embodiments, R⁵ is -(5- to 7-membered heterocyclyl)-C₁-alkyl.

In some embodiments, R⁵ is -(5- to 7-membered heterocyclyl)-C₁-alkyl, wherein the alkyl or heterocyclyl represented by R⁵ is optionally substituted by one or more C₁₋₆ alkyl.

In some embodiments, R⁵ is -(5- to 7-membered heterocyclyl)-C₁-alkyl, wherein the alkyl or heterocyclyl represented by R⁵ is substituted by one or more C₁₋₆ alkyl.

In some embodiments, R⁵ is -(5- to 7-membered heterocyclyl)-C₂-alkyl.

In some embodiments, R⁵ is -(5- to 7-membered heterocyclyl)-C₂-alkyl, wherein the alkyl or heterocyclyl represented by R⁵ is optionally substituted by one or more C₁₋₆ alkyl.

In some embodiments, R⁵ is -(5- to 7-membered heterocyclyl)-C₂-alkyl, wherein the alkyl or heterocyclyl represented by R⁵ is substituted by one or more C₁₋₆ alkyl.

In some embodiments, R⁵ is -(5- to 7-membered heterocyclyl)-C₃-alkyl.

In some embodiments, R⁵ is -(5- to 7-membered heterocyclyl)-C₃-alkyl, wherein the alkyl or heterocyclyl represented by R⁵ is optionally substituted by one or more C₁₋₆ alkyl.

In some embodiments, R⁵ is -(5- to 7-membered heterocyclyl)-C₃-alkyl, wherein the alkyl or heterocyclyl represented by R⁵ is substituted by one or more C₁₋₆ alkyl.

In some embodiments, R⁵ is -(5- to 7-membered heterocyclyl)-C₄-alkyl.

In some embodiments, R⁵ is -(5- to 7-membered heterocyclyl)-C₄-alkyl, wherein the alkyl or heterocyclyl represented by R⁵ is optionally substituted by one or more C₁₋₆ alkyl.

In some embodiments, R⁵ is -(5- to 7-membered heterocyclyl)-C₄-alkyl, wherein the alkyl or heterocyclyl represented by R⁵ is substituted by one or more C₁₋₆ alkyl.

In some embodiments, R⁵ is -(5- to 7-membered heterocyclyl)-C₅-alkyl.

In some embodiments, R⁵ is -(5- to 7-membered heterocyclyl)-C₅-alkyl, wherein the alkyl or heterocyclyl represented by R⁵ is optionally substituted by one or more C₁₋₆ alkyl.

In some embodiments, R⁵ is -(5- to 7-membered heterocyclyl)-C₅-alkyl, wherein the alkyl or heterocyclyl represented by R⁵ is substituted by one or more C₁₋₆ alkyl.

In some embodiments, R⁵ is -(5- to 7-membered heterocyclyl)-C₆-alkyl.

In some embodiments, R⁵ is -(5- to 7-membered heterocyclyl)-C₆-alkyl, wherein the alkyl or heterocyclyl represented by R⁵ is optionally substituted by one or more C₁₋₆ alkyl.

In some embodiments, R⁵ is -(5- to 7-membered heterocyclyl)-C₆-alkyl, wherein the alkyl or heterocyclyl represented by R⁵ is substituted by one or more C₁₋₆ alkyl.

In some embodiments, R⁵ is —(C₃₋₆ cycloalkyl)-C₀₋₆-alkyl.

In some embodiments, R⁵ is —(C₃₋₆ cycloalkyl)-C₀₋₆-alkyl, wherein the alkyl or cycloalkyl represented by R⁵ is optionally substituted by one or more C₁₋₆ alkyl.

In some embodiments, R⁵ is —(C₃₋₆ cycloalkyl)-C₀₋₆-alkyl, wherein the alkyl or cycloalkyl represented by R⁵ is substituted by one or more C₁₋₆ alkyl.

In some embodiments, R⁵ is —(C₃ cycloalkyl)-C₀₋₆-alkyl.

In some embodiments, R⁵ is —(C₃ cycloalkyl)-C₀₋₆-alkyl, wherein the alkyl or cycloalkyl represented by R⁵ is optionally substituted by one or more C₁₋₆ alkyl.

In some embodiments, R⁵ is —(C₃ cycloalkyl)-C₀₋₆-alkyl, wherein the alkyl or cycloalkyl represented by R⁵ is substituted by one or more C₁₋₆ alkyl.

In some embodiments, R⁵ is —(C₄ cycloalkyl)-C₀₋₆-alkyl.

In some embodiments, R⁵ is —(C₄ cycloalkyl)-C₀₋₆-alkyl, wherein the alkyl or cycloalkyl represented by R⁵ is optionally substituted by one or more C₁₋₆ alkyl.

In some embodiments, R⁵ is —(C₄ cycloalkyl)-C₀₋₆-alkyl, wherein the alkyl or cycloalkyl represented by R⁵ is substituted by one or more C₁₋₆ alkyl.

In some embodiments, R⁵ is —(C₅ cycloalkyl)-C₀₋₆-alkyl.

In some embodiments, R⁵ is —(C₅ cycloalkyl)-C₀₋₆-alkyl, wherein the alkyl or cycloalkyl represented by R⁵ is optionally substituted by one or more C₁₋₆ alkyl.

In some embodiments, R⁵ is —(C₅ cycloalkyl)-C₀₋₆-alkyl, wherein the alkyl or cycloalkyl represented by R⁵ is substituted by one or more C₁₋₆ alkyl.

In some embodiments, R⁵ is —(C₆ cycloalkyl)-C₀₋₆-alkyl.

In some embodiments, R⁵ is —(C₆ cycloalkyl)-C₀₋₆-alkyl, wherein the alkyl or cycloalkyl represented by R⁵ is optionally substituted by one or more C₁₋₆ alkyl.

In some embodiments, R⁵ is —(C₆ cycloalkyl)-C₀₋₆-alkyl, wherein the alkyl or cycloalkyl represented by R⁵ is substituted by one or more C₁₋₆ alkyl.

In some embodiments, R⁵ is C₃₋₆ cycloalkyl.

In some embodiments, R⁵ is C₃₋₆ cycloalkyl, optionally substituted by one or more C₁₋₆ alkyl.

In some embodiments, R⁵ is C₃₋₆ cycloalkyl, substituted by one or more C₁₋₆ alkyl.

In some embodiments, R⁵ is —(C₃₋₆ cycloalkyl)-C₁-alkyl.

In some embodiments, R⁵ is —(C₃₋₆ cycloalkyl)-C₁-alkyl, wherein the alkyl or cycloalkyl represented by R⁵ is optionally substituted by one or more C₁₋₆ alkyl.

In some embodiments, R⁵ is —(C₃₋₆ cycloalkyl)-C₁-alkyl, wherein the alkyl or cycloalkyl represented by R⁵ is substituted by one or more C₁₋₆ alkyl.

In some embodiments, R⁵ is —(C₃₋₆ cycloalkyl)-C₂-alkyl.

In some embodiments, R⁵ is —(C₃₋₆ cycloalkyl)-C₂-alkyl, wherein the alkyl or cycloalkyl represented by R⁵ is optionally substituted by one or more C₁₋₆ alkyl.

In some embodiments, R⁵ is —(C₃₋₆ cycloalkyl)-C₂-alkyl, wherein the alkyl or cycloalkyl represented by R⁵ is substituted by one or more C₁₋₆ alkyl.

In some embodiments, R⁵ is —(C₃₋₆ cycloalkyl)-C₃-alkyl.

In some embodiments, R⁵ is —(C₃₋₆ cycloalkyl)-C₃-alkyl, wherein the alkyl or cycloalkyl represented by R⁵ is optionally substituted by one or more C₁₋₆ alkyl.

In some embodiments, R⁵ is —(C₃₋₆ cycloalkyl)-C₃-alkyl, wherein the alkyl or cycloalkyl represented by R⁵ is substituted by one or more C₁₋₆ alkyl.

In some embodiments, R⁵ is —(C₃₋₆ cycloalkyl)-C₄-alkyl.

In some embodiments, R⁵ is —(C₃₋₆ cycloalkyl)-C₄-alkyl, wherein the alkyl or cycloalkyl represented by R⁵ is optionally substituted by one or more C₁₋₆ alkyl.

In some embodiments, R⁵ is —(C₃₋₆ cycloalkyl)-C₄-alkyl, wherein the alkyl or cycloalkyl represented by R⁵ is substituted by one or more C₁₋₆ alkyl.

In some embodiments, R⁵ is —(C₃₋₆ cycloalkyl)-C₅-alkyl.

In some embodiments, R⁵ is —(C₃₋₆ cycloalkyl)-C₅-alkyl, wherein the alkyl or cycloalkyl represented by R⁵ is optionally substituted by one or more C₁₋₆ alkyl.

In some embodiments, R⁵ is —(C₃₋₆ cycloalkyl)-C₅-alkyl, wherein the alkyl or cycloalkyl represented by R⁵ is substituted by one or more C₁₋₆ alkyl.

In some embodiments, R⁵ is —(C₃₋₆ cycloalkyl)-C₆-alkyl.

In some embodiments, R⁵ is —(C₃₋₆ cycloalkyl)-C₆-alkyl, wherein the alkyl or cycloalkyl represented by R⁵ is optionally substituted by one or more C₁₋₆ alkyl.

In some embodiments, R⁵ is —(C₃₋₆ cycloalkyl)-C₆-alkyl, wherein the alkyl or cycloalkyl represented by R⁵ is substituted by one or more C₁₋₆ alkyl.

In some embodiments, R⁵ is —C₀₋₆-alkyl-(5- to 7-membered heterocyclyl) or —C₀₋₆-alkyl-(C₃₋₆ cycloalkyl).

In some embodiments, R⁵ is —C₀₋₆-alkyl-(5- to 7-membered heterocyclyl) or —C₀₋₆-alkyl-(C₃₋₆ cycloalkyl), wherein the alkyl, heterocyclyl, or cycloalkyl represented by R⁵ is optionally substituted by one or more C₁₋₆ alkyl.

In some embodiments, R⁵ is —C₀₋₆-alkyl-(5- to 7-membered heterocyclyl) or —C₀₋₆-alkyl-(C₃₋₆ cycloalkyl), wherein the alkyl, heterocyclyl, or cycloalkyl represented by R⁵ is substituted by one or more C₁₋₆ alkyl.

In some embodiments, R⁵ is —C₀₋₆-alkyl-(5- to 7-membered heterocyclyl).

In some embodiments, R⁵ is —C₀₋₆-alkyl-(5- to 7-membered heterocyclyl), wherein the alkyl or heterocyclyl represented by R⁵ is optionally substituted by one or more C₁₋₆ alkyl.

In some embodiments, R⁵ is —C₀₋₆-alkyl-(5- to 7-membered heterocyclyl), wherein the alkyl or heterocyclyl represented by R⁵ is substituted by one or more C₁₋₆ alkyl.

In some embodiments, R⁵ is —C₀₋₆-alkyl-(5-membered heterocyclyl).

In some embodiments, R⁵ is —C₀₋₆-alkyl-(5-membered heterocyclyl), wherein the alkyl or heterocyclyl represented by R⁵ is optionally substituted by one or more C₁₋₆ alkyl.

In some embodiments, R⁵ is —C₀₋₆-alkyl-(5-membered heterocyclyl), wherein the alkyl or heterocyclyl represented by R⁵ is substituted by one or more C₁₋₆ alkyl.

In some embodiments, R⁵ is —C₀₋₆-alkyl-(6-membered heterocyclyl).

In some embodiments, R⁵ is —C₀₋₆-alkyl-(6-membered heterocyclyl), wherein the alkyl or heterocyclyl represented by R⁵ is optionally substituted by one or more C₁₋₆ alkyl.

In some embodiments, R⁵ is —C₀₋₆-alkyl-(6-membered heterocyclyl), wherein the alkyl or heterocyclyl represented by R⁵ is substituted by one or more C₁₋₆ alkyl.

In some embodiments, R⁵ is —C₀₋₆-alkyl-(7-membered heterocyclyl).

In some embodiments, R⁵ is —C₀₋₆-alkyl-(7-membered heterocyclyl), wherein the alkyl or heterocyclyl represented by R⁵ is optionally substituted by one or more C₁₋₆ alkyl.

In some embodiments, R⁵ is —C₀₋₆-alkyl-(7-membered heterocyclyl), wherein the alkyl or heterocyclyl represented by R⁵ is substituted by one or more C₁₋₆ alkyl.

In some embodiments, R⁵ is —C₁-alkyl-(5- to 7-membered heterocyclyl).

In some embodiments, R⁵ is —C₁-alkyl-(5- to 7-membered heterocyclyl), wherein the alkyl or heterocyclyl represented by R⁵ is optionally substituted by one or more C₁₋₆ alkyl.

In some embodiments, R⁵ is —C₁-alkyl-(5- to 7-membered heterocyclyl), wherein the alkyl or heterocyclyl represented by R⁵ is substituted by one or more C₁₋₆ alkyl.

In some embodiments, R⁵ is —C₂-alkyl-(5- to 7-membered heterocyclyl).

In some embodiments, R⁵ is —C₂-alkyl-(5- to 7-membered heterocyclyl), wherein the alkyl or heterocyclyl represented by R⁵ is optionally substituted by one or more C₁₋₆ alkyl.

In some embodiments, R⁵ is —C₂-alkyl-(5- to 7-membered heterocyclyl), wherein the alkyl or heterocyclyl represented by R⁵ is substituted by one or more C₁₋₆ alkyl.

In some embodiments, R⁵ is —C₃-alkyl-(5- to 7-membered heterocyclyl).

In some embodiments, R⁵ is —C₃-alkyl-(5- to 7-membered heterocyclyl), wherein the alkyl or heterocyclyl represented by R⁵ is optionally substituted by one or more C₁₋₆ alkyl.

In some embodiments, R⁵ is —C₃-alkyl-(5- to 7-membered heterocyclyl), wherein the alkyl or heterocyclyl represented by R⁵ is substituted by one or more C₁₋₆ alkyl.

In some embodiments, R⁵ is —C₄-alkyl-(5- to 7-membered heterocyclyl).

In some embodiments, R⁵ is —C₄-alkyl-(5- to 7-membered heterocyclyl), wherein the alkyl or heterocyclyl represented by R⁵ is optionally substituted by one or more C₁₋₆ alkyl.

In some embodiments, R⁵ is —C₄-alkyl-(5- to 7-membered heterocyclyl), wherein the alkyl or heterocyclyl represented by R⁵ is substituted by one or more C₁₋₆ alkyl.

In some embodiments, R⁵ is —C₅-alkyl-(5- to 7-membered heterocyclyl).

In some embodiments, R⁵ is —C₅-alkyl-(5- to 7-membered heterocyclyl), wherein the alkyl or heterocyclyl represented by R⁵ is optionally substituted by one or more C₁₋₆ alkyl.

In some embodiments, R⁵ is —C₅-alkyl-(5- to 7-membered heterocyclyl), wherein the alkyl or heterocyclyl represented by R⁵ is substituted by one or more C₁₋₆ alkyl.

In some embodiments, R⁵ is —C₆-alkyl-(5- to 7-membered heterocyclyl).

In some embodiments, R⁵ is —C₆-alkyl-(5- to 7-membered heterocyclyl), wherein the alkyl or heterocyclyl represented by R⁵ is optionally substituted by one or more C₁₋₆ alkyl.

In some embodiments, R⁵ is —C₆-alkyl-(5- to 7-membered heterocyclyl), wherein the alkyl or heterocyclyl represented by R⁵ is substituted by one or more C₁₋₆ alkyl.

In some embodiments, R⁵ is —C₀₋₆-alkyl-(C₃₋₆ cycloalkyl).

In some embodiments, R⁵ is —C₀₋₆-alkyl-(C₃₋₆ cycloalkyl), wherein the alkyl or cycloalkyl represented by R⁵ is optionally substituted by one or more C₁₋₆ alkyl.

In some embodiments, R⁵ is —C₀₋₆-alkyl-(C₃₋₆ cycloalkyl), wherein the alkyl or cycloalkyl represented by R⁵ is substituted by one or more C₁₋₆ alkyl.

In some embodiments, R⁵ is —C₀₋₆-alkyl-(C₃ cycloalkyl).

In some embodiments, R⁵ is —C₀₋₆-alkyl-(C₃ cycloalkyl), wherein the alkyl or cycloalkyl represented by R⁵ is optionally substituted by one or more C₁₋₆ alkyl.

In some embodiments, R⁵ is —C₀₋₆-alkyl-(C₃ cycloalkyl), wherein the alkyl or cycloalkyl represented by R⁵ is substituted by one or more C₁₋₆ alkyl.

In some embodiments, R⁵ is —C₀₋₆-alkyl-(C₄ cycloalkyl).

In some embodiments, R⁵ is —C₀₋₆-alkyl-(C₄ cycloalkyl), wherein the alkyl or cycloalkyl represented by R⁵ is optionally substituted by one or more C₁₋₆ alkyl.

In some embodiments, R⁵ is —C₀₋₆-alkyl-(C₄ cycloalkyl), wherein the alkyl or cycloalkyl represented by R⁵ is substituted by one or more C₁₋₆ alkyl.

In some embodiments, R⁵ is —C₀₋₆-alkyl-(C₅ cycloalkyl).

In some embodiments, R⁵ is —C₀₋₆-alkyl-(C₅ cycloalkyl), wherein the alkyl or cycloalkyl represented by R⁵ is optionally substituted by one or more C₁₋₆ alkyl.

In some embodiments, R⁵ is —C₀₋₆-alkyl-(C₅ cycloalkyl), wherein the alkyl or cycloalkyl represented by R⁵ is substituted by one or more C₁₋₆ alkyl.

In some embodiments, R⁵ is —C₀₋₆-alkyl-(C₆ cycloalkyl).

In some embodiments, R⁵ is —C₀₋₆-alkyl-(C₆ cycloalkyl), wherein the alkyl or cycloalkyl represented by R⁵ is optionally substituted by one or more C₁₋₆ alkyl.

In some embodiments, R⁵ is —C₀₋₆-alkyl-(C₆ cycloalkyl), wherein the alkyl or cycloalkyl represented by R⁵ is substituted by one or more C₁₋₆ alkyl.

In some embodiments, R⁵ is —C₁-alkyl-(C₃₋₆ cycloalkyl).

In some embodiments, R⁵ is —C₁-alkyl-(C₃₋₆ cycloalkyl), wherein the alkyl or cycloalkyl represented by R⁵ is optionally substituted by one or more C₁₋₆ alkyl.

In some embodiments, R⁵ is —C₁-alkyl-(C₃₋₆ cycloalkyl), wherein the alkyl or cycloalkyl represented by R⁵ is substituted by one or more C₁₋₆ alkyl.

In some embodiments, R⁵ is —C₂-alkyl-(C₃₋₆ cycloalkyl).

In some embodiments, R⁵ is —C₂-alkyl-(C₃₋₆ cycloalkyl), wherein the alkyl or cycloalkyl represented by R⁵ is optionally substituted by one or more C₁₋₆ alkyl.

In some embodiments, R⁵ is —C₂-alkyl-(C₃₋₆ cycloalkyl), wherein the alkyl or cycloalkyl represented by R⁵ is substituted by one or more C₁₋₆ alkyl.

In some embodiments, R⁵ is —C₃-alkyl-(C₃₋₆ cycloalkyl).

In some embodiments, R⁵ is —C₃-alkyl-(C₃₋₆ cycloalkyl), wherein the alkyl or cycloalkyl represented by R⁵ is optionally substituted by one or more C₁₋₆ alkyl.

In some embodiments, R⁵ is —C₃-alkyl-(C₃₋₆ cycloalkyl), wherein the alkyl or cycloalkyl represented by R⁵ is substituted by one or more C₁₋₆ alkyl.

In some embodiments, R⁵ is —C₄-alkyl-(C₃₋₆ cycloalkyl).

In some embodiments, R⁵ is —C₄-alkyl-(C₃₋₆ cycloalkyl), wherein the alkyl or cycloalkyl represented by R⁵ is optionally substituted by one or more C₁₋₆ alkyl.

In some embodiments, R⁵ is —C₄-alkyl-(C₃₋₆ cycloalkyl), wherein the alkyl or cycloalkyl represented by R⁵ is substituted by one or more C₁₋₆ alkyl.

In some embodiments, R⁵ is —C₅-alkyl-(C₃₋₆ cycloalkyl).

In some embodiments, R⁵ is —C₅-alkyl-(C₃₋₆ cycloalkyl), wherein the alkyl or cycloalkyl represented by R⁵ is optionally substituted by one or more C₁₋₆ alkyl.

In some embodiments, R⁵ is —C₅-alkyl-(C₃₋₆ cycloalkyl), wherein the alkyl or cycloalkyl represented by R⁵ is substituted by one or more C₁₋₆ alkyl.

In some embodiments, R⁵ is —C₆-alkyl-(C₃₋₆ cycloalkyl).

In some embodiments, R⁵ is —C₆-alkyl-(C₃₋₆ cycloalkyl), wherein the alkyl or cycloalkyl represented by R⁵ is optionally substituted by one or more C₁₋₆ alkyl.

In some embodiments, R⁵ is —C₆-alkyl-(C₃₋₆ cycloalkyl), wherein the alkyl or cycloalkyl represented by R⁵ is substituted by one or more C₁₋₆ alkyl.

In some embodiments, each R is independently H, C₁₋₆ alkyl, C₂₋₆ alkenyl, C₂₋₆ alkynyl, or C₃₋₆ cycloalkyl.

In some embodiments, each R is independently H.

In some embodiments, each R is independently C₁₋₆ alkyl, C₂₋₆ alkenyl, C₂₋₆ alkynyl, or C₃₋₆ cycloalkyl.

In some embodiments, each R is independently C₁₋₆ alkyl, C₂₋₆ alkenyl, or C₂₋₆ alkynyl.

In some embodiments, R is C₁₋₆ alkyl. In some embodiments, R is C₁ alkyl. In some embodiments, R is C₂ alkyl. In some embodiments, R is C₃ alkyl. In some embodiments, R is C₄ alkyl. In some embodiments, R is C₅ alkyl. In some embodiments, R is C₆ alkyl.

In some embodiments, R is methyl. In some embodiments, R is ethyl. In some embodiments, R is propyl. In some embodiments, R is isopropyl. In some embodiments, R is butyl. In some embodiments, R is isobutyl. In some embodiments, R is sec-butyl. In some embodiments, R is tert-butyl. In some embodiments, R is pentyl. In some embodiments, R is hexyl.

In some embodiments, R is C₂₋₆ alkenyl. In some embodiments, R is C₂₋₆ alkynyl.

In some embodiments, each R is independently C₃₋₆ cycloalkyl.

In some embodiments, R is C₃ cycloalkyl. In some embodiments, R is C₄ cycloalkyl.

In some embodiments, R is C₅ cycloalkyl. In some embodiments, R is C₆ cycloalkyl.

In some embodiments, R is cyclopropyl. In some embodiments, R is cyclobutyl. In some embodiments, R is cyclopentyl. In some embodiments, R is cyclohexyl.

It is understood that, for a compound of Formula I′ or Formula I, X, Y, Z, R¹, R^{1′}, R², R³, R⁴, R^{4′}, R⁵, or n can each be, where applicable, selected from the groups described herein, and any group described herein for any of X, Y, Z, R¹, R^{1′}, R², R³, R⁴, R^{4′}, R⁵, or n can be combined, where applicable, with any group described herein for one or more of the remainder of X, Y, Z, R¹, R^{1′}, R², R³, R⁴, R^{4′}, R⁵, or n. In some embodiments, any one of the variables above can be combined with any one of the other variables above.

In some embodiments, the compound of Formula I′ or Formula I is of Formula Ia, Ib, Ic, Id, Id′, Ie, If, Ig, or Ih:

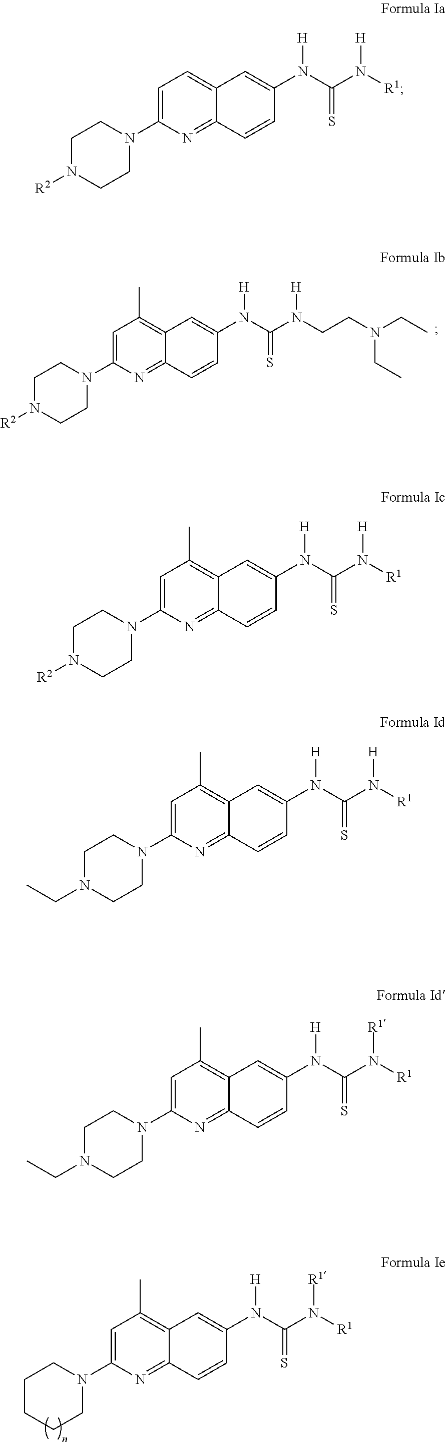

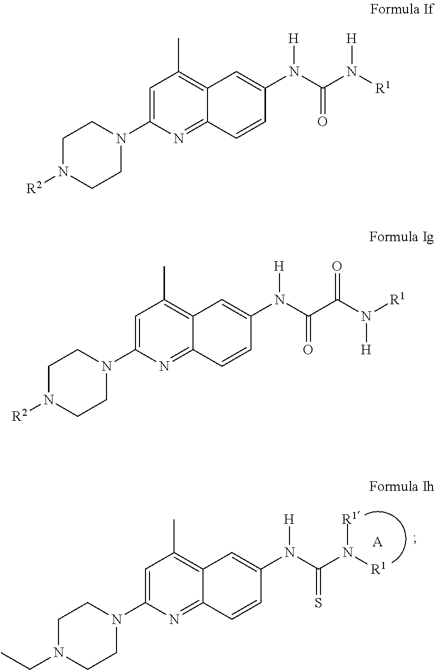

or a pharmaceutically acceptable salt thereof, wherein R¹, R^{1′}, R², and n are as described herein, and wherein ring A is a 5- to 6-membered heterocyclyl optionally substituted with one or more R⁵.

In some embodiments, the compound is of Formula Ia or a prodrug, solvate, or a pharmaceutically acceptable salt thereof.

In some embodiments, the compound is of Formula Ib or a prodrug, solvate, or a pharmaceutically acceptable salt thereof.

In some embodiments, the compound is of Formula Ic or a prodrug, solvate, or a pharmaceutically acceptable salt thereof.

In some embodiments, the compound is of Formula Id or a prodrug, solvate, or a pharmaceutically acceptable salt thereof.

In some embodiments, the compound is of Formula Id′ or a prodrug, solvate, or a pharmaceutically acceptable salt thereof.

In some embodiments, the compound is of Formula Ie or a prodrug, solvate, or a pharmaceutically acceptable salt thereof.

In some embodiments, the compound is of Formula If or a prodrug, solvate, or a pharmaceutically acceptable salt thereof.

In some embodiments, the compound is of Formula Ig or a prodrug, solvate, or pharmaceutically acceptable salt thereof.

In some embodiments, the compound is of Formula Ih or a prodrug, solvate, or a pharmaceutically acceptable salt thereof.

In some embodiments, a compound of Formula I′ is a compound of Formula Ia, Formula Ib, Formula Ic, Formula Id, Formula Id′, Formula Ie, Formula If, Formula Ig, or Formula Ih or a prodrug, solvate, or a pharmaceutically acceptable salt thereof.

In some embodiments, a compound of Formula I′ is a compound of Formula Ia, Formula Ib, Formula Ic, Formula Id, Formula Id′, Formula Ie, Formula If, Formula Ig, or Formula Ih or a pharmaceutically acceptable salt thereof.

In some embodiments, a compound of Formula I′ is a compound of Formula Ia, Formula Ib, Formula Ic, Formula Id, Formula Id′, Formula Ie, Formula If, Formula Ig, or Formula Ih.

In some embodiments, the compound of Formula Ia is selected from Table 1:

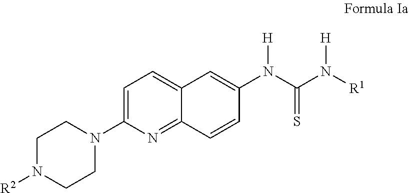

TABLE 1
Compounds of Formula Ia

| | | | MS |
|---|---|---|---|
| Name | R¹ | R² | (M + H)⁺ |
| 0001 | 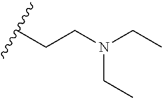 [SMILES: *CCN(CC)CC] | H | 387.6 |
| 0002 | 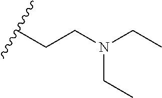 [SMILES: *CCN(CC)CC] | CH₃ | 401.6 |
| 0003 | 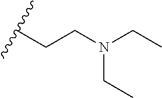 [SMILES: *CCN(CC)CC] | 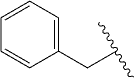 [SMILES: *Cc1ccccc1] | 477.7 |
| 0004 | 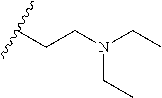 [SMILES: *CCN(CC)CC] | 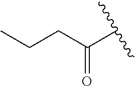 [SMILES: *C(=O)CCC] | 457.7 |
| 0005 | 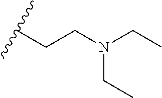 [SMILES: *CCN(CC)CC] | 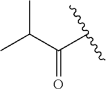 [SMILES: *C(=O)C(C)C] | 457.7 |
| 0006 | 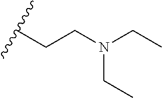 [SMILES: *CCN(CC)CC] | 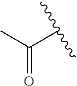 [SMILES: *C(C)=O] | 429.6 |
| 0007 | 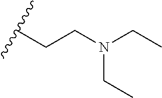 [SMILES: *CCN(CC)CC] | 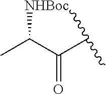 [SMILES: *C(=O)[C@H](C)NC(=O)OC(C)(C)C] | 559.8 |
| 0008 | 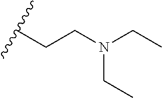 [SMILES: *CCN(CC)CC] | 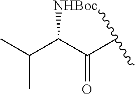 [SMILES: *C(=O)[C@@H](NC(=O)OC(C)(C)C)C(C)C] | 586.9 |
| 0009 | 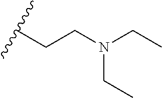 [SMILES: *CCN(CC)CC] | 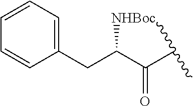 [SMILES: *C(=O)[C@H](Cc1ccccc1)NC(=O)OC(C)(C)C] | 634.9 |
| 0010 | 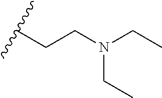 [SMILES: *CCN(CC)CC] | 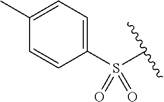 [SMILES: *S(=O)(=O)c1ccc(C)cc1] | 541.7 |
| 0011 | 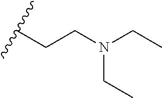 [SMILES: *CCN(CC)CC] | 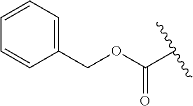 [SMILES: *C(=O)OCc1ccccc1] | 521.8 |
| 0012 | 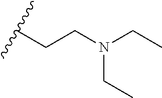 [SMILES: *CCN(CC)CC] | 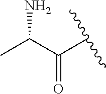 [SMILES: *C(=O)[C@H](C)N] | 458.7 |
| 0013 | 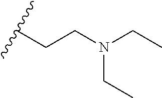 [SMILES: *CCN(CC)CC] | 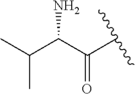 [SMILES: *C(=O)[C@@H](N)C(C)C] | 486.8 |
| 0014 | 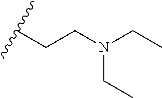 [SMILES: *CCN(CC)CC] | 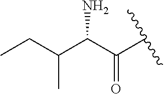 [SMILES: *C(=O)[C@@H](N)C(C)CC] | 500.8 |
| 0015 | 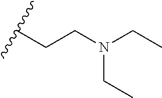 [SMILES: *CCN(CC)CC] | 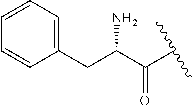 [SMILES: *C(=O)[C@@H](N)Cc1ccccc1] | 534.8 |
| 0016 | 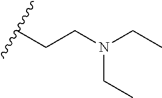 [SMILES: *CCN(CC)CC] | 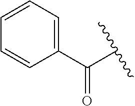 [SMILES: *C(=O)c1ccccc1] | 491.7 |
| 0017 | 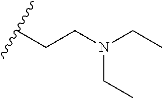 [SMILES: *CCN(CC)CC] | 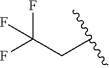 [SMILES: *CC(F)(F)F] | 469.7 |
| 0018 | 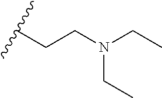 [SMILES: *CCN(CC)CC] | 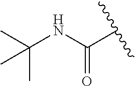 [SMILES: *C(=O)NC(C)(C)C] | 486.7 |
| 0019 | 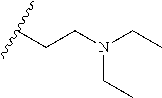 [SMILES: *CCN(CC)CC] | 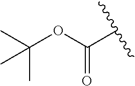 [SMILES: *C(=O)OC(C)(C)C] | 487.7 |
| 0020 | 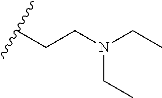 [SMILES: *CCN(CC)CC] | 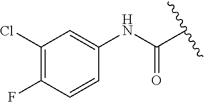 [SMILES: *C(=O)Nc1ccc(F)c(Cl)c1] | 558.7 |
| 0021 | 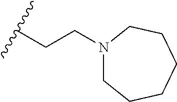 [SMILES: *CCN1CCCCCC1] | H | 413.6 |
| 0022 | 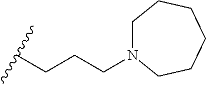 [SMILES: *CCCN1CCCCCC1] | H | 427.7 |
| 0023 | 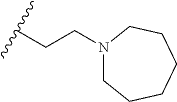 [SMILES: *CCN1CCCCCC1] | Boc | 513.8 |
| 0024 | 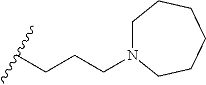 [SMILES: *CCCN1CCCCCC1] | Boc | 527.8 |
| 0040 | 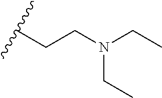 [SMILES: *CCN(CC)CC] | 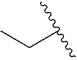 [SMILES: *CC] | 415.7 |

In some embodiments, the compounds of Formula Ib are selected from Table 2:

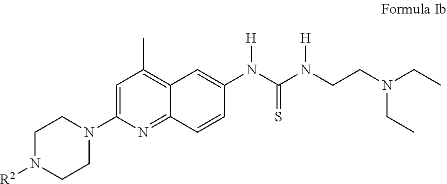

TABLE 2
Compounds of Formula 1b

| | | MS |
|---|---|---|
| Name | R² | (M + H)⁺ |
| 0048 | H— | 401.6 |
| 0049 | 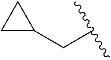 [SMILES: *CC1CC1] | 455.8 |
| 0050 | 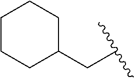 [SMILES: *CC1CCCCC1] | 497.8 |
| 0051 | 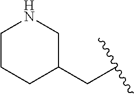 [SMILES: *CC1CCCNC1] | 498.8 |
| 0052 | 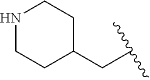 [SMILES: *CC1CCNCC1] | 498.8 |
| 0053 | 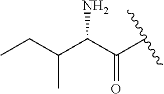 [SMILES: *C(=O)[C@@H](N)C(C)CC] | 514.9 |
| 0054 | 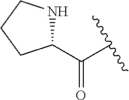 [SMILES: *C(=O)[C@@H]1CCCN1] | 498.7 |
| 0055 | 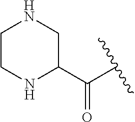 [SMILES: *C(=O)C1CNCCN1] | 513.8 |
| 0080 | 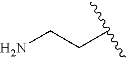 [SMILES: *CCN] | 444.7 |
| 0082 | 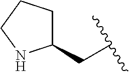 [SMILES: *C[C@@H]1CCCN1] | 484.8 |
| 0083 | 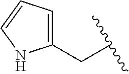 [SMILES: *Cc1ccc[nH]1] | 480.8 |
| 0084 | 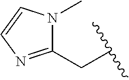 [SMILES: *Cc1nccn1C] | 495.8 |
| 0085 | 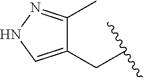 [SMILES: *Cc1c[nH]nc1C] | 495.8 |
| 0086 | 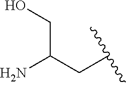 [SMILES: *CC(N)CO] | 474.8 |
| 0087 | 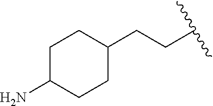 [SMILES: *CCC1CCC(N)CC1] | 526.9 |

In some embodiments, the compounds of Formula Ic are selected from Table 3:

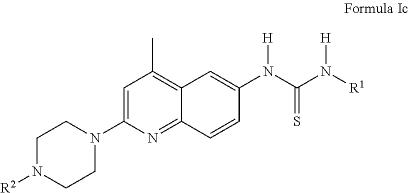

TABLE 3
Compounds of Formula Ic

| | | | MS |
|---|---|---|---|
| Name | R² | R¹ | (M + H)⁺ |
| 0059 | 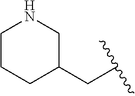 [SMILES: *CC1CCCNC1] | 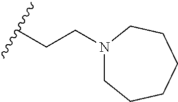 [SMILES: *CCN1CCCCCC1] | 524.9 |
| 0060 | 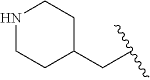 [SMILES: *CC1CCNCC1] | 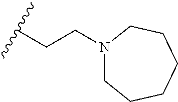 [SMILES: *CCN1CCCCCC1] | 524.9 |
| 0061 | 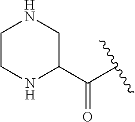 [SMILES: *C(=O)C1CNCCN1] | 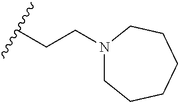 [SMILES: *CCN1CCCCCC1] | 537.9 |
| 0062 | 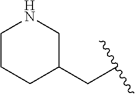 [SMILES: *CC1CCCNC1] | 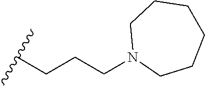 [SMILES: *CCCN1CCCCCC1] | 538.9 |
| 0063 | 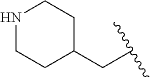 [SMILES: *CC1CCNCC1] | 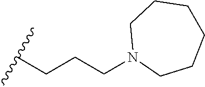 [SMILES: *CCCN1CCCCCC1] | 538.9 |
| 0064 | 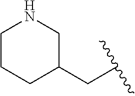 [SMILES: *CC1CCCNC1] | 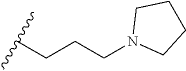 [SMILES: *CCCN1CCCC1] | 510.9 |
| 0065 | 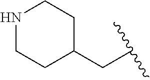 [SMILES: *CC1CCNCC1] | 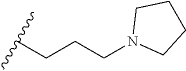 [SMILES: *CCCN1CCCC1] | 510.9 |
| 0108 | 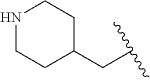 [SMILES: *CC1CCNCC1] | 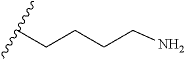 [SMILES: *CCCCN] | 470.8 |
| 0109 | 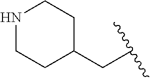 [SMILES: *CC1CCNCC1] | 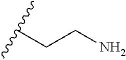 [SMILES: *CCN] | 442.8 |
| 0110 | 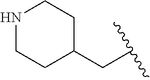 [SMILES: *CC1CCNCC1] | 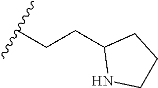 [SMILES: *CCC1CCCN1] | 496.8 |
| 0112 | 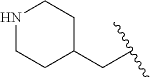 [SMILES: *CC1CCNCC1] | 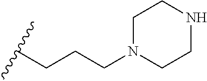 [SMILES: *CCCN1CCNCC1] | 525.9 |

In some embodiments, the compounds of Formula Id′ are selected from Table 4:

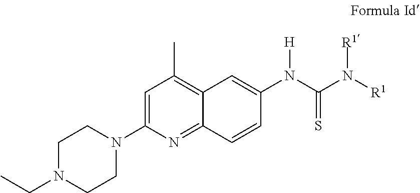

TABLE 4
Compounds of Formula Id′

| | | | MS |
|---|---|---|---|
| Name | R¹ | R^{1′} | (M + H)⁺ |
| 0025 | 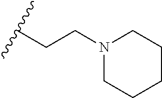 [SMILES: *CCN1CCCCC1] | H | 441.6 |
| 0026 | 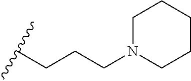 [SMILES: *CCCN1CCCCC1] | H | 455.7 |
| 0027 | 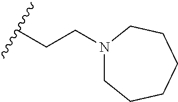 [SMILES: *CCN1CCCCCC1] | H | 455.7 |
| 0028 | 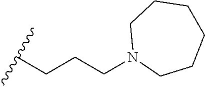 [SMILES: *CCCN1CCCCCC1] | H | 469.7 |
| 0029 | 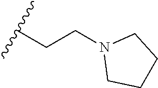 [SMILES: *CCN1CCCC1] | H | 427.7 |
| 0030 | 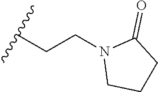 [SMILES: *CCN1CCCC1=O] | H | 441.7 |
| 0031 | 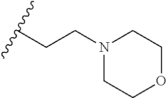 [SMILES: *CCN1CCOCC1] | H | 443.7 |
| 0032 | 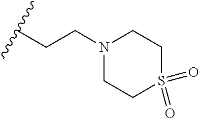 [SMILES: *CCN1CCS(=O)(=O)CC1] | H | 491.7 |
| 0033 | 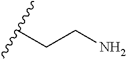 [SMILES: *CCN] | H | 373.6 |
| 0034 | 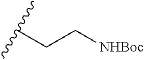 [SMILES: *CCNC(=O)OC(C)(C)C] | H | 473.7 |
| 0035 | 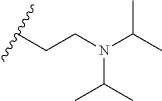 [SMILES: *CCN(C(C)C)C(C)C] | H | 457.7 |
| 0036 | 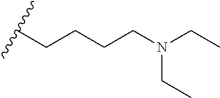 [SMILES: *CCCCN(CC)CC] | H | 457.7 |
| 0037 | —H | H | 330.5 |
| 0041 | 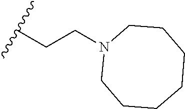 [SMILES: *CCN1CCCCCCC1] | H | 469.7 |
| 0042 | 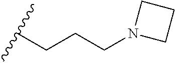 [SMILES: *CCCN1CCC1] | H | 427.7 |
| 0043 | 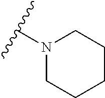 [SMILES: *N1CCCCC1] | H | 413.7 |
| 0044 | 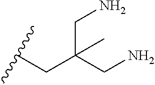 [SMILES: *CC(C)(CN)CN] | H | 430.7 |
| 0045 | 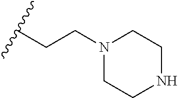 [SMILES: *CCN1CCNCC1] | H | 442.7 |
| 0046 | 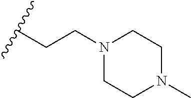 [SMILES: *CCN1CCN(C)CC1] | H | 456.7 |
| 0047 | 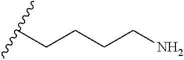 [SMILES: *CCCCN] | H | 401.6 |
| 0056 | 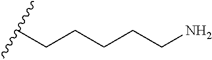 [SMILES: *CCCCCN] | H | 415.7 |
| 0057 | 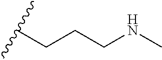 [SMILES: *CCCNC] | H | 401.6 |
| 0058 | 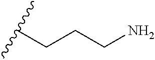 [SMILES: *CCCN] | CH₃ | 401.6 |
| 0066 | 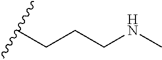 [SMILES: *CCCNC] | CH₃ | 415.7 |
| 0067 | 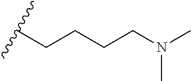 [SMILES: *CCCCN(C)C] | H | 429.7 |
| 0068 | 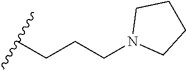 [SMILES: *CCCN1CCCC1] | H | 441.7 |
| 0069 | 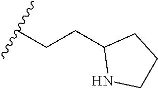 [SMILES: *CCC1CCCN1] | H | 427.6 |
| 0073 | 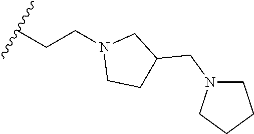 [SMILES: *CCN1CCC(CN2CCCC2)C1] | H | 510.9 |
| 0074 | 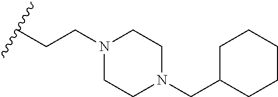 [SMILES: *CCN1CCN(CC2CCCCC2)CC1] | H | 538.9 |
| 0075 | 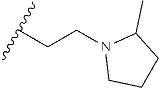 [SMILES: *CCN1CCCC1C] | H | 441.7 |
| 0076 | 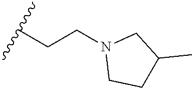 [SMILES: *CCN1CCC(C)C1] | H | 441.6 |
| 0077 | 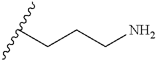 [SMILES: *CCCN] | H | 387.6 |
| 0078 | 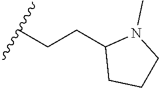 [SMILES: *CCC1CCCN1C] | H | 441.7 |
| 0079 | 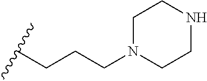 [SMILES: *CCCN1CCNCC1] | H | 456.8 |

In some embodiments, the compounds of Formula Ie are selected from Table 5:

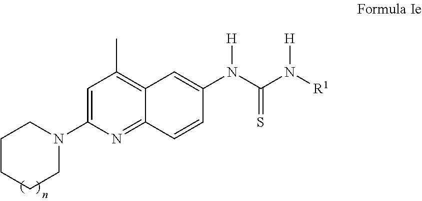

TABLE 5
Compounds of Formula Ie

| | | | MS |
|---|---|---|---|
| Name | n | R¹ | (M + H)⁺ |
| 0088 | 0 | 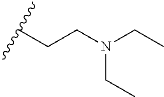 [SMILES: *CCN(CC)CC] | 386.7 |
| 0089 | 0 | 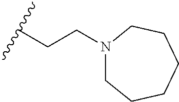 [SMILES: *CCN1CCCCCC1] | 412.7 |
| 0090 | 0 | 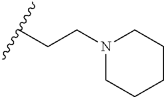 [SMILES: *CCN1CCCCC1] | 398.7 |
| 0091 | 0 | 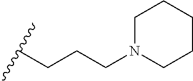 [SMILES: *CCCN1CCCCC1] | 412.7 |
| 0092 | 0 | 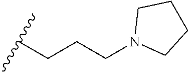 [SMILES: *CCCN1CCCC1] | 398.7 |
| 0093 | 0 | 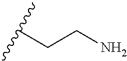 [SMILES: *CCN] | 330.6 |
| 0094 | 0 | 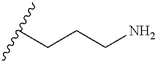 [SMILES: *CCCN] | 344.6 |
| 0095 | 0 | 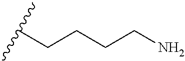 [SMILES: *CCCCN] | 358.6 |
| 0096 | 0 | 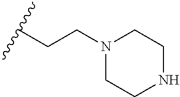 [SMILES: *CCN1CCNCC1] | 399.7 |
| 0097 | 0 | 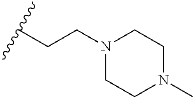 [SMILES: *CCN1CCN(C)CC1] | 413.7 |
| 0098 | 1 | 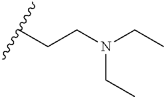 [SMILES: *CCN(CC)CC] | 400.7 |
| 0099 | 1 | 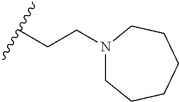 [SMILES: *CCN1CCCCCC1] | 426.7 |
| 0100 | 1 | 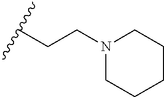 [SMILES: *CCN1CCCCC1] | 412.7 |
| 0101 | 1 | 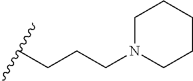 [SMILES: *CCCN1CCCCC1] | 426.7 |
| 0102 | 1 | 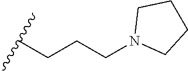 [SMILES: *CCCN1CCCC1] | 412.7 |
| 0103 | 1 | 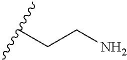 [SMILES: *CCN] | 344.6 |
| 0104 | 1 | 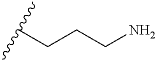 [SMILES: *CCCN] | 358.6 |
| 0105 | 1 | 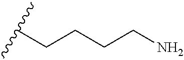 [SMILES: *CCCCN] | 372.6 |
| 0106 | 1 | 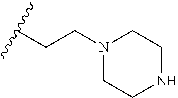 [SMILES: *CCN1CCNCC1] | 413.7 |
| 0107 | 1 | 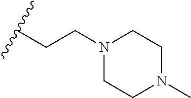 [SMILES: *CCN1CCN(C)CC1] | 427.7 |

In some embodiments, the compounds of Formula If are selected from Table 6:

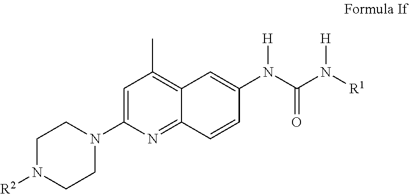

TABLE 6
Compounds of Formula If

| | | | MS |
|---|---|---|---|
| Name | R¹ | R² | (M + H)⁺ |
| 0038 | 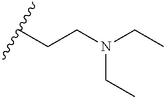 [SMILES: *CCN(CC)CC] | 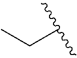 [SMILES: *CC] | 413.7 |
| 0039 | 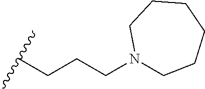 [SMILES: *CCCN1CCCCCC1] | 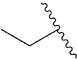 [SMILES: *CC] | 453.8 |
| 0113 | 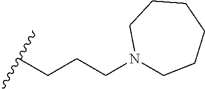 [SMILES: *CCCN1CCCCCC1] | 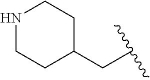 [SMILES: *CC1CCNCC1] | 522.9 |

In some embodiments, the compounds of Formula Ig are selected from Table 7:

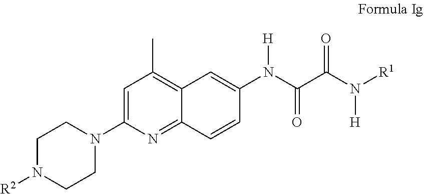

TABLE 7
Compounds of Formula Ig

| | | | MS |
|---|---|---|---|
| Name | R¹ | R² | (M + H)⁺ |
| 0114 | 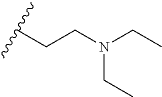 [SMILES: *CCN(CC)CC] | 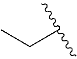 [SMILES: *CC] | 441.8 |

In some embodiments, the compounds of Formula Ih are selected from Table 8:

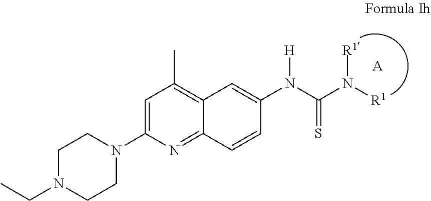

TABLE 8
Compounds of Formula Ih

| Name | 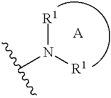 | MS (M + H)⁺ |
|---|---|---|
| 0070 | 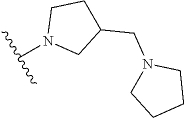 [SMILES: *N1CCC(CN2CCCC2)C1] | 467.7 |
| 0071 | 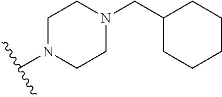 [SMILES: *N1CCN(CC2CCCCC2)CC1] | 495.8 |
| 0072 | 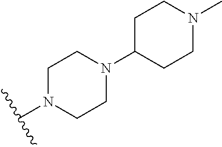 [SMILES: *N1CCN(C2CCN(C)CC2)CC1] | 496.8 |

In some embodiments, the compound is selected from the compounds in Table 9.

In some embodiments, the compound is selected from the compounds described in Table 9 and prodrugs and pharmaceutically acceptable salts thereof.

In some embodiments, the compound is selected from the compounds described in Table 9 and pharmaceutically acceptable salts thereof.

In some embodiments, the compound is selected from the prodrugs of compounds described in Table 9 and pharmaceutically acceptable salts thereof.

In some embodiments, the compound is selected from the compounds described in Table 9.

TABLE 9

| Compound | |
|---|---|
| No. | Structure |
| 0001 | 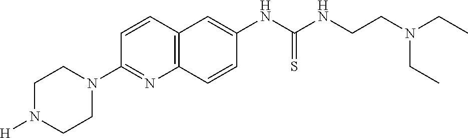 [SMILES: CCN(CC)CCNC(=S)Nc1ccc2nc(N3CCNCC3)ccc2c1] |
| 0002 | 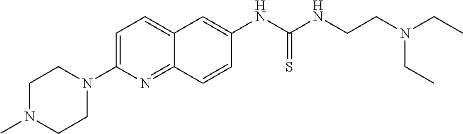 [SMILES: CCN(CC)CCNC(=S)Nc1ccc2nc(N3CCN(C)CC3)ccc2c1] |
| 0003 | 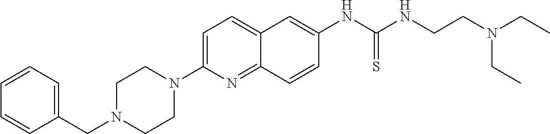 [SMILES: CCN(CC)CCNC(=S)Nc1ccc2nc(N3CCN(Cc4ccccc4)CC3)ccc2c1] |
| 0004 | 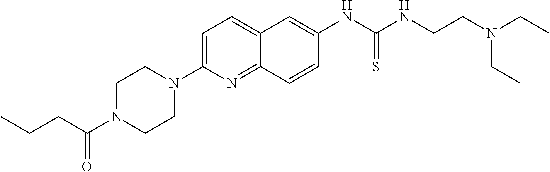 [SMILES: CCCC(=O)N1CCN(c2ccc3cc(NC(=S)NCCN(CC)CC)ccc3n2)CC1] |
| 0005 | 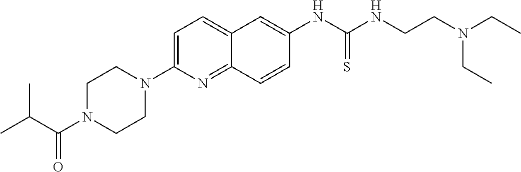 [SMILES: CCN(CC)CCNC(=S)Nc1ccc2nc(N3CCN(C(=O)C(C)C)CC3)ccc2c1] |
| 0006 | 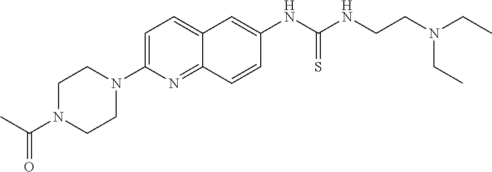 [SMILES: CCN(CC)CCNC(=S)Nc1ccc2nc(N3CCN(C(C)=O)CC3)ccc2c1] |
| 0007 | 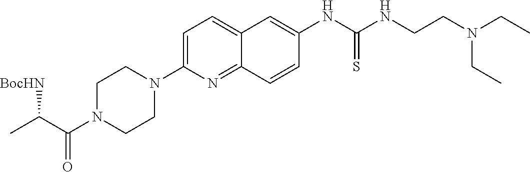 [SMILES: CCN(CC)CCNC(=S)Nc1ccc2nc(N3CCN(C(=O)[C@H](C)NC(=O)OC(C)(C)C)CC3)ccc2c1] |
| 0008 | 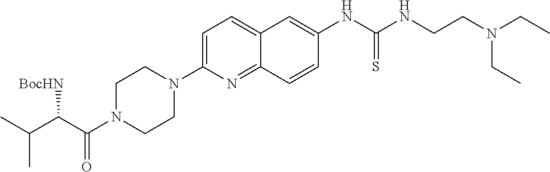 [SMILES: CCN(CC)CCNC(=S)Nc1ccc2nc(N3CCN(C(=O)[C@@H](NC(=O)OC(C)(C)C)C(C)C)CC3)ccc2c1] |
| 0009 | 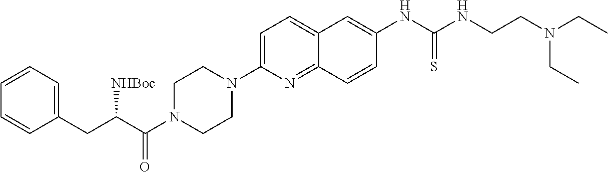 [SMILES: CCN(CC)CCNC(=S)Nc1ccc2nc(N3CCN(C(=O)[C@H](Cc4ccccc4)NC(=O)OC(C)(C)C)CC3)ccc2c1] |
| 0010 | 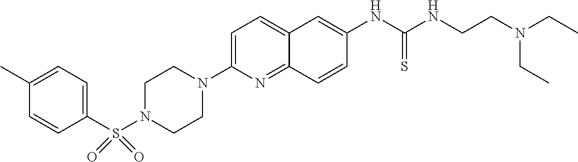 [SMILES: CCN(CC)CCNC(=S)Nc1ccc2nc(N3CCN(S(=O)(=O)c4ccc(C)cc4)CC3)ccc2c1] |
| 0011 | 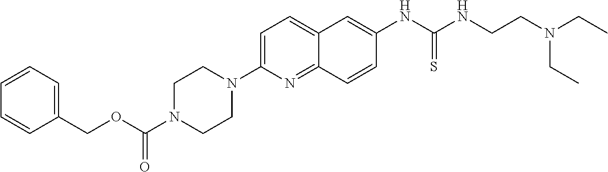 [SMILES: CCN(CC)CCNC(=S)Nc1ccc2nc(N3CCN(C(=O)OCc4ccccc4)CC3)ccc2c1] |
| 0012 | 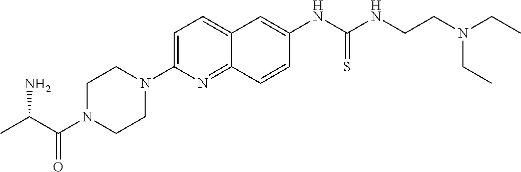 [SMILES: CCN(CC)CCNC(=S)Nc1ccc2nc(N3CCN(C(=O)[C@H](C)N)CC3)ccc2c1] |
| 0013 | 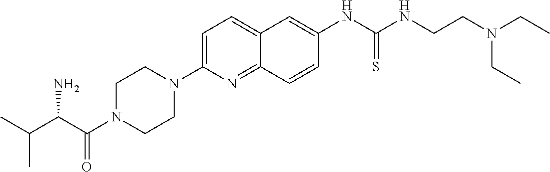 [SMILES: CCN(CC)CCNC(=S)Nc1ccc2nc(N3CCN(C(=O)[C@@H](N)C(C)C)CC3)ccc2c1] |
| 0014 | 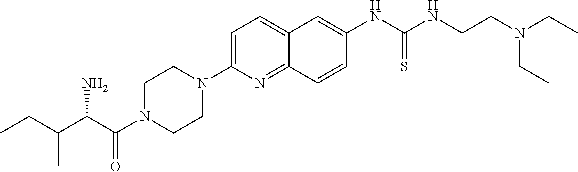 [SMILES: CCC(C)[C@H](N)C(=O)N1CCN(c2ccc3cc(NC(=S)NCCN(CC)CC)ccc3n2)CC1] |
| 0015 | 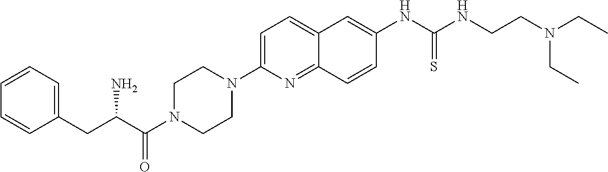 [SMILES: CCN(CC)CCNC(=S)Nc1ccc2nc(N3CCN(C(=O)[C@@H](N)Cc4ccccc4)CC3)ccc2c1] |
| 0016 | 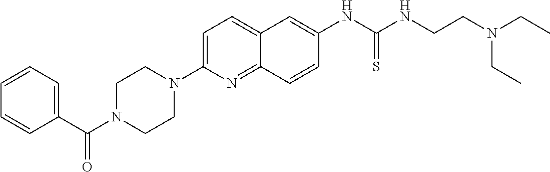 [SMILES: CCN(CC)CCNC(=S)Nc1ccc2nc(N3CCN(C(=O)c4ccccc4)CC3)ccc2c1] |
| 0017 | 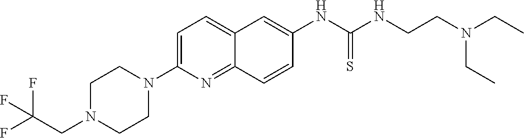 [SMILES: CCN(CC)CCNC(=S)Nc1ccc2nc(N3CCN(CC(F)(F)F)CC3)ccc2c1] |
| 0018 | 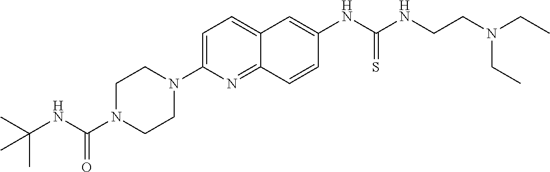 [SMILES: CCN(CC)CCNC(=S)Nc1ccc2nc(N3CCN(C(=O)NC(C)(C)C)CC3)ccc2c1] |
| 0019 | 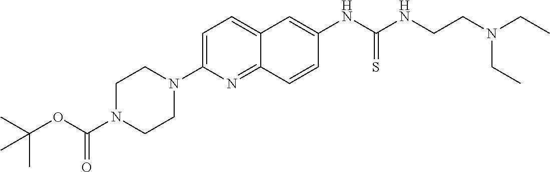 [SMILES: CCN(CC)CCNC(=S)Nc1ccc2nc(N3CCN(C(=O)OC(C)(C)C)CC3)ccc2c1] |
| 0020 | 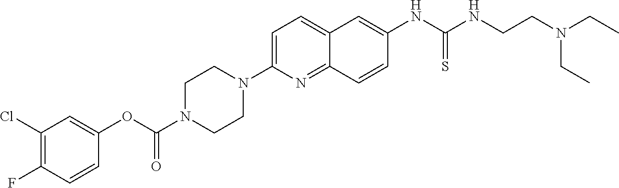 [SMILES: CCN(CC)CCNC(=S)Nc1ccc2nc(N3CCN(C(=O)Oc4ccc(F)c(Cl)c4)CC3)ccc2c1] |
| 0021 | 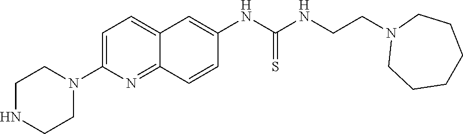 [SMILES: S=C(NCCN1CCCCCC1)Nc1ccc2nc(N3CCNCC3)ccc2c1] |
| 0022 | 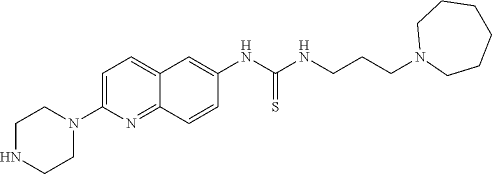 [SMILES: S=C(NCCCN1CCCCCC1)Nc1ccc2nc(N3CCNCC3)ccc2c1] |
| 0023 | 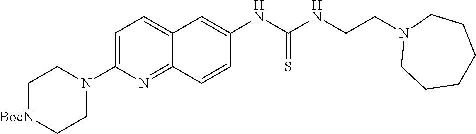 [SMILES: CC(C)(C)OC(=O)N1CCN(c2ccc3cc(NC(=S)NCCN4CCCCCC4)ccc3n2)CC1] |
| 0024 | 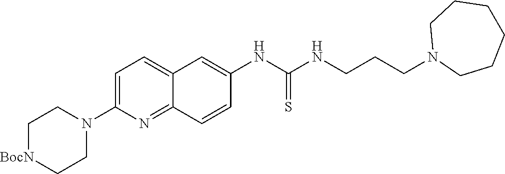 [SMILES: CC(C)(C)OC(=O)N1CCN(c2ccc3cc(NC(=S)NCCCN4CCCCCC4)ccc3n2)CC1] |
| 0025 | 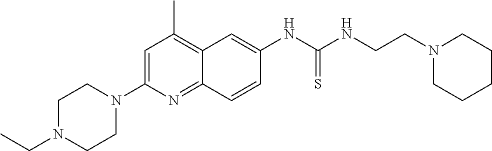 [SMILES: CCN1CCN(c2cc(C)c3cc(NC(=S)NCCN4CCCCC4)ccc3n2)CC1] |
| 0026 | 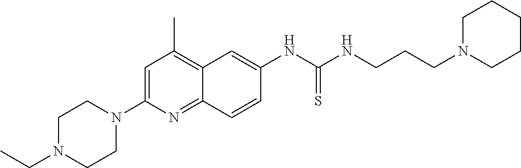 [SMILES: CCN1CCN(c2cc(C)c3cc(NC(=S)NCCCN4CCCCC4)ccc3n2)CC1] |
| 0027 | 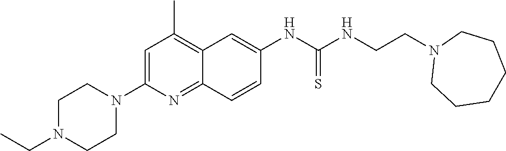 [SMILES: CCN1CCN(c2cc(C)c3cc(NC(=S)NCCN4CCCCCC4)ccc3n2)CC1] |
| 0028 | 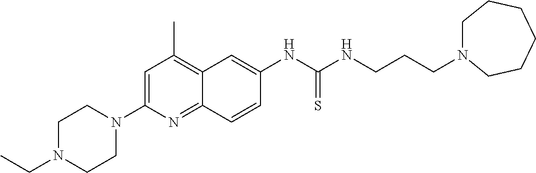 [SMILES: CCN1CCN(c2cc(C)c3cc(NC(=S)NCCCN4CCCCCC4)ccc3n2)CC1] |
| 0029 | 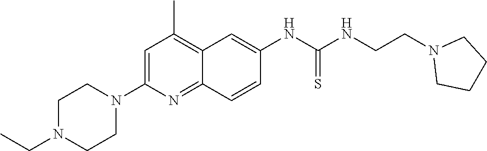 [SMILES: CCN1CCN(c2cc(C)c3cc(NC(=S)NCCN4CCCC4)ccc3n2)CC1] |
| 0030 | 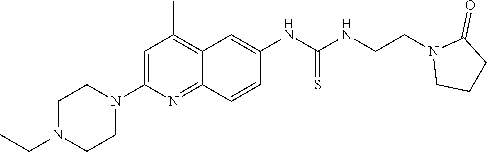 [SMILES: CCN1CCN(c2cc(C)c3cc(NC(=S)NCCN4CCCC4=O)ccc3n2)CC1] |
| 0031 | 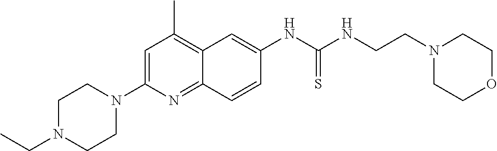 [SMILES: CCN1CCN(c2cc(C)c3cc(NC(=S)NCCN4CCOCC4)ccc3n2)CC1] |
| 0032 | 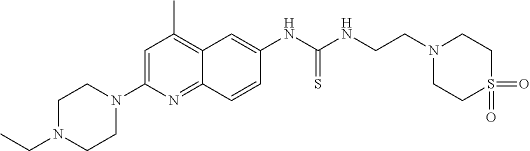 [SMILES: CCN1CCN(c2cc(C)c3cc(NC(=S)NCCN4CCS(=O)(=O)CC4)ccc3n2)CC1] |
| 0033 | 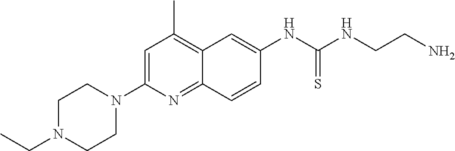 [SMILES: CCN1CCN(c2cc(C)c3cc(NC(=S)NCCN)ccc3n2)CC1] |
| 0034 | 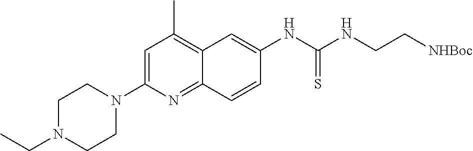 [SMILES: CCN1CCN(c2cc(C)c3cc(NC(=S)NCCNC(=O)OC(C)(C)C)ccc3n2)CC1] |
| 0035 | 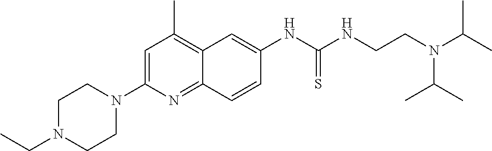 [SMILES: CCN1CCN(c2cc(C)c3cc(NC(=S)NCCN(C(C)C)C(C)C)ccc3n2)CC1] |
| 0036 | 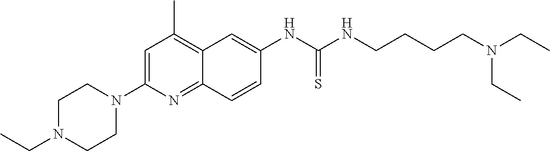 [SMILES: CCN(CC)CCCCNC(=S)Nc1ccc2nc(N3CCN(CC)CC3)cc(C)c2c1] |
| 0037 | 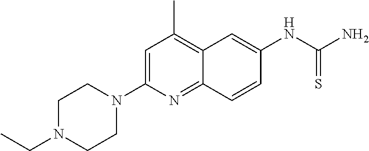 [SMILES: CCN1CCN(c2cc(C)c3cc(NC(N)=S)ccc3n2)CC1] |
| 0038 | 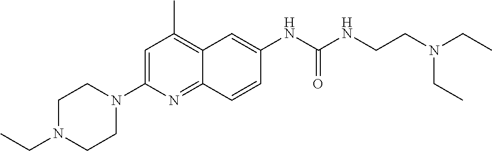 [SMILES: CCN(CC)CCNC(=O)Nc1ccc2nc(N3CCN(CC)CC3)cc(C)c2c1] |
| 0039 | 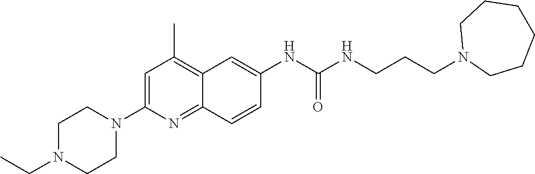 [SMILES: CCN1CCN(c2cc(C)c3cc(NC(=O)NCCCN4CCCCCC4)ccc3n2)CC1] |
| 0040 | 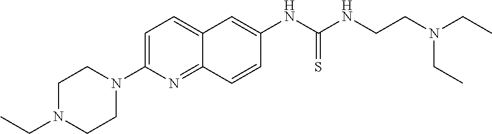 [SMILES: CCN(CC)CCNC(=S)Nc1ccc2nc(N3CCN(CC)CC3)ccc2c1] |
| 0041 | 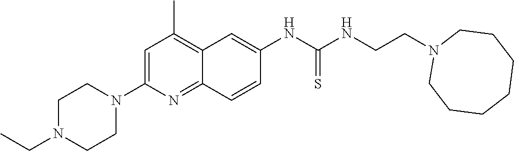 [SMILES: CCN1CCN(c2cc(C)c3cc(NC(=S)NCCN4CCCCCCC4)ccc3n2)CC1] |
| 0042 | 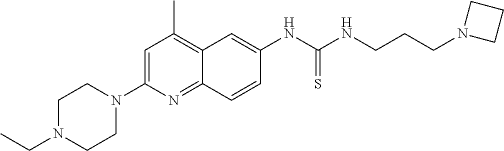 [SMILES: CCN1CCN(c2cc(C)c3cc(NC(=S)NCCCN4CCC4)ccc3n2)CC1] |
| 0043 | 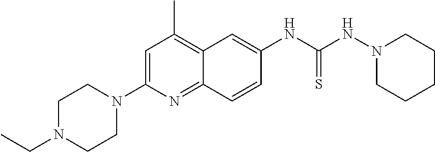 [SMILES: CCN1CCN(c2cc(C)c3cc(NC(=S)NN4CCCCC4)ccc3n2)CC1] |
| 0044 | 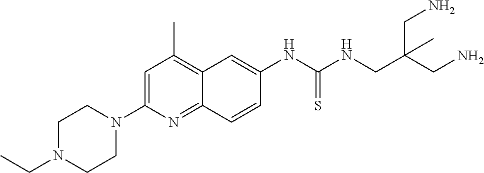 [SMILES: CCN1CCN(c2cc(C)c3cc(NC(=S)NCC(C)(CN)CN)ccc3n2)CC1] |
| 0045 | 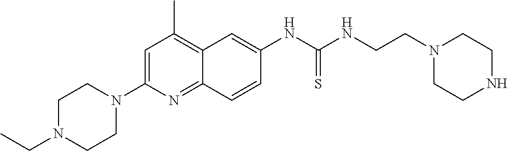 [SMILES: CCN1CCN(c2cc(C)c3cc(NC(=S)NCCN4CCNCC4)ccc3n2)CC1] |
| 0046 | 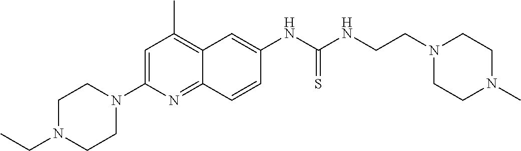 [SMILES: CCN1CCN(c2cc(C)c3cc(NC(=S)NCCN4CCN(C)CC4)ccc3n2)CC1] |
| 0047 | 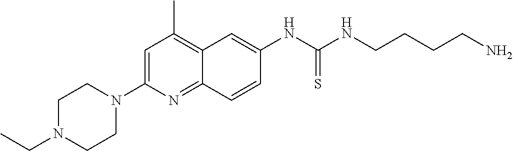 [SMILES: CCN1CCN(c2cc(C)c3cc(NC(=S)NCCCCN)ccc3n2)CC1] |
| 0048 | 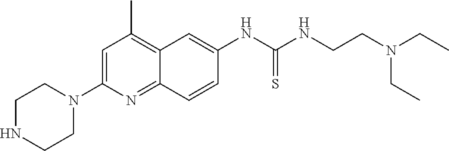 [SMILES: CCN(CC)CCNC(=S)Nc1ccc2nc(N3CCNCC3)cc(C)c2c1] |
| 0049 | 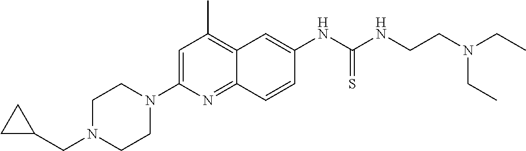 [SMILES: CCN(CC)CCNC(=S)Nc1ccc2nc(N3CCN(CC4CC4)CC3)cc(C)c2c1] |
| 0050 | 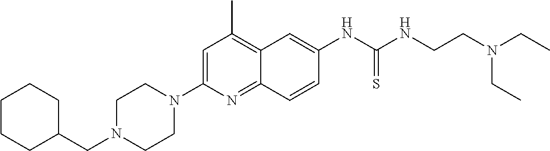 [SMILES: CCN(CC)CCNC(=S)Nc1ccc2nc(N3CCN(CC4CCCCC4)CC3)cc(C)c2c1] |
| 0051 | 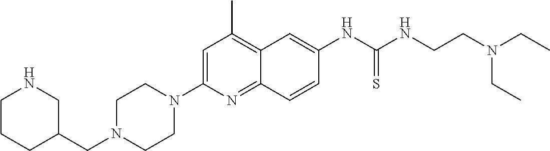 [SMILES: CCN(CC)CCNC(=S)Nc1ccc2nc(N3CCN(CC4CCCNC4)CC3)cc(C)c2c1] |
| 0052 | 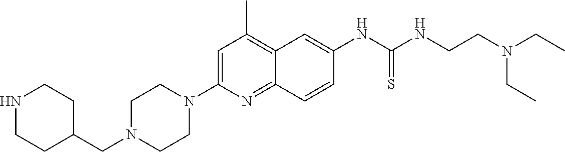 [SMILES: CCN(CC)CCNC(=S)Nc1ccc2nc(N3CCN(CC4CCNCC4)CC3)cc(C)c2c1] |
| 0053 | 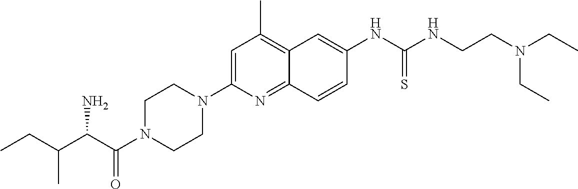 [SMILES: CCC(C)[C@H](N)C(=O)N1CCN(c2cc(C)c3cc(NC(=S)NCCN(CC)CC)ccc3n2)CC1] |
| 0054 | 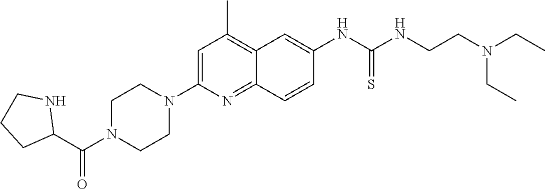 [SMILES: CCN(CC)CCNC(=S)Nc1ccc2nc(N3CCN(C(=O)C4CCCN4)CC3)cc(C)c2c1] |
| 0055 | 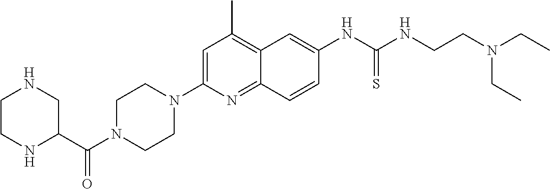 [SMILES: CCN(CC)CCNC(=S)Nc1ccc2nc(N3CCN(C(=O)C4CNCCN4)CC3)cc(C)c2c1] |
| 0056 | 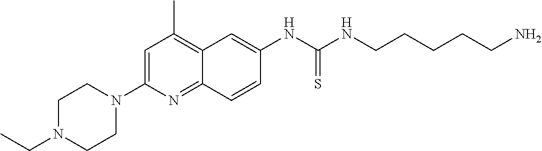 [SMILES: CCN1CCN(c2cc(C)c3cc(NC(=S)NCCCCCN)ccc3n2)CC1] |
| 0057 | 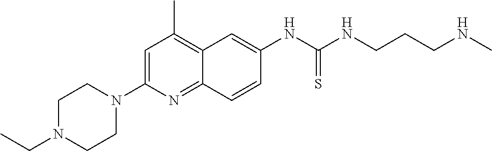 [SMILES: CCN1CCN(c2cc(C)c3cc(NC(=S)NCCCNC)ccc3n2)CC1] |
| 0058 | 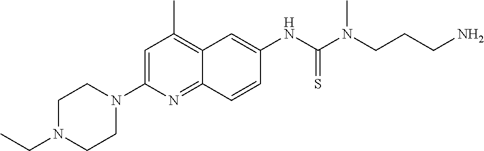 [SMILES: CCN1CCN(c2cc(C)c3cc(NC(=S)N(C)CCCN)ccc3n2)CC1] |
| 0059 | 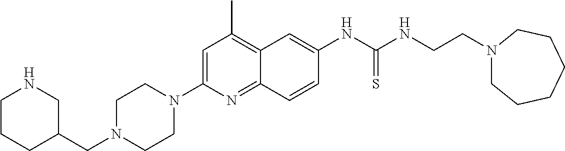 [SMILES: Cc1cc(N2CCN(CC3CCCNC3)CC2)nc2ccc(NC(=S)NCCN3CCCCCC3)cc12] |
| 0060 | 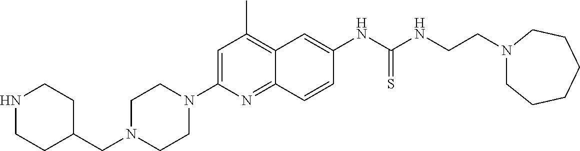 [SMILES: Cc1cc(N2CCN(CC3CCNCC3)CC2)nc2ccc(NC(=S)NCCN3CCCCCC3)cc12] |
| 0061 | 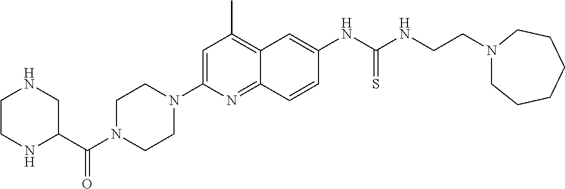 [SMILES: Cc1cc(N2CCN(C(=O)C3CNCCN3)CC2)nc2ccc(NC(=S)NCCN3CCCCCC3)cc12] |
| 0062 | 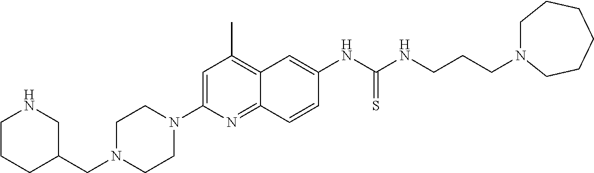 [SMILES: Cc1cc(N2CCN(CC3CCCNC3)CC2)nc2ccc(NC(=S)NCCCN3CCCCCC3)cc12] |
| 0063 | 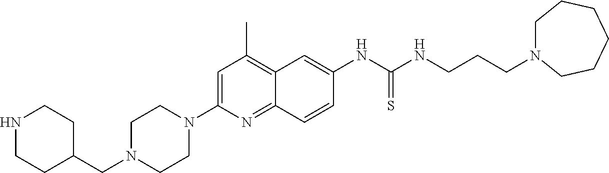 [SMILES: Cc1cc(N2CCN(CC3CCNCC3)CC2)nc2ccc(NC(=S)NCCCN3CCCCCC3)cc12] |
| 0064 | 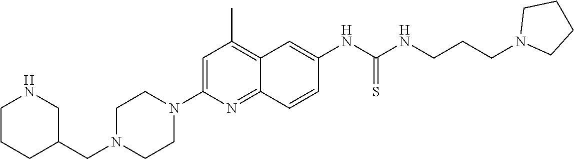 [SMILES: Cc1cc(N2CCN(CC3CCCNC3)CC2)nc2ccc(NC(=S)NCCCN3CCCC3)cc12] |
| 0065 | 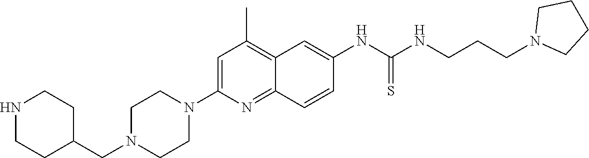 [SMILES: Cc1cc(N2CCN(CC3CCNCC3)CC2)nc2ccc(NC(=S)NCCCN3CCCC3)cc12] |
| 0066 | 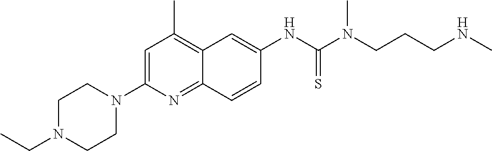 [SMILES: CCN1CCN(c2cc(C)c3cc(NC(=S)N(C)CCCNC)ccc3n2)CC1] |
| 0067 | 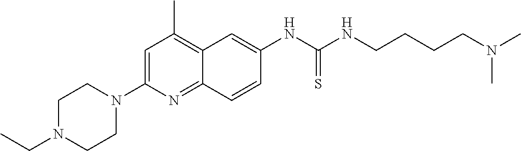 [SMILES: CCN1CCN(c2cc(C)c3cc(NC(=S)NCCCCN(C)C)ccc3n2)CC1] |
| 0068 | 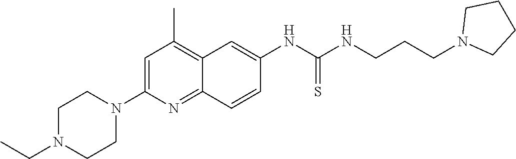 [SMILES: CCN1CCN(c2cc(C)c3cc(NC(=S)NCCCN4CCCC4)ccc3n2)CC1] |
| 0069 | 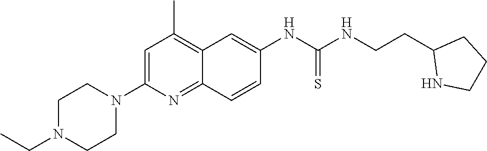 [SMILES: CCN1CCN(c2cc(C)c3cc(NC(=S)NCCC4CCCN4)ccc3n2)CC1] |
| 0070 | 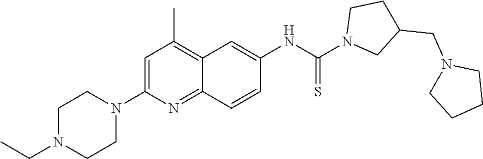 [SMILES: CCN1CCN(c2cc(C)c3cc(NC(=S)N4CCC(CN5CCCC5)C4)ccc3n2)CC1] |
| 0071 | 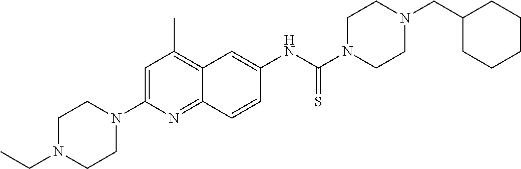 [SMILES: CCN1CCN(c2cc(C)c3cc(NC(=S)N4CCN(CC5CCCCC5)CC4)ccc3n2)CC1] |
| 0072 | 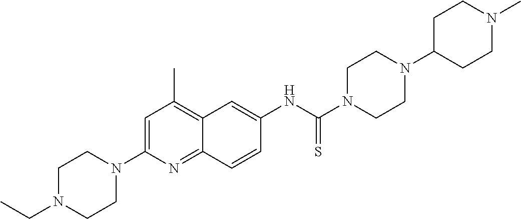 [SMILES: CCN1CCN(c2cc(C)c3cc(NC(=S)N4CCN(C5CCN(C)CC5)CC4)ccc3n2)CC1] |
| 0073 | 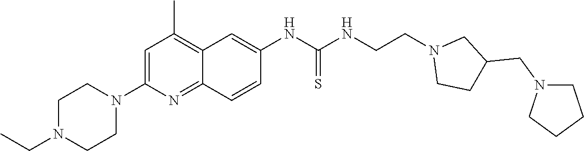 [SMILES: CCN1CCN(c2cc(C)c3cc(NC(=S)NCCN4CCC(CN5CCCC5)C4)ccc3n2)CC1] |
| 0074 | 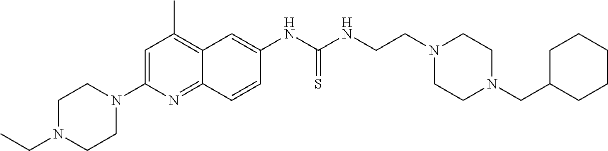 [SMILES: CCN1CCN(c2cc(C)c3cc(NC(=S)NCCN4CCN(CC5CCCCC5)CC4)ccc3n2)CC1] |
| 0075 | 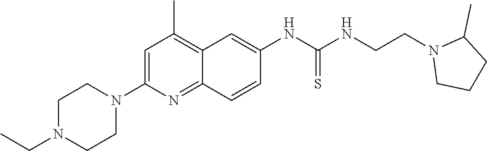 [SMILES: CCN1CCN(c2cc(C)c3cc(NC(=S)NCCN4CCCC4C)ccc3n2)CC1] |
| 0076 | 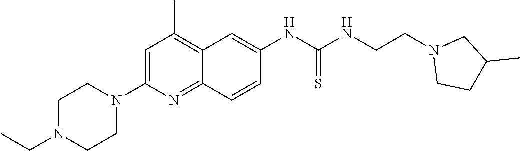 [SMILES: CCN1CCN(c2cc(C)c3cc(NC(=S)NCCN4CCC(C)C4)ccc3n2)CC1] |
| 0077 | 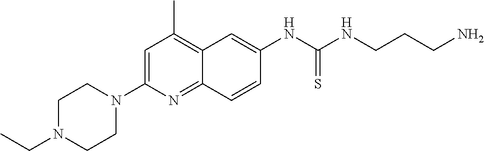 [SMILES: CCN1CCN(c2cc(C)c3cc(NC(=S)NCCCN)ccc3n2)CC1] |
| 0078 | 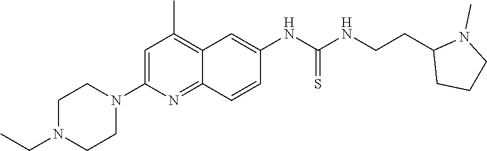 [SMILES: CCN1CCN(c2cc(C)c3cc(NC(=S)NCCC4CCCN4C)ccc3n2)CC1] |
| 0079 | 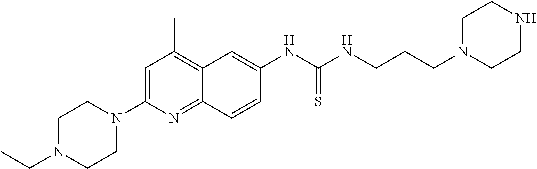 [SMILES: CCN1CCN(c2cc(C)c3cc(NC(=S)NCCCN4CCNCC4)ccc3n2)CC1] |
| 0080 | 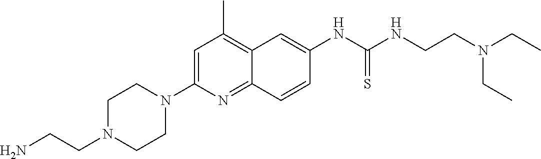 [SMILES: CCN(CC)CCNC(=S)Nc1ccc2nc(N3CCN(CCN)CC3)cc(C)c2c1] |
| 0082 | 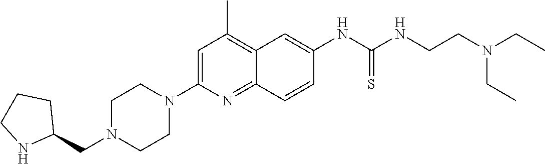 [SMILES: CCN(CC)CCNC(=S)Nc1ccc2nc(N3CCN(C[C@@H]4CCCN4)CC3)cc(C)c2c1] |
| 0083 | 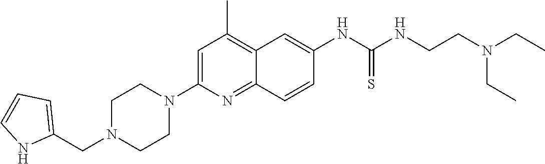 [SMILES: CCN(CC)CCNC(=S)Nc1ccc2nc(N3CCN(Cc4ccc[nH]4)CC3)cc(C)c2c1] |
| 0084 | 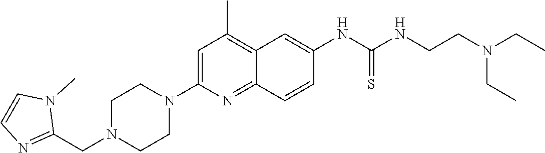 [SMILES: CCN(CC)CCNC(=S)Nc1ccc2nc(N3CCN(Cc4nccn4C)CC3)cc(C)c2c1] |
| 0085 | 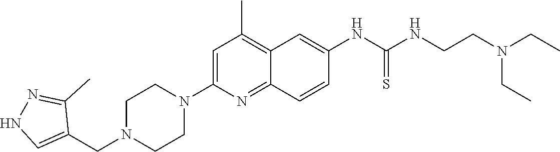 [SMILES: CCN(CC)CCNC(=S)Nc1ccc2nc(N3CCN(Cc4c[nH]nc4C)CC3)cc(C)c2c1] |
| 0086 | 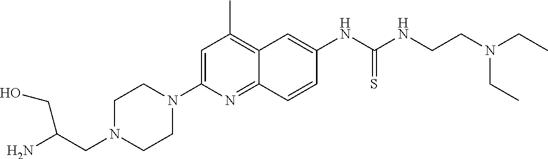 [SMILES: CCN(CC)CCNC(=S)Nc1ccc2nc(N3CCN(CC(N)CO)CC3)cc(C)c2c1] |
| 0087 | 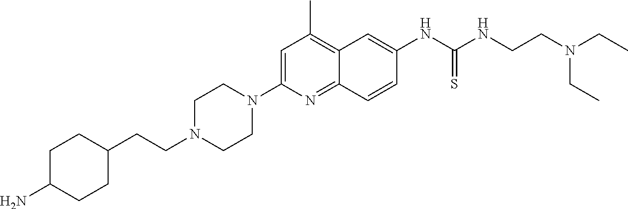 [SMILES: CCN(CC)CCNC(=S)Nc1ccc2nc(N3CCN(CCC4CCC(N)CC4)CC3)cc(C)c2c1] |
| 0088 | 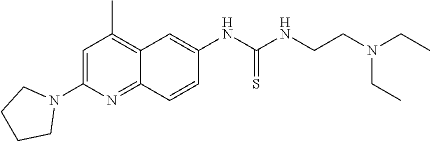 [SMILES: CCN(CC)CCNC(=S)Nc1ccc2nc(N3CCCC3)cc(C)c2c1] |
| 0089 | 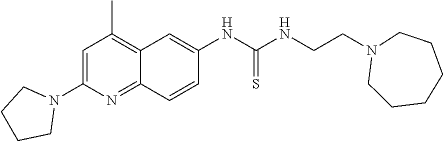 [SMILES: Cc1cc(N2CCCC2)nc2ccc(NC(=S)NCCN3CCCCCC3)cc12] |
| 0090 | 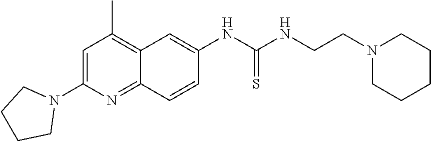 [SMILES: Cc1cc(N2CCCC2)nc2ccc(NC(=S)NCCN3CCCCC3)cc12] |
| 0091 | 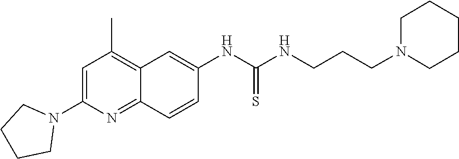 [SMILES: Cc1cc(N2CCCC2)nc2ccc(NC(=S)NCCCN3CCCCC3)cc12] |
| 0092 | 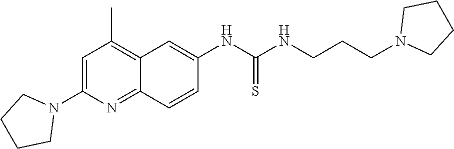 [SMILES: Cc1cc(N2CCCC2)nc2ccc(NC(=S)NCCCN3CCCC3)cc12] |
| 0093 | 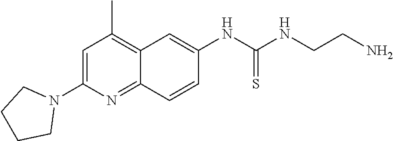 [SMILES: Cc1cc(N2CCCC2)nc2ccc(NC(=S)NCCN)cc12] |
| 0094 | 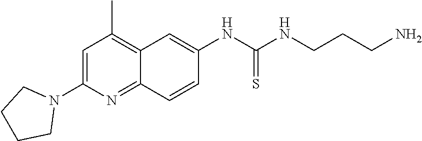 [SMILES: Cc1cc(N2CCCC2)nc2ccc(NC(=S)NCCCN)cc12] |
| 0095 | 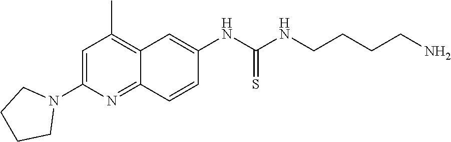 [SMILES: Cc1cc(N2CCCC2)nc2ccc(NC(=S)NCCCCN)cc12] |
| 0096 | 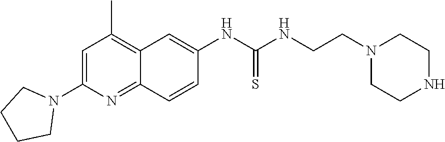 [SMILES: Cc1cc(N2CCCC2)nc2ccc(NC(=S)NCCN3CCNCC3)cc12] |
| 0097 | 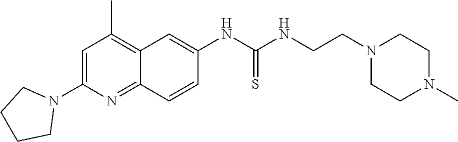 [SMILES: Cc1cc(N2CCCC2)nc2ccc(NC(=S)NCCN3CCN(C)CC3)cc12] |
| 0098 | 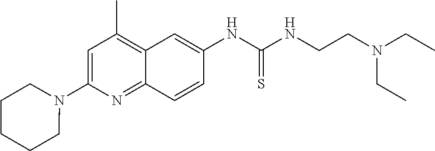 [SMILES: CCN(CC)CCNC(=S)Nc1ccc2nc(N3CCCCC3)cc(C)c2c1] |
| 0099 | 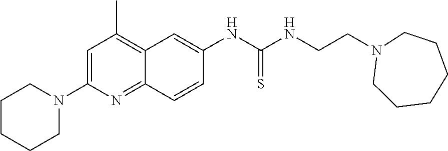 [SMILES: Cc1cc(N2CCCCC2)nc2ccc(NC(=S)NCCN3CCCCCC3)cc12] |
| 0100 | 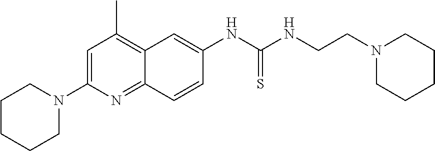 [SMILES: Cc1cc(N2CCCCC2)nc2ccc(NC(=S)NCCN3CCCCC3)cc12] |
| 0101 | 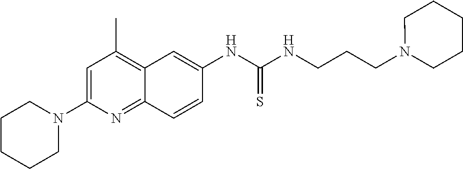 [SMILES: Cc1cc(N2CCCCC2)nc2ccc(NC(=S)NCCCN3CCCCC3)cc12] |
| 0102 | 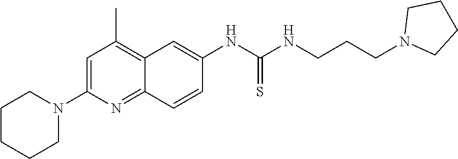 [SMILES: Cc1cc(N2CCCCC2)nc2ccc(NC(=S)NCCCN3CCCC3)cc12] |
| 0103 | 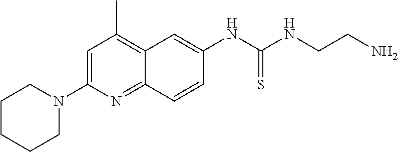 [SMILES: Cc1cc(N2CCCCC2)nc2ccc(NC(=S)NCCN)cc12] |
| 0104 | 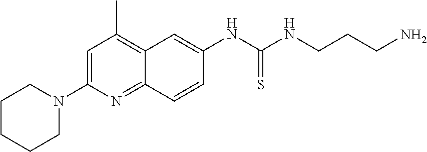 [SMILES: Cc1cc(N2CCCCC2)nc2ccc(NC(=S)NCCCN)cc12] |
| 0105 | 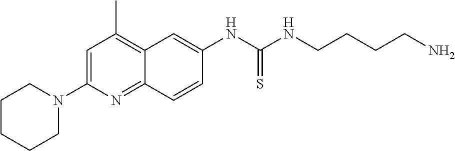 [SMILES: Cc1cc(N2CCCCC2)nc2ccc(NC(=S)NCCCCN)cc12] |
| 0106 | 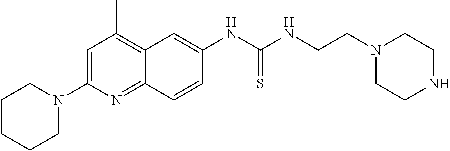 [SMILES: Cc1cc(N2CCCCC2)nc2ccc(NC(=S)NCCN3CCNCC3)cc12] |
| 0107 | 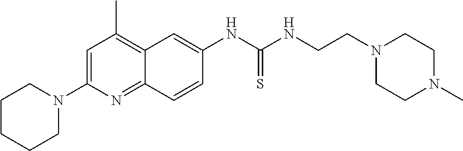 [SMILES: Cc1cc(N2CCCCC2)nc2ccc(NC(=S)NCCN3CCN(C)CC3)cc12] |
| 0108 | 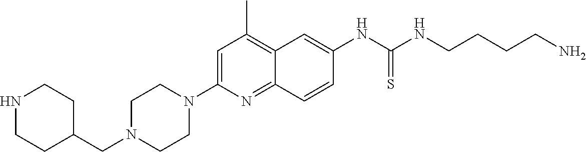 [SMILES: Cc1cc(N2CCN(CC3CCNCC3)CC2)nc2ccc(NC(=S)NCCCCN)cc12] |
| 0109 | 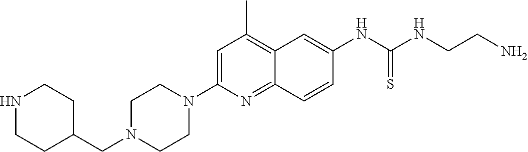 [SMILES: Cc1cc(N2CCN(CC3CCNCC3)CC2)nc2ccc(NC(=S)NCCN)cc12] |
| 0110 | 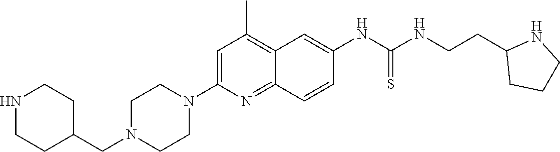 [SMILES: Cc1cc(N2CCN(CC3CCNCC3)CC2)nc2ccc(NC(=S)NCCC3CCCN3)cc12] |
| 0112 | 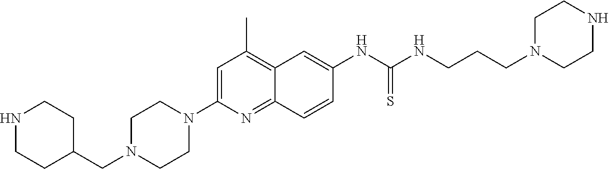 [SMILES: Cc1cc(N2CCN(CC3CCNCC3)CC2)nc2ccc(NC(=S)NCCCN3CCNCC3)cc12] |
| 0113 | 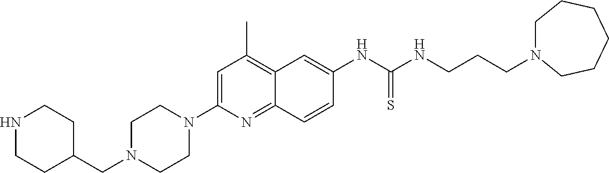 [SMILES: Cc1cc(N2CCN(CC3CCNCC3)CC2)nc2ccc(NC(=S)NCCCN3CCCCCC3)cc12] |
| 0114 | 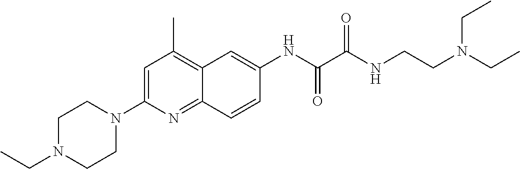 [SMILES: CCN(CC)CCNC(=O)C(=O)Nc1ccc2nc(N3CCN(CC)CC3)cc(C)c2c1] |

In some embodiments, the compound is a pharmaceutically acceptable salt of any one of the compounds described in Tables 1-9.

In some embodiments, the compound is a pharmaceutically acceptable salt of any one of the compounds described in Table 9.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 9 and prodrugs and pharmaceutically acceptable salts thereof.

In some embodiments, the compound is an isotopic derivative of any one of the compounds described in Table 9 and pharmaceutically acceptable salts thereof.

In some embodiments, the compound is an isotopic derivative of any one of prodrugs of the compounds described in Table 9 and pharmaceutically acceptable salts thereof.

In some embodiments, the compound is an isotopic derivative of any one of the compounds described in Table 9.

It is understood that the isotopic derivative can be prepared using any of a variety of art-recognized techniques. For example, the isotopic derivative can generally be prepared by carrying out the procedures disclosed in the Scheme and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula I′ or Formula I is isotopically enriched with regard to, or labelled with, one or more isotopes as compared to the corresponding compound of Formula I′ or Formula I. In some embodiments, the isotopic derivative is enriched with regard to, or labelled with, one or more atoms selected from ²H, ¹³C, ¹⁴C, ¹⁵N, ¹⁸O, ²⁹Si, ³¹P and ³⁴S. In some embodiments, the isotopic derivative is a deuterium labeled compound (i.e., being enriched with ²H with regard to one or more atoms thereof). In some embodiments, the compound is a ¹⁸F labeled compound. In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof. In some embodiments, the compound is a ³³S labeled compound, a ³⁴S labeled compound, a ³⁵S labeled compound, a ³⁶S labeled compound, or any combination thereof.

It is understood that the ¹⁸F, ¹²³J, ¹²⁴J, ¹²⁵J, ¹²⁹J, ¹³¹J, ¹³⁵I, ³²S, ³⁴S, ³⁵S, and/or ³⁶S labeled compound, can be prepared using any of a variety of art-recognized techniques. For example, the deuterium labeled compound can generally be prepared by carrying out the procedures disclosed in the Scheme and/or in the Examples described herein, by substituting a ¹⁸F, ¹²³I, ¹²⁴I, ¹²⁵I, ¹²⁹I, ¹³¹I, ¹³⁵I, ³S, ³⁴S, ³⁵S, and/or ³⁶S labeled reagent for a non-isotope labeled reagent.

A compound of the invention or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³²S, ³⁴S, ³⁵S, and ³⁶S atom(s) is within the scope of the invention. Further, substitution with isotope (e.g., ¹⁸F, ¹²³I, ¹²⁴I, ¹²⁵I, ¹²⁹I, ¹³¹I, ¹³⁵I, ³S, ³⁴S, ³⁵S, and/or ³⁶S) may afford certain therapeutic advantages resulting from greater metabolic stability, e.g., increased in vivo half-life or reduced dosage requirements.

The compounds described herein can possess one or more stereocenters, and each stereocenter can exist independently in either the (R) or (S) configuration. In some embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In some embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In some embodiments, the compounds described herein exist in solvated forms with pharmaceutically acceptable solvents such as water, and ethanol. In other embodiments, the compounds described herein exist in unsolvated form.

In some embodiments, the compound(s) described herein can exist as tautomers. All tautomers are included within the scope of the compounds presented herein.

In some embodiments, compounds described herein are prepared as prodrugs. A “prodrug” refers to an agent that is converted into the parent drug in vivo. In some embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In some embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In some embodiments, the appropriate substituent to decrease or eliminate the susceptibility of the aromatic ring to metabolic reactions is, by way of example only, a deuterium, a halogen, or an alkyl group.

In some embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In some embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In some embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups may be useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

In some embodiments, a blocking/protecting groups may be selected from:

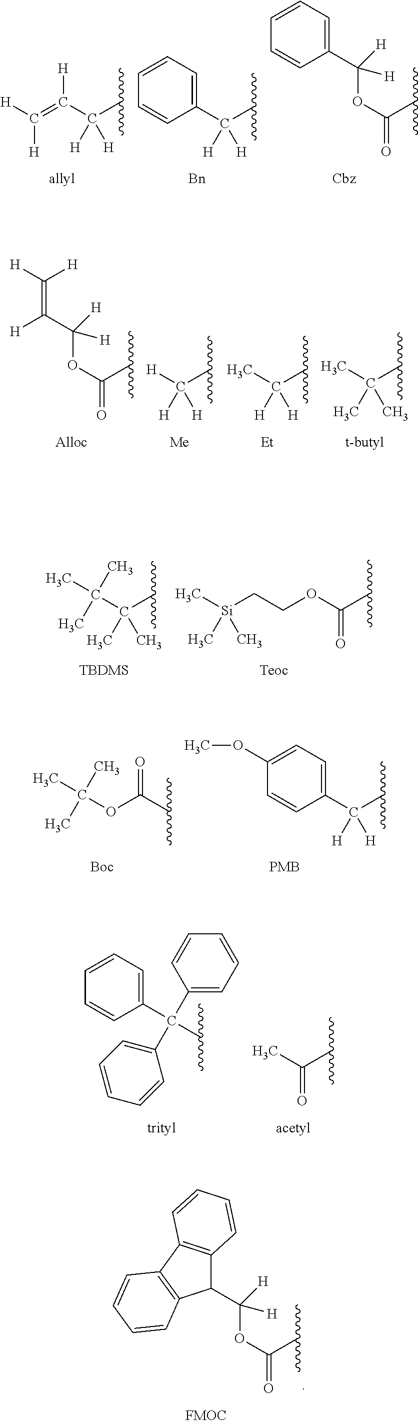

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays are may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological assay is a cell viability assay, a clonogenic survival assay, a surface plasmon resonance assay, a fluorescence-quenching assay, a D-loop formation assay, or an acridine orange displacement assay.

In some embodiments, the biological assay is described in the Examples herein.

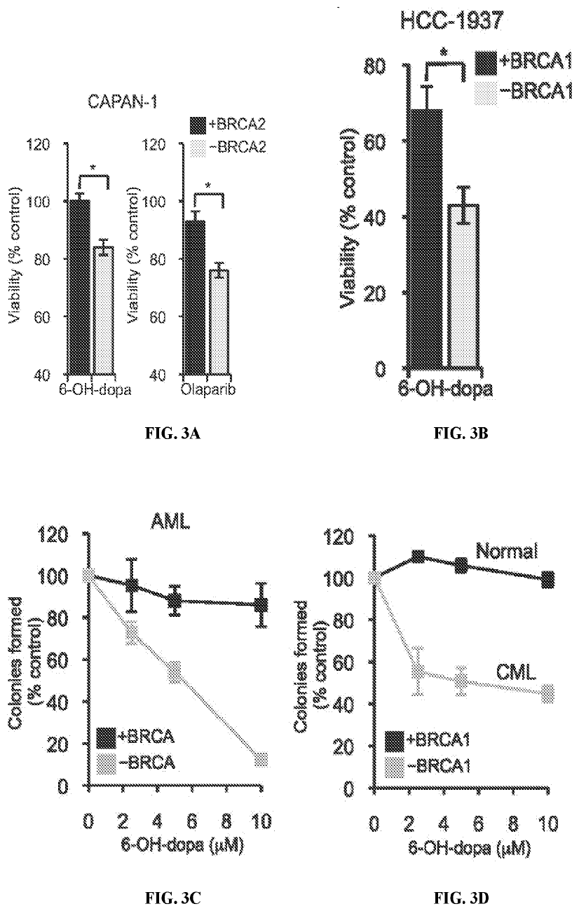

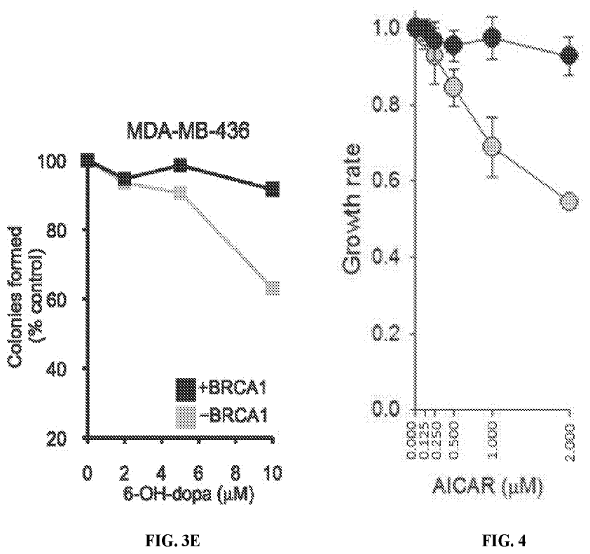

The applicability of RAD52 inhibitors in BRCA-deficient cancers can be seen in Chandramouly, Gurushankar et al. “Small-Molecule Disruption of RAD52 Rings as a Mechanism for Precision Medicine in BRCA-Deficient Cancers.” Chemistry & Biology vol. 22, 11 (2015): 1491-1504, wherein FIG. 3A depicts viability of BRCA proficient (black) and deficient (grey) cells from the pancreatic adenocarcinoma cancer cell line CAPAN-1 following treatment with 10 μM 6-hydroxydopamine (6-OH-dopa). FIG. 3B depicts viability of BRCA proficient (black) and deficient (grey) cells from the BRCA1 deficient triple negative breast cancer cell line HCC1937 following treatment with 5 μM 6-OH-dopa. FIG. 3C depicts clonogenic survival of acute myeloid leukemia (AML) cells from patients with low expression of BRCA1/2 following treatments with 6-OH-dopa. FIG. 3D depicts clonogenic survival of chronic myelogenous leukemia (CML) cells from patients with low expression of BRCA1 following treatments with 6-OH-dopa. FIG. 3E depicts clonogenic survival of BRCA1-deficient breast cancer cells from the cell line MDA-MB-436 following treatments with 6-OH-dopa. FIGS. 3A, 3B, 3C, 3D, and 3E are adapted from.

The applicability of RAD52 inhibitors is shown in FIG. 4 which depicts the growth of BRCA1-null HCC1937 cells (grey dots) and their BRCA1-reconstitued counterparts (black dots) in the presence of indicated concentrations of 5-aminoimidazole-4-carboxamide ribonucleotide (adapted from Sullivan, Katherine et al. “Identification of a Small Molecule Inhibitor of RAD52 by Structure-Based Selection.” PloS one vol. 11, 1 e0147230. 19 Jan. 2016).

### Methods of Treatment

In some aspects, the present disclosure provides a method of modulating RAD52 activity (e.g., in vitro or in vivo), comprising contacting a cell with an effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some embodiments, the disease or disorder is associated with an implicated RAD52 activity. In some embodiments, the disease or disorder is a disease or disorder in which RAD52 activity is implicated.

In some embodiments, the disease or disorder is a cancer.

In some aspects, the present disclosure provides a method of treating or preventing a cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing breast cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating breast cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing ovarian cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating ovarian cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in modulating RAD52 activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing a cancer in a subject in need thereof.

In some aspects, the present disclosure provides a compound of the present disclosure or a pharmaceutically acceptable salt thereof for use in treating a cancer in a subject in need thereof.

In some aspects, the present disclosure provides a compound of the present disclosure or a pharmaceutically acceptable salt thereof for use in treating or preventing breast cancer in a subject in need thereof.

In some aspects, the present disclosure provides a compound of the present disclosure or a pharmaceutically acceptable salt thereof for use in treating breast cancer in a subject in need thereof.

In some aspects, the present disclosure provides a compound of the present disclosure or a pharmaceutically acceptable salt thereof for use in treating or preventing ovarian cancer in a subject in need thereof.

In some aspects, the present disclosure provides a compound of the present disclosure or a pharmaceutically acceptable salt thereof for use in treating ovarian cancer in a subject in need thereof.

In some aspects, the present disclosure provides use of a compound of the present disclosure or a pharmaceutically acceptable salt thereof in the manufacture of a medicament for modulating RAD52 activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing a cancer in a subject in need thereof.

In some aspects, the present disclosure provides use of a compound of the present disclosure or a pharmaceutically acceptable salt thereof in the manufacture of a medicament for treating a cancer in a subject in need thereof.

In some aspects, the present disclosure provides use of a compound of the present disclosure or a pharmaceutically acceptable salt thereof in the manufacture of a medicament for treating or preventing breast cancer in a subject in need thereof.

In some aspects, the present disclosure provides use of a compound of the present disclosure or a pharmaceutically acceptable salt thereof in the manufacture of a medicament for treating breast cancer in a subject in need thereof.

In some aspects, the present disclosure provides use of a compound of the present disclosure or a pharmaceutically acceptable salt thereof in the manufacture of a medicament for treating or preventing ovarian cancer in a subject in need thereof.

In some aspects, the present disclosure provides use of a compound of the present disclosure or a pharmaceutically acceptable salt thereof in the manufacture of a medicament for treating ovarian cancer in a subject in need thereof.

The present disclosure provides compounds that function as modulators of RAD52 activity.

In some embodiments, the compounds of the present disclosure inhibit RAD52.

In some embodiments, modulation is inhibition.

Effectiveness of compounds of the disclosure can be determined by industry-accepted assays/disease models according to standard practices of elucidating the same as described in the art and are found in the current general knowledge.

The present disclosure also provides a method of treating a disease or disorder in which RAD52 activity is implicated in a patient in need of such treatment, said method comprising administering to said patient a therapeutically effective amount of a compound, or a pharmaceutically acceptable salt thereof, or a pharmaceutical composition as defined herein.

The disclosure includes a method of treating cancer using the compounds of Formula I′ or Formula I. The disclosure includes a method of treating cancer using the compounds of Formula I′. The disclosure includes a method of treating cancer using the compounds of Formula I. In some embodiments, the method includes inhibiting RAD52 in a subject in need thereof, thereby treating cancer in the subject.

In some embodiments, the cancer has a dysfunctional RAD52 activity.

In some embodiments, the cancer has a dysfunctional BRCA1, BRCA2, PALB2, or RAD51 paralog (e.g., RAD51D or XRCC3) activity. In some embodiments, the cancer has a dysfunctional BRCA1 activity. In some embodiments, the cancer has a dysfunctional BRCA2 activity. In some embodiments, the cancer has a dysfunctional PALB2 activity. In some embodiments, the cancer has a dysfunctional RAD51 paralog (e.g., RAD51D or XRCC3) activity.

In some embodiments, the dysfunction is a mutation. In some embodiments, the dysfunction is constitutive downregulation.

In some embodiments, the cancer is squamous cell cancer, lung cancer, vulval cancer, thyroid cancer, adenocarcinoma of the lung, squamous carcinoma of the lung, cancer of the peritoneum, hepatocellular cancer, gastric or stomach cancer including gastrointestinal cancer, gastroesophageal, pancreatic cancer, brain cancer, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, biliary tract, anal carcinoma, penile carcinoma, leukemia, lymphoma, melanoma, or head and neck cancer.

In some embodiments, the lung cancer is small-cell lung cancer or non-small cell lung cancer.

In some embodiments, the cancer is a solid tumor.

In some embodiments, the cancer is ovarian cancer. In some embodiments, the ovarian cancer has a BRCA1 and/or BRCA2 mutation. In some embodiments, the ovarian cancer has a BRCA1 and BRCA2 mutation. In some embodiments, the ovarian cancer has a BRCA1 or BRCA2 mutation. In some embodiments, the ovarian cancer has a BRCA1 mutation. In some embodiments, the ovarian cancer has a BRCA2 mutation.

In some embodiments, the cancer is breast cancer. In some embodiments, the breast cancer has a BRCA1 and/or BRCA2 mutation. In some embodiments, the breast cancer has a BRCA1 and BRCA2 mutation. In some embodiments, the breast cancer has a BRCA1 or BRCA2 mutation. In some embodiments, the breast cancer has a BRCA1 mutation. In some embodiments, the breast cancer has a BRCA2 mutation. In some embodiments, the breast cancer is triple negative breast cancer.

In some embodiments, the cancer is pancreatic cancer. In some embodiments, the pancreatic cancer is pancreatic adenocarcinoma.

In some embodiments, the cancer is brain cancer. In some embodiments, the brain cancer is glioblastoma.

In some embodiments, the cancer is leukemia. In some embodiments the leukemia is acute myeloid leukemia, chronic myelogenous leukemia, or chronic myelogenous leukemia.

In some embodiments the leukemia is acute myeloid leukemia. In some embodiments the leukemia is chronic myelogenous leukemia. In some embodiments the leukemia is chronic myelogenous leukemia.

In some embodiments, the cancer is a gastric cancer. In some embodiments, the cancer is pancreatic cancer.

In some embodiments, the subject has a BRCA1 and/or BRCA2 mutation.

In some embodiments, the subject has a BRCA1 and BRCA2 mutation. In some embodiments, the subject has a BRCA1 or BRCA2 mutation. In some embodiments, the subject has a BRCA1 mutation. In some embodiments, the subject has a BRCA2 mutation.

The Examples described herein demonstrate that inhibition of RAD52 caused the death of BRCA-1 and/or BRCA-2 deficient cells.

In some embodiments, the cancer comprises PARP inhibitor resistant cells.

In some embodiments, the subject has previously been administered a PARP inhibitor. In some embodiments, the subject is resistant to PARP inhibitors.

In some embodiments, the cancer comprises a homologous recombination DNA damage repair mutation.

In some embodiments, a method of treating a RAD52 related disease or disorder in a subject in need thereof includes administering at least one compound of Formula I′ or Formula I. In some embodiments, a method of treating a RAD52 related disease or disorder in a subject in need thereof includes administering at least one compound of Formula I′. In some embodiments, a method of treating a RAD52 related disease or disorder in a subject in need thereof includes administering at least one compound of Formula I. In some embodiments, the RAD52 related disease or disorder is any of the cancers described herein.

The methods described herein include administering to the subject a therapeutically effective amount of at least one compound described herein, which is optionally formulated in a pharmaceutical composition. In some embodiments, a therapeutically effective amount of at least one compound described herein present in a pharmaceutical composition is the only therapeutically active compound in a pharmaceutical composition. In some embodiments, the method further comprises administering to the subject an additional therapeutic agent that treats cancer.

In some embodiments, administering the compound(s) described herein to the subject allows for administering a lower dose of the additional therapeutic agent as compared to the dose of the additional therapeutic agent alone that is required to achieve similar results in treating a cancer in the subject. For example, in some embodiments, the compound(s) described herein enhance(s) the activity of the additional therapeutic compound, thereby allowing for a lower dose of the additional therapeutic compound to provide the same effect.

In some embodiments, the compound(s) described herein and the therapeutic agent are co-administered to the subject. In other embodiments, the compound(s) described herein and the therapeutic agent are coformulated and co-administered to the subject.

In some embodiments, the subject is a mammal. In other embodiments, the mammal is a human.

### Combination Therapies

The compounds useful within the methods described herein can be used in combination with one or more additional therapeutic agents useful for treating cancer. These additional therapeutic agents may comprise compounds that are commercially available or synthetically accessible to those skilled in the art. These additional therapeutic agents are known to treat or reduce the symptoms, of cancer.

In non-limiting examples, the compounds useful within the invention may be used in combination with one or more of the following therapeutic agents: Erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), pemetrexed (ALIMTA®, Eli Lilly), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, rapamycin, oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®).

In some embodiments, the compounds described herein can be used in combination with radiation therapy. In other embodiments, the combination of administration of the compounds described herein and application of radiation therapy is more effective in treating or preventing cancer than application of radiation therapy by itself. In yet other embodiments, the combination of administration of the compounds described herein and application of radiation therapy allows for use of lower amount of radiation therapy in treating the subject.

In some embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat cancer in the patient. An effective amount of the therapeutic compound necessary to achieve a therapeutic effect may vary according to factors such as the state of the disease or disorder in the patient; the age, sex, and weight of the patient; and the ability of the therapeutic compound to treat cancer in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In some embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In some embodiments, the compositions described herein are formulated using one or more pharmaceutically acceptable excipients or carriers. In some embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In some embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In some embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

In some embodiments, suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

Oral Administration

In some embodiments for oral application, tablets, dragees, liquids, drops, suppositories, or capsules, caplets and gelcaps are suitable. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

In some embodiments, suitable dispersing agents include, but are not limited to, potato starch, sodium starch glycolate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

In some embodiments, suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

In some embodiments, suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

In some embodiments, suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

In some embodiments, suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, optionally in their polyoxyethylated versions. These oil solutions or suspensions may also contain a long-chain alcohol diluent or dispersant, such as Ph. Helv or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In some embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some embodiments, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein.

Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds.

The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In some embodiments, the compound(s) described herein are administered to a patient, alone or in combination with another pharmaceutical agent, using a sustained release formulation. In some embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of cancer in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In some embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### Methods of Synthesis

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

### EXAMPLES

Some embodiments of the present application can be better understood by reference to the following Examples which are offered by way of illustration. The scope of the present application is not limited to the Examples given herein.

For exemplary purpose, neutral compounds of Formula I′ or Formula I are synthesized and tested in the examples. It is understood that the neutral compounds of Formula I′ or Formula I may be converted to the corresponding pharmaceutically acceptable salts of the compounds using routine techniques in the art.

Abbreviations:

BOC tert-butyl carbamate

BSA bovine serum albumin

DCE 1,2-dichloroethane

DCM dichloromethane

DIEA/DIPEA N,N-diisopropylethylamine

ds(DNA) double-stranded DNA

DTT dithiothreitol

EtOAc ethyl acetate

EDTA ethylenediaminetetraacetic acid

Et₃N triethylamine

HEPES 4-(2-hydroxyethyl)-1-piperazineethanesulfonic acid

HOBT hydroxy benzotriazole

HPLC high-performance liquid chromatography

HTS high-throughput screening

LC liquid chromatography

MeCN acetonitrile

MS mass spectrometry

NMR nuclear magnetic resonance

rt room temperature

SDS sodium dodecyl sulfate

ssDNA single-stranded DNA

TFA trifluoroacetic acid

THE tetrahydrofuran

TMS tetramethylsilane

Tris tris(hydroxymethyl)aminomethane

UV Ultraviolet

UV-VIS Ultraviolet/Visible

### Example 1. Synthesis of the Compound of the Present Disclosure

### General Synthetic Schemes for Preparation of Compounds of Formula I

The reagents used in the preparation of the compounds described herein can be commercially obtained or prepared by standard procedures described in the literature. Compounds of Formula I′ or Formula I can be produced by one of the following reaction schemes.

The first aspect of the process of the present disclosure relates to a process for preparing quinolines having the Formula I′ or Formula I. Compounds of Formula I′ or Formula I can be prepared according to the process outlined in Schemes 1-17.

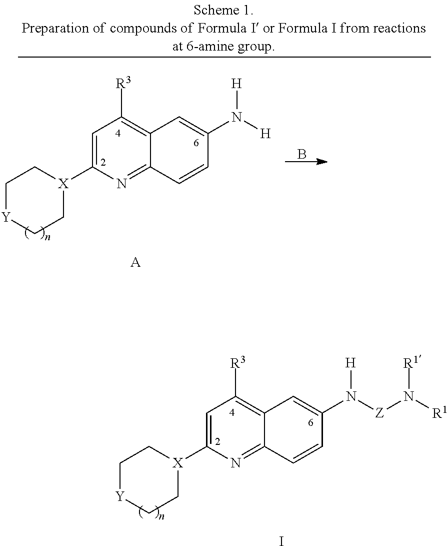

Accordingly, a suitably substituted compound of the formula (A), a known compound or compound prepared by known methods, is reacted with a compound B, a known compound or a compound prepared by known methods, optionally in the presence an organic solvent such as methylene chloride, dichloroethane, tetrahydrofuran, 1,4-dioxane, N,N-dimethyl formamide, and the like, optionally with cooling or heating (optionally with microwave irradiation), to provide a compound of the Formula I′ or Formula I. Compound B can be but is not limited to, a thio-isocyanate (Scheme 2), an isocyanate (Scheme 3), a carbamic chloride (Scheme 4), a substituted amino-2-oxoacetyl chloride (Scheme 5), or a phenyl N′-cyano-N,N—R¹R^{1′}-carbamimidate (Scheme 6).

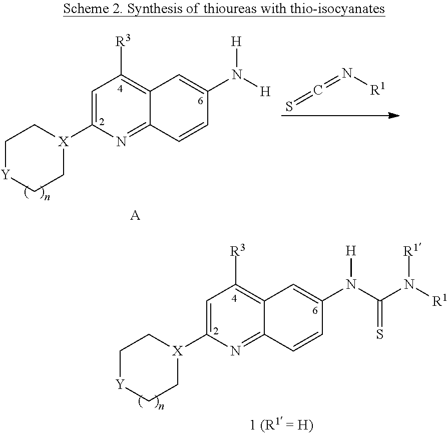

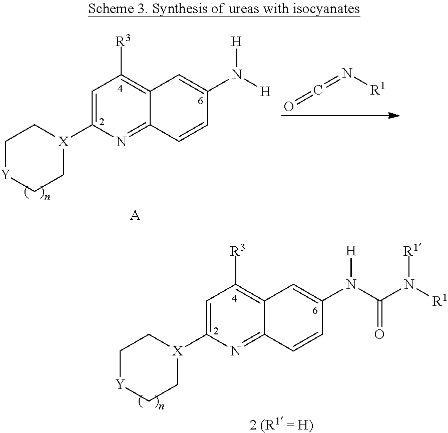

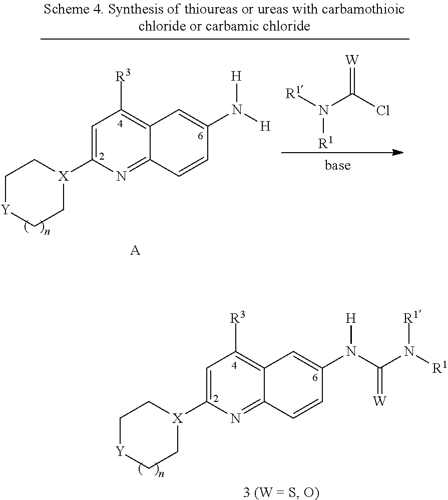

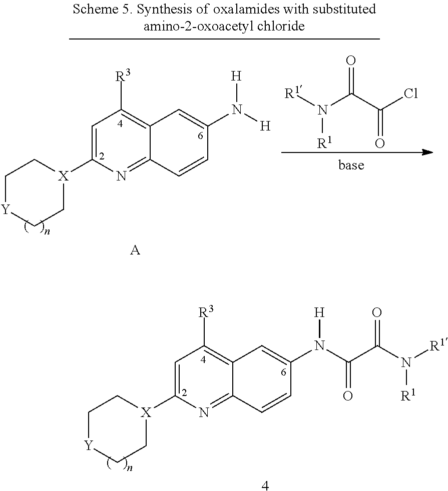

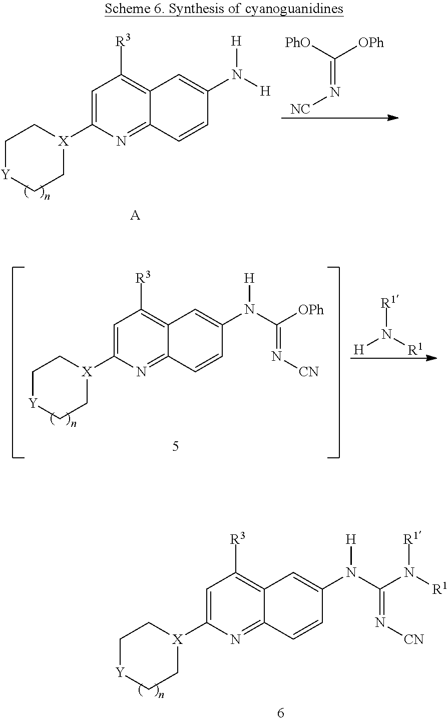

Alternatively, a suitably substituted compound of the formula (A), a known compound or compound prepared by known methods, is activated to form an active intermediate, such as thio-isocyanate 7 (Scheme 7), or isocyanate 8 (Scheme 8), or amino-2-oxoacetyl chloride 9 (Scheme 9), which can further react with an amine to provide the corresponding products of the formula 1-2 and 4, in the presence of an organic solvent such as tetrahydrofuran, 1,4-dioxane, N,N-dimethylformamide, methylene chloride, dichloroethane, methanol, ethanol, and the like, optionally in the presence of a base such as triethylamine, diisopropylethylamine, pyridine, 2,6-lutidine, and the like, optionally in the presence of 4-N,N-dimethylaminopyridine, optionally with heating, optionally with microwave irradiation.

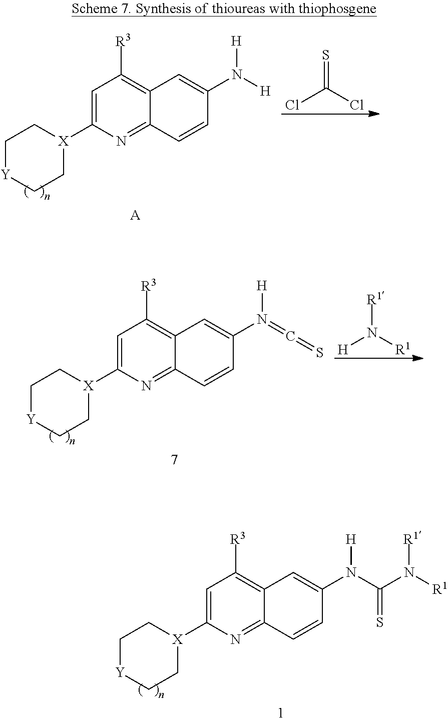

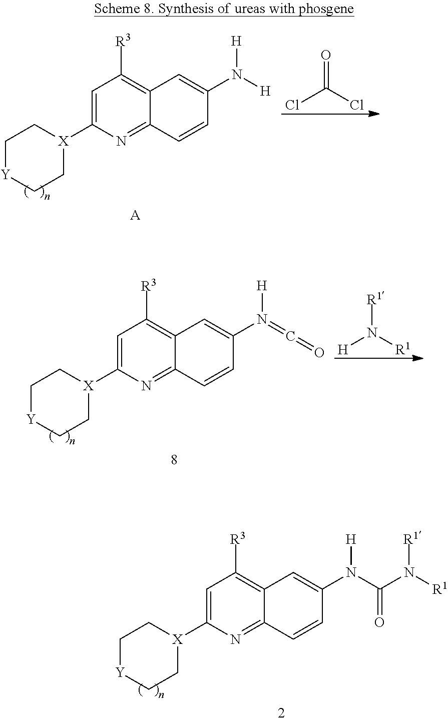

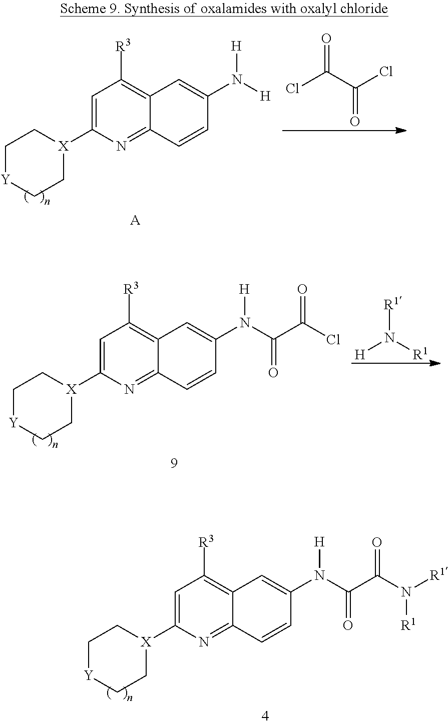

A compound of the formula (B, Y═N), a known compound or a compound prepared by known methods wherein Q is selected from the group consisting of nitro, or the corresponding moieties at position 6 of quinoline in the compounds 1-6., is reacted with a known compound or a compound prepared by known methods, such as alkyl halide (Scheme 10), aldehyde (Scheme 11), chloroformate (Scheme 12), acid chloride (Scheme 13), isocyanate (Scheme 14), sulfonyl chloride (Scheme 15), optionally in the presence an organic solvent such as methylene chloride, dichloroethane, tetrahydrofuran, 1,4-dioxane, N,N-dimethyl formamide, and the like, optionally with heating, optionally with microwave irradiation, to provide compounds 10-15 (as seen in Scheme 10-15, below).

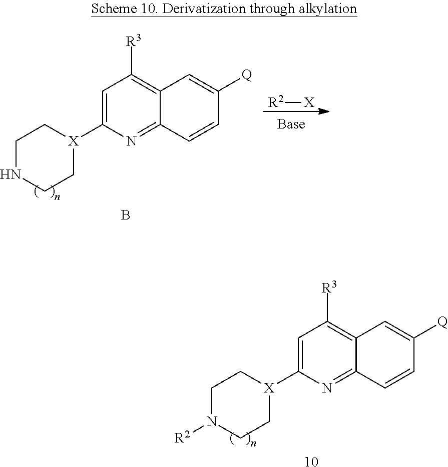

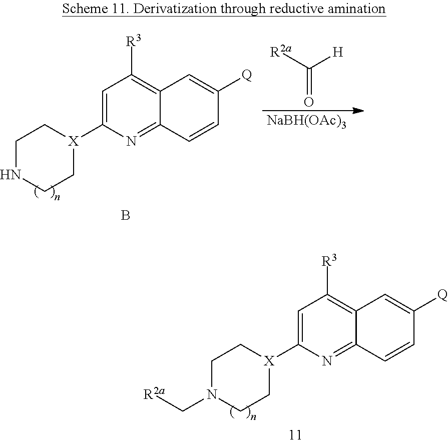

wherein R^{2a} is H, C₁₋₄ alkyl, —C₀₋₄ alkyl-C₃₋₆ cycloalkyl, —C₀₋₄ alkyl-(3- to 7-membered heterocyclyl), —C₀₋₄ alkyl-(C₆₋₁₀ aryl), or —C₀₋₄ alkyl-(3- to 7-membered heteroaryl), wherein the alkyl, cycloalkyl, heterocyclyl, aryl, heteroaryl represented by R² is optionally substituted with one or more —OH, —NH₂, —NH—C(═O)—O—(C₁₋₆ alkyl), halogen, C₁₋₆ alkyl, or phenyl.

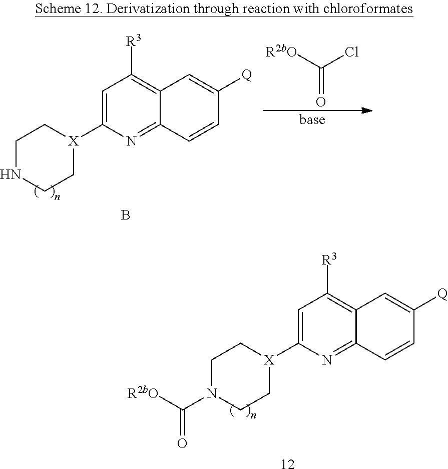

wherein R^{2b} is C₁₋₆ alkyl, optionally substituted with one or more —OH, —NH₂, —NH—C(═O)—O—(C₁₋₆ alkyl), halogen, C₁₋₆ alkyl, or phenyl.

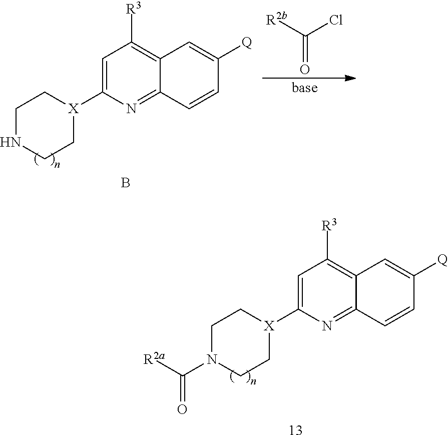

wherein R^{2b} is as described herein.

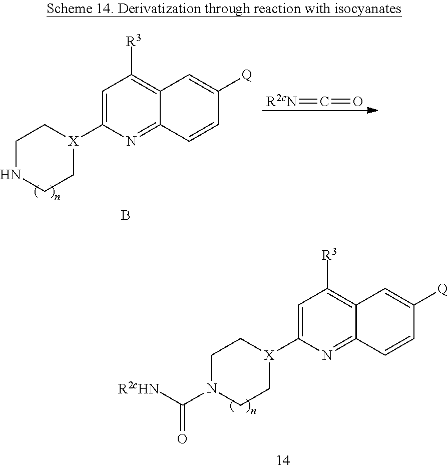

wherein R^{2c} is —C₁₋₆ alkyl, or —C₆₋₁₀ aryl, wherein the alkyl or aryl represented by R^{2c} is optionally substituted with one or more —OH, —NH₂, —NH—C(═O)—O—(C₁₋₆ alkyl), halogen, C₁₋₆ alkyl, or phenyl.

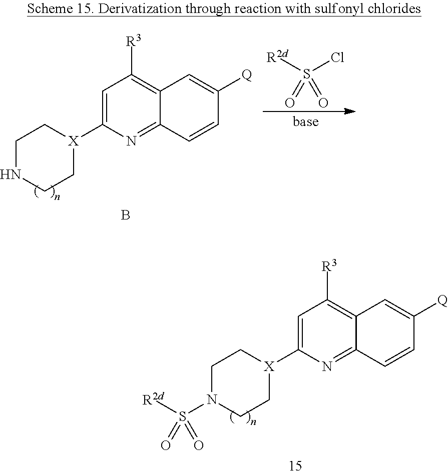

wherein R^{2d} is C₆₋₁₀ aryl optionally substituted with one or more —OH, —NH₂, —NH—C(═O)—O—(C₁₋₆ alkyl), halogen, C₁₋₆ alkyl, or phenyl.

A compound of the formula (B) is reacted with an aromatic halide or heteroaromatic halide (11), a known compound or a compound prepared by known methods, in the presence of a palladium catalyst such as palladium (II) acetate, tetrakis(triphenylphosphine)palladium(0), dichlorobis (triphenyl phosphine)palladium(II), bis(acetonitrile)dichloropalladium(II), tris(dibenzylideneacetone) dipalladium(0), and the like, in the presence of a ligand and a base such as sodium carbonate, potassium carbonate, lithium carbonate, sodium bicarbonate, potassium bicarbonate, cesium carbonate. lithium bicarbonate, triethylamine, diisopropylethylamine, pyridine, and the like, optionally in the presence of water, in a solvent such as tetrahydrofuran, 1,4-dioxane, acetonitrile, N,N-dimethyl formamide, N,N-dimethylacetamide, methylene chloride, 1,2-dichloroethane, and the like, optionally with heating, optionally with microwave irradiation to provide a compound of the formula (16). Or, a compound of the formula (B) is reacted with an aromatic boronic acid or heteroaromatic boronic acid, a known compound or a compound prepared by known methods, in the presence of a copper catalyst such as copper (II) acetate, and the like, in the presence of a base to make the compound of the formula (16).

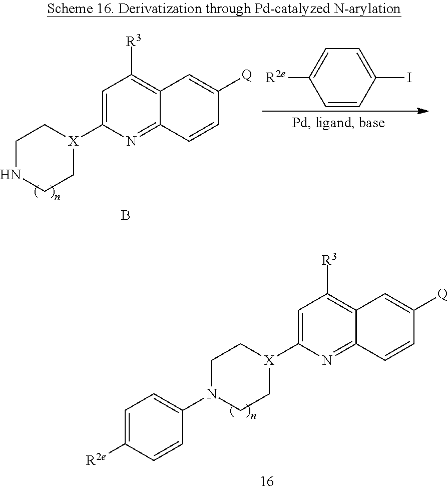

wherein R^{2e} is —OH, —NH₂, —NH—C(═O)—O—(C₁₋₆ alkyl), halogen, C₁₋₆ alkyl, or phenyl.

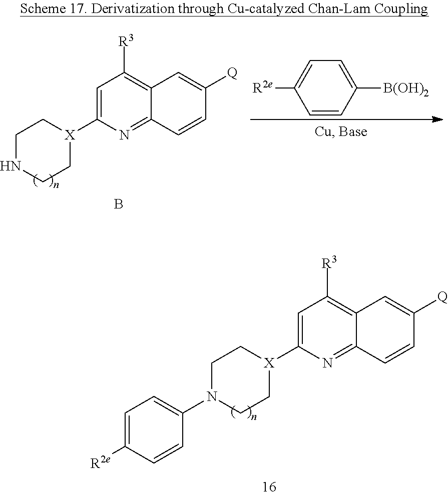

### General Chemistry Methods

All reagents and solvents were used as purchased from commercial sources. Reactions were carried out under argon atmosphere. Flash column chromatography was performed on either CombiFlash Rf+ or CombiFlash Companion using the appropriate size Teledyne ISCO columns (20-40 microns or 40-60 microns) and prepacked silica filled cartridges. Preparative high-performance liquid chromatography (HPLC) was performed using a Gilson 331 and 332 pumps with a UV/VIS-155 detector and GX-271 liquid handler. Column was Phenomenex Luna LC Column (5 μm C18 100 Å, 150×21.2 mm). ¹H NMR spectra were recorded on a 300 MHz INOVA VARIAN spectrometer. Chemical shifts values are given in ppm and referred as the internal standard to TMS (tetramethylsilane). The peak patterns are indicated as follows: s, singlet; d, doublet; t, triplet; q, quadruplet; m, multiplet; and dd, doublet of doublets. The coupling constants (J) are reported in Hertz (Hz). Mass Spectra were obtained on an Agilent 6120 mass spectrometer with electrospray ionization source (1200 Aligent LC-MS spectrometer, Positive). Mobile phase flow was 1.0 mL/min with a 3.0 min gradient from 20% aqueous media (0.1% formic acid) to 95% CH₃CN (0.1% formic acid) and a 9.0 min total acquisition time. All the tested compounds possess a purity of at least 90%, which was determined by LC/MS Data recorded using an Agilent 1200 liquid chromatography and Agilent 6120 mass spectrometer, and further supported by clean NMR spectra.

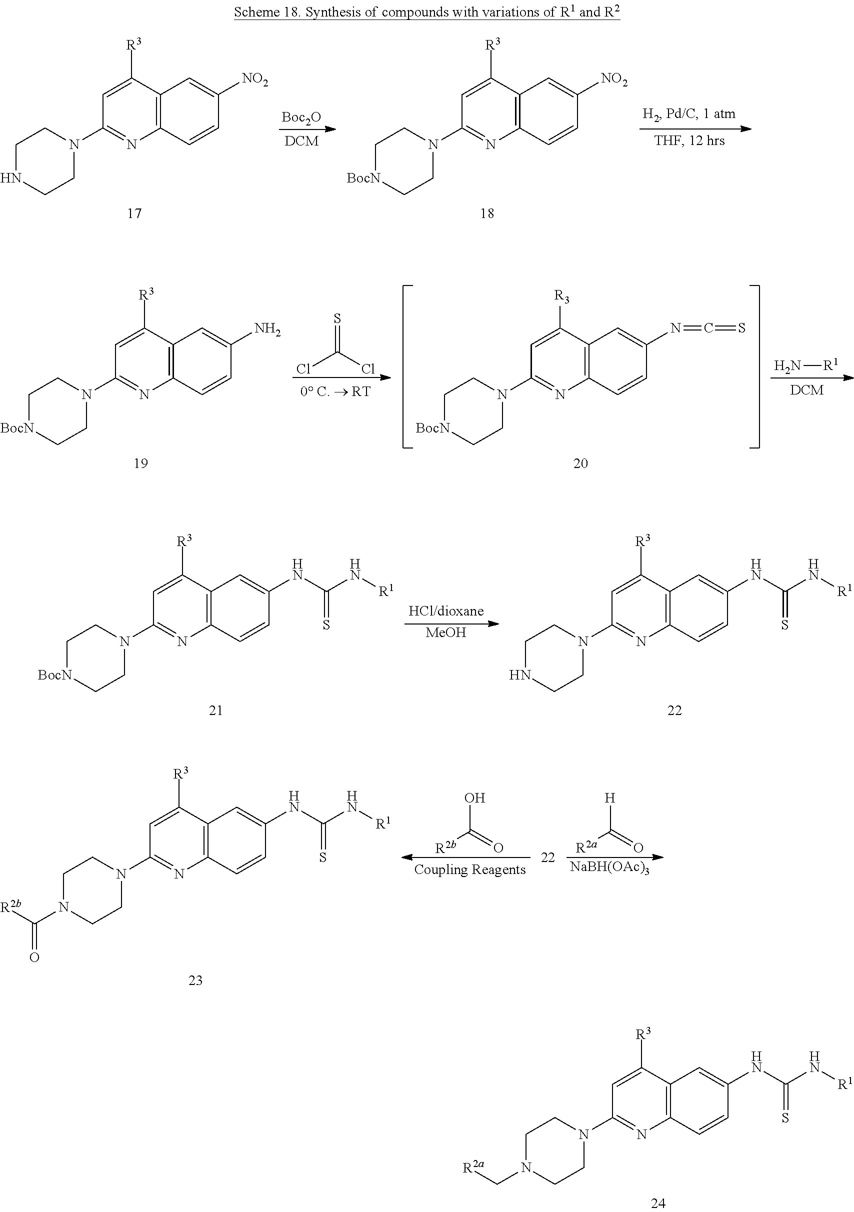

The starting material 17 was dissolved in DCM, and Boc₂O (1 eq.) in DCM was added dropwise. After letting the reaction stir overnight, it was concentrated down and re-dissolved in THF. Pd/C was added and the reaction was put in the hydrogenator at around 1.0-1.5 atm H₂ and left overnight. The mixture was then filtered through celite and the filtrate was collected and concentrated down to provide intermediate 19 for the next step. 19 was dissolved in DCM, and 4 eq. NaHCO₃ dissolved in an equal amount of water was added to it. The reaction was put in an ice bath to cool down to 0° C., and thiophosgene (1.5 eq.) in DCM was added dropwise. After 10 min, the reaction was taken off the ice bath and allowed to proceed at room temperature overnight. The organic and aqueous phases were then separated, and the organic layer was washed with brine, dried with solid Na₂SO₄, and concentrated down. The intermediate 20 was redissolved in DCM, and Et₃N and N,N-diethylethylenediamine (or a different amine) (1.2 eq.) was added. After overnight, it was diluted in EtOAc, washed with brine, and concentrated. Then it was purified by HPLC in 0.1% TFA of H₂O:MeCN to afford 21. The Boc was then removed by stirring the intermediate 21 in 1:1 4M HCl in dioxane and MeOH for 2 hours. Afterwards, it was concentrated and co-evaporated with MeOH 3 times to get rid of excess of HCl.

Then the resulting intermediate 22 can react with either an aldehyde or carboxylic acid. For the carboxylic acid (1 eq.), the acid, 6, EDC HCl (1 eq.), HOBT H₂O (1 eq.), and Et₃N were stirred in DCM overnight. The reaction mixture was concentrated and purified by HPLC; For the aldehyde, the aldehyde, 22, NaBH(OAc)₃ (3 eq.), were dissolved in 1,2-dichloroethane (DCE) with a few drops of Et₃N to neutralize the acid salt. After the reaction stirred overnight, it was quenched with aqueous NaHCO₃ and stirred vigorously for several hours. The phases were separated and the aqueous phase was extracted with DCM twice. The organic phases were combined and dried with solid Na₂SO₄ and then purified with HPLC.

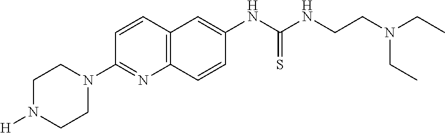 [SMILES: CCN(CC)CCNC(=S)Nc1ccc2nc(N3CCNCC3)ccc2c1]

1-(2-(diethylamino)ethyl)-3-(2-(piperazin-1-yl)quinolin-6-yl)thiourea. This compound was prepared according to synthetic Scheme 18. LC-MS, (M+H)⁺@ 387.6.

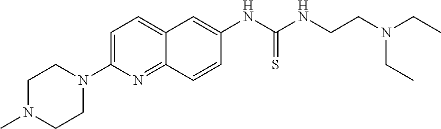 [SMILES: CCN(CC)CCNC(=S)Nc1ccc2nc(N3CCN(C)CC3)ccc2c1]

1-(2-(diethylamino)ethyl)-3-(2-(4-methylpiperazin-1-yl)quinolin-6-yl)thiourea. This compound was prepared according to synthetic Scheme 18. LC-MS, (M+H)⁺@ 401.6.

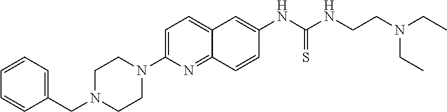 [SMILES: CCN(CC)CCNC(=S)Nc1ccc2nc(N3CCN(Cc4ccccc4)CC3)ccc2c1]

1-(2-(4-benzylpiperazin-1-yl)quinolin-6-yl)-3-(2-(diethylamino)ethyl)thiourea. This compound was prepared according to synthetic Scheme 18. LC-MS, (M+H)⁺@ 477.7; ¹H NMR (δ, ppm, CD₃OD) 8.24-2.15 (m, 1H), 7.87-7.81 (m, 1H), 7.81-7.74 (m, 1H), 7.70-7.62 (m, 1H), 7.62-7.50 (m, 5H), 7.40-7.32 (m, 1H), 5.06-4.80 (m, 6H), 4.42 (s, 2H), 4.24-3.96 (m, 4H), 3.50-3.34 (m, 6H), 1.46-1.28 (m, 6H).

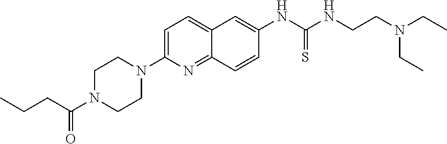 [SMILES: CCCC(=O)N1CCN(c2ccc3cc(NC(=S)NCCN(CC)CC)ccc3n2)CC1]

1-(2-(4-butyrylpiperazin-1-yl)quinolin-6-yl)-3-(2-(diethylamino)ethyl)thiourea. This compound was prepared according to synthetic Scheme 18. LC-MS, (M+H)⁺ @ 457.7; ¹H NMR (δ, ppm, CD₃OD) 8.42-8.34 (m, 1H), 8.09-8.03 (m, 1H), 7.94-7.86 (m, 2H), 7.53-7.45 (m, 1H), 4.07-3.92 (m, 6H), 3.92-3.84 (m, 4H), 3.50-3.41 (m, 2H), 3.40-3.20 (m, 4H), 2.50-2.40 (m, 2H), 1.76-1.60 (m, 2H), 1.42-1.32 (m, 6H), 1.05-0.96 (m, 3H).

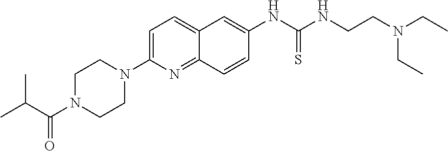 [SMILES: CCN(CC)CCNC(=S)Nc1ccc2nc(N3CCN(C(=O)C(C)C)CC3)ccc2c1]

1-(2-(diethylamino)ethyl)-3-(2-(4-isobutyrylpiperazin-1-yl)quinolin-6-yl)thiourea. This compound was prepared according to synthetic Scheme 18. LC-MS, (M+H)⁺@ 457.7; ¹H NMR (δ, ppm, CD₃OD) 8.40-8.33 (m, 1H), 8.07-8.01 (m, 1H), 7.92-7.82 (m, 2H), 7.52-7.44 (m, 1H), 4.08-3.99 (m, 4H), 3.99-3.90 (m, 4H), 3.90-3.82 (m, 2H), 3.49-3.41 (m, 2H), 3.40-3.20 (m, 4H), 3.10-2.95 (m, 1H), 1.42-1.32 (m, 6H), 1.20-1.11 (m, 6H)

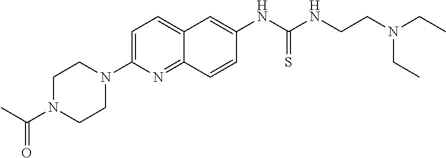 [SMILES: CCN(CC)CCNC(=S)Nc1ccc2nc(N3CCN(C(C)=O)CC3)ccc2c1]

1-(2-(4-acetylpiperazin-1-yl)quinolin-6-yl)-3-(2-(diethylamino)ethyl)thiourea. This compound was prepared according to synthetic Scheme 18. LC-MS, (M+H)⁺@ 429.6; ¹H NMR (δ, ppm, CD₃OD) 8.40-8.32 (m, 1H), 8.06-8.00 (m, 1H), 7.92-7.82 (m, 2H), 7.52-7.44 (m, 1H), 4.08-3.99 (m, 4H), 3.99-3.92 (m, 2H), 3.92-3.82 (m, 4H), 3.50-3.42 (m, 2H), 3.42-2.30 (m, 4H), 2.19 (s, 3H), 1.44-1.32 (m, 6H)

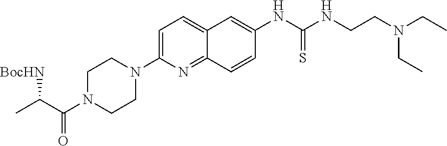 [SMILES: CCN(CC)CCNC(=S)Nc1ccc2nc(N3CCN(C(=O)[C@H](C)NC(=O)OC(C)(C)C)CC3)ccc2c1]

(S)-tert-butyl (1-(4-(6-(3-(2-(diethylamino)ethyl)thioureido)quinolin-2-yl)piperazin-1-yl)-1-oxopropan-2-yl)carbamate. This compound was prepared according to synthetic Scheme 18. LC-MS, (M+H)⁺@ 559.8.

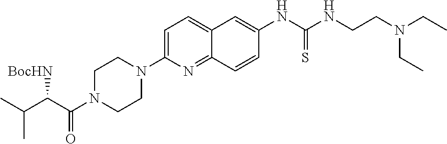 [SMILES: CCN(CC)CCNC(=S)Nc1ccc2nc(N3CCN(C(=O)[C@@H](NC(=O)OC(C)(C)C)C(C)C)CC3)ccc2c1]

(S)-tert-butyl (1-(4-(6-(3-(2-(diethylamino)ethyl)thioureido)quinolin-2-yl)piperazin-1-yl)-3-methyl-1-oxobutan-2-yl)carbamate. This compound was prepared according to synthetic Scheme 18. LC-MS, (M+H)⁺ @ 586.9.

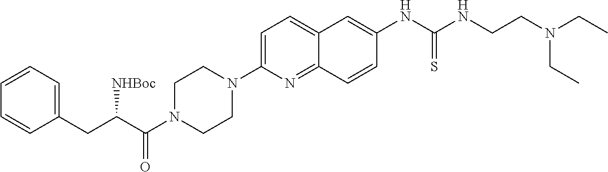 [SMILES: CCN(CC)CCNC(=S)Nc1ccc2nc(N3CCN(C(=O)[C@H](Cc4ccccc4)NC(=O)OC(C)(C)C)CC3)ccc2c1]

(S)-tert-butyl (1-(4-(6-(3-(2-(diethylamino)ethyl)thioureido)quinolin-2-yl)piperazin-1-yl)-1-oxo-3-phenylpropan-2-yl)carbamate. This compound was prepared according to synthetic Scheme 18. LC-MS, (M+H)⁺ @ 634.9.

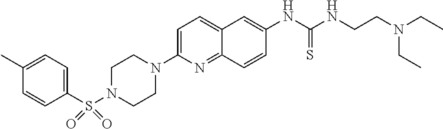 [SMILES: CCN(CC)CCNC(=S)Nc1ccc2nc(N3CCN(S(=O)(=O)c4ccc(C)cc4)CC3)ccc2c1]

1-(2-(diethylamino)ethyl)-3-(2-(4-tosylpiperazin-1-yl)quinolin-6-yl)thiourea. This compound was prepared according to synthetic Scheme 18. LC-MS, (M+H)⁺ @ 541.7; ¹H NMR (δ, ppm, CD₃OD) 8.26-8.20 (m, 1H), 7.92-7.88 (m, 1H), 7.81-7.68 (m, 4H), 7.46-7.40 (m, 2H), 7.40-7.32 (m, 1H), 4.05-3.90 (m, 6H), 3.46-3.38 (m, 2H), 3.28-3.14 (m, 6H), 2.41 (s, 3H), 1.42-1.32 (m, 6H).

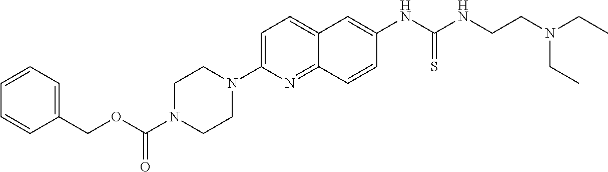 [SMILES: CCN(CC)CCNC(=S)Nc1ccc2nc(N3CCN(C(=O)OCc4ccccc4)CC3)ccc2c1]

Benzyl 4-(6-(3-(2-(diethylamino)ethyl)thioureido)quinolin-2-yl)piperazine-1-carboxylate. This compound was prepared according to synthetic Scheme 18. LC-MS, (M+H)⁺@ 521.8.

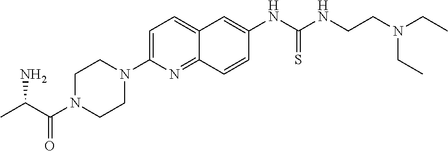 [SMILES: CCN(CC)CCNC(=S)Nc1ccc2nc(N3CCN(C(=O)[C@H](C)N)CC3)ccc2c1]

(S)-1-(2-(4-(2-aminopropanoyl)piperazin-1-yl)quinolin-6-yl)-3-(2-(diethylamino)ethyl)thiourea. This compound was prepared according to synthetic Scheme 18. LC-MS, (M+H)⁺@ 458.7; ¹H NMR (δ, ppm, CD₃OD) 8.50-8.40 (m, 1H), 8.12 (s, 1H), 8.06-7.97 (m, 1H), 7.97-7.88 (m, 1H), 7.60-7.50 (m, 1H), 4.60-4.50 (m, 1H), 4.20-3.80 (m, 10H), 3.52-3.42 (m, 2H), 3.42-3.20 (m, 4H), 1.58-1.46 (m, 3H), 1.46-1.32 (m, 6H).

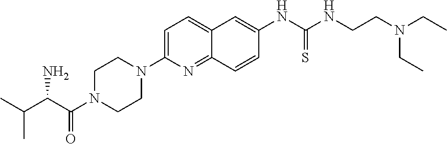 [SMILES: CCN(CC)CCNC(=S)Nc1ccc2nc(N3CCN(C(=O)[C@@H](N)C(C)C)CC3)ccc2c1]

(S)-1-(2-(4-(2-amino-3-methylbutanoyl)piperazin-1-yl)quinolin-6-yl)-3-(2-(diethylamino)ethyl)thiourea. This compound was prepared according to synthetic Scheme 18. LC-MS, (M+H)⁺@ 486.8; ¹H NMR (δ, ppm, CD₃OD) 8.48-8.41 (m, 1H), 8.14-8.08 (m, 1H), 8.05-7.98 (m, 1H), 7.95-7.88 (m, 1H), 7.58-7.50 (m, 1H), 4.48-4.40 (m, 1H), 4.22-3.75 (m, 10H), 3.50-3.42 (m, 2H), 3.42-3.20 (m, 4H), 2.34-2.18 (m, 1H), 1.44-1.34 (m, 6H), 1.20-1.11 (m, 3H), 1.11-1.02 (m, 3H).

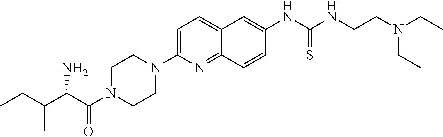 [SMILES: CCC(C)[C@H](N)C(=O)N1CCN(c2ccc3cc(NC(=S)NCCN(CC)CC)ccc3n2)CC1]

1-(2-(4-((2S)-2-amino-3-methylpentanoyl)piperazin-1-yl)quinolin-6-yl)-3-(2-(diethylamino)ethyl)thiourea. This compound was prepared according to synthetic Scheme 18. LC-MS, (M+H)⁺@ 500.8; ¹H NMR (δ, ppm, CD₃OD) 8.49-8.40 (m, 1H), 8.11 (s, 1H), 8.04-7.96 (m, 1H), 7.96-7.88 (m, 1H), 7.58-7.50 (m, 1H), 4.50-4.42 (m, 1H), 4.20-3.96 (m, 10H), 3.50-3.20 (m, 6H), 2.06-1.94 (m, 1H), 1.70-1.48 (m, 2H), 1.46-1.34 (m, 6H), 1.18-1.10 (m, 3H), 1.10-0.96 (m, 3H).

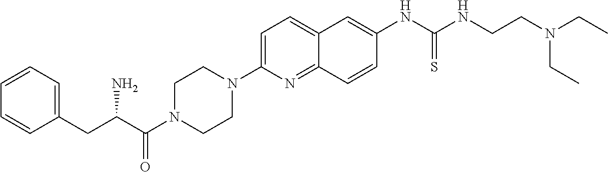 [SMILES: CCN(CC)CCNC(=S)Nc1ccc2nc(N3CCN(C(=O)[C@@H](N)Cc4ccccc4)CC3)ccc2c1]

(S)-1-(2-(4-(2-amino-3-phenylpropanoyl)piperazin-1-yl)quinolin-6-yl)-3-(2-(diethylamino)ethyl)thiourea. This compound was prepared according to synthetic Scheme 18. LC-MS, (M+H)⁺@ 534.8; ¹H NMR (δ, ppm, CD₃OD) 8.44-8.38 (m, 1H), 8.12-8.06 (m, 1H), 8.00-7.86 (m, 2H), 7.46-7.26 (m, 6H), 4.82-4.70 (m, 2H), 4.10-3.92 (m, 4H), 4.92-3.60 (m, 7H), 3.50-3.42 (m, 2H), 3.27-3.10 (m, 4H), 1.44-1.34 (m, 6H).

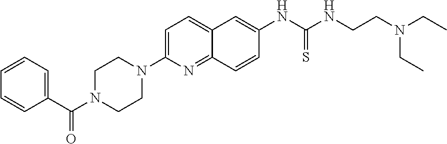 [SMILES: CCN(CC)CCNC(=S)Nc1ccc2nc(N3CCN(C(=O)c4ccccc4)CC3)ccc2c1]

1-(2-(4-benzoylpiperazin-1-yl)quinolin-6-yl)-3-(2-(diethylamino)ethyl)thiourea. This compound was prepared according to synthetic Scheme 18. LC-MS, (M+H)⁺@ 491.7; ¹H NMR (δ, ppm, CD₃OD) 8.41-8.34 (m, 1H), 8.06-8.02 (m, 1H), 7.90-7.85 (m, 2H), 7.57-7.49 (m, 6H), 4.16-3.72 (m, 10H), 3.50-3.40 (m, 2H), 3.40-3.20 (m, 4H), 1.44-1.33 (m, 6H).

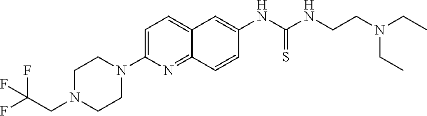 [SMILES: CCN(CC)CCNC(=S)Nc1ccc2nc(N3CCN(CC(F)(F)F)CC3)ccc2c1]

1-(2-(diethylamino)ethyl)-3-(2-(4-(2,2,2-trifluoroethyl)piperazin-1-yl)quinolin-6-yl)thiourea. This compound was prepared according to synthetic Scheme 18. LC-MS, (M+H)⁺@ 469.7; ¹H NMR (δ, ppm, CD₃OD) 8.39-8.32 (m, 1H), 8.06-8.00 (m, 1H), 7.90-7.84 (m, 2H), 7.55-7.48 (m, 1H), 4.06-3.99 (m, 2H), 3.99-3.90 (m, 4H), 3.49-3.40 (m, 2H), 3.28-3.16 (m, 4H), 3.00-2.90 (m, 4H), 1.42-1.33 (m, 6H).

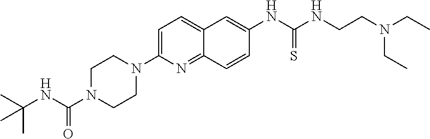 [SMILES: CCN(CC)CCNC(=S)Nc1ccc2nc(N3CCN(C(=O)NC(C)(C)C)CC3)ccc2c1]

N-(tert-butyl)-4-(6-(3-(2-(diethylamino)ethyl)thioureido)quinolin-2-yl)piperazine-1-carboxamide. This compound was prepared according to synthetic Scheme 18. LC-MS, (M+H)⁺@ 486.7; ¹H NMR (δ, ppm, CD₃OD) 8.40-8.33 (m, 1H), 8.06-8.02 (m, 1H), 7.92-7.84 (m, 2H), 7.54-7.46 (m, 1H), 4.07-3.90 (m, 6H), 3.72-3.62 (m, 4H), 3.49-3.41 (m, 2H), 3.41-3.20 (m, 4H), 1.42-1.32 (m, 15H).

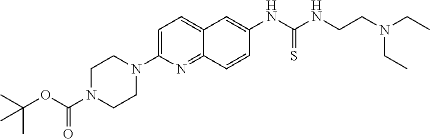 [SMILES: CCN(CC)CCNC(=S)Nc1ccc2nc(N3CCN(C(=O)OC(C)(C)C)CC3)ccc2c1]

tert-butyl 4-(6-(3-(2-(diethylamino)ethyl)thioureido)quinolin-2-yl)piperazine-1-carboxylate. This compound was prepared according to synthetic Scheme 18. LC-MS, (M+H)⁺@ 487.7; ¹H NMR (δ, ppm, CD₃OD) 8.40-8.32 (m, 1H), 8.06-8.02 (m, 1H), 7.90-7.84 (m, 2H), 7.53-7.46 (m, 1H), 4.06-3.90 (m, 6H), 3.76-3.66 (m, 4H), 3.49-3.40 (m, 2H), 3.40-3.20 (m, 4H), 1.52-1.46 (m, 9H), 1.41-1.32 (m, 6H).

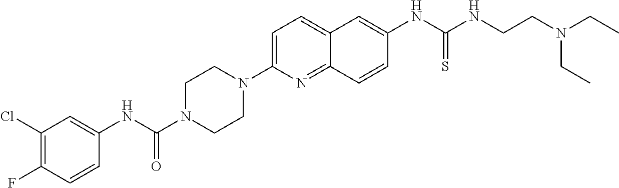 [SMILES: CCN(CC)CCNC(=S)Nc1ccc2nc(N3CCN(C(=O)Nc4ccc(F)c(Cl)c4)CC3)ccc2c1]

N-(3-chloro-4-fluorophenyl)-4-(6-(3-(2-(diethylamino)ethyl)thioureido)quinolin-2-yl)piperazine-1-carboxamide. This compound was prepared according to synthetic Scheme 18. LC-MS, (M+H)⁺@ 558.7; ¹H NMR (δ, ppm, CD₃OD) 8.38-8.30 (m, 1H), 8.03-7.96 (m, 1H), 7.92-7.80 (m, 2H), 7.66-7.60 (m, 1H), 7.52-7.45 (m, 1H), 7.36-7.28 (m, 1H), 7.21-7.12 (m, 1H), 4.07-3.98 (m, 6H), 3.88-3.80 (m, 4H), 3.48-3.40 (m, 2H), 3.40-3.20 (m, 4H), 1.42-1.33 (m, 6H).

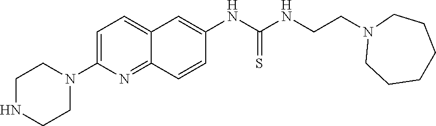 [SMILES: S=C(NCCN1CCCCCC1)Nc1ccc2nc(N3CCNCC3)ccc2c1]

1-(2-(azepan-1-yl)ethyl)-3-(2-(piperazin-1-yl)quinolin-6-yl)thiourea. This compound was prepared according to synthetic Scheme 18. LC-MS, (M+H)⁺@ 413.6; ¹H NMR (δ, ppm, CD₃OD) 8.52-8.44 (m, 1H), 8.16-8.09 (m, 1H), 8.04-7.87 (m, 2H), 7.60-7.50 (m, 1H), 4.26-4.14 (m, 4H), 4.10-4.00 (m, 2H), 3.70-3.40 (m, 10H), 2.06-1.84 (m, 4H), 1.82-1.66 (m, 4H).

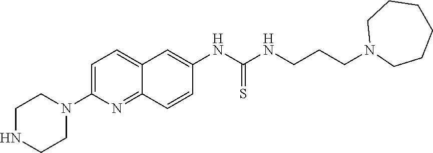 [SMILES: S=C(NCCCN1CCCCCC1)Nc1ccc2nc(N3CCNCC3)ccc2c1]

1-(3-(azepan-1-yl)propyl)-3-(2-(piperazin-1-yl)quinolin-6-yl)thiourea. This compound was prepared according to synthetic Scheme 18. LC-MS, (M+H)⁺ @ 427.7; ¹H NMR (δ, ppm, CD₃OD) 8.50-8.40 (m, 1H), 8.14-8.04 (m, 1H), 8.00-7.84 (m, 2H), 7.58-7.50 (m, 1H), 4.25-4.13 (m, 4H), 3.80-3.70 (m, 2H), 3.70-3.62 (m, 2H), 3.62-3.40 (m, 8H), 2.18-2.06 (m, 2H), 2.06-1.86 (m, 4H), 1.82-1.70 (m, 4H).

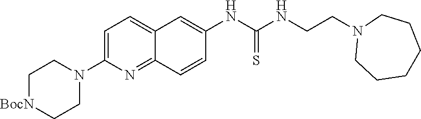 [SMILES: CC(C)(C)OC(=O)N1CCN(c2ccc3cc(NC(=S)NCCN4CCCCCC4)ccc3n2)CC1]

tert-butyl 4-(6-(3-(2-(azepan-1-yl)ethyl)thioureido)quinolin-2-yl)piperazine-1-carboxylate. This compound was prepared according to synthetic Scheme 18. LC-MS, (M+H)⁺@ 513.8.

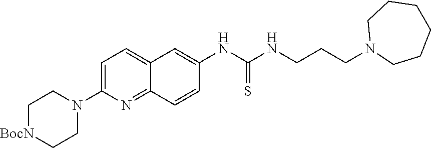 [SMILES: CC(C)(C)OC(=O)N1CCN(c2ccc3cc(NC(=S)NCCCN4CCCCCC4)ccc3n2)CC1]

tert-butyl 4-(6-(3-(3-(azepan-1-yl)propyl)thioureido)quinolin-2-yl)piperazine-1-carboxylate. This compound was prepared according to synthetic Scheme 18. LC-MS, (M+H)⁺@ 527.8.

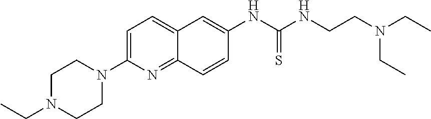 [SMILES: CCN(CC)CCNC(=S)Nc1ccc2nc(N3CCN(CC)CC3)ccc2c1]

1-(2-(diethylamino)ethyl)-3-(2-(4-ethylpiperazin-1-yl)quinolin-6-yl)thiourea. This compound was prepared according to synthetic Scheme 18. LC-MS, (M+H)⁺@ 415.7.

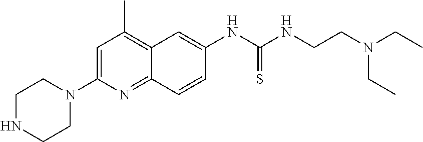 [SMILES: CCN(CC)CCNC(=S)Nc1ccc2nc(N3CCNCC3)cc(C)c2c1]

1-(2-(diethylamino)ethyl)-3-(4-methyl-2-(piperazin-1-yl)quinolin-6-yl)thiourea. This compound was prepared according to synthetic Scheme 18. LC-MS, (M+H)⁺@ 401.6.

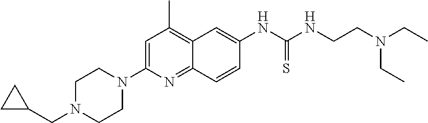 [SMILES: CCN(CC)CCNC(=S)Nc1ccc2nc(N3CCN(CC4CC4)CC3)cc(C)c2c1]

1-(2-(4-(cyclopropylmethyl)piperazin-1-yl)-4-methylquinolin-6-yl)-3-(2-(diethylamino)ethyl)thiourea. This compound was prepared according to synthetic Scheme 18. LC-MS, (M+H)⁺@ 455.8; ¹H NMR (δ, ppm, CDCl₃) 8.22 (s, 1H), 7.95-7.88 (m, 1H), 7.84-7.76 (m, 1H), 6.91 (s, 1H), 4.30-4.14 (m, 3H), 4.08-4.00 (m, 2H), 3.56-3.44 (m, 3H), 3.44-3.36 (m, 3H), 3.26-3.10 (m, 5H), 3.02-2.94 (m, 2H), 2.65 (s, 3H), 1.40-1.30 (m, 6H), 1.18-1.04 (m, 1H), 0.82-0.72 (m, 2H), 0.44-0.34 (m, 2H).

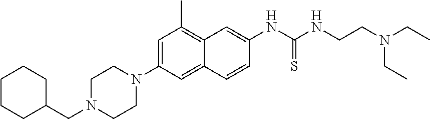 [SMILES: CCN(CC)CCNC(=S)Nc1ccc2cc(N3CCN(CC4CCCCC4)CC3)cc(C)c2c1]

1-(6-(4-(cyclohexylmethyl)piperazin-1-yl)-8-methylnaphthalen-2-yl)-3-(2-(diethylamino)ethyl)thiourea. This compound was prepared according to synthetic Scheme 18. LC-MS, (M+H)⁺@ 497.8.

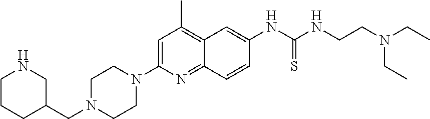 [SMILES: CCN(CC)CCNC(=S)Nc1ccc2nc(N3CCN(CC4CCCNC4)CC3)cc(C)c2c1]

1-(2-(diethylamino)ethyl)-3-(4-methyl-2-(4-(piperidin-3-ylmethyl)piperazin-1-yl)quinolin-6-yl)thiourea. This compound was prepared according to synthetic Scheme 18. LC-MS, (M+H)⁺@ 498.8; ¹H NMR (δ, ppm, CD₃OD) 8.36-8.32 (m, 1H), 8.05-7.98 (m, 1H), 7.98-7.92 (m, 1H), 7.52 (s, 1H), 4.08-4.02 (m, 2H), 3.78-3.67 (m, 4H), 3.67-3.62 (m, 6H), 3.60-3.54 (m, 2H), 3.50-3.40 (m, 3H), 3.40-3.30 (m, 3H), 3.02-2.82 (m, 2H), 2.78 (s, 3H), 2.58-2.44 (m, 1H), 2.16-1.80 (m, 4H), 1.42-1.34 (m, 6H).

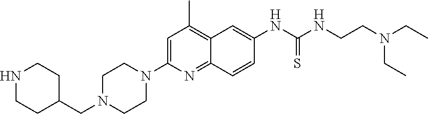 [SMILES: CCN(CC)CCNC(=S)Nc1ccc2nc(N3CCN(CC4CCNCC4)CC3)cc(C)c2c1]

1-(2-(diethylamino)ethyl)-3-(4-methyl-2-(4-(piperidin-4-ylmethyl)piperazin-1-yl)quinolin-6-yl)thiourea. This compound was prepared according to synthetic Scheme 18. LC-MS, (M+H)⁺@ 498.8; ¹H NMR (δ, ppm, CD₃OD) 8.38-8.32 (m, 1H), 8.06-7.99 (m, 1H), 7.99-7.91 (m, 1H), 7.53 (s, 1H), 4.10-4.00 (m, 3H), 3.98-3.80 (m, 2H), 3.77-3.71 (m, 1H), 3.70-3.62 (m, 4H), 3.62-3.52 (m, 2H), 3.52-3.40 (m, 6H), 3.18-3.00 (m, 4H), 2.78 (s, 3H), 2.46-2.30 (m, 1H), 2.28-2.16 (m, 2H), 1.68-1.50 (m, 2H), 1.44-1.34 (m, 6H).

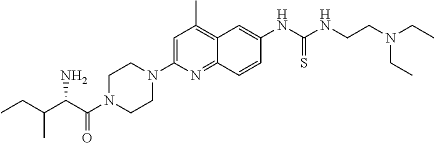 [SMILES: CCC(C)[C@H](N)C(=O)N1CCN(c2cc(C)c3cc(NC(=S)NCCN(CC)CC)ccc3n2)CC1]

1-(2-(4-((2S)-2-amino-3-methylpentanoyl)piperazin-1-yl)-4-methylquinolin-6-yl)-3-(2-(diethylamino)ethyl)thiourea. This compound was prepared according to synthetic Scheme 18. LC-MS, (M+H)⁺@ 514.9.

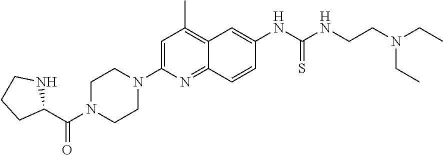 [SMILES: CCN(CC)CCNC(=S)Nc1ccc2nc(N3CCN(C(=O)[C@@H]4CCCN4)CC3)cc(C)c2c1]

1-(2-(diethylamino)ethyl)-3-(4-methyl-2-(4-(pyrrolidine-2-carbonyl)piperazin-1-yl)quinolin-6-yl)thiourea. This compound was prepared according to synthetic Scheme 18. LC-MS, (M+H)⁺@ 498.7.

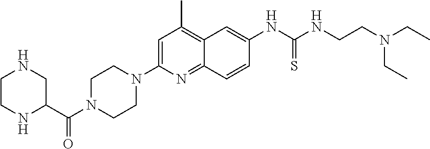 [SMILES: CCN(CC)CCNC(=S)Nc1ccc2nc(N3CCN(C(=O)C4CNCCN4)CC3)cc(C)c2c1]

1-(2-(diethylamino)ethyl)-3-(4-methyl-2-(4-(piperazine-2-carbonyl)piperazin-1-yl)quinolin-6-yl)thiourea. This compound was prepared according to synthetic Scheme 18. LC-MS, (M+H)⁺@ 513.8.

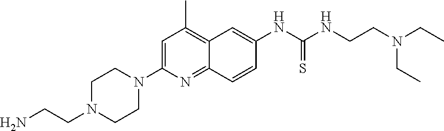 [SMILES: CCN(CC)CCNC(=S)Nc1ccc2nc(N3CCN(CCN)CC3)cc(C)c2c1]

1-(2-(4-(2-aminoethyl)piperazin-1-yl)-4-methylquinolin-6-yl)-3-(2-(diethylamino)ethyl)thiourea. This compound was prepared according to synthetic Scheme 18. LC-MS, (M+H)⁺@ 444.7.

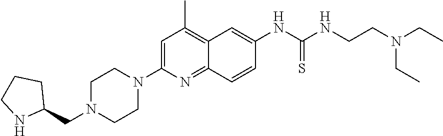 [SMILES: CCN(CC)CCNC(=S)Nc1ccc2nc(N3CCN(C[C@@H]4CCCN4)CC3)cc(C)c2c1]

(S)-1-(2-(diethylamino)ethyl)-3-(4-methyl-2-(4-(pyrrolidin-2-ylmethyl)piperazin-1-yl)quinolin-6-yl)thiourea. This compound was prepared according to synthetic Scheme 18. LC-MS, (M+H)⁺@ 484.8.

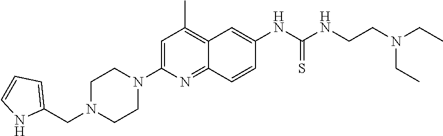 [SMILES: CCN(CC)CCNC(=S)Nc1ccc2nc(N3CCN(Cc4ccc[nH]4)CC3)cc(C)c2c1]

1-(2-(4-((1H-pyrrol-2-yl)methyl)piperazin-1-yl)-4-methylquinolin-6-yl)-3-(2-(diethylamino)ethyl)thiourea. This compound was prepared according to synthetic Scheme 18. LC-MS, (M+H)⁺@ 480.8; ¹H NMR (δ, ppm, CDCl₃) 8.08-7.98 (m, 1H), 7.76-7.60 (m, 2H), 6.90-6.76 (m, 2H), 6.24-6.14 (m, 1H), 6.12-6.02 (m, 1H), 4.24-4.14 (m, 2H), 4.00-3.98 (m, 2H), 3.30-3.21 (m, 8H), 3.20-3.08 (m, 6H), 2.58-2.51 (m, 3H), 1.33-1.23 (m, 6H).

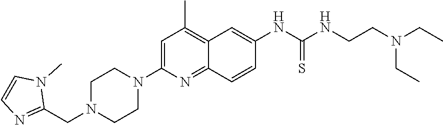 [SMILES: CCN(CC)CCNC(=S)Nc1ccc2nc(N3CCN(Cc4nccn4C)CC3)cc(C)c2c1]

1-(2-(diethylamino)ethyl)-3-(4-methyl-2-(4-((1-methyl-1H-imidazol-2-yl)methyl)piperazin-1-yl)quinolin-6-yl)thiourea. This compound was prepared according to synthetic Scheme 18. LC-MS, (M+H)⁺@ 495.8.

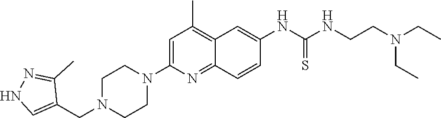 [SMILES: CCN(CC)CCNC(=S)Nc1ccc2nc(N3CCN(Cc4c[nH]nc4C)CC3)cc(C)c2c1]

1-(2-(diethylamino)ethyl)-3-(4-methyl-2-(4-((3-methyl-1H-pyrazol-4-yl)methyl)piperazin-1-yl)quinolin-6-yl)thiourea. This compound was prepared according to synthetic Scheme 18. LC-MS, (M+H)⁺ @ 495.8; ¹H NMR (δ, ppm, CDCl₃) 8.16-8.10 (m, 1H), 7.86-7.80 (m, 1H), 7.76-7.70 (m, 1H), 7.62-7.56 (m, 1H), 6.90 (s, 1H), 4.14-4.06 (m, 4H), 4.04-3.98 (m, 2H), 3.42-3.34 (m, 4H), 3.34-3.24 (m, 4H), 3.24-3.13 (m, 4H), 2.70-2.50 (m, 3H), 2.26 (s, 3H), 1.38-1.29 (m, 6H).

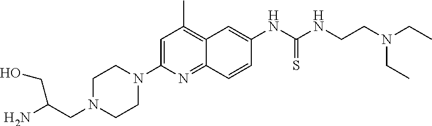 [SMILES: CCN(CC)CCNC(=S)Nc1ccc2nc(N3CCN(CC(N)CO)CC3)cc(C)c2c1]

1-(2-(4-(2-amino-3-hydroxypropyl)piperazin-1-yl)-4-methylquinolin-6-yl)-3-(2-(diethylamino)ethyl)thiourea. This compound was prepared according to synthetic Scheme 18. LC-MS, (M+H)⁺@ 474.8.

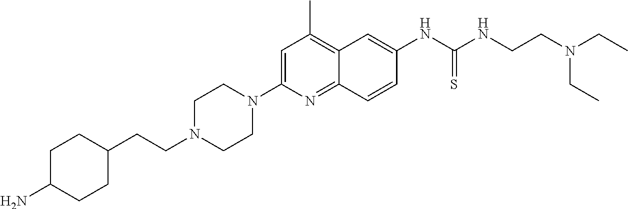 [SMILES: CCN(CC)CCNC(=S)Nc1ccc2nc(N3CCN(CCC4CCC(N)CC4)CC3)cc(C)c2c1]

1-(2-(4-(2-(4-aminocyclohexyl)ethyl)piperazin-1-yl)-4-methylquinolin-6-yl)-3-(2-(diethylamino)ethyl)thiourea. This compound was prepared according to synthetic Scheme 18. LC-MS, (M+H)⁺@ 526.9.

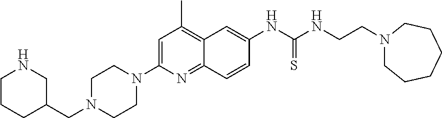 [SMILES: Cc1cc(N2CCN(CC3CCCNC3)CC2)nc2ccc(NC(=S)NCCN3CCCCCC3)cc12]

1-(2-(azepan-1-yl)ethyl)-3-(4-methyl-2-(4-(piperidin-3-ylmethyl)piperazin-1-yl)quinolin-6-yl)thiourea. This compound was prepared according to synthetic Scheme 18. LC-MS, (M+H)⁺@ 524.9.

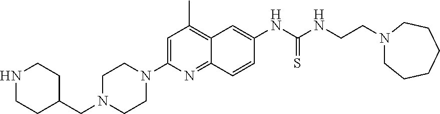 [SMILES: Cc1cc(N2CCN(CC3CCNCC3)CC2)nc2ccc(NC(=S)NCCN3CCCCCC3)cc12]

1-(2-(azepan-1-yl)ethyl)-3-(4-methyl-2-(4-(piperidin-4-ylmethyl)piperazin-1-yl)quinolin-6-yl)thiourea. This compound was prepared according to synthetic Scheme 18. LC-MS, (M+H)⁺@ 524.9.

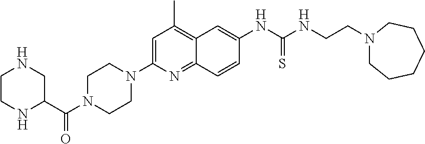 [SMILES: Cc1cc(N2CCN(C(=O)C3CNCCN3)CC2)nc2ccc(NC(=S)NCCN3CCCCCC3)cc12]

1-(2-(azepan-1-yl)ethyl)-3-(4-methyl-2-(4-(piperazine-2-carbonyl)piperazin-1-yl)quinolin-6-yl)thiourea. This compound was prepared according to synthetic Scheme 18. LC-MS, (M+H)⁺@ 537.9.

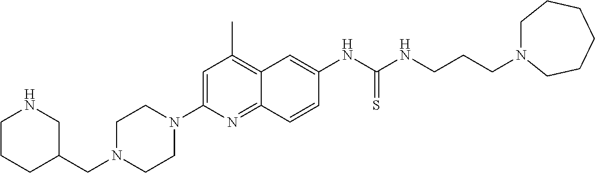 [SMILES: Cc1cc(N2CCN(CC3CCCNC3)CC2)nc2ccc(NC(=S)NCCCN3CCCCCC3)cc12]

1-(3-(azepan-1-yl)propyl)-3-(4-methyl-2-(4-(piperidin-3-ylmethyl)piperazin-1-yl)quinolin-6-yl)thiourea. This compound was prepared according to synthetic Scheme 18. LC-MS, (M+H)⁺@ 538.9.

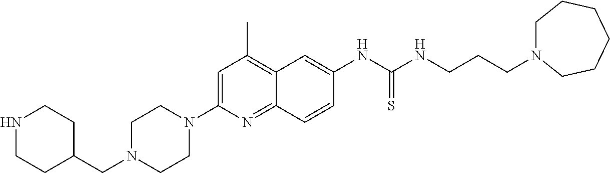 [SMILES: Cc1cc(N2CCN(CC3CCNCC3)CC2)nc2ccc(NC(=S)NCCCN3CCCCCC3)cc12]

1-(3-(azepan-1-yl)propyl)-3-(4-methyl-2-(4-(piperidin-4-ylmethyl)piperazin-1-yl)quinolin-6-yl)thiourea. This compound was prepared according to synthetic Scheme 18. LC-MS, (M+H)⁺@ 538.9.

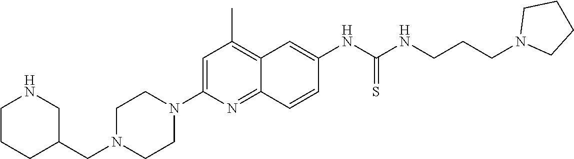 [SMILES: Cc1cc(N2CCN(CC3CCCNC3)CC2)nc2ccc(NC(=S)NCCCN3CCCC3)cc12]

1-(4-methyl-2-(4-(piperidin-3-ylmethyl)piperazin-1-yl)quinolin-6-yl)-3-(3-(pyrrolidin-1-yl)propyl)thiourea. This compound was prepared according to synthetic Scheme 18. LC-MS, (M+H)⁺@ 510.9.

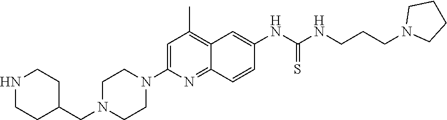 [SMILES: Cc1cc(N2CCN(CC3CCNCC3)CC2)nc2ccc(NC(=S)NCCCN3CCCC3)cc12]

1-(4-methyl-2-(4-(piperidin-4-ylmethyl)piperazin-1-yl)quinolin-6-yl)-3-(3-(pyrrolidin-1-yl)propyl)thiourea. This compound was prepared according to synthetic Scheme 18. LC-MS, (M+H)⁺@ 510.9.

### Procedure for Compounds 0108-0112

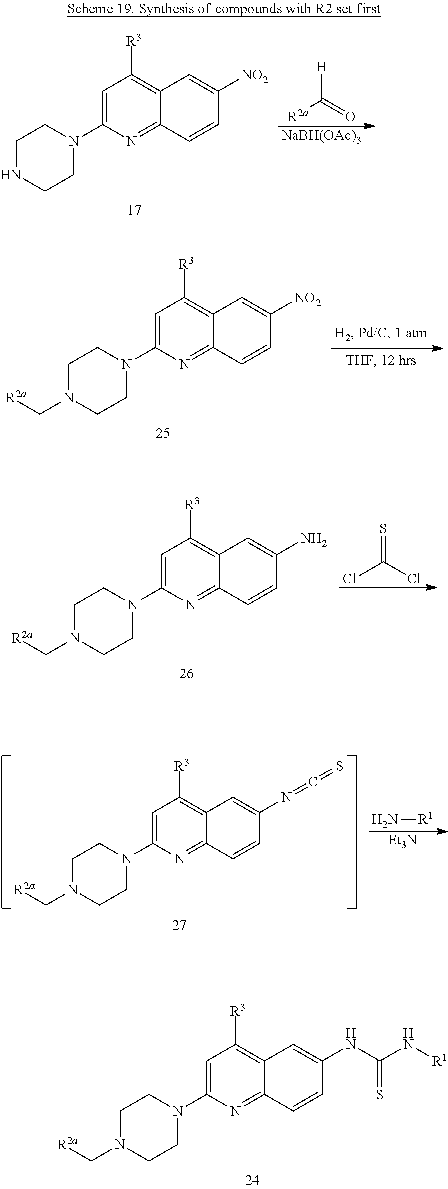

The starting material 17 in 1,2-dichloroethane (DCE) was added dropwise to a stirring reaction of aldehyde (1 eq.) and NaBH(OAc)₃ (3 eq.) in DCE. After the reaction stirred overnight, it was quenched with saturated aqueous NaHCO₃ and stirred vigorously for several hours. The phases were separated, and the aqueous phase was extracted with DCM twice. The organic phases were combined and concentrated. Then, the intermediate 25 was dissolved in THE and reduced under Pd/C at 1.5 atm H₂ and left overnight. The mixture was filtered through celite and the filtrate was collected and concentrated. The intermediate 26 was dissolved in DCM, and NaHCO₃(4 eq.) dissolved in an equal amount of water was added to it. The reaction was put in an ice bath, and thiophosgene (1.5 eq.) was added dropwise. After 10 min, the reaction was taken off the ice bath and allowed to warm to room temperature, at which it was stirred overnight. The organic and aqueous phases were separated if still immiscible at the end of the reaction, and the organic layer was washed with brine and concentrated down; if the phases were miscible, then the whole reaction mixture was concentrated down and used for the next step. The resulting intermediate was re-dissolved in DCM, and Et₃N and an amine (1.2 eq.) was added. Once the reaction was concluded, it was diluted with EtOAc, washed with brine, and concentrated down. Then it was dissolved in MeOH to be purified by HPLC in 0.1% TFA H₂O:MeCN. Any Boc present from the aldehyde or amine was cleaved off by stirring in 1:1 4M HCl in dioxane and MeOH for 2 hours. Afterwards, it was concentrated and co-evaporated with MeOH 3 times to get rid of excess HCl.

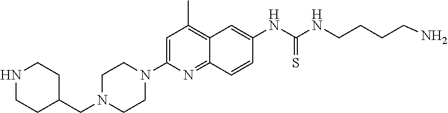 [SMILES: Cc1cc(N2CCN(CC3CCNCC3)CC2)nc2ccc(NC(=S)NCCCCN)cc12]

1-(4-aminobutyl)-3-(4-methyl-2-(4-(piperidin-4-ylmethyl)piperazin-1-yl)quinolin-6-yl)thiourea. This compound was prepared according to synthetic Scheme 19. LC-MS, (M+H)⁺@ 470.8.

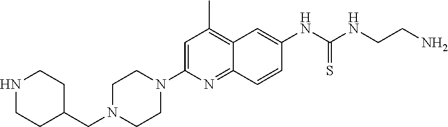 [SMILES: Cc1cc(N2CCN(CC3CCNCC3)CC2)nc2ccc(NC(=S)NCCN)cc12]

1-(2-aminoethyl)-3-(4-methyl-2-(4-(piperidin-4-ylmethyl)piperazin-1-yl)quinolin-6-yl)thiourea. This compound was prepared according to synthetic Scheme 19. LC-MS, (M+H)⁺@ 442.8.

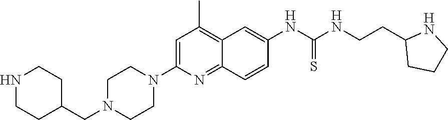 [SMILES: Cc1cc(N2CCN(CC3CCNCC3)CC2)nc2ccc(NC(=S)NCCC3CCCN3)cc12]

1-(4-methyl-2-(4-(piperidin-4-ylmethyl)piperazin-1-yl)quinolin-6-yl)-3-(2-(pyrrolidin-2-yl)ethyl)thiourea. This compound was prepared according to synthetic Scheme 19. LC-MS, (M+H)⁺@ 496.8.

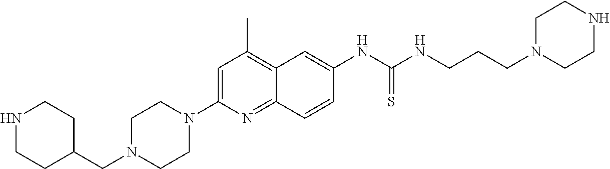 [SMILES: Cc1cc(N2CCN(CC3CCNCC3)CC2)nc2ccc(NC(=S)NCCCN3CCNCC3)cc12]

1-(4-methyl-2-(4-(piperidin-4-ylmethyl)piperazin-1-yl)quinolin-6-yl)-3-(3-(piperazin-1-yl)propyl)thiourea. This compound was prepared according to synthetic Scheme 19. LC-MS, (M+H)⁺@ 525.9.

### Procedure for Starting Material 2

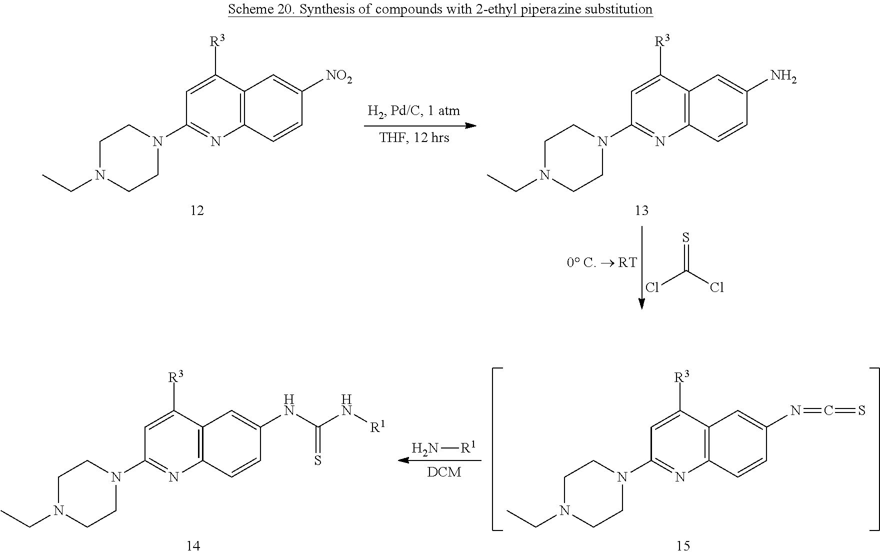

The starting material 12 was reduced under Pd/C at around 1.0-1.5 atm H₂ and left overnight. The mixture was then filtered through celite and the filtrate was collected and concentrated to provide the amine 13, which was dissolved in DCM, and 4 eq. NaHCO₃ dissolved in an equal amount of water was added to it. The reaction was put in an ice bath to cool down to 0° C., and thiophosgene (1.5 eq.) in DCM was added dropwise. After 10 min, the reaction was taken off the ice bath and allowed to proceed at room temperature overnight. The organic and aqueous phases were then separated, and the organic layer was washed with brine, dried with solid Na₂SO₄, and concentrated. The thio-isocyanate intermediate 14 was redissolved in DCM, and Et₃N and an amine (R¹NH₂, 1.2 eq.) was added. After overnight, it was diluted in EtOAc, washed with brine, and concentrated. Then it was purified by HPLC in 0.1% TFA of H₂O. MeCN to afford 15.

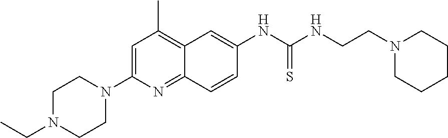 [SMILES: CCN1CCN(c2cc(C)c3cc(NC(=S)NCCN4CCCCC4)ccc3n2)CC1]

1-(2-(4-ethylpiperazin-1-yl)-4-methylquinolin-6-yl)-3-(2-(piperidin-1-yl)ethyl)thiourea. This compound was prepared according to synthetic Scheme 20. LC-MS, (M+H)⁺@ 441.6.

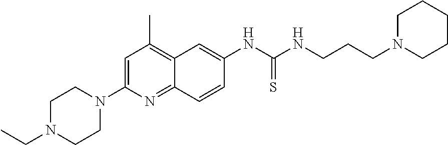 [SMILES: CCN1CCN(c2cc(C)c3cc(NC(=S)NCCCN4CCCCC4)ccc3n2)CC1]

1-(2-(4-ethylpiperazin-1-yl)-4-methylquinolin-6-yl)-3-(3-(piperidin-1-yl)propyl)thiourea. This compound was prepared according to synthetic Scheme 20. LC-MS, (M+H)⁺@ 455.7.

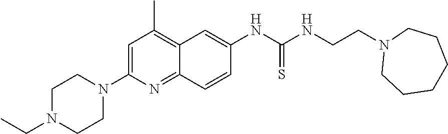 [SMILES: CCN1CCN(c2cc(C)c3cc(NC(=S)NCCN4CCCCCC4)ccc3n2)CC1]

1-(2-(azepan-1-yl)ethyl)-3-(2-(4-ethylpiperazin-1-yl)-4-methylquinolin-6-yl)thiourea. This compound was prepared according to synthetic Scheme 20. LC-MS, (M+H)⁺@ 455.7.

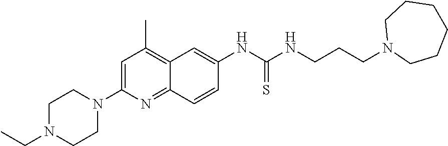 [SMILES: CCN1CCN(c2cc(C)c3cc(NC(=S)NCCCN4CCCCCC4)ccc3n2)CC1]

1-(3-(azepan-1-yl)propyl)-3-(2-(4-ethylpiperazin-1-yl)-4-methylquinolin-6-yl)thiourea. This compound was prepared according to synthetic Scheme 20. LC-MS, (M+H)⁺@ 469.7.

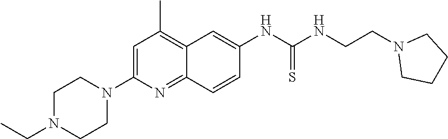 [SMILES: CCN1CCN(c2cc(C)c3cc(NC(=S)NCCN4CCCC4)ccc3n2)CC1]

1-(2-(4-ethylpiperazin-1-yl)-4-methylquinolin-6-yl)-3-(2-(pyrrolidin-1-yl)ethyl)thiourea. This compound was prepared according to synthetic Scheme 20. LC-MS, (M+H)⁺@ 427.7.

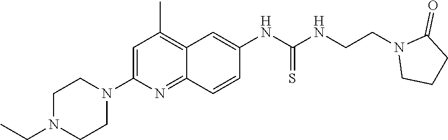 [SMILES: CCN1CCN(c2cc(C)c3cc(NC(=S)NCCN4CCCC4=O)ccc3n2)CC1]

1-(2-(4-ethylpiperazin-1-yl)-4-methylquinolin-6-yl)-3-(2-(2-oxopyrrolidin-1-yl)ethyl)thiourea. This compound was prepared according to synthetic Scheme 20. LC-MS, (M+H)⁺@ 441.7.

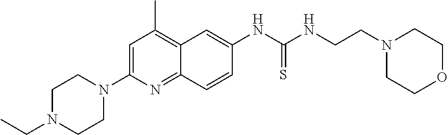 [SMILES: CCN1CCN(c2cc(C)c3cc(NC(=S)NCCN4CCOCC4)ccc3n2)CC1]

1-(2-(4-ethylpiperazin-1-yl)-4-methylquinolin-6-yl)-3-(2-morpholinoethyl)thiourea. This compound was prepared according to synthetic Scheme 20. LC-MS, (M+H)⁺@ 443.7.

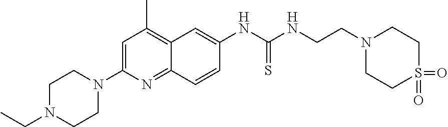 [SMILES: CCN1CCN(c2cc(C)c3cc(NC(=S)NCCN4CCS(=O)(=O)CC4)ccc3n2)CC1]

1-(2-(1,1-dioxidothiomorpholino)ethyl)-3-(2-(4-ethylpiperazin-1-yl)-4-methylquinolin-6-yl)thiourea. This compound was prepared according to synthetic Scheme 20. LC-MS, (M+H)⁺@ 491.7; ¹H NMR (δ, ppm, CD₃OD) 8.30-8.22 (m, 1H), 7.90-7.80 (m, 2H), 7.44-7.36 (m, 2H), 3.92-3.80 (m, 2H), 3.62-3.45 (m, 4H), 3.45-3.35 (m, 4H), 3.35-3.26 (m, 6H), 3.26-3.13 (m, 4H), 3.13-3.02 (m, 2H), 2.73 (s, 3H), 1.46-1.46 (m, 3H).

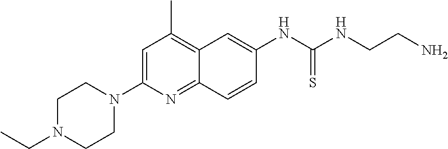 [SMILES: CCN1CCN(c2cc(C)c3cc(NC(=S)NCCN)ccc3n2)CC1]

1-(2-aminoethyl)-3-(2-(4-ethylpiperazin-1-yl)-4-methylquinolin-6-yl)thiourea. This compound was prepared according to synthetic Scheme 20. LC-MS, (M+H)⁺@ 373.6.

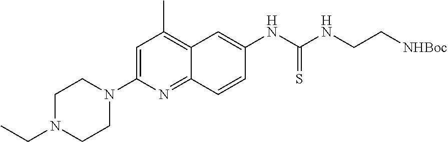 [SMILES: CCN1CCN(c2cc(C)c3cc(NC(=S)NCCNC(=O)OC(C)(C)C)ccc3n2)CC1]

tert-butyl (2-(3-(2-(4-ethylpiperazin-1-yl)-4-methylquinolin-6-yl)thioureido)ethyl)carbamate. This compound was prepared according to synthetic Scheme 20. LC-MS, (M+H)⁺@ 473.7.

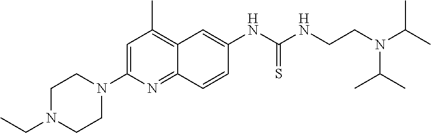 [SMILES: CCN1CCN(c2cc(C)c3cc(NC(=S)NCCN(C(C)C)C(C)C)ccc3n2)CC1]

1-(2-(diisopropylamino)ethyl)-3-(2-(4-ethylpiperazin-1-yl)-4-methylquinolin-6-yl)thiourea. This compound was prepared according to synthetic Scheme 20. LC-MS, (M+H)⁺@ 457.7.

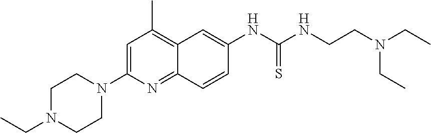 [SMILES: CCN(CC)CCNC(=S)Nc1ccc2nc(N3CCN(CC)CC3)cc(C)c2c1]

1-(4-(diethylamino)butyl)-3-(2-(4-ethylpiperazin-1-yl)-4-methylquinolin-6-yl)thiourea. This compound was prepared according to synthetic Scheme 20. LC-MS, (M+H)⁺@ 457.7.

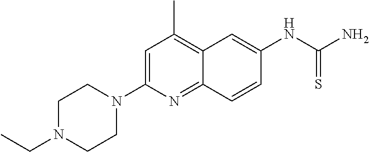 [SMILES: CCN1CCN(c2cc(C)c3cc(NC(N)=S)ccc3n2)CC1]

1-(2-(4-ethylpiperazin-1-yl)-4-methylquinolin-6-yl)thiourea. This compound was prepared according to synthetic Scheme 20. LC-MS, (M+H)⁺@ 330.5.

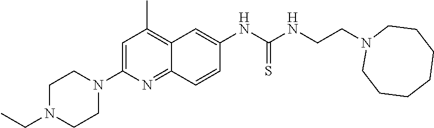 [SMILES: CCN1CCN(c2cc(C)c3cc(NC(=S)NCCN4CCCCCCC4)ccc3n2)CC1]

1-(2-(azocan-1-yl)ethyl)-3-(2-(4-ethylpiperazin-1-yl)-4-methylquinolin-6-yl)thiourea. This compound was prepared according to synthetic Scheme 20. LC-MS, (M+H)⁺@ 469.7.

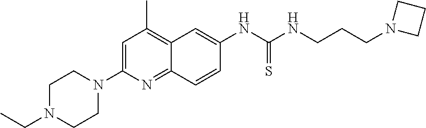 [SMILES: CCN1CCN(c2cc(C)c3cc(NC(=S)NCCCN4CCC4)ccc3n2)CC1]

1-(3-(azetidin-1-yl)propyl)-3-(2-(4-ethylpiperazin-1-yl)-4-methylquinolin-6-yl)thiourea. This compound was prepared according to synthetic Scheme 20. LC-MS, (M+H)⁺@ 427.7.

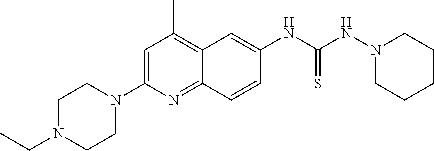 [SMILES: CCN1CCN(c2cc(C)c3cc(NC(=S)NN4CCCCC4)ccc3n2)CC1]

1-(2-(4-ethylpiperazin-1-yl)-4-methylquinolin-6-yl)-3-(piperidin-1-yl)thiourea. This compound was prepared according to synthetic Scheme 20. LC-MS, (M+H)⁺@ 413.7.

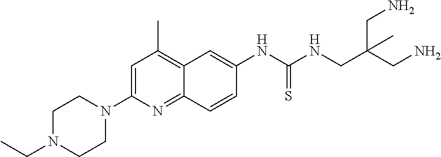 [SMILES: CCN1CCN(c2cc(C)c3cc(NC(=S)NCC(C)(CN)CN)ccc3n2)CC1]

1-(3-amino-2-(aminomethyl)-2-methylpropyl)-3-(2-(4-ethylpiperazin-1-yl)-4-methylquinolin-6-yl)thiourea. This compound was prepared according to synthetic Scheme 20. LC-MS, (M+H)⁺@ 430.7.

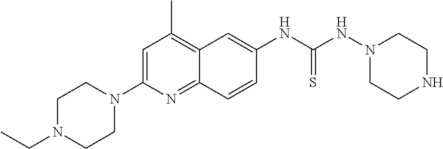 [SMILES: CCN1CCN(c2cc(C)c3cc(NC(=S)NN4CCNCC4)ccc3n2)CC1]

1-(2-(4-ethylpiperazin-1-yl)-4-methylquinolin-6-yl)-3-(2-(piperazin-1-yl)ethyl)thiourea. This compound was prepared according to synthetic Scheme 20. LC-MS, (M+H)⁺@ 442.7.

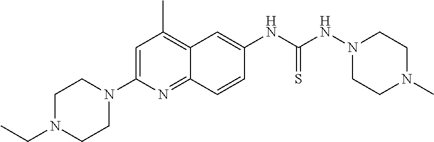 [SMILES: CCN1CCN(c2cc(C)c3cc(NC(=S)NN4CCN(C)CC4)ccc3n2)CC1]

1-(2-(4-ethylpiperazin-1-yl)-4-methylquinolin-6-yl)-3-(2-(4-methylpiperazin-1-yl)ethyl)thiourea. This compound was prepared according to synthetic Scheme 20. LC-MS, (M+H)⁺@ 456.7.

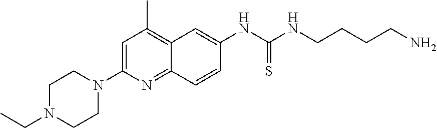 [SMILES: CCN1CCN(c2cc(C)c3cc(NC(=S)NCCCCN)ccc3n2)CC1]

1-(4-aminobutyl)-3-(2-(4-ethylpiperazin-1-yl)-4-methylquinolin-6-yl)thiourea. This compound was prepared according to synthetic Scheme 20. LC-MS, (M+H)⁺@ 401.6.

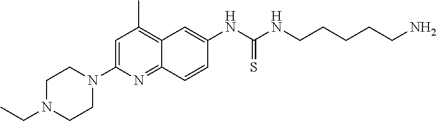 [SMILES: CCN1CCN(c2cc(C)c3cc(NC(=S)NCCCCCN)ccc3n2)CC1]

1-(5-aminopentyl)-3-(2-(4-ethylpiperazin-1-yl)-4-methylquinolin-6-yl)thiourea. This compound was prepared according to synthetic Scheme 20. LC-MS, (M+H)⁺@ 415.7.

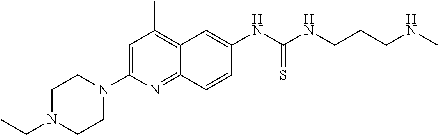 [SMILES: CCN1CCN(c2cc(C)c3cc(NC(=S)NCCCNC)ccc3n2)CC1]

1-(2-(4-ethylpiperazin-1-yl)-4-methylquinolin-6-yl)-3-(3-(methylamino)propyl)thiourea. This compound was prepared according to synthetic Scheme 20. LC-MS, (M+H)⁺@ 401.6.

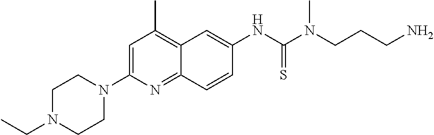 [SMILES: CCN1CCN(c2cc(C)c3cc(NC(=S)N(C)CCCN)ccc3n2)CC1]

1-(3-aminopropyl)-3-(2-(4-ethylpiperazin-1-yl)-4-methylquinolin-6-yl)-1-methylthiourea. This compound was prepared according to synthetic Scheme 20. LC-MS, (M+H)⁺@ 401.6.

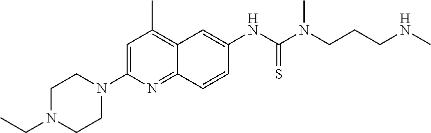 [SMILES: CCN1CCN(c2cc(C)c3cc(NC(=S)N(C)CCCNC)ccc3n2)CC1]

3-(2-(4-ethylpiperazin-1-yl)-4-methylquinolin-6-yl)-1-methyl-1-(3-(methylamino)propyl)thiourea. This compound was prepared according to synthetic Scheme 20. LC-MS, (M+H)⁺@ 415.7.

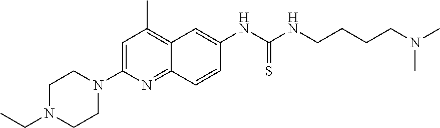 [SMILES: CCN1CCN(c2cc(C)c3cc(NC(=S)NCCCCN(C)C)ccc3n2)CC1]

1-(4-(dimethylamino)butyl)-3-(2-(4-ethylpiperazin-1-yl)-4-methylquinolin-6-yl)thiourea. This compound was prepared according to synthetic Scheme 20. LC-MS, (M+H)⁺@ 429.7; ¹H NMR (δ, ppm, CDCl₃) 8.26-8.20 (m, 1H), 7.81-7.75 (m, 1H), 7.72-7.65 (m, 1H), 6.94 (s, 1H), 3.68-3.58 (m, 2H), 3.40-3.26 (m, 8H), 3.15-3.00 (m, 4H), 2.80-2.74 (m, 6H), 2.60 (s, 3H), 1.86-1.73 (m, 2H), 1.73-1.60 (m, 2H), 1.38-1.28 (m, 3H).

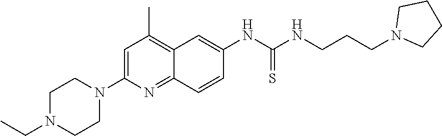 [SMILES: CCN1CCN(c2cc(C)c3cc(NC(=S)NCCCN4CCCC4)ccc3n2)CC1]

1-(2-(4-ethylpiperazin-1-yl)-4-methylquinolin-6-yl)-3-(3-(pyrrolidin-1-yl)propyl)thiourea. This compound was prepared according to synthetic Scheme 20. LC-MS, (M+H)⁺@ 441.7.

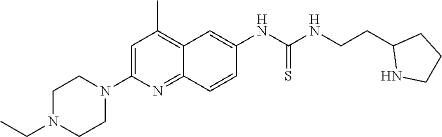 [SMILES: CCN1CCN(c2cc(C)c3cc(NC(=S)NCCC4CCCN4)ccc3n2)CC1]

1-(2-(4-ethylpiperazin-1-yl)-4-methylquinolin-6-yl)-3-(2-(pyrrolidin-2-yl)ethyl)thiourea. This compound was prepared according to synthetic Scheme 20. LC-MS, (M+H)⁺@ 427.6.

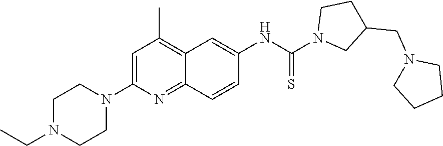 [SMILES: CCN1CCN(c2cc(C)c3cc(NC(=S)N4CCC(CN5CCCC5)C4)ccc3n2)CC1]

N-(2-(4-ethylpiperazin-1-yl)-4-methylquinolin-6-yl)-3-(pyrrolidin-1-ylmethyl)pyrrolidine-1-carbothioamide. This compound was prepared according to synthetic Scheme 20. LC-MS, (M+H)⁺@ 467.7; ¹H NMR (δ, ppm, CDCl₃) 8.02-7.98 (m, 1H), 7.84-7.78 (m, 2H), 6.96 (broad s, 1H), 4.18-4.00 (m, 4H), 4.00-3.60 (m, 3H), 3.54-3.42 (m, 1H), 3.25-3.15 (m, 4H), 3.15-3.05 (m, 4H), 2.94-2.80 (m, 2H), 2.80-2.63 (m, 2H), 2.60 (s, 3H), 2.30-2.16 (m, 1H), 2.12-2.00 (m, 5H), 1.70-1.50 (m, 1H), 1.36-1.28 (m, 3H).

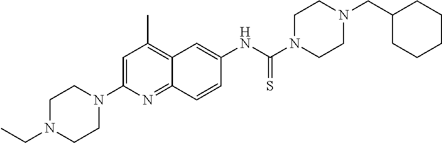 [SMILES: CCN1CCN(c2cc(C)c3cc(NC(=S)N4CCN(CC5CCCCC5)CC4)ccc3n2)CC1]

4-(cyclohexylmethyl)-N-(2-(4-ethylpiperazin-1-yl)-4-methylquinolin-6-yl)piperazine-1-carbothioamide. This compound was prepared according to synthetic Scheme 20. LC-MS, (M+H)⁺@ 495.8.

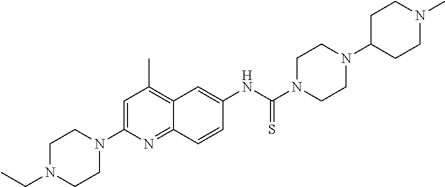 [SMILES: CCN1CCN(c2cc(C)c3cc(NC(=S)N4CCN(C5CCN(C)CC5)CC4)ccc3n2)CC1]

N-(2-(4-ethylpiperazin-1-yl)-4-methylquinolin-6-yl)-4-(1-methylpiperidin-4-yl)piperazine-1-carbothioamide. This compound was prepared according to synthetic Scheme 20. LC-MS, (M+H)⁺@ 496.8.

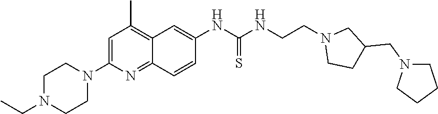 [SMILES: CCN1CCN(c2cc(C)c3cc(NC(=S)NCCN4CCC(CN5CCCC5)C4)ccc3n2)CC1]

1-(2-(4-ethylpiperazin-1-yl)-4-methylquinolin-6-yl)-3-(2-(3-(pyrrolidin-1-ylmethyl)pyrrolidin-1-yl)ethyl)thiourea. This compound was prepared according to synthetic Scheme 20. LC-MS, (M+H)⁺@ 510.9.

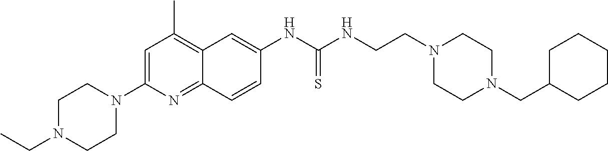 [SMILES: CCN1CCN(c2cc(C)c3cc(NC(=S)NCCN4CCN(CC5CCCCC5)CC4)ccc3n2)CC1]

1-(2-(4-(cyclohexylmethyl)piperazin-1-yl)ethyl)-3-(2-(4-ethylpiperazin-1-yl)-4-methylquinolin-6-yl)thiourea. This compound was prepared according to synthetic Scheme 20. LC-MS, (M+H)⁺@ 538.9.

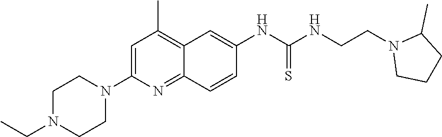 [SMILES: CCN1CCN(c2cc(C)c3cc(NC(=S)NCCN4CCCC4C)ccc3n2)CC1]

1-(2-(4-ethylpiperazin-1-yl)-4-methylquinolin-6-yl)-3-(2-(2-methylpyrrolidin-1-yl)ethyl)thiourea. This compound was prepared according to synthetic Scheme 20. LC-MS, (M+H)⁺@ 441.7; ¹H NMR (δ, ppm, CDCl₃) 8.12-8.06 (m, 1H), 7.82-7.76 (m, 1H), 7.72-7.65 (m, 1H), 6.92-6.88 (m, 1H), 4.01-3.95 (m, 2H), 3.46-3.38 (m, 2H), 3.35-3.26 (m, 8H), 3.12-3.07 (m, 2H), 2.61 (s, 3H), 2.32-2.40 (m, 3H), 2.28-2.14 (m, 2H), 1.78-1.60 (m, 2H), 1.38-1.31 (m, 3H), 1.14-1.08 (m, 3H).

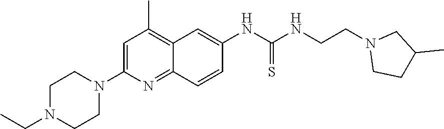 [SMILES: CCN1CCN(c2cc(C)c3cc(NC(=S)NCCN4CCC(C)C4)ccc3n2)CC1]

1-(2-(4-ethylpiperazin-1-yl)-4-methylquinolin-6-yl)-3-(2-(3-methylpyrrolidin-1-yl)ethyl)thiourea. This compound was prepared according to synthetic Scheme 20. LC-MS, (M+H) @ 441.6.

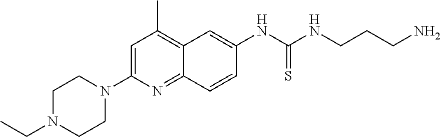 [SMILES: CCN1CCN(c2cc(C)c3cc(NC(=S)NCCCN)ccc3n2)CC1]

1-(3-aminopropyl)-3-(2-(4-ethylpiperazin-1-yl)-4-methylquinolin-6-yl)thiourea. This compound was prepared according to synthetic Scheme 20. LC-MS, (M+H)⁺@ 387.6.

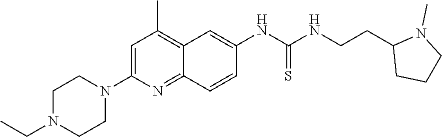 [SMILES: CCN1CCN(c2cc(C)c3cc(NC(=S)NCCC4CCCN4C)ccc3n2)CC1]

1-(2-(4-ethylpiperazin-1-yl)-4-methylquinolin-6-yl)-3-(2-(1-methylpyrrolidin-2-yl)ethyl)thiourea. This compound was prepared according to synthetic Scheme 20. LC-MS, (M+H)⁺@ 441.7.

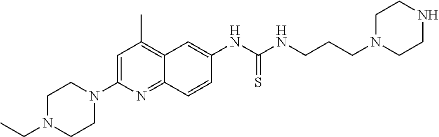 [SMILES: CCN1CCN(c2cc(C)c3cc(NC(=S)NCCCN4CCNCC4)ccc3n2)CC1]

1-(2-(4-ethylpiperazin-1-yl)-4-methylquinolin-6-yl)-3-(3-(piperazin-1-yl)propyl)thiourea. This compound was prepared according to synthetic Scheme 20. LC-MS, (M+H)⁺@ 456.8.

### Procedure for Starting Materials 4 and 5

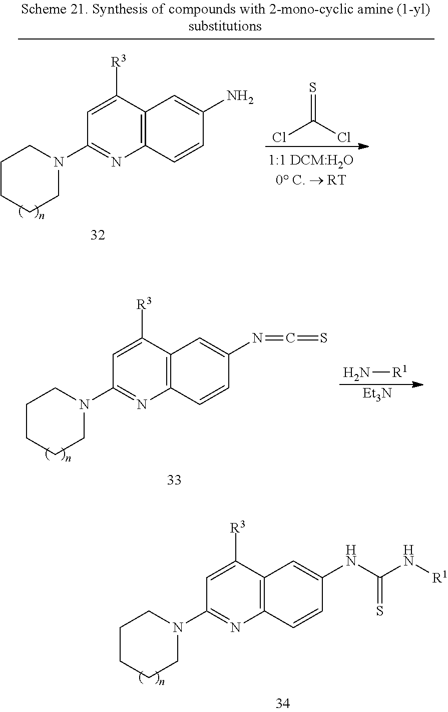

The starting material 32 was dissolved in DCM, and 4 eq. NaHCO₃ dissolved in an equal amount of water was added to it. The reaction was put in an ice bath to cool down to 0° C., and thiophosgene (1.5 eq.) in DCM was added dropwise. After 10 min, the reaction was taken off the ice bath and allowed to proceed at room temperature overnight. The organic and aqueous phases were then separated, and the organic layer was washed with brine, dried with solid Na₂SO₄, and concentrated. The thio-isocyanate intermediate 33 was re-dissolved in DCM, and Et₃N and an amine (R¹NH₂, 1.2 eq.) was added. After overnight, it was diluted in EtOAc, washed with brine, and concentrated. Then it was purified by HPLC in 0.1% TFA of H₂O. MeCN to afford 34.

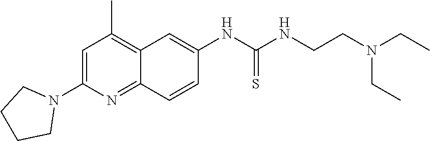 [SMILES: CCN(CC)CCNC(=S)Nc1ccc2nc(N3CCCC3)cc(C)c2c1]

1-(2-(diethylamino)ethyl)-3-(4-methyl-2-(pyrrolidin-1-yl)quinolin-6-yl)thiourea. This compound was prepared according to synthetic Scheme 21. LC-MS, (M+H)⁺@ 386.7; ¹H NMR (δ, ppm, CDCl₃) 8.19-8.14 (m, 1H), 7.98-7.92 (m, 1H), 7.78-7.71 (m, 1H), 6.65 (s, 1H), 4.08-3.99 (m, 2H), 3.90-3.56 (m, 4H), 3.43-3.36 (m, 2H), 3.26-3.16 (m, 4H), 2.58 (s, 3H), 2.18-2.10 (m, 4H), 1.40-1.32 (m, 6H).

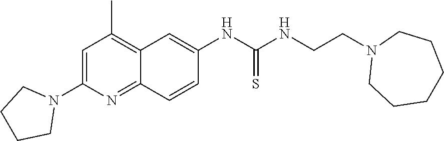 [SMILES: Cc1cc(N2CCCC2)nc2ccc(NC(=S)NCCN3CCCCCC3)cc12]

1-(2-(azepan-1-yl)ethyl)-3-(4-methyl-2-(pyrrolidin-1-yl)quinolin-6-yl)thiourea. This compound was prepared according to synthetic Scheme 21. LC-MS, (M+H)⁺@ 412.7; ¹H NMR (δ, ppm, CDCl₃) 8.20-8.14 (m, 1H), 7.93-7.87 (m, 1H), 7.78-7.72 (m, 1H), 6.65 (s, 1H), 4.06-3.98 (m, 2H), 3.68-3.48 (m, 4H) 3.42-3.32 (m, 4H), 3.16-3.04 (m, 2H), 2.57 (s, 3H), 2.17-2.08 (m, 4H), 1.96-1.84 (m, 4H), 1.80-1.60 (m, 4H).

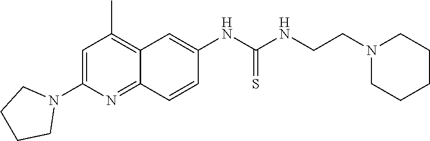 [SMILES: Cc1cc(N2CCCC2)nc2ccc(NC(=S)NCCN3CCCCC3)cc12]

1-(4-methyl-2-(pyrrolidin-1-yl)quinolin-6-yl)-3-(2-(piperidin-1-yl)ethyl)thiourea. This compound was prepared according to synthetic Scheme 21. LC-MS, (M+H)⁺@ 398.7; ¹H NMR (δ, ppm, CDCl₃) 8.20-1.14 (m, 1H), 7.94-7.86 (m, 1H), 7.80-7.72 (m, 1H), 6.64 (s, 1H), 4.09-3.98 (m, 2H), 3.86-3.54 (m, 6H), 3.38-3.28 (m, 2H), 2.86-2.72 (m, 2H), 2.70-2.40 (m, 4H), 2.13 (s, 3H), 1.98-1.78 (m, 6H).

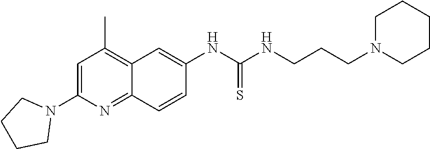 [SMILES: Cc1cc(N2CCCC2)nc2ccc(NC(=S)NCCCN3CCCCC3)cc12]

1-(4-methyl-2-(pyrrolidin-1-yl)quinolin-6-yl)-3-(3-(piperidin-1-yl)propyl)thiourea. This compound was prepared according to synthetic Scheme 21. LC-MS, (M+H)⁺@ 412.7.

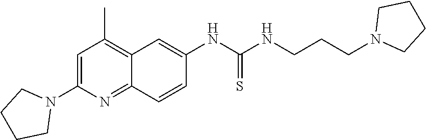 [SMILES: Cc1cc(N2CCCC2)nc2ccc(NC(=S)NCCCN3CCCC3)cc12]

1-(4-methyl-2-(pyrrolidin-1-yl)quinolin-6-yl)-3-(3-(pyrrolidin-1-yl)propyl)thiourea. This compound was prepared according to synthetic Scheme 21. LC-MS, (M+H)⁺@ 398.7; ¹H NMR (δ, ppm, CDCl₃) 8.26-8.20 (m, 1H), 7.82-7.76 (m, 1H), 7.74-7.68 (m, 1H), 6.63 (s, 1H), 3.78-3.58 (m, 8H), 3.25-3.17 (m, 2H), 2.98-2.86 (m, 2H), 2.53 (s, 3H), 2.17-2.04 (m, 10H).

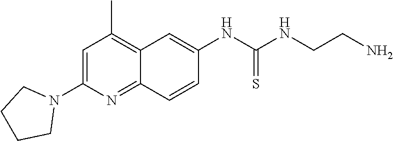 [SMILES: Cc1cc(N2CCCC2)nc2ccc(NC(=S)NCCN)cc12]

1-(2-aminoethyl)-3-(4-methyl-2-(pyrrolidin-1-yl)quinolin-6-yl)thiourea. This compound was prepared according to synthetic Scheme 21. LC-MS, (M+H)⁺@ 330.6; ¹H NMR (δ, ppm, CDCl₃) 8.24-8.18 (m, 1H), 7.86-7.80 (m, 1H), 7.74-7.67 (m, 1H), 6.66 (s, 1H), 3.94-3.86 (m, 2H), 3.41-3.38 (m, 3H), 3.38-3.34 (m, 1H), 3.24-3.18 (m, 2H), 2.58 (s, 3H), 2.18-2.10 (m, 4H).

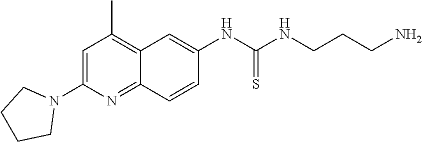 [SMILES: Cc1cc(N2CCCC2)nc2ccc(NC(=S)NCCCN)cc12]

1-(3-aminopropyl)-3-(4-methyl-2-(pyrrolidin-1-yl)quinolin-6-yl)thiourea. This compound was prepared according to synthetic Scheme 21. LC-MS, (M+H)⁺ @ 344.6; ¹H NMR (δ, ppm, CDCl₃) 8.25-8.20 (m, 1H), 7.84-7.77 (m, 1H), 7.72-7.64 (m, 1H), 6.66 (s, 1H), 3.78-3.70 (m, 2H), 3.42-3.39 (m, 2H), 3.39-3.34 (m, 2H), 3.04-2.96 (m, 2H), 2.58 (s, 3H), 2.18-2.10 (m, 4H), 2.02-1.92 (m, 2H).

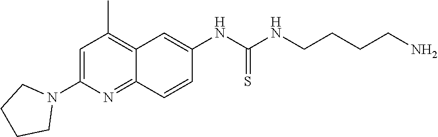 [SMILES: Cc1cc(N2CCCC2)nc2ccc(NC(=S)NCCCCN)cc12]

1-(4-aminobutyl)-3-(4-methyl-2-(pyrrolidin-1-yl)quinolin-6-yl)thiourea. This compound was prepared according to synthetic Scheme 21. LC-MS, (M+H)⁺ @ 358.6; ¹H NMR (δ, ppm, CDCl₃) 8.02-7.96 (m, 1H), 7.68-7.64 (m, 1H), 7.48-7.40 (m, 1H), 6.74-6.70 (m, 1H), 3.86-3.70 (m, 2H), 3.40-3.36 (m, 2H), 3.36-3.28 (m, 2H), 2.65 (s, 3H), 2.62-2.56 (m, 2H), 2.20-2.10 (m, 4H), 1.78-1.70 (m, 4H).

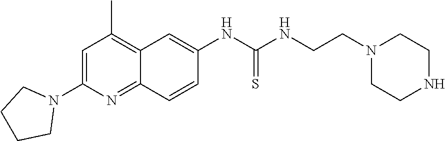 [SMILES: Cc1cc(N2CCCC2)nc2ccc(NC(=S)NCCN3CCNCC3)cc12]

1-(4-methyl-2-(pyrrolidin-1-yl)quinolin-6-yl)-3-(2-(piperazin-1-yl)ethyl)thiourea. This compound was prepared according to synthetic Scheme 21. LC-MS, (M+H)⁺@ 399.7; ¹H NMR (δ, ppm, CDCl₃) 8.26-8.22 (m, 1H), 7.90-7.85 (m, 2H), 7.13 (s, 1H), 4.12-4.05 (m, 2H), 3.84-3.58 (m, 12H), 3.50-3.42 (m, 2H), 2.71 (s, 3H), 2.25-2.14 (m, 4H).

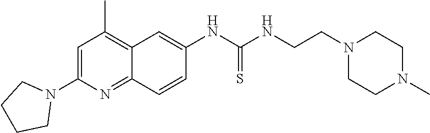 [SMILES: Cc1cc(N2CCCC2)nc2ccc(NC(=S)NCCN3CCN(C)CC3)cc12]

1-(4-methyl-2-(pyrrolidin-1-yl)quinolin-6-yl)-3-(2-(4-methylpiperazin-1-yl)ethyl)thiourea. This compound was prepared according to synthetic Scheme 21. LC-MS, (M+H)⁺@ 413.7.

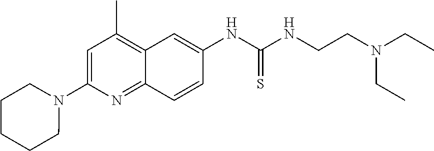 [SMILES: CCN(CC)CCNC(=S)Nc1ccc2nc(N3CCCCC3)cc(C)c2c1]

1-(2-(diethylamino)ethyl)-3-(4-methyl-2-(piperidin-1-yl)quinolin-6-yl)thiourea. This compound was prepared according to synthetic Scheme 21. LC-MS, (M+H)⁺@ 400.7; ¹H NMR (δ, ppm, CDCl₃) 8.19-8.15 (m, 1H), 7.98-7.88 (m, 1H), 7.77-7.70 (m, 1H), 6.90-6.85 (m, 1H), 4.00-3.94 (m, 2H), 3.80-3.74 (m, 4H), 3.39-3.12 (m, 8H), 2.60 (s, 3H), 1.80-1.60 (m, 6H), 1.35-1.25 (m, 6H).

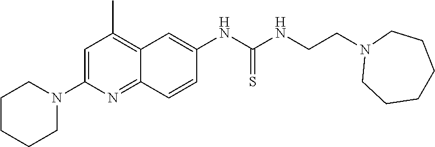 [SMILES: Cc1cc(N2CCCCC2)nc2ccc(NC(=S)NCCN3CCCCCC3)cc12]

1-(2-(azepan-1-yl)ethyl)-3-(4-methyl-2-(piperidin-1-yl)quinolin-6-yl)thiourea. This compound was prepared according to synthetic Scheme 21. LC-MS, (M+H)⁺@ 426.7; ¹H NMR (δ, ppm, CDCl₃) 8.20-8.12 (m, 1H), 7.98-7.90 (m, 1H), 7.78-7.69 (m, 1H), 6.90-6.84 (m, 1H), 4.08-3.98 (m, 2H), 3.86-3.74 (m, 4H), 3.63-3.48 (m, 2H), 3.42-3.34 (m, 2H), 3.18-3.02 (m, 2H), 2.58 (s, 3H), 2.00-1.60 (m, 14H).

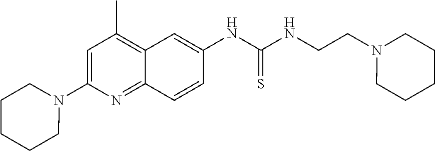 [SMILES: Cc1cc(N2CCCCC2)nc2ccc(NC(=S)NCCN3CCCCC3)cc12]

1-(4-methyl-2-(piperidin-1-yl)quinolin-6-yl)-3-(2-(piperidin-1-yl)ethyl)thiourea. This compound was prepared according to synthetic Scheme 21. LC-MS, (M+H)⁺@ 412.7; ¹H NMR (δ, ppm, CDCl₃) 8.19-1.13 (m, 1H), 7.97-7.90 (m, 1H), 7.77-7.70 (m, 1H), 6.87 (s, 1H), 4.18-4.00 (m, 2H), 3.85-3.75 (m, 4H), 3.68-3.56 (m, 2H), 3.38-3.30 (m, 2H), 2.57 (s, 3H), 1.96-1.80 (m, 6H), 1.80-1.70 (m, 6H).

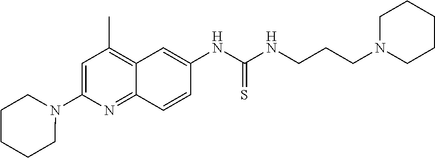 [SMILES: Cc1cc(N2CCCCC2)nc2ccc(NC(=S)NCCCN3CCCCC3)cc12]

1-(4-methyl-2-(piperidin-1-yl)quinolin-6-yl)-3-(3-(piperidin-1-yl)propyl)thiourea. This compound was prepared according to synthetic Scheme 21. LC-MS, (M+H)⁺@ 426.7; ¹H NMR (δ, ppm, CDCl₃) 8.23-8.17 (m, 1H), 7.89-7.82 (m, 1H), 7.74-7.67 (m, 1H), 6.87 (s, 1H), 3.84-3.70 (m, 6H), 3.58-3.48 (m, 2H), 3.16-3.06 (m, 2H), 2.78-2.64 (m, 2H), 2.56 (s, 3H), 2.16-2.04 (m, 2H), 1.94-1.80 (m, 6H), 1.80-1.70 (m, 6H).

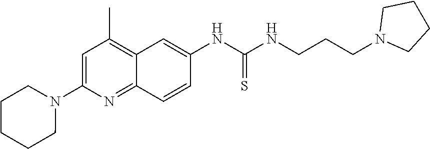 [SMILES: Cc1cc(N2CCCCC2)nc2ccc(NC(=S)NCCCN3CCCC3)cc12]

1-(4-methyl-2-(piperidin-1-yl)quinolin-6-yl)-3-(3-(pyrrolidin-1-yl)propyl)thiourea. This compound was prepared according to synthetic Scheme 21. LC-MS, (M+H)⁺@ 412.7.

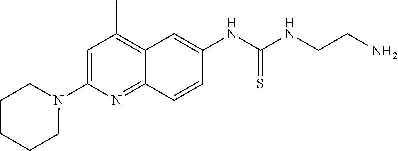 [SMILES: Cc1cc(N2CCCCC2)nc2ccc(NC(=S)NCCN)cc12]

1-(2-aminoethyl)-3-(4-methyl-2-(piperidin-1-yl)quinolin-6-yl)thiourea. This compound was prepared according to synthetic Scheme 21. LC-MS, (M+H)⁺@ 344.6.

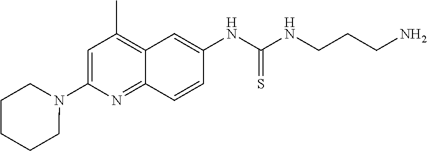 [SMILES: Cc1cc(N2CCCCC2)nc2ccc(NC(=S)NCCCN)cc12]

1-(3-aminopropyl)-3-(4-methyl-2-(piperidin-1-yl)quinolin-6-yl)thiourea. This compound was prepared according to synthetic Scheme 21. LC-MS, (M+H)⁺@ 358.6; ¹H NMR (δ, ppm, CDCl₃) 8.20-8.14 (m, 1H), 7.83-7.76 (m, 1H), 7.67-7.60 (m, 1H), 6.88 (s, 1H), 3.85-3.70 (m, 6H), 3.05-2.95 (m, 2H), 2.55 (s, 3H), 2.04-1.92 (m, 2H), 1.82-1.72 (m, 6H).

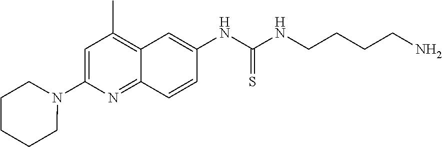 [SMILES: Cc1cc(N2CCCCC2)nc2ccc(NC(=S)NCCCCN)cc12]

1-(4-aminobutyl)-3-(4-methyl-2-(piperidin-1-yl)quinolin-6-yl)thiourea. This compound was prepared according to synthetic Scheme 21. LC-MS, (M+H)⁺ @ 372.6.

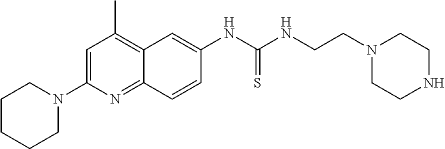 [SMILES: Cc1cc(N2CCCCC2)nc2ccc(NC(=S)NCCN3CCNCC3)cc12]

1-(4-methyl-2-(piperidin-1-yl)quinolin-6-yl)-3-(2-(piperazin-1-yl)ethyl)thiourea. This compound was prepared according to synthetic Scheme 21. LC-MS, (M+H)⁺@ 413.7; ¹H NMR (δ, ppm, CD₃OD) 8.26-8.22 (m, 1H), 7.88-7.85 (m, 2H), 7.41 (s, 1H), 4.11-4.02 (m, 2H), 3.93-3.85 (m, 4H), 3.75-3.35 (m, 10H), 2.73-2.69 (m, 3H), 1.88-1.78 (m, 6H).

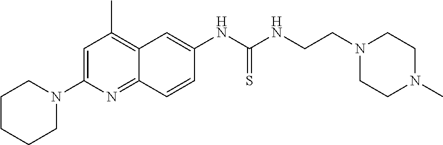 [SMILES: Cc1cc(N2CCCCC2)nc2ccc(NC(=S)NCCN3CCN(C)CC3)cc12]

1-(4-methyl-2-(piperidin-1-yl)quinolin-6-yl)-3-(2-(4-methylpiperazin-1-yl)ethyl)thiourea. This compound was prepared according to synthetic Scheme 21. LC-MS, (M+H)⁺@ 427.7; ¹H NMR (δ, ppm, CDCl₃) 8.30-8.24 (m, 1H), 7.85-7.78 (m, 1H), 7.73-7.66 (m, 1H), 6.87 (s, 1H), 3.92-3.83 (m, 2H), 3.83-3.74 (m, 4H), 3.40-3.30 (m, 4H), 3.26-2.14 (m, 4H), 3.08-3.00 (m, 2H), 2.80 (s, 3H), 2.57 (s, 3H), 1.80-1.72 (m, 6H).

### Procedure for Compounds 0038, 0039, and 0113

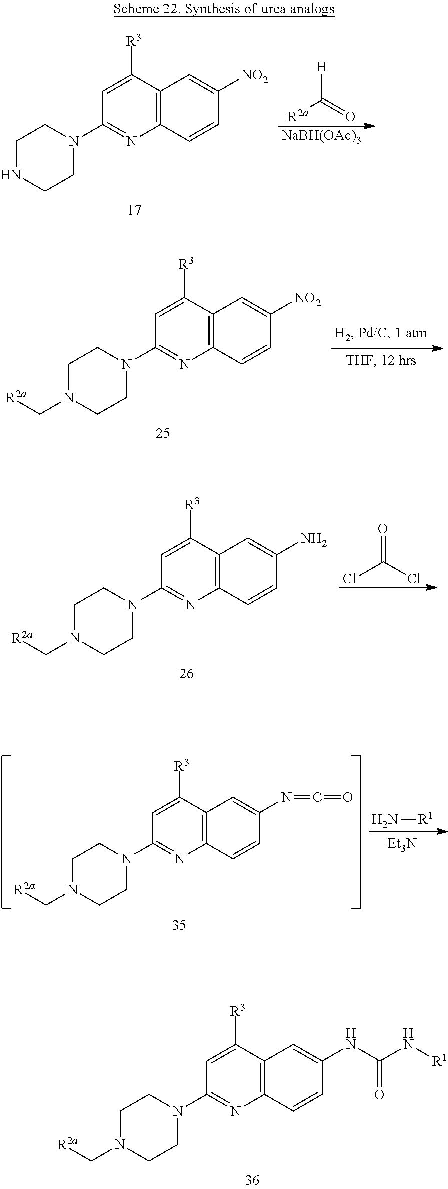

The aniline 26 was dissolved in chloroform under argon gas, and DIPEA (6 eq) and 3 eq. of phosgene was added to it. After 2 hours, the reaction was stirred under vacuum to get rid of excess phosgene, and then concentrated down completely. The isocyanate intermediate 35 was immediately re-dissolved in DCM, and Et₃N (2 eq) and an amine (R¹NH₂, 1.1 eq.) was added. After overnight, it was diluted in EtOAc, washed with brine, and concentrated. Then it was purified by HPLC in 0.1% TFA of H₂O. MeCN to afford 36.

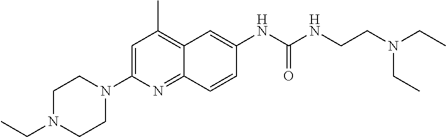 [SMILES: CCN(CC)CCNC(=O)Nc1ccc2nc(N3CCN(CC)CC3)cc(C)c2c1]

1-(2-(diethylamino)ethyl)-3-(2-(4-ethylpiperazin-1-yl)-4-methylquinolin-6-yl)urea. This compound was prepared according to synthetic Scheme 22. LC-MS, (M+H)⁺@ 413.7.

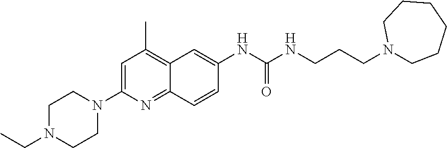 [SMILES: CCN1CCN(c2cc(C)c3cc(NC(=O)NCCCN4CCCCCC4)ccc3n2)CC1]

1-(3-(azepan-1-yl)propyl)-3-(2-(4-ethylpiperazin-1-yl)-4-methylquinolin-6-yl)urea. This compound was prepared according to synthetic Scheme 22. LC-MS, (M+H)⁺@ 453.8.

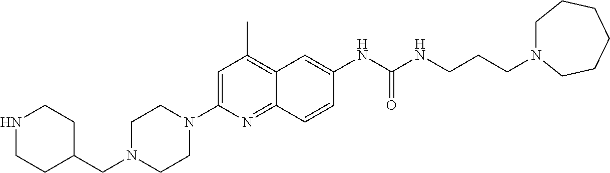 [SMILES: Cc1cc(N2CCN(CC3CCNCC3)CC2)nc2ccc(NC(=O)NCCCN3CCCCCC3)cc12]

1-(3-(azepan-1-yl)propyl)-3-(4-methyl-2-(4-(piperidin-4-ylmethyl)piperazin-1-yl)quinolin-6-yl)urea. This compound was prepared according to synthetic Scheme 22. LC-MS, (M+H)⁺@ 522.9; ¹H NMR (δ, ppm, CD₃OD) 8.30-8.26 (m, 1H), 8.01-7.95 (m, 1H), 7.86-7.79 (m, 1H), 7.51 (s, 1H), 3.55-3.40 (m, 4H), 3.40-3.33 (m, 1H), 3.33-3.30 (m, 4H), 3.30-3.02 (m, 10H), 2.76 (s, 3H), 2.26-2.14 (m, 2H), 2.10-1.82 (m, 8H), 1.80-1.50 (m, 5H).

### Procedure for Compound 0114

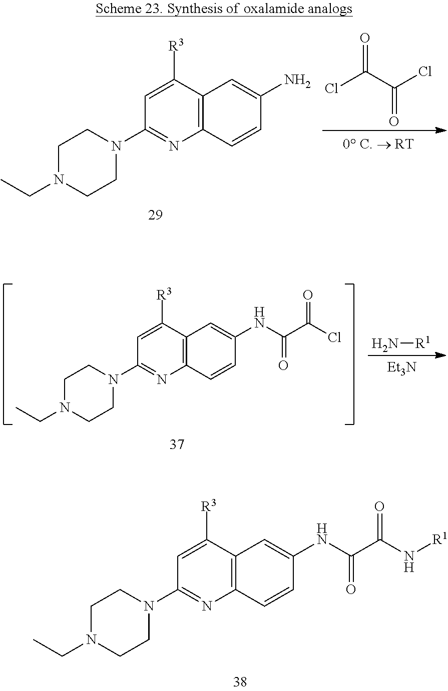

1.2 eq. of oxalyl chloride was dissolved in DCM under argon gas, and put on an ice bath to cool down to 0° C. The starting material 29 was dissolved in DCM, and added dropwise to the oxalyl chloride. After 10 min, the reaction was taken off the ice bath and allowed to proceed at room temperature overnight. The reaction was then concentrated down, and the intermediate 37 was re-dissolved in DCM, and Et₃N (2 eq) and an amine (R¹NH₂, 1.1 eq.) was added. After overnight, it was diluted in EtOAc, washed with brine, and concentrated. Then it was purified by HPLC in 0.1% TFA of H₂O:MeCN to afford 38.

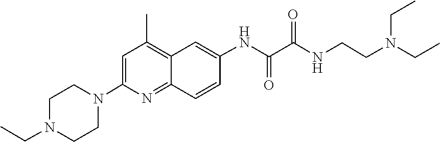 [SMILES: CCN(CC)CCNC(=O)C(=O)Nc1ccc2nc(N3CCN(CC)CC3)cc(C)c2c1]

N¹-(2-(diethylamino)ethyl)-N²-(2-(4-ethylpiperazin-1-yl)-4-methylquinolin-6-yl)oxalamide. This compound was prepared according to synthetic Scheme 23. LC-MS, (M+H)⁺@ 441.8; ¹H NMR (δ, ppm, CD₃OD) 8.50-8.45 (m, 1H), 8.06-7.98 (m, 1H), 7.86-7.79 (m, 1H), 7.36-7.30 (m, 1H), 3.76-3.69 (m, 2H), 3.58-3.43 (m, 3H), 3.43-3.33 (m, 5H), 3.33-3.22 (m, 8H), 2.71 (s, 3H), 1.42-1.31 (m, 9H).

### Biochemical Assays

The biological activity of the compounds of the present disclosure was determined utilizing the assay described herein

### Example 2. Cellular Assays

### Cell Lines:

1. BRCA1 deficient/proficient cell line: UWB1.298/UWB1.298 (BRCA1+).

2. BRCA2 deficient/proficient cell line: Capan-1/Capan-1 (BRCA2+)

### Protocol:

Capan-1 and Capan-1 (BRCA2+) cells were kept in IMDM (ATCC) media containing 20% FBS (GIBCO). UWB1.298 and UWB1.298 (BRCA1+) cells were kept in 48.5% RPMI1640 (ATCC), 48.5% MEGM (Clonetics/Lonza, MEGM kit, CC-3150) and 3% FBS (GIBCO) respectively. Cells in log-phase were harvested and 100 μl cell suspensions were replated in a 96-well plate with a final density of 4000 cells/well. After overnight growth, cells were treated with indicated concentrations of compounds. Media containing the invariant concentration of compounds were refreshed every 3 days until cells were finally lysed by 30 l/well of Promega CellTiter-Glo reagents and read on a Promega GloMax 96 reader on day 10 (9 days exposure).

### Example 3. Clonogenic Survival Assay

MDA-MB-436 cells were cultured in RPMI+10% FBS. BRCA-proficient and BRCA-deficient cells were plated on day 0 in triplicate at 5,000 cells/well. Cells were counted on day 4 on a hemocytometer, using Trypan Blue exclusion, and immediately were plated in a clonogenic assay at a density of 500 cells/well in a 6 well plate, in RPMI+10% FBS. After two weeks, the colonies were fixed/stained with 0.05% of 10 mg/ml ethidium bromide in 50% ethanol and visualized with Alphaimager gel imager (Alpha Innotech).

### Example 4. CML Viability Assay

Lin-CD34+ primary CML and normal cells were obtained by magnetic sorting using the EasySep negative selection human progenitor cell enrichment cocktail followed by treatment with human CD34 positive selection cocktail (StemCell Technologies), and were subsequently cultured in StemSpan H3000 media (StemCell Technologies) supplemented with a cocktail of growth factors (100 ng/ml stem cell factor, 20 ng/ml interleukin3 [IL-3], 100 ng/ml fms-related tyrosine kinase 3 ligand, 20 ng/ml granulocyte colony-stimulating factor, 20 ng/ml IL-6).

### Example 5. Chemicals, Proteins, and DNA

Cisplatin was purchased from Sigma-Aldrich. Human RAD52 and RAD51 were purified as described (Bugreev et al., 2005, Mol. Cell. Biol. 33, 387-395). The oligonucleotides (Table A) were purchased from IDT, Inc and further purified by electrophoresis (Rossi et al., 2010, Methods 51, 336-346). Supercoiled pUC19 plasmid DNA was purified using Qiagen kits. All DNA concentrations are expressed as moles of nucleotide.

TABLE A
Sequences of the oligonucleotides

| | Length, | |
|---|---|---|
| N | nt | Sequence (5′→3′) |
| 337- | 60 | FLU-CACTGTGATGCACGATGATCGACGACAGTAGTCAGT |
| FLU | | GCTGGGTCAACATCTGTATGCAGG (FLU-SEQ ID NO: 1) |
| 1337- | 39 | AGCACTGACTACTGTCGTCGATCATCGTGCATCACAGTG- |
| BHQ1 | | BHQ1 (SEQ ID NO: 2-BHQ1) |
| 265-55 | 55 | ATACAGATGTTGACCCAGCACTGACTACTGTCGTCAATCAT |
| | | CGTGCATCACAGTG (SEQ ID NO: 3) |
| 90 | 90 | CGGGTGTCGGGGCTGGCTTAACTATGCGGCATCAGAGCAGA |
| | | TTGTACT GAG AGT GCA CCA TAT GCG GTG TGA AAT ACC |
| | | GCA CAG ATG CGT (SEQ ID NO:4) |

Note:
“FLU” and “BHQ1” denote Fluorescein and Black Hole Quencher 1, respectively.

### Example 6. Measurement of Compound Binding to RAD52 by SPR

Experiments were performed using the ProteOn XPR36 SPR array system (Bio-Rad). ProteOn GLH sensor chips were preconditioned with two short pulses each (10 s) of 50 mM NaOH, 100 mM HCl, and 0.5% SDS. Then the system was equilibrated with PBS-T buffer (20 mM Na-phosphate, 150 mM NaCl, and 0.1% polysorbate 20, pH 7.4). Individual ligand flow channels were activated for 5 min at 25° C. with a mixture of 1-ethyl-3-[3-dimethylamino propyl carbodiimide hydrochloride) (0.2 μM) and sulfo-N-hydroxy succinimide (0.05 M). Immediately after chip activation, either RAD52 (100 μg·ml-1 in 25 mM Tris-Acetate, 20 mM KCl, 0.3 mM magnesium acetate, pH 7.5) or the anti-HIV mAb 2F5 (100 μg·ml-1 in 10 mM sodium acetate, pH 5.0) was injected across ligand flow channels for 5 min at a flow rate of 30 ρL·min⁻¹.

Excess active ester groups on the sensor surface were capped by a 5-min injection of 1 M ethanolamine HCl (pH 8.5). This resulted in the coupling of RAD52 and 2F5 at a density of 9,000 RUs (response unit, which is an arbitrary unit that corresponds to 1 μg/mm²). The standard deviation in the immobilization level from the six spots within each channel was less than 4%.

Specific regeneration of the surfaces between injections was not needed owing to the nature of the interaction. Data were analyzed using the ProteOn Manager Software version 3.0 (Bio-Rad). The responses of a buffer injection and responses from the reference flow cell were subtracted to account for nonspecific binding. Experimental data were fitted globally to a simple 1:1 binding model. The average kinetic parameters (association [ka] and dissociation [kd] rates) generated from three data sets were used to define the equilibrium dissociation constant (KD). Data that could not be adequately fit to a binding model were analyzed using equilibrium analysis, plotting the response at equilibrium versus concentration and fitting to a steady state model.

### Example 7. Measuring the Effect of Inhibitors on GFP-RAD52 and RAD51 Foci Formation

GFP-RAD52 foci formation was measured in BCR-ABL1-positive BRCA1-deficient 32Dcl3 murine hematopoietic cell line that expresses GFP-RAD52 (Cramer-Morales et al., 2013, Blood 122, 1293-1304). RAD51 foci formation was measured in parental 32Dcl3. Both cell lines were cultured in IMDM plus 10% FBS.

### Example 8. Fluorescence-Quenching Assay for RAD52 DNA Annealing

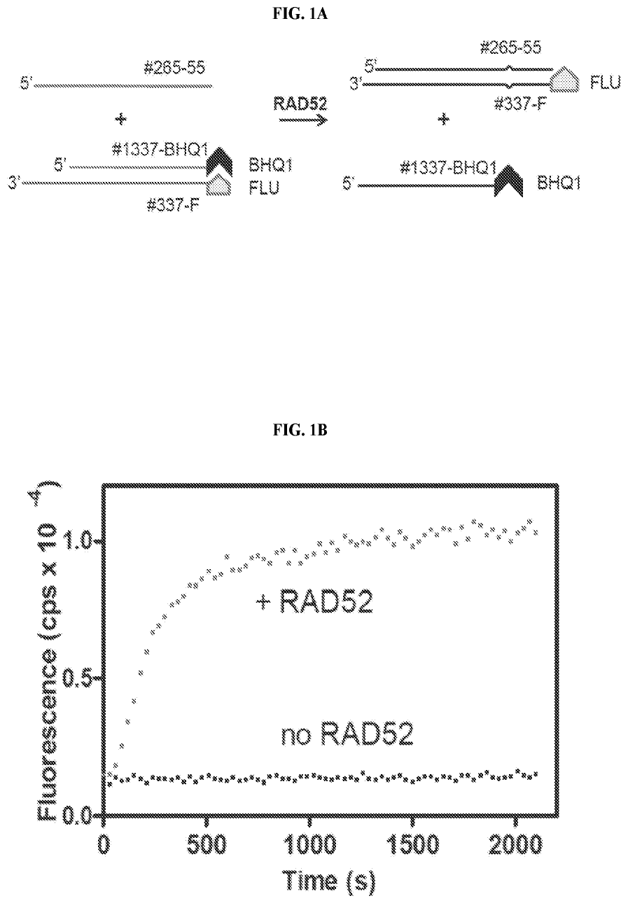

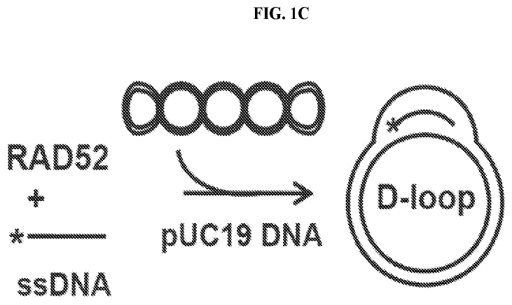

As shown in FIG. 1A and FIG. 1B, tailed dsDNA substrate was prepared by thermal annealing of ssDNA oligonucleotides 337-F and 1337-BHQ1 (Table A) containing Fluorescein and Black Hole Quencher 1 residues at the 5′ and 3′ end, respectively. DNA annealing was initiated by adding RAD52 (20 nM) to the mixture of ssDNA oligonucleotide 265-55 (Table A) (5 nM, molecules) and tailed dsDNA 337-F/1337-BHQ1 (Table A) (5 nM, molecules) in buffer containing 25 mM Tris-acetate pH 7.5, 100 μg·ml-1 BSA and 1 mM DTT. The fluorescence intensity was measured in a 3-mm quartz cuvette (Starna Cells) using a FluoroMax-3 (HORIBA) fluorimeter with 492 nm excitation wavelength and 520 nm emission wavelength at 30° C. for at least 2000 s.

### Example 9. HTS for RAD52 Inhibitors

The fluorescence-quenching assay for RAD52-promoted DNA annealing was optimized to a 4 μl 1536 well protocol using 25 nM RAD52 and 8 nM (molecules) DNA in buffer containing 25 mM Tris-acetate pH 7.5, 100 μg·mL-1 BSA, 1 mM DTT, and 0.01% Pluronic F-68. Wells containing no RAD52 were used as a positive control to estimate the activity of fully inhibited protein; wells in which the compounds were replaced with only the vehicle (DMSO) were used as neutral control. The HTS was performed using the 8 channel BioRAPTR 1536 (Beckman) for reagent dispensing. The reactions were carried out for 30 minutes followed by measurement of an endpoint fluorescence (485 nm excitation, 535 nm emission) using an EnVision multimode plate reader (Perkin Elmer). Wells containing no RAD52 enzyme were used to as positive control, and data were analyzed using Genedata. The compounds with an inhibitory effect of 30% or greater were tested further by measuring the concentration dependence (in a range from 1 nM to 100 μM) of their inhibition of RAD51. The most potent inhibitory compounds were analyzed further using non-fluorescent assays. Detailed methods for RAD52 screening are in PubMed: pubchem dot ncbi dot nlm dot nih dot gov/assay/assay dot cgi?aid=651660.

### Example 10. D-Loop Formation by RAD52 or RAD51

As shown in FIG. 1C, to form RAD52 nucleoprotein complexes, RAD52 (0.45 PM) was incubated with a ³²P-labeled ssDNA (oligo 90/SEQ ID NO: 4) (3 μM, nt) in buffer containing 25 mM Tris-Acetate, pH 7.5, 100 μg·mL-1 BSA, 0.3 mM magnesium acetate, and 2 mM DTT at 37° C. for 15 min. To form RAD51 nucleoprotein filament, RAD51 (1 μM) was incubated with ³²P-labeled ssDNA (3 μM, nt) in buffer containing 25 mM Tris-Acetate, pH 7.5, 100 μg·ml-1 BSA, 1 mM calcium chloride, 1 mM ATP and 2 mM DTT for 15 min at 37° C. Then inhibitors were added to both reactions and incubation continued for 15 min at 37° C. D-Loop formation was initiated by addition of supercoiled pUC19 DNA (50 μM, nucleotides) and was carried out 15 min at 37° C. The reactions were stopped and deproteinized by the addition of 1.5% SDS and proteinase K (0.8 mg/ml) for 15 min at 37° C., mixed with a 0.10 volume of loading buffer (70% glycerol, 0.1% bromphenol blue), and analyzed by electrophoresis in 1% agarose gels in TAE buffer (40 mM Tris acetate, pH 8.3, and 1 mM EDTA) at 5 V/cm for 3 h. The gels were dried on DEAE-81 paper (Whatman) and the yield of D-loops quantified using a Storm 840 PhosphorImager and ImageQuant 5.2 (GE Healthcare). The D-loop yield was expressed as a percentage of plasmid DNA carrying D-loops relative to the total plasmid DNA. The results of the D-loop experiment are shown below in Table B.

TABLE B
Activities of exemplary compounds at 10 μM in D-Loop experiment

| Compound | D-Loop | Compound | D-Loop | Compound | D-Loop |
|---|---|---|---|---|---|
| No. | (% @10 μM) | No. | (% @10 μM) | No. | (% @10 μM) |
| 0021 | 40.29 | 0050 | 62.82 | 0077 | 21.2 |
| 0022 | 37.7 | 0051 | 10.13 | 0079 | 25.15 |
| 0025 | 36.08 | 0052 | 4.07 | 0080 | 35.63 |
| 0026 | 11.42 | 0053 | 54.13 | 0084 | 68.84 |
| 0027 | 23.26 | 0054 | 53.78 | 0086 | 4.44 |
| 0028 | 46.57 | 0055 | 27.42 | 0087 | 0 |
| 0029 | 46.67 | 0056 | 0.31 | 0088 | 8.25 |
| 0033 | 12.77 | 0057 | 52.1 | 0089 | 3.48 |
| 0035 | 64.68 | 0058 | 25.89 | 0090 | 2.93 |
| 0036 | 64.62 | 0059 | 1.3 | 0091 | 3.47 |
| 0038 | 77.7 | 0060 | 0.66 | 0092 | 5.98 |
| 0039 | 93.38 | 0061 | 39.75 | 0093 | 4.86 |
| 0041 | 62.62 | 0062 | 1.89 | 0094 | 4.26 |
| 0042 | 53.70 | 0063 | 1.58 | 0095 | 4.51 |
| 0044 | 50.05 | 0064 | 1.5 | 0096 | 3.99 |
| 0045 | 55.68 | 0065 | 1.04 | 0097 | 4.72 |
| 0046 | 18.73 | 0067 | 35.65 | 0098 | 70.99 |
| 0047 | 7.92 | 0068 | 30.62 | 0099 | 67.50 |
| 0048 | 15.65 | 0069 | 20.7 | 0113 | 0.34 |
| 0049 | 44.4 | 0073 | 16.42 | 0114 | 16 |

### Calculation of the IC₅₀ Value for RAD52 Inhibitors

IC₅₀ values were calculated using GraphPad Prism V5.0 software. The data were obtained from three independent repeats of experiments.

### Example 11. Luminescent Cell Viability Assay

BxPC3 cells were kept in RPMI 1640 (ATCC) media supplemented with 10% FBS (Gibco); Capan-1 cells were kept in IMDM (ATCC) media containing 20% FBS (GIBCO); UWB1.298 and UWB1.298 (BRCA1+) cells were kept in 48.5% RPMI1640 (ATCC), 48.5% MEGM (Clonetics/Lonza, MEGM kit, CC-3150) and 3% FBS (GIBCO) respectively. Cells in log-phase were harvested and 100 μL cell suspensions were replated in a 96-well plate with a final density of 4000 cells/well. After overnight growth, cells were treated with indicated concentrations of compounds. Media containing the invariant concentration of compounds were refreshed every 3 days until cells were finally lysed by 30 l/well of Promega CellTiter-Glo reagents and read on a Promega GloMax 96 reader on day 10 (9 days exposure).

### Example 12. Luminescent Cell Viability Assay for DLD1 BRCA2^{+/+} and BRCA2^{-/-} Cells

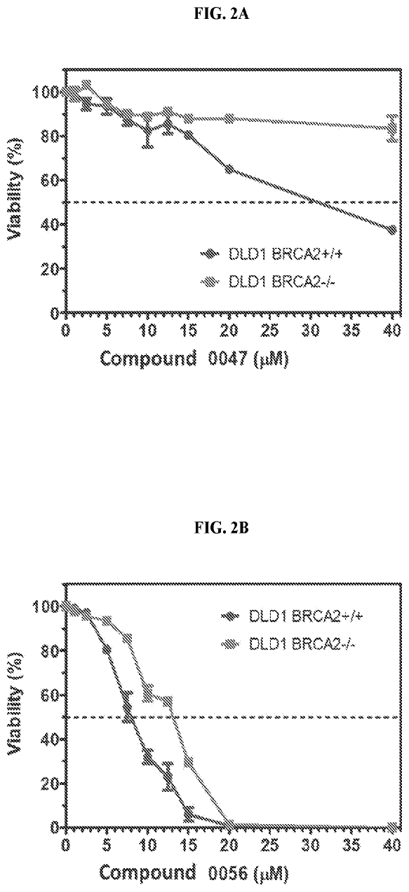

A 96-well plate was seeded with DLD1 BRCA2+/+(200 cells/well) and BRCA2−/− cells (600 cells/well) in triplicate for each test compound. The plate was incubated for 24 h at 37° C. to ensure that cells adhere to the surface of the well. On days 1,4, and 7, medium was replaced with fresh medium and test compound (premixed, 100 μL), followed by incubation for 72 h at 37° C. On day 10, 30 μL of CellTiterGlo reagent directly to each well. The plate was covered with aluminum foil and placed on a shaker for 2 min at 0.8 setting to ensure proper mixing. The plate was incubated at room temperature for 10 min to stabilize the luminescence signal, and then read on a Promega GloMax 96 reader. The results of this assay for compounds 0047 and 0056 are shown in FIG. 2A and FIG. 2B, respectively.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following embodiments are provided, the numbering of which is not to be construed as designating levels of importance:

Embodiment 1 provides a compound of Formula I′:

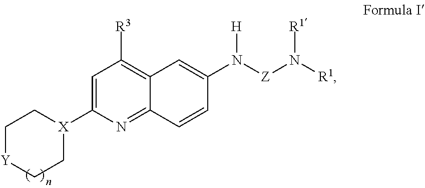

and pharmaceutically acceptable salts and solvates thereof, wherein:

X is CH or N;

Y is CH₂ or N—R²;

Z is

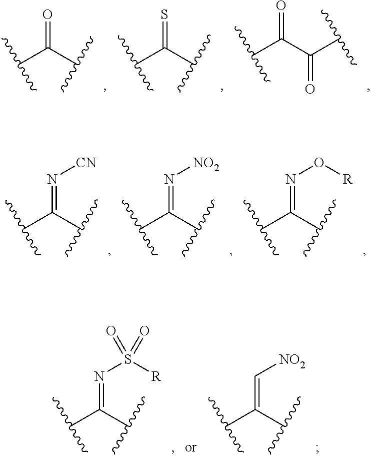

R¹ is H, C₁₋₆ alkyl optionally substituted with one or more N(R⁴)(R^{4′}), —C₀₋₆ alkyl-(4- to 8-membered heterocyclyl), or -(4- to 8-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl;

R^{1′} is H or C₁₋₆ alkyl, or

R¹ and R^{1′}, together with the nitrogen atom to which R¹ and R^{1′} are attached, form a 5- to 6-membered heterocyclyl optionally substituted with one or more R⁵;

R² is H, C₁₋₆ alkyl, —C₃₋₆ cycloalkyl-C₀₋₆ alkyl, —C₀₋₆ alkyl-C₃₋₆ cycloalkyl, -(3- to 7-membered heterocyclyl)-C₀₋₆ alkyl, —C₀₋₅ alkyl-(3- to 7-membered heterocyclyl), —(C₆₋₁₀ aryl)-C₀₋₆ alkyl, —C₀₋₅ alkyl-(C₆₋₁₀ aryl), -(3- to 7-membered heteroaryl)-C₀₋₆ alkyl, —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl, cycloalkyl, heterocyclyl, aryl, heteroaryl represented by R² is optionally substituted with one or more —OH, —NH₂, —NH—C(═O)—O—(C₁₋₆ alkyl), halogen, C₁₋₆ alkyl, or phenyl;

R³ is H, C₁₋₆ alkyl, C₂₋₆ alkenyl, C₂₋₆ alkynyl, C₁₋₆ haloalkyl, halogen, —CN, —NO₂, —OR, —SR, —S(═O)₂R, —C(═O)R, —OC(═O)R, —NR₂, or —CO₂R;

each R⁴ and R^{4′} is independently H, —C(═O)—O—(C₁₋₆ alkyl), C₁₋₆ alkyl, C₂₋₆ alkenyl, C₂₋₆ alkynyl, or C₃₋₆ cycloalkyl;

R⁵ is -(5- to 7-membered heterocyclyl)-C₀₋₆-alkyl, —C₀₋₆-alkyl-(5- to 7-membered heterocyclyl), —(C₃₋₆ cycloalkyl)-C₀₋₆-alkyl, or —C₀₋₆-alkyl-(C₃₋₆ cycloalkyl), wherein the alkyl, heterocyclyl, or cycloalkyl represented by R⁵ is optionally substituted by one or more C₁₋₆ alkyl;

each R is independently H, C₁₋₆ alkyl, C₂₋₆ alkenyl, C₂₋₆ alkynyl, or C₃₋₆ cycloalkyl; and

n is 0 or 1,

provided that R² and R³ are not simultaneously CH₃.

Embodiment 2 provides the compound embodiment 1, wherein:

X is N;

Y is CH₂ or N—R²;

Z is

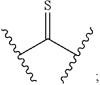 [SMILES: *C(*)=S]

R¹ is H, C₁₋₆ alkyl optionally substituted with one or more N(R⁴)(R^{4′}), or —C₀₋₆ alkyl-(4- to 8-membered heterocyclyl), wherein the heterocyclyl is optionally substituted with one or more oxo, —C₀₋₂ alkyl-(4- to 8-membered heterocyclyl), —C₀₋₆ alkyl-(C₃₋₇ cycloalkyl), or C₁₋₃ alkyl;

R^{1′} is H or —CH₃, or

R¹ and R^{1′}, together with the nitrogen atom to which R¹ and R^{1′} are attached, form a 5- to 6-membered heterocyclyl optionally substituted with one or more R⁵;

R² is H, C₁₋₅ alkyl, —C₀₋₅ alkyl-C₃₋₆ cycloalkyl, —C₀₋₅ alkyl-(3- to 7-membered heterocyclyl), —C₀₋₅ alkyl-(C₆₋₁₀ aryl), —C₀₋₅ alkyl-(3- to 7-membered heteroaryl), —C(═O)—C₁₋₅ alkyl, —C(═O)—(C₆₋₁₀ aryl), —C(═O)-(5- to 7-membered heterocyclyl), —C(═O)—O—C₁₋₅ alkyl, —SO₂—(C₆₋₁₀ aryl), —C(═O)—NH—C₁₋₅ alkyl, or —C(═O)—NH—(C₆₋₁₀ aryl), wherein the alkyl, cycloalkyl, heterocyclyl, aryl, heteroaryl represented by R² is optionally substituted with one or more —OH, —NH₂, —NH—C(═O)—O—(C₁₋₅ alkyl), halogen, C₁₋₃ alkyl, or phenyl;

R³ is H or C₁₋₆ alkyl;

each R⁴ and R^{4′} is independently H, —C(═O)—O—(C₁₋₅ alkyl), or C₁₋₆ alkyl;

R⁵ is —C₀₋₆-alkyl-(5- to 7-membered heterocyclyl) or —C₀₋₆-alkyl-(C₃₋₆ cycloalkyl), wherein the alkyl, heterocyclyl, or cycloalkyl represented by R⁵ is optionally substituted by one or more C₁₋₆ alkyl;

each R is independently H or C₁₋₆ alkyl; and

n is 0 or 1,

provided that R² and R³ are not simultaneously CH₃.

Embodiment 3 provides the compound of any one of the preceding embodiments, wherein R² is H, C₂₋₅ alkyl, —C₃₋₆ cycloalkyl-C₀₋₅ alkyl, —C₀₋₅ alkyl-C₃₋₆ cycloalkyl, -(3- to 7-membered heterocyclyl)-C₀₋₅ alkyl, —C₀₋₅ alkyl-(3- to 7-membered heterocyclyl), —(C₆₋₁₀ aryl)-C₀₋₅ alkyl, —C₀₋₅ alkyl-(C₆₋₁₀ aryl), -(3- to 7-membered heteroaryl)-C₀₋₅ alkyl, —C₀₋₅ alkyl-(3- to 7-membered heteroaryl), —C(═O)—C₁₋₅ alkyl, —C(═O)—(C₆₋₁₀ aryl), —C(═O)-(5- to 7-membered heterocyclyl), —C(═O)—O—C₁₋₅ alkyl, —SO₂—(C₆₋₁₀ aryl), —C(═O)—NH—C₁₋₅ alkyl, or —C(═O)—NH—(C₆₋₁₀ aryl), wherein the alkyl, cycloalkyl, heterocyclyl, aryl, heteroaryl represented by R² is optionally substituted with one or more —OH, —NH₂, —NH—C(═O)—O—(C₁₋₅ alkyl), halogen, C₁₋₃ alkyl, or phenyl and R³ is CH₃.

Embodiment 4 provides the compound of any one of the preceding embodiments, wherein R² is CH₃ and R³ is H, C₂₋₆ alkyl, C₂₋₆ alkenyl, C₂₋₆ alkynyl, C₁₋₆ haloalkyl, halogen, —CN, —NO₂, —OR, —SR, —S(═O)₂R, —C(═O)R, —OC(═O)R, —NR₂, and —CO₂R.

Embodiment 5 provides the compound of any one of the preceding embodiments, wherein the compound of Formula I is not 1-(2-(diethylamino)ethyl)-3-(4-methyl-2-(4-ethylpiperazin-1-yl)quinolin-6-yl)thiourea, 1-isopropyl-3-(4-methyl-2-(pyrrolidin-1-yl)quinolin-6-yl)thiourea, 1-(4-Ethyl-phenyl)-3-[2-(4-ethyl-piperazin-1-yl)-4-methyl-quinolin-6-yl]-1-propyl-thiourea, 1-Benzyl-3-[2-(4-ethyl-piperazin-1-yl)-4-methyl-quinolin-6-yl]-1-methyl-thiourea, 1-(4-Ethoxy-phenyl)-1-ethyl-3-[2-(4-ethyl-piperazin-1-yl)-4-methyl-quinolin-6-yl]-thiourea, 1-[2-(4-Ethyl-piperazin-1-yl)-4-methyl-quinolin-6-yl]-3-thiophen-2-ylmethyl-thiourea, 1-[2-(4-Ethyl-piperazin-1-yl)-4-methyl-quinolin-6-yl]-3-(2-methoxy-benzyl)-thiourea, 1-[2-(4-Ethyl-piperazin-1-yl)-4-methyl-quinolin-6-yl]-3-(4-fluoro-benzyl)-thiourea, 1-[2-(4-Ethyl-piperazin-1-yl)-4-methyl-quinolin-6-yl]-3-furan-2-ylmethyl-thiourea, 1-Ethyl-3-[2-(4-ethyl-piperazin-1-yl)-4-methyl-quinolin-6-yl]-1-(4-fluoro-phenyl)-thiourea, 1-(2-Ethyl-phenyl)-3-[2-(4-ethyl-piperazin-1-yl)-4-methyl-quinolin-6-yl]-1-methyl-thiourea, 1-Benzo[1,3]dioxol-5-ylmethyl-3-[2-(4-ethyl-piperazin-1-yl)-4-methyl-quinolin-6-yl]-thiourea, 1-(2-(dimethylamino)ethyl)-3-(4-methyl-2-(pyrrolidin-1-yl)quinolin-6-yl)thiourea, 1-(3-(3,5-dimethylpiperidin-1-yl)propyl)-3-(2-(4-ethylpiperazin-1-yl)-4-methylquinolin-6-yl)thiourea, N-(2-(4-ethylpiperazin-1-yl)-4-methylquinolin-6-yl)-[1,4′-bipiperidine]-1′-carbothioamide, 1-(2-(4-ethylpiperazin-1-yl)-4-methylquinolin-6-yl)-3-(3-(2-ethylpiperidin-1-yl)propyl)thiourea, or 1-((1-benzylpiperidin-4-yl)methyl)-3-(2-(piperazin-1-yl)quinolin-6-yl)thiourea.

Embodiment 6 provides the compound of any one of the preceding embodiments, wherein X is N.

Embodiment 7 provides the compound of any one of the preceding embodiments, wherein n is 1.

Embodiment 8 provides the compound of any one of the preceding embodiments, wherein Z is

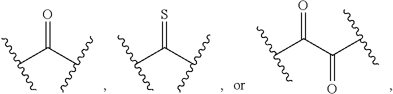

Embodiment 9 provides the compound of any one of the preceding embodiments, wherein Z is

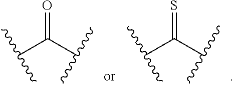

Embodiment 10 provides the compound of any one of the preceding embodiments, wherein Z is

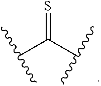 [SMILES: *C(*)=S]

Embodiment 11 provides the compound of any one of the preceding embodiments, wherein Y is N—R².

Embodiment 12 provides the compound of any one of the preceding embodiments, wherein R¹ is H.

Embodiment 13 provides the compound of any one of the preceding embodiments, wherein R¹ is C₁₋₆ alkyl optionally substituted with one or more N(R⁴)(R^{4′}), —C₀₋₆ alkyl-(4- to 8-membered heterocyclyl), or -(4- to 8-membered heterocyclyl)-C₀₋₆ alkyl, wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

Embodiment 14 provides the compound of any one of the preceding embodiments, wherein R¹ is C₁₋₆ alkyl optionally substituted with one or more N(R⁴)(R^{4′}) or —C₀₋₆ alkyl-(4- to 8-membered heterocyclyl), wherein the heterocyclyl is optionally substituted with one or more oxo, —C₀₋₆ alkyl-(4- to 8-membered heterocyclyl), —C₀₋₆ alkyl-(C₃₋₇ cycloalkyl), or C₁₋₆ alkyl.

Embodiment 15 provides the compound of any one of the preceding embodiments, wherein R¹ is C₁₋₆ alkyl optionally substituted with one or more N(R⁴)(R^{4′}).

Embodiment 16 provides the compound of any one of the preceding embodiments, wherein R¹ is —C₀₋₆ alkyl-(4- to 8-membered heterocyclyl) or -(4- to 8-membered heterocyclyl)-C₀₋₆ alkyl.

Embodiment 17 provides the compound of any one of the preceding embodiments, wherein R¹ is —C₀₋₆ alkyl-(4- to 8-membered heterocyclyl) or -(4- to 8-membered heterocyclyl)-C₀₋₆ alkyl, wherein the heterocyclyl is optionally substituted with one or more oxo, —C₀₋₆ alkyl-(4- to 8-membered heterocyclyl), —C₀₋₆ alkyl-(C₃₋₇ cycloalkyl), or C₁₋₆ alkyl.

Embodiment 18 provides the compound of any one of the preceding embodiments, wherein R¹ is —C₀₋₆ alkyl-(4- to 8-membered heterocyclyl).

Embodiment 19 provides the compound of any one of the preceding embodiments, wherein R¹ is —C₀₋₆ alkyl-(4- to 8-membered heterocyclyl), wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

Embodiment 20 provides the compound of any one of the preceding embodiments, wherein R^{1′} is H or methyl.

Embodiment 21 provides the compound of any one of the preceding embodiments, wherein R^{1′} is H.

Embodiment 22 provides the compound of any one of the preceding embodiments, wherein R^{1′} is methyl.

Embodiment 23 provides the compound of any one of the preceding embodiments, wherein R¹ is H,

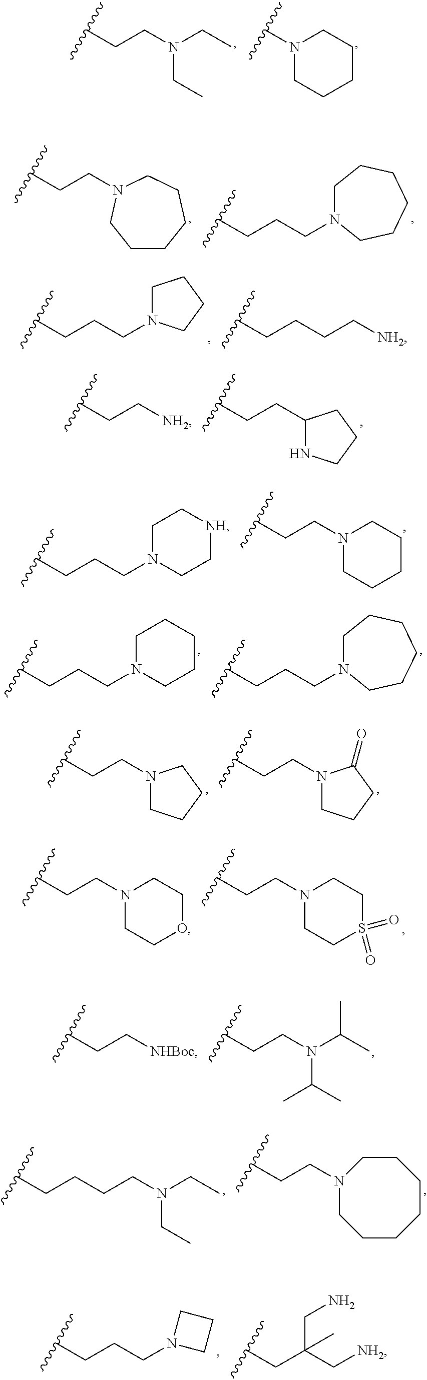

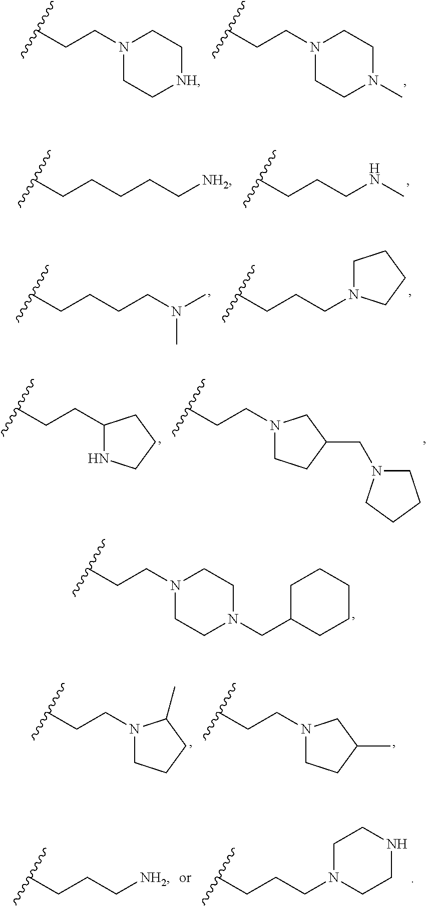

Embodiment 24 provides the compound of any one of the preceding embodiments, wherein R¹ and R^{1′}, together with the nitrogen atom to which R¹ and R^{1′} are attached, form a 5-membered heterocyclyl.

Embodiment 25 provides the compound of any one of the preceding embodiments, wherein R¹ and R^{1′}, together with the nitrogen atom to which R¹ and R^{1′} are attached, form a 6-membered heterocyclyl.

Embodiment 26 provides the compound of any one of the preceding embodiments, wherein R¹ and R^{1′}, together with the nitrogen atom to which R¹ and R^{1′} are attached, form a 5-membered heterocyclyl optionally substituted with one or more R⁵.

Embodiment 27 provides the compound of any one of the preceding embodiments, wherein R¹ and R^{1′}, together with the nitrogen atom to which R¹ and R^{1′} are attached, form a 6-membered heterocyclyl optionally substituted with one or more R⁵.

Embodiment 28 provides the compound of any one of the preceding embodiments, wherein R¹ and R^{1′}, together with the nitrogen atom to which R¹ and R^{1′} are attached, form a heterocycle selected from the group consisting of consisting of

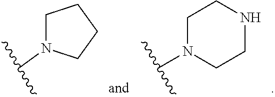 [SMILES: *N1CCCC1 ; *N1CCNCC1]

Embodiment 29 provides the compound of any one of the preceding embodiments, wherein R¹ and R^{1′}, together with the nitrogen atom to which R¹ and R^{1′} are attached, form a heterocycle selected from

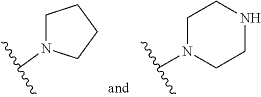 [SMILES: *N1CCCC1 ; *N1CCNCC1]

wherein the heterocycle is optionally substituted with one or more R⁵.

Embodiment 30 provides the compound of any one of the preceding embodiments, wherein R¹ and R^{1′}, together with the nitrogen atom to which R¹ and R^{1′} are attached, form a substituted heterocycle selected from

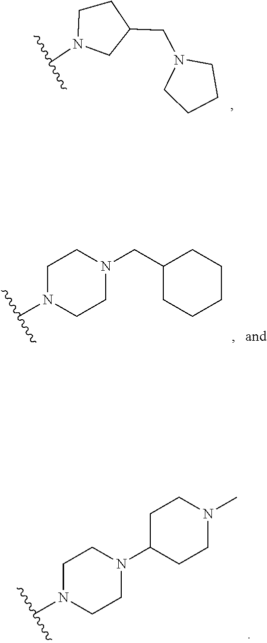 [SMILES: *N1CCC(CN2CCCC2)C1 ; *N1CCN(CC2CCCCC2)CC1 ; *N1CCN(C2CCN(C)CC2)CC1]

Embodiment 31 provides the compound of any one of the preceding embodiments, wherein R² is H.

Embodiment 32 provides the compound of any one of the preceding embodiments, wherein R² is C₁₋₆ alkyl, —C₃₋₆ cycloalkyl-C₀₋₆ alkyl, —C₀₋₆ alkyl-C₃₋₆ cycloalkyl, -(3- to 7-membered heterocyclyl)-C₀₋₆ alkyl, —C₀₋₆ alkyl-(3- to 7-membered heterocyclyl), —(C₆₋₁₀ aryl)-C₀₋₆ alkyl, —C₀₋₆ alkyl-(C₆₋₁₀ aryl), -(3- to 7-membered heteroaryl)-C₀₋₆ alkyl, —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl).

Embodiment 33 provides the compound of any one of the preceding embodiments, wherein R² is C₁₋₆ alkyl, —C₃₋₆ cycloalkyl-C₀₋₆ alkyl, —C₀₋₆ alkyl-C₃₋₆ cycloalkyl, -(3- to 7-membered heterocyclyl)-C₀₋₆ alkyl, —C₀₋₆ alkyl-(3- to 7-membered heterocyclyl), —(C₆₋₁₀ aryl)-C₀₋₆ alkyl, —C₀₋₆ alkyl-(C₆₋₁₀ aryl), -(3- to 7-membered heteroaryl)-C₀₋₆ alkyl, —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl, cycloalkyl, heterocyclyl, aryl, heteroaryl represented by R² is optionally substituted with one or more —OH, —NH₂, —NH—C(═O)—O—(C₁₋₅ alkyl), halogen, C₁₋₆ alkyl, or phenyl.

Embodiment 34 provides the compound of any one of the preceding embodiments, wherein R² is H, C₁₋₆ alkyl, —C₀₋₆ alkyl-C₃₋₆ cycloalkyl, —C₀₋₆ alkyl-(3- to 7-membered heterocyclyl), —C₀₋₆ alkyl-(C₆₋₁₀ aryl), —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl).

Embodiment 35 provides the compound of any one of the preceding embodiments, wherein R² is H, C₁₋₆ alkyl, —C₀₋₆ alkyl-C₃₋₆ cycloalkyl, —C₀₋₆ alkyl-(3- to 7-membered heterocyclyl), —C₀₋₆ alkyl-(C₆₋₁₀ aryl), —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl or aryl represented by R² is optionally substituted with one or more —OH, —NH₂, —NH—C(═O)—O—(C₁₋₆ alkyl), halogen, C₁₋₆ alkyl, or phenyl.

Embodiment 36 provides the compound of any one of the preceding embodiments, wherein R² is C₁₋₆ alkyl optionally substituted with one or more —OH, —NH₂, —NH—C(═O)—O—(C₁₋₆ alkyl), halogen, C₁₋₆ alkyl, or phenyl.

Embodiment 37 provides the compound of any one of the preceding embodiments, wherein R² is —C₀₋₆ alkyl-C₃₋₆ cycloalkyl or —C₀₋₆ alkyl-(3- to 7-membered heterocyclyl).

Embodiment 38 provides the compound of any one of the preceding embodiments, wherein R² is —C₀₋₆ alkyl-C₃₋₆ cycloalkyl or —C₀₋₆ alkyl-(3- to 7-membered heterocyclyl), wherein the alkyl, cycloalkyl, or heterocyclyl represented by R² is optionally substituted with one or more —OH, —NH₂, —NH—C(═O)—O—(C₁₋₆ alkyl), halogen, C₁₋₆ alkyl, or phenyl.

Embodiment 39 provides the compound of any one of the preceding embodiments, wherein R² is —C₀₋₆ alkyl-(C₆₋₁₀ aryl) or —C₀₋₆ alkyl-(3- to 7-membered heteroaryl).

Embodiment 40 provides the compound of any one of the preceding embodiments, wherein R² is —C₀₋₆ alkyl-(C₆₋₁₀ aryl) or —C₀₋₆ alkyl-(3- to 7-membered heteroaryl), wherein the alkyl, aryl, or heteroaryl represented by R² is optionally substituted with one or more —OH, —NH₂, —NH—C(═O)—O—(C₁₋₆ alkyl), halogen, C₁₋₆ alkyl, or phenyl.

Embodiment 41 provides the compound of any one of the preceding embodiments, wherein R² is —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl).

Embodiment 42 provides the compound of any one of the preceding embodiments, wherein R² is —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl, heterocyclyl, or aryl represented by R² is optionally substituted with one or more —OH, —NH₂, —NH—C(═O)—O—(C₁₋₆ alkyl), halogen, C₁₋₆ alkyl, or phenyl.

Embodiment 43 provides the compound of any one of the preceding embodiments, wherein R² is H, CH₃,

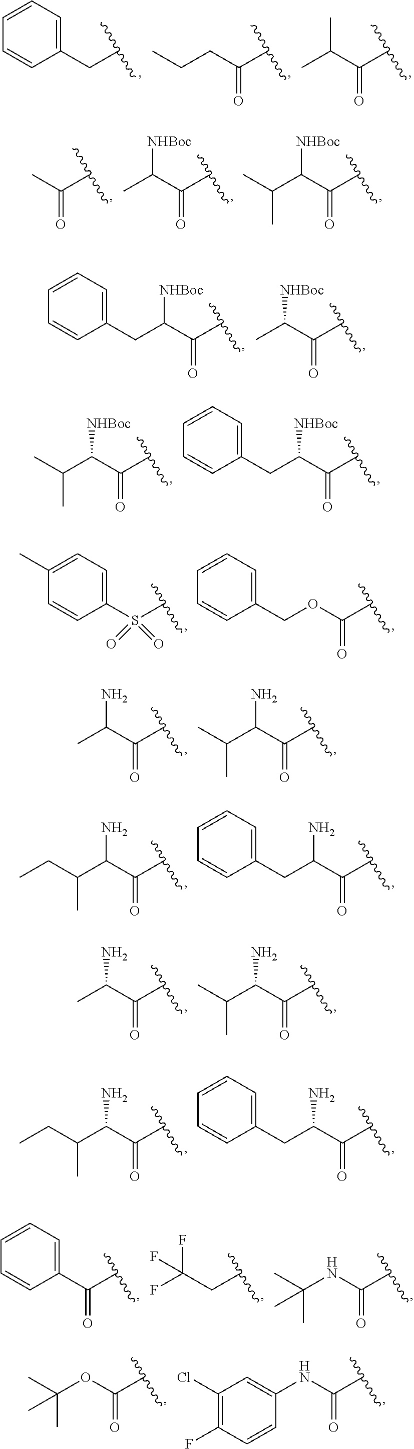

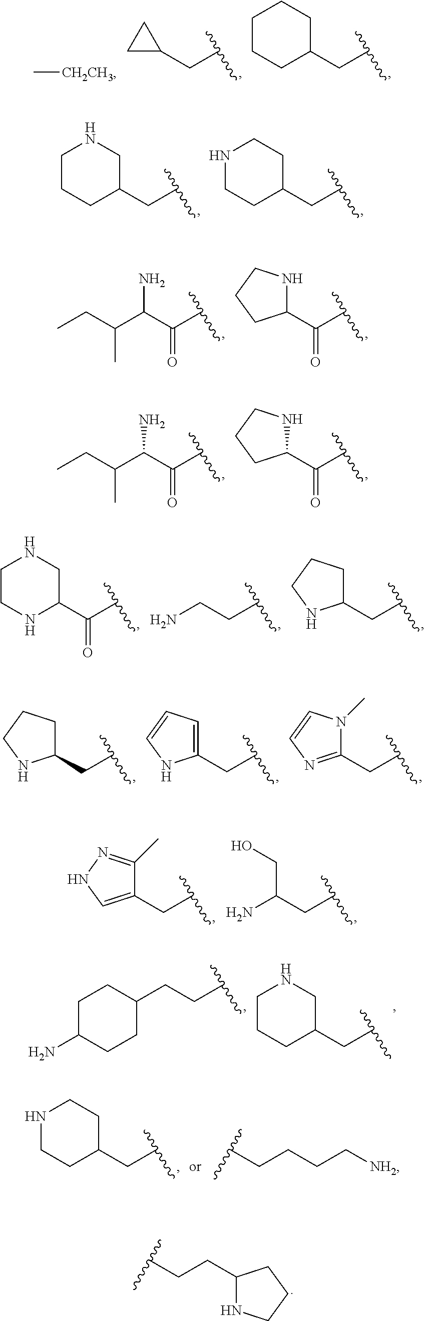

Embodiment 44 provides the compound of any one of the preceding embodiments, wherein R³ is H.

Embodiment 45 provides the compound of any one of the preceding embodiments, wherein R³ is C₁₋₆ alkyl, C₂₋₆ alkenyl, C₂₋₆ alkynyl, C₁₋₆ haloalkyl, halogen, —CN, —NO₂, —OR, —SR, —S(═O)₂R, —C(═O)R, —OC(═O)R, —NR₂, or —CO₂R.

Embodiment 46 provides the compound of any one of the preceding embodiments, wherein R⁴ is H.

Embodiment 47 provides the compound of any one of the preceding embodiments, wherein R⁴ is —C(═O)—O—(C₁₋₆ alkyl), C₁₋₆ alkyl, C₂₋₆ alkenyl, C₂₋₆ alkynyl, or C₃₋₆ cycloalkyl.

Embodiment 48 provides the compound of any one of the preceding embodiments, wherein R^{4′} is H.

Embodiment 49 provides the compound of any one of the preceding embodiments, wherein R^{4′} is —C(═O)—O—(C₁₋₆ alkyl), C₁₋₆ alkyl, C₂₋₆ alkenyl, C₂₋₆ alkynyl, or C₃₋₆ cycloalkyl.

Embodiment 50 provides the compound of any one of the preceding embodiments, wherein R⁴ and R^{4′}, together with the nitrogen atom to which R⁴ and R^{4′} are attached, form a C₅₋₆ heterocyclyl ring.

Embodiment 51 provides the compound of any one of the preceding embodiments, wherein R⁵ is —C₀₋₆-alkyl-(5- to 7-membered heterocyclyl) or —C₀₋₆-alkyl-(C₃₋₆ cycloalkyl).

Embodiment 52 provides the compound of any one of the preceding embodiments, wherein R⁵ is —C₀₋₆-alkyl-(5- to 7-membered heterocyclyl) or —C₀₋₆-alkyl-(C₃₋₆ cycloalkyl), wherein the alkyl, heterocyclyl, or cycloalkyl represented by R⁵ is optionally substituted by one or more C₁₋₆ alkyl.

Embodiment 53 provides the compound of any one of the preceding embodiments, wherein each R is independently H.

Embodiment 54 provides the compound of any one of the preceding embodiments, wherein each R is independently C₁₋₆ alkyl, C₂₋₆ alkenyl, C₂₋₆ alkynyl, or C₃₋₆ cycloalkyl.

Embodiment 55 provides the compound of any one of the preceding embodiments, wherein the compound of Formula I′ or Formula I is of Formula Ia, Ib, Ic, Id, Id′, Ie, If, Ig, or Ih:

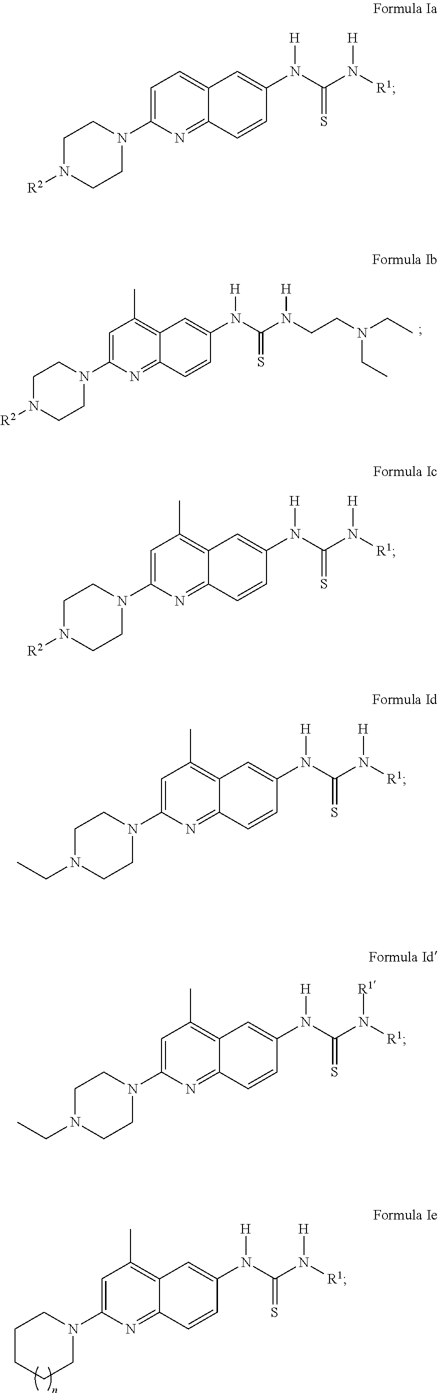

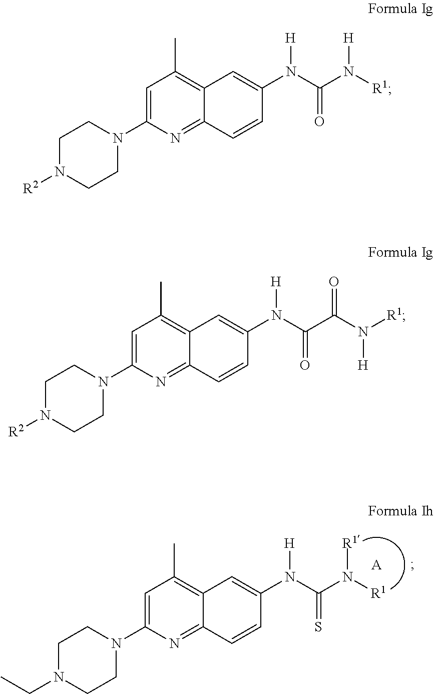

or a pharmaceutically acceptable salt thereof, wherein R¹, R^{1′}, R², and n are as described herein, and wherein ring A is a 5- to 6-membered heterocyclyl optionally substituted with one or more R⁵.

Embodiment 56 provides the compound of any one of the preceding embodiments, wherein the compound is selected from the compounds described in Tables 1-9 and prodrugs and pharmaceutically acceptable salts thereof.

Embodiment 57 provides the compound of any one of the preceding embodiments, wherein the compound is selected from the compounds described in Table 1-9 and pharmaceutically acceptable salts thereof.

Embodiment 58 provides the compound of any one of the preceding embodiments, wherein the compound is selected from the compounds described in Table 1-9.

Embodiment 59 provides s compound obtainable by, or obtained by, a method described herein; optionally, the method comprises one or more steps described in Schemes 1-23.

Embodiment 60 provides a pharmaceutical composition comprising the compound of any one of embodiments 1-59, or a pharmaceutically acceptable salt thereof, and a pharmaceutically acceptable diluent or carrier.

Embodiment 61 provides the pharmaceutical composition of embodiments 60, wherein the compound is selected from the compounds described in Table 1-9.

Embodiment 62 provides a method of modulating RAD52 activity (e.g., in vitro or in vivo), comprising contacting a cell with an effective amount of a compound of any one of embodiments 1-59 or a pharmaceutically acceptable salt of embodiment 60 or 61.

Embodiment 63 provides a method of treating or preventing a disease or disorder disclosed herein in a subject in need thereof, comprising administering to the subject a therapeutically effective amount of a compound of any one of embodiments 1-59 or a pharmaceutically acceptable salt of embodiment 60 or 61, or a pharmaceutical composition thereof.

Embodiment 64 provides the compound of any one of embodiments 1-59 or a pharmaceutically acceptable salt of embodiment 60 or 61 for use in modulating RAD52 activity (e.g., in vitro or in vivo).

Embodiment 65 provides the compound of any one of embodiments 1-59 or a pharmaceutically acceptable salt of embodiment 60 or 61 for use in treating or preventing a disease or disorder disclosed herein.

Embodiment 66 provides use of a compound of any one of embodiments 1-59 or a pharmaceutically acceptable salt of embodiment 60 or 61 in the manufacture of a medicament for modulating RAD52 activity (e.g., in vitro or in vivo).

Embodiment 67 provides use of a compound of any one of embodiments 1-59 or a pharmaceutically acceptable salt of embodiment 60 or 61 in the manufacture of a medicament for treating or preventing a disease or disorder disclosed herein.

Embodiment 68 provides the method, compound, pharmaceutical composition, or use of any one of embodiments 62-67, wherein the disease or disorder is associated with an implicated RAD52 activity.

Embodiment 69 provides the method, compound, pharmaceutical composition, or use of any one of embodiments 62-67, wherein the disease or disorder is a cancer.

Embodiment 70 provides the method, compound, pharmaceutical composition, or use of any one of embodiments 62-67, wherein the cancer has a dysfunctional BRCA1, BRCA2, PALB2, or RAD51 paralog (e.g., RAD51D or XRCC3) activity.

Embodiment 71 provides the method, compound, pharmaceutical composition, or use of any one of embodiments 62-67, wherein the cancer is squamous cell cancer, lung cancer, vulval cancer, thyroid cancer, adenocarcinoma of the lung, squamous carcinoma of the lung, cancer of the peritoneum, hepatocellular cancer, gastric or stomach cancer including gastrointestinal cancer, gastroesophageal, pancreatic cancer, brain cancer, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, biliary tract, anal carcinoma, penile carcinoma, leukemia, lymphoma, melanoma, or head and neck cancer.

Embodiment 72 provides the method, compound, pharmaceutical composition, or use of any one of embodiments 62-71, wherein the cancer is ovarian cancer.

Embodiment 73 the method, compound, pharmaceutical composition, or use of embodiment 72, wherein the ovarian cancer has a BRCA1 and/or BRCA2 mutation.

Embodiment 74 provides the method, compound, pharmaceutical composition, or use of any one of embodiments 62-71, wherein the cancer is breast cancer.

Embodiment 75 the method, compound, pharmaceutical composition, or use of embodiment 74, wherein the breast cancer has a BRCA1 and/or BRCA2 mutation.

Embodiment A provides a compound of Formula I, or a pharmaceutically acceptable salt or solvate thereof:

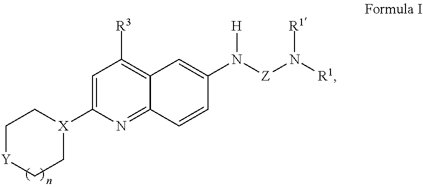

wherein

X is CH or N;

Y is CH₂ or N—R²;

Z is selected from the group consisting of

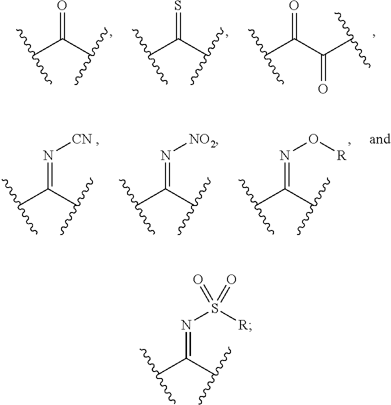

R¹ is selected from the group consisting of H, C₁₋₆ alkyl optionally substituted with one or more N(R⁴)(R^{4′}), C₀₋₆ alkyl-C₄₋₈ heterocyclyl optionally substituted on the heterocyclyl, and C₄₋₈ heterocyclyl-C₀₋₆ alkyl optionally substituted on the heterocyclyl, wherein the optional substitutions on the heterocycle are selected from the group consisting of ═O, heterocyclyl-C₀₋₂ alkyl, cycloalkyl-C₀₋₆ alkyl, C₀₋₂ alkyl-heterocyclyl, C₀₋₆ alkyl-cycloalkyl, CH₃, and CH₂CH₃;

R^{1′} is H or CH₃, or

R¹ and R^{1′}, together with the nitrogen to which R¹ and R^{1′} are connected, form a C₅₋₆ heterocyclyl optionally substituted with R₅;

R² is selected from the group consisting of H, Boc, optionally substituted C₁₋₅ alkyl, optionally substituted C₃₋₆ cycloalkyl-C₁₋₅ alkyl, optionally substituted C₃₋₇ heterocyclyl-C₀₋₅ alkyl, optionally substituted aryl-C₁₋₅ alkyl, optionally substituted heteroaryl-C₁₋₅ alkyl, optionally substituted C(═O)—C₁₋₅ alkyl, optionally substituted C(═O)—C₅₋₇ heterocyclyl, optionally substituted C(═O)—O—C₁₋₅ alkyl, optionally substituted SO₂—(C₆₋₁₀ aryl), optionally substituted C(═O)—NH—(C₆₋₁₀ aryl), optionally substituted C(═O)—NH—C₁₋₅ alkyl, optionally substituted C₀₋₅ alkyl-C₃₋₇ heterocyclyl, optionally substituted C₁₋₅ alkyl-(C₆₋₁₀ aryl), and optionally substituted C₁₋₅ alkyl-heteroaryl, wherein the optional substitution is from 1 to 4 substituents independently selected from the group consisting of OH, NH₂, NHBoc, halogen, C₁₋₃ alkyl, and phenyl;

R³ is selected from the group consisting of H, C₁₋₆ alkyl, C₁₋₆ haloalkyl, C₁₋₆ heteroalkyl, F, Cl, Br, I, CN, NO₂, OR, SR, S(═O)₂R, C(═O)R, OC(═O)R, NR₂, and CO₂R;

R⁴ and R^{4′} are each independently selected from the group consisting of H, Boc, and C₁₋₆ hydrocarbyl, or R⁴ and R^{4′}, together with the nitrogen to which R⁴ and R^{4′} are connected, form a C₅₋₇ heterocyclyl ring;

R₅ is selected from the group consisting of heterocyclyl-C₀₋₆-alkyl, cycloalkyl-C₀₋₆-alkyl, C₀₋₆-alkyl-heterocyclyl and C₀₋₆-alkyl-cycloalkyl;

each occurrence of R is independently selected from the group consisting of C₁₋₁₀ hydrocarbyl and H; and

n is 0 or 1; and

provided that R² and R³ are not both CH₃, and

provided that the compound is not 1-(2-(diethylamino)ethyl)-3-(4-methyl-2-(4-ethylpiperazin-1-yl)quinolin-6-yl)thiourea, 1-isopropyl-3-(4-methyl-2-(pyrrolidin-1-yl)quinolin-6-yl)thiourea, 1-(4-Ethyl-phenyl)-3-[2-(4-ethyl-piperazin-1-yl)-4-methyl-quinolin-6-yl]-1-propyl-thiourea, 1-Benzyl-3-[2-(4-ethyl-piperazin-1-yl)-4-methyl-quinolin-6-yl]-1-methyl-thiourea, 1-(4-Ethoxy-phenyl)-1-ethyl-3-[2-(4-ethyl-piperazin-1-yl)-4-methyl-quinolin-6-yl]-thiourea, 1-[2-(4-Ethyl-piperazin-1-yl)-4-methyl-quinolin-6-yl]-3-thiophen-2-ylmethyl-thiourea, 1-[2-(4-Ethyl-piperazin-1-yl)-4-methyl-quinolin-6-yl]-3-(2-methoxy-benzyl)-thiourea, 1-[2-(4-Ethyl-piperazin-1-yl)-4-methyl-quinolin-6-yl]-3-(4-fluoro-benzyl)-thiourea, 1-[2-(4-Ethyl-piperazin-1-yl)-4-methyl-quinolin-6-yl]-3-furan-2-ylmethyl-thiourea, 1-Ethyl-3-[2-(4-ethyl-piperazin-1-yl)-4-methyl-quinolin-6-yl]-1-(4-fluoro-phenyl)-thiourea, 1-(2-Ethyl-phenyl)-3-[2-(4-ethyl-piperazin-1-yl)-4-methyl-quinolin-6-yl]-1-methyl-thiourea, 1-Benzo[1,3]dioxol-5-ylmethyl-3-[2-(4-ethyl-piperazin-1-yl)-4-methyl-quinolin-6-yl]-thiourea, 1-(2-(dimethylamino)ethyl)-3-(4-methyl-2-(pyrrolidin-1-yl)quinolin-6-yl)thiourea, 1-(3-(3,5-dimethylpiperidin-1-yl)propyl)-3-(2-(4-ethylpiperazin-1-yl)-4-methylquinolin-6-yl)thiourea, N-(2-(4-ethylpiperazin-1-yl)-4-methylquinolin-6-yl)-[1,4′-bipiperidine]-1′-carbothioamide, 1-(2-(4-ethylpiperazin-1-yl)-4-methylquinolin-6-yl)-3-(3-(2-ethylpiperidin-1-yl)propyl)thiourea, or 1-((1-benzylpiperidin-4-yl)methyl)-3-(2-(piperazin-1-yl)quinolin-6-yl)thiourea.

Embodiment B provides the compound of embodiment A, wherein X is N.

Embodiment C provides the compound of any one of embodiments A, wherein n is 1.

Embodiment D provides the compound of any one of embodiments A-C, wherein Z is selected from the group consisting of

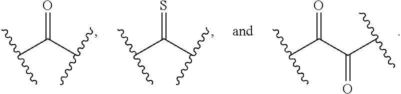

Embodiment E provides the compound of any one of embodiments A-C, wherein Z is

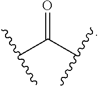 [SMILES: *C(*)=O]

Embodiment F provides the compound of any one of embodiments A-C, wherein Z is

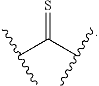 [SMILES: *C(*)=S]

Embodiment G provides the compound of any one of embodiments A-C, wherein Z is

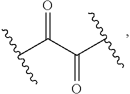 [SMILES: *C(=O)C(*)=O]

Embodiment H provides the compound of any one of embodiments A-G, wherein Y is CH₂.

Embodiment I provides the compound of any one of embodiments A-G, wherein Y is N—R².

Embodiment J provides the compound of any one of embodiments A-I, wherein R¹ is C₁₋₆ alkyl-N(R⁴)(R^{4′}).

Embodiment K provides the compound of any one of embodiments A-I, wherein R¹ is C₀₋₆ alkyl-C₄₋₈ heterocyclyl.

Embodiment L provides the compound of any one of embodiments A-K, wherein R¹ is selected from the group consisting of H,

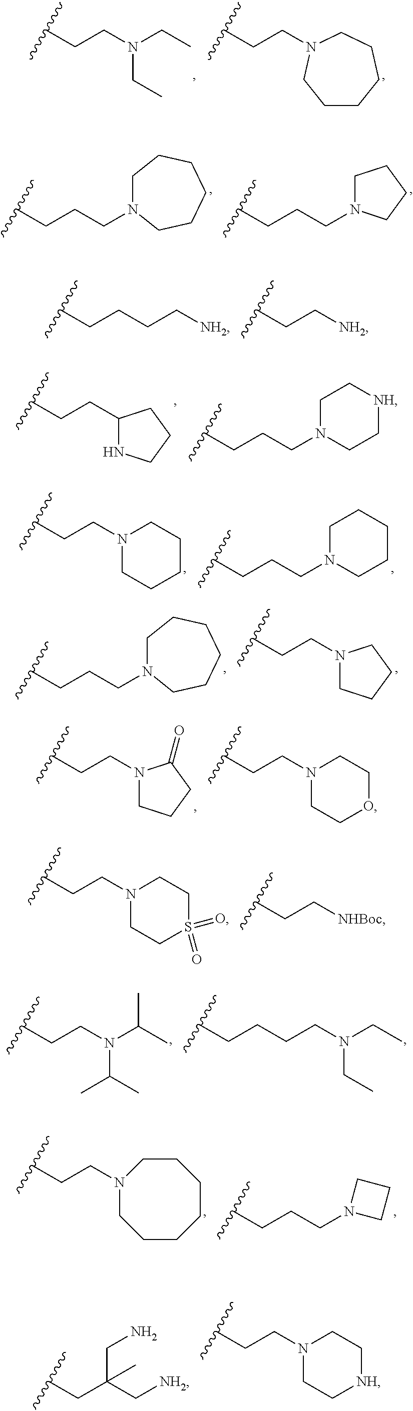

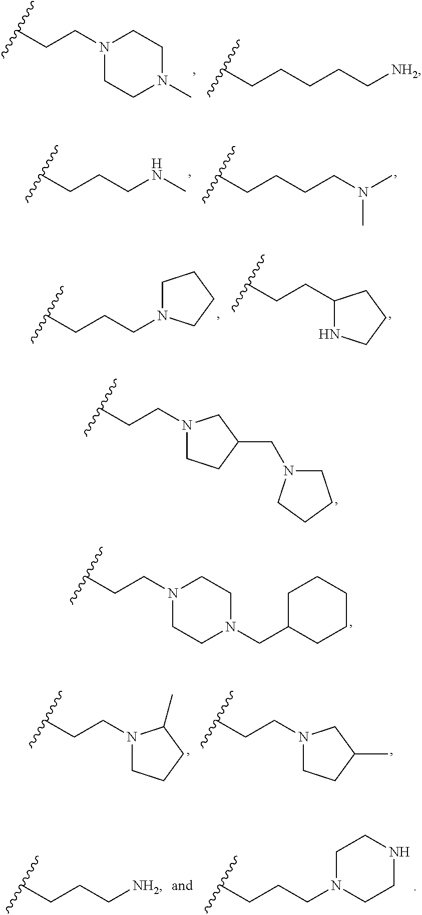

Embodiment M provides the compound of any one of embodiments A-H, wherein R¹ is

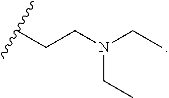 [SMILES: *CCN(CC)CC]

Embodiment N provides the compound of any one of embodiments A-M, wherein R¹ and R^{1′}, together with the nitrogen to which R¹ and R^{1′} are connected, form a C₅₋₆ heterocyclyl optionally substituted with R₅.

Embodiment O provides the compound of any one of embodiments A-N, wherein R¹ and R^{1′}, together with the nitrogen to which R¹ and R^{1′} are connected, form an unsubstituted C₅₋₆ heterocyclyl.

Embodiment P provides the compound of any one of embodiments A-O, wherein R¹ and R^{1′}, together with the nitrogen to which R¹ and R^{1′} are connected, form an unsubstituted heterocycle which is

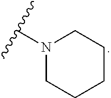 [SMILES: *N1CCCCC1]

Embodiment Q provides the compound of any one of embodiments A-I, wherein R¹ and R^{1′}, together with the nitrogen to which R¹ and R^{1′} are connected, form a heterocycle selected from the group consisting of consisting of

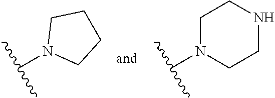 [SMILES: *N1CCCC1 ; *N1CCNCC1]

and wherein the heterocycle is optionally further substituted with R₅.

Embodiment R provides the compound of any one of embodiments A-Q, wherein R¹ and R^{1′}, together with the nitrogen to which R¹ and R^{1′} are connected, form a substituted heterocycle selected from the group consisting of

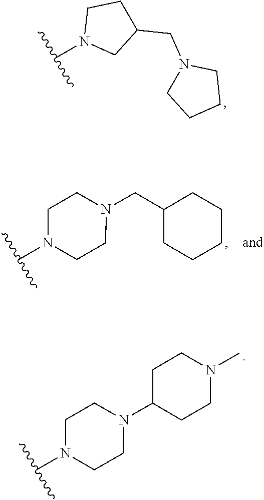 [SMILES: *N1CCC(CN2CCCC2)C1 ; *N1CCN(CC2CCCCC2)CC1 ; *N1CCN(C2CCN(C)CC2)CC1]

Embodiment S provides the compound of any one of embodiments A-R, wherein R² is selected from the group consisting of H, CH₃,

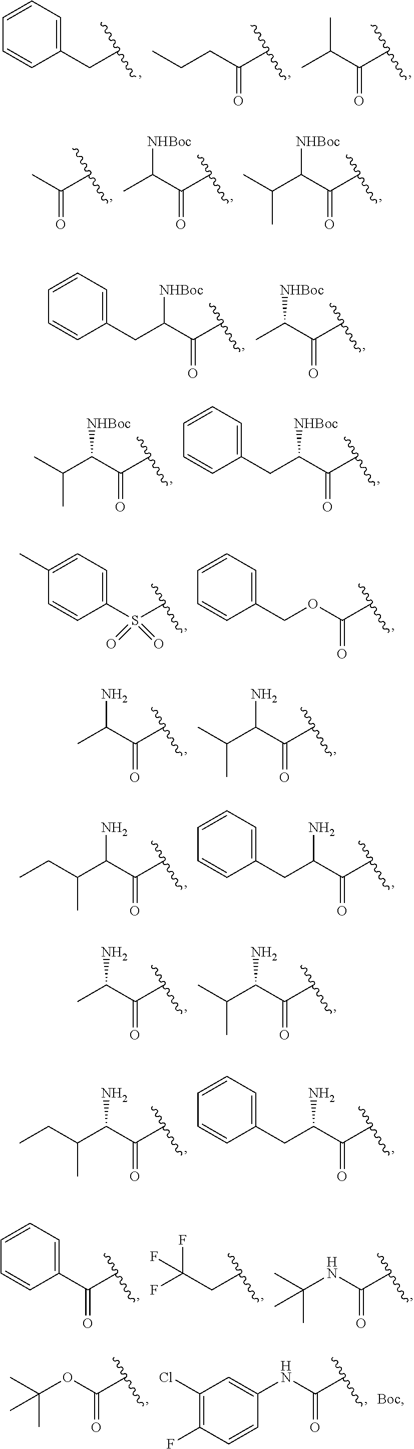

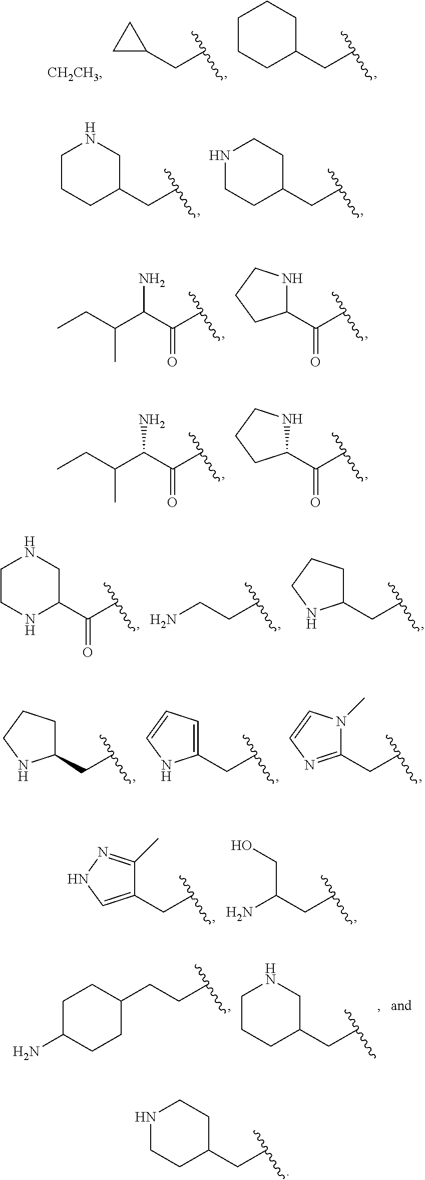

Embodiment T provides the compound of any one of embodiments A-S, wherein the compound has a structure selected from the group consisting of

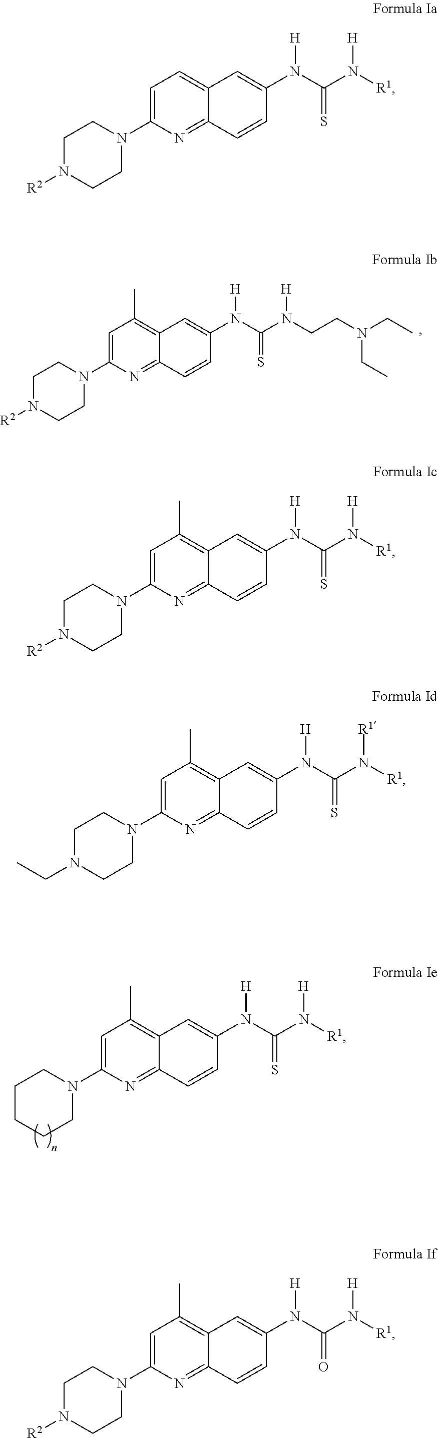

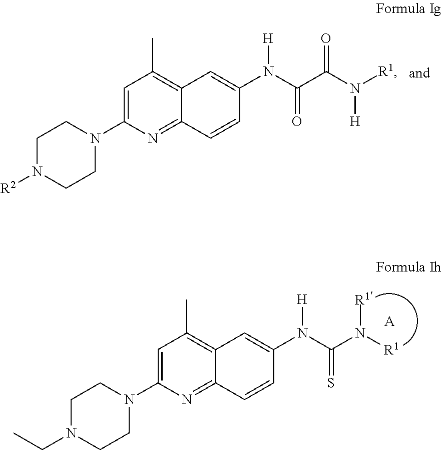

Embodiment U provides a pharmaceutical composition comprising at least one compound of any one of embodiments A-T and at least one pharmaceutically acceptable carrier.

Embodiment V provides composition of embodiment U, further comprising at least one additional therapeutic agent that treats cancer.

Embodiment W provides a method of treating a cancer in a subject in need thereof, the method comprising administering to the subject a therapeutically effective amount of at least one compound of any one of embodiments A-V.

Embodiment X provides the method of embodiment W, wherein the method further comprises administering to the subject at least one additional therapeutic agent that treats cancer.

Embodiment Y provides the method of any one of embodiments W-X, wherein the at least one compound and the at least one additional therapeutic agent are co-administered to the subject.

Embodiment Z provides the method of any one of embodiments W-Y, wherein the subject is human.

Embodiment AA provides the method of any one of embodiments W-Z, wherein the cancer is selected from the group consisting of squamous cell cancer, lung cancer including small-cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer.

Embodiment BB provides the method of any one of embodiments W-AA, wherein the cancer is ovarian cancer or breast cancer.

Embodiment CC provides the method of any one of embodiments W-BB, wherein the human has mutations in BRCA1 and/or BRCA2.

Embodiment DD provides a method of treating a RAD52 related disease or disorder in a subject in need thereof, the method comprising administering to the subject a therapeutically effective amount of at least one compound of any one of embodiments A-V.

Embodiment EE provides the method of embodiment 30, wherein the RAD52 related disease or disorder comprises cancer.

Embodiment FF provides the method of any one of embodiments DD-EE, the method further comprising administering to the subject at least one additional therapeutic agent that treats cancer.

Embodiment GG provides the method of any one of embodiments DD-FF, wherein the at least one compound and the at least one additional therapeutic agent are co-administered to the subject.

Embodiment HH provides the method of any one of embodiments DD-GG, wherein the at least one compound and the at least one additional therapeutic agent are coformulated.

Embodiment II provides the method of any one of embodiments DD-HH, wherein the subject is human.

Embodiment JJ provides the method of any one of embodiments DD-II, wherein the cancer is selected from the group consisting of squamous cell cancer, lung cancer including small-cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer.

Embodiment KK provides the method of any one of embodiments DD-JJ, wherein the cancer is ovarian cancer or breast cancer.

Embodiment LL provides the method of any one of embodiments DD-KK, wherein the subject has mutations in BRCA1 and/or BRCA2.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula I′: 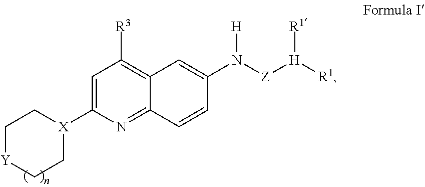
wherein:
X is CH or N;
Y is CH₂ or N—R²;
Z is 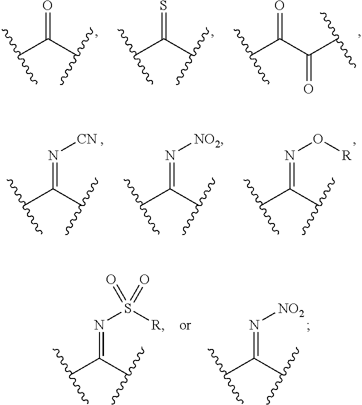
R¹ is H, C₁₋₆ alkyl optionally substituted with one or more N(R⁴)(R^{4′}), —C₀₋₆ alkyl-(4- to 8-membered heterocyclyl), or -(4- to 8-membered heterocyclyl)-C₀₋₆ alkyl,
wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl;
R^{1′} is H or C₁₋₆ alkyl,
or R¹ and R^{1′}, together with the nitrogen atom to which R¹ and R^{1′} are attached, form a 5- to 6-membered heterocyclyl optionally substituted with one or more R⁵;
R² is H, C₁₋₆ alkyl, —C₃₋₆ cycloalkyl-C₀₋₆ alkyl, —C₀₋₆ alkyl-C₃₋₆ cycloalkyl, -(3- to 7-membered heterocyclyl)-C₀₋₆ alkyl, —C₀₋₅ alkyl-(3- to 7-membered heterocyclyl), —(C₆₋₁₀ aryl)-C₀₋₆ alkyl, —C₀₋₅ alkyl-(C₆₋₁₀ aryl), -(3- to 7-membered heteroaryl)-C₀₋₆ alkyl, —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl),
wherein the alkyl, cycloalkyl, heterocyclyl, aryl, or heteroaryl represented by R² is optionally substituted with one or more —OH, —NH₂, —NH—C(═O)—O—(C₁₋₆ alkyl), halogen, C₁₋₆ alkyl, or phenyl;
R³ is H, C₁₋₆ alkyl, C₂₋₆ alkenyl, C₂₋₆ alkynyl, C₁₋₆ haloalkyl, halogen, —CN, —NO₂, —OR, —SR, —S(═O)₂R, —C(═O)R, —OC(═O)R, —NR₂, or —CO₂R;
each R⁴ and R^{4′} is independently H, —C(═O)—O—(C₁₋₆ alkyl), C₁₋₆ alkyl, C₂₋₆ alkenyl, C₂₋₆ alkynyl, or C₃₋₆ cycloalkyl;
R⁵ is -(5- to 7-membered heterocyclyl)-C₀₋₆-alkyl, —C₀₋₆-alkyl-(5- to 7-membered heterocyclyl), —(C₃₋₆ cycloalkyl)-C₀₋₆-alkyl, or —C₀₋₆-alkyl-(C₃₋₆ cycloalkyl),
wherein the alkyl, heterocyclyl, or cycloalkyl represented by R⁵ is optionally substituted by one or more C₁₋₆ alkyl;
each R is independently H, C₁₋₆ alkyl, C₂₋₆ alkenyl, C₂₋₆ alkynyl, or C₃₋₆ cycloalkyl; and
n is 0 or 1,
provided that R² and R³ are not simultaneously CH₃,
or a pharmaceutically acceptable salt or solvate thereof.

2. The compound of claim 1, wherein:
X is N;
Y is CH₂ or N—R²;
Z is 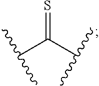 [SMILES: *C(*)=S]
R¹ is H, C₁₋₆ alkyl optionally substituted with one or more N(R⁴)(R^{4′}), or —C₀₋₆ alkyl-(4- to 8-membered heterocyclyl),
wherein the heterocyclyl is optionally substituted with one or more oxo, —C₀₋₂ alkyl-(4- to 8-membered heterocyclyl), —C₀₋₆ alkyl-(C₃₋₇ cycloalkyl), or C₁₋₃ alkyl;
R^{1′} is H or —CH₃,
or R¹ and R^{1′}, together with the nitrogen atom to which R¹ and R^{1′} are attached, form a 5- to 6-membered heterocyclyl optionally substituted with one or more R⁵;
R² is H, C₁₋₅ alkyl, —C₀₋₅ alkyl-C₃₋₆ cycloalkyl, —C₀₋₅ alkyl-(3- to 7-membered heterocyclyl), —C₀₋₅ alkyl-(C₆₋₁₀ aryl), —C₀₋₅ alkyl-(3- to 7-membered heteroaryl), —C(═O)—C₁₋₅ alkyl, —C(═O)—(C₆₋₁₀ aryl), —C(═O)-(5- to 7-membered heterocyclyl), —C(═O)—O—C₁₋₅ alkyl, —SO₂—(C₆₋₁₀ aryl), —C(═O)—NH—C₁₋₅ alkyl, or —C(═O)—NH—(C₆₋₁₀ aryl),
wherein the alkyl, cycloalkyl, heterocyclyl, aryl, or heteroaryl represented by R² is optionally substituted with one or more —OH, —NH₂, —NH—C(═O)—O—(C₁₋₅ alkyl), halogen, C₁₋₃ alkyl, or phenyl;
R³ is H or C₁₋₆ alkyl;
each R⁴ and R^{4′} is independently H, —C(═O)—O—(C₁₋₅ alkyl), or C₁₋₆ alkyl;
R⁵ is —C₀₋₆-alkyl-(5- to 7-membered heterocyclyl) or —C₀₋₆-alkyl-(C₃₋₆ cycloalkyl),
wherein the alkyl, heterocyclyl, or cycloalkyl represented by R⁵ is optionally substituted by one or more C₁₋₆ alkyl;
each R is independently H or C₁₋₆ alkyl; and
n is 0 or 1,
provided that R² and R³ are not simultaneously CH₃.

3. The compound of claim 1, wherein the compound of Formula I′ is not 1-(2-(diethylamino)ethyl)-3-(4-methyl-2-(4-ethylpiperazin-1-yl)quinolin-6-yl)thiourea, 1-isopropyl-3-(4-methyl-2-(pyrrolidin-1-yl)quinolin-6-yl)thiourea, 1-(4-Ethyl-phenyl)-3-[2-(4-ethyl-piperazin-1-yl)-4-methyl-quinolin-6-yl]-1-propyl-thiourea, 1-Benzyl-3-[2-(4-ethyl-piperazin-1-yl)-4-methyl-quinolin-6-yl]-1-methyl-thiourea, 1-(4-Ethoxy-phenyl)-1-ethyl-3-[2-(4-ethyl-piperazin-1-yl)-4-methyl-quinolin-6-yl]-thiourea, 1-[2-(4-Ethyl-piperazin-1-yl)-4-methyl-quinolin-6-yl]-3-thiophen-2-ylmethyl-thiourea, 1-[2-(4-Ethyl-piperazin-1-yl)-4-methyl-quinolin-6-yl]-3-(2-methoxy-benzyl)-thiourea, 1-[2-(4-Ethyl-piperazin-1-yl)-4-methyl-quinolin-6-yl]-3-(4-fluoro-benzyl)-thiourea, 1-[2-(4-Ethyl-piperazin-1-yl)-4-methyl-quinolin-6-yl]-3-furan-2-ylmethyl-thiourea, 1-Ethyl-3-[2-(4-ethyl-piperazin-1-yl)-4-methyl-quinolin-6-yl]-1-(4-fluoro-phenyl)-thiourea, 1-(2-Ethyl-phenyl)-3-[2-(4-ethyl-piperazin-1-yl)-4-methyl-quinolin-6-yl]-1-methyl-thiourea, 1-Benzo[1,3]dioxol-5-ylmethyl-3-[2-(4-ethyl-piperazin-1-yl)-4-methyl-quinolin-6-yl]-thiourea, 1-(2-(dimethylamino)ethyl)-3-(4-methyl-2-(pyrrolidin-1-yl)quinolin-6-yl)thiourea, 1-(3-(3,5-dimethylpiperidin-1-yl)propyl)-3-(2-(4-ethylpiperazin-1-yl)-4-methylquinolin-6-yl)thiourea, N-(2-(4-ethylpiperazin-1-yl)-4-methylquinolin-6-yl)-[1,4′-bipiperidine]-1′-carbothioamide, 1-(2-(4-ethylpiperazin-1-yl)-4-methylquinolin-6-yl)-3-(3-(2-ethylpiperidin-1-yl)propyl)thiourea, or 1-((1-benzylpiperidin-4-yl)methyl)-3-(2-(piperazin-1-yl)quinolin-6-yl)thiourea.

4. The compound of claim 1, wherein X is N.

5. The compound of claim 1, wherein n is 1.

6. The compound of claim 1, wherein Z is

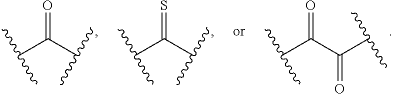

7. The compound of claim 1, wherein Y is N—R².

8. The compound of claim 1, wherein R¹ is H.

9. The compound of claim 1,
wherein R¹ is C₁₋₆ alkyl optionally substituted with one or more N(R⁴)(R^{4′}), —C₀₋₆ alkyl-(4- to 8-membered heterocyclyl), or -(4- to 8-membered heterocyclyl)-C₀₋₆ alkyl,
wherein the heterocyclyl is optionally substituted with one or more oxo, -(4- to 8-membered heterocyclyl)-C₀₋₆ alkyl, —(C₃₋₇ cycloalkyl)-C₀₋₆ alkyl, —C₀₋₆ alkyl-(4- to 8-membered heterocyclyl), —C₀₋₆ alkyl-(C₃₋₇ cycloalkyl), or C₁₋₆ alkyl.

10. The compound of claim 1, wherein R^{1′} is H or methyl.

11. The compound of claim 1, wherein R¹ is H,

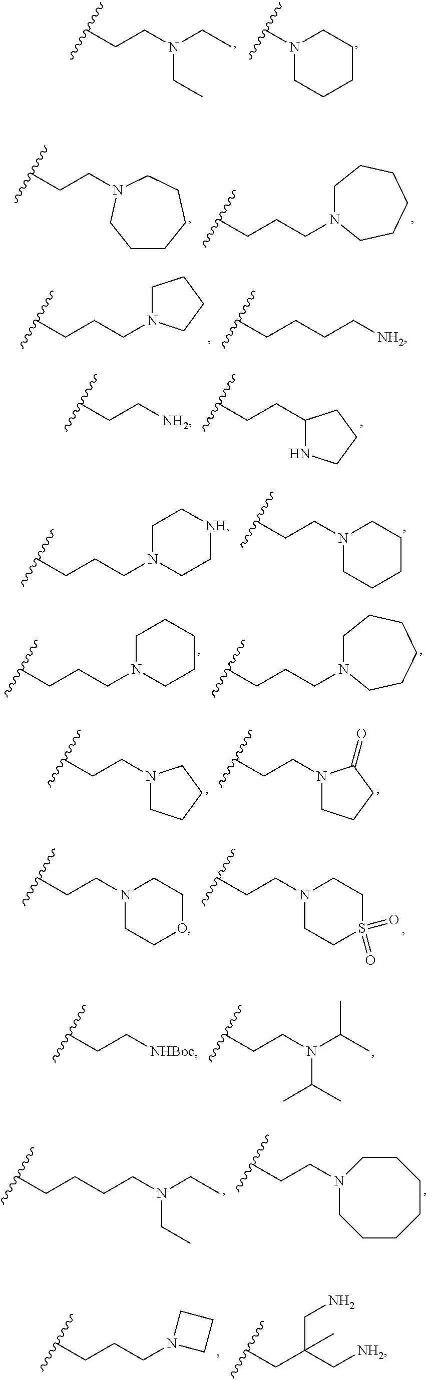

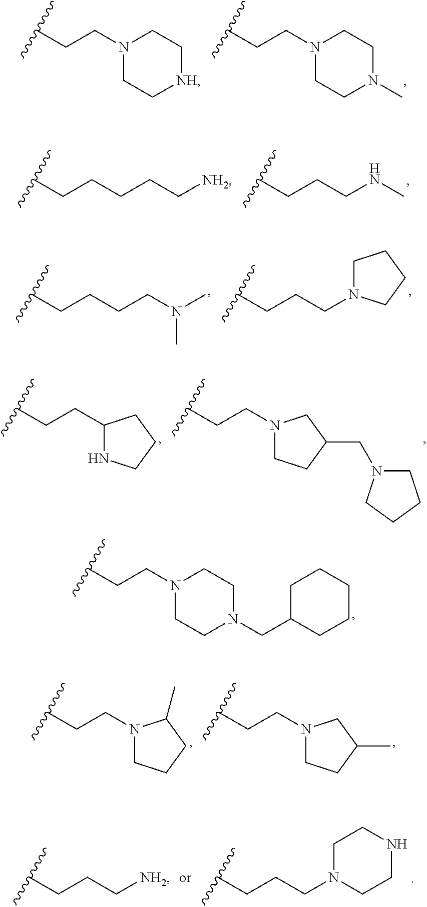

12. The compound of claim 1, wherein R¹ and R^{1′}, together with the nitrogen atom to which R¹ and R^{1′} are attached, form a 5-membered heterocyclyl optionally substituted with one or more R⁵.

13. The compound of claim 1, wherein R¹ and R^{1′}, together with the nitrogen atom to which R¹ and R^{1′} are attached, form a 6-membered heterocyclyl optionally substituted with one or more R⁵.

14. The compound of claim 1, wherein R¹ and R^{1′}, together with the nitrogen atom to which R¹ and R^{1′} are attached, form a heterocycle selected from 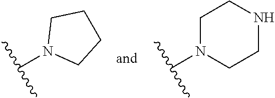 [SMILES: *N1CCCC1 ; *N1CCNCC1]
and wherein the heterocycle is optionally substituted with one or more R⁵.

15. The compound of claim 1, wherein R¹ and R^{1′}, together with the nitrogen atom to which R¹ and R^{1′} are attached, form a substituted heterocycle selected from

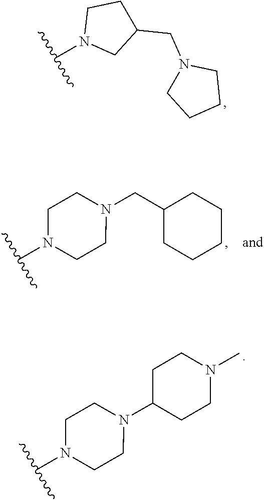 [SMILES: *N1CCC(CN2CCCC2)C1 ; *N1CCN(CC2CCCCC2)CC1 ; *N1CCN(C2CCN(C)CC2)CC1]

16. (canceled)

17. The compound of claim 1, wherein R² is H, C₁₋₆ alkyl, —C₀₋₆ alkyl-C₃₋₆ cycloalkyl, —C₀₋₆ alkyl-(3- to 7-membered heterocyclyl), —C₀₋₆ alkyl-(C₆₋₁₀ aryl), —C₀₋₆ alkyl-(3- to 7-membered heteroaryl), —C(═O)—C₁₋₆ alkyl, —C(═O)—(C₆₋₁₀ aryl), —C(═O)-(5- to 7-membered heterocyclyl), —C(═O)—O—C₁₋₆ alkyl, —SO₂—(C₆₋₁₀ aryl), —C(═O)—NH—C₁₋₆ alkyl, or —C(═O)—NH—(C₆₋₁₀ aryl), wherein the alkyl, cycloalkyl, heterocyclyl, aryl, heteroaryl represented by R² is optionally substituted with one or more —OH, —NH₂, —NH—C(═O)—O—(C₁₋₅ alkyl), halogen, C₁₋₆ alkyl, or phenyl.

18. The compound of claim 1, wherein R² is H, CH₃,

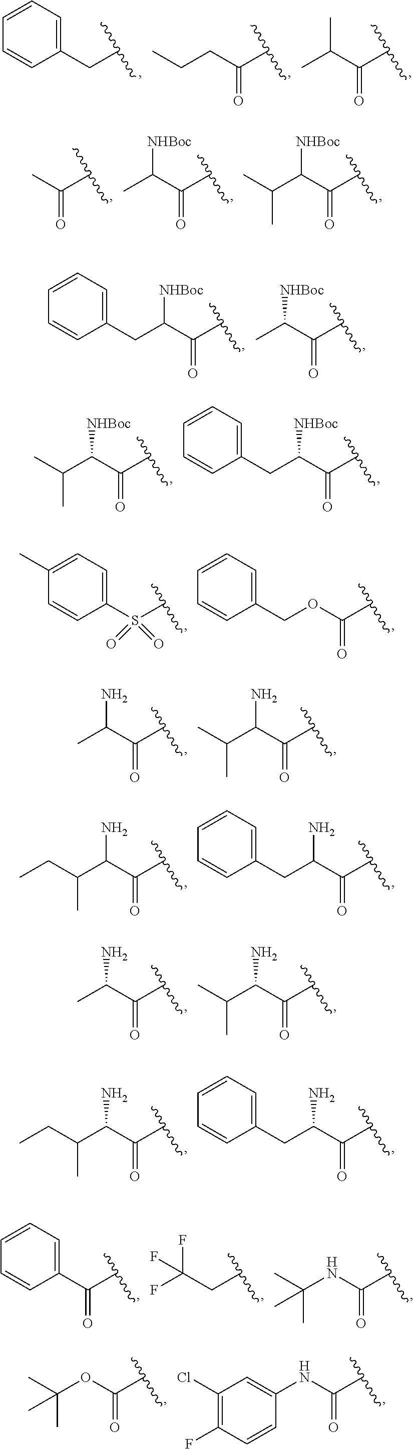

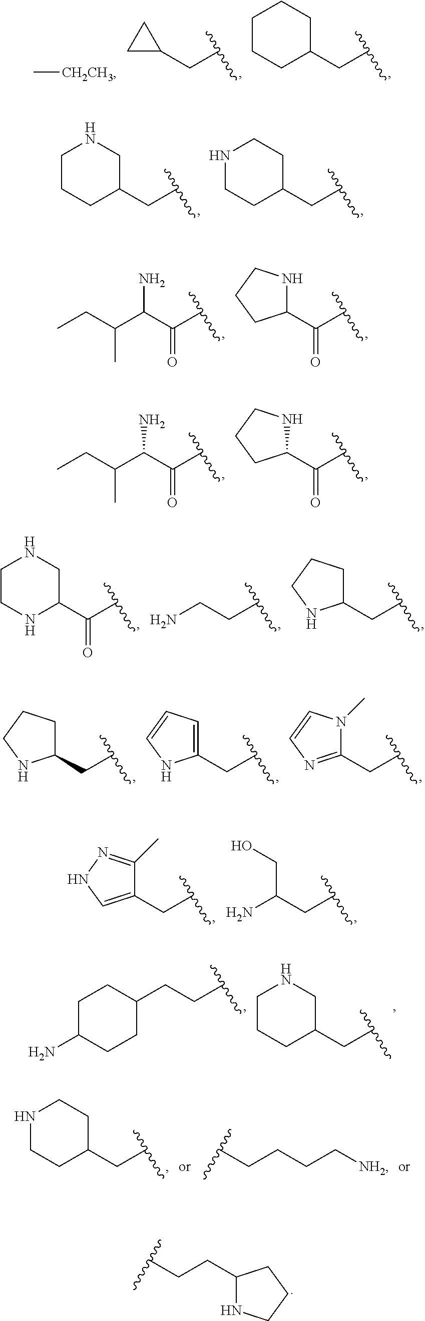

19. (canceled)

20. The compound of claim 1, wherein R³ is H, C₁₋₆ alkyl, C₂₋₆ alkenyl, C₂₋₆ alkynyl, C₁₋₆ haloalkyl, halogen, —CN, —NO₂, —OR, —SR, —S(═O)₂R, —C(═O)R, —OC(═O)R, —NR₂, or —CO₂R.

21. (canceled)

22. The compound of claim 1, wherein R⁴ or R^{4′} is independently H, —C(═O)—O—(C₁₋₆ alkyl), C₁₋₆ alkyl, C₂₋₆ alkenyl, C₂₋₆ alkynyl, or C₃₋₆ cycloalkyl.

23. The compound of claim 1, wherein R⁴ and R^{4′}, together with the nitrogen atom to which R⁴ and R^{4′} are attached, form a C₅₋₆ heterocyclyl ring.

24. The compound of claim 1, wherein R⁵ is —C₀₋₆-alkyl-(5- to 7-membered heterocyclyl) or —C₀₋₆-alkyl-(C₃₋₆ cycloalkyl), wherein the alkyl, heterocyclyl, or cycloalkyl represented by R⁵ is optionally substituted by one or more C₁₋₆ alkyl.

25. (canceled)

26. The compound of claim 1, wherein each R is independently H, C₁₋₆ alkyl, C₂₋₆ alkenyl, C₂₋₆ alkynyl, or C₃₋₆ cycloalkyl.

27. The compound of claim 1, wherein the compound of Formula I′ is of Formula Ia, Ib, Ic, Id, Id′, Ie, If, Ig, or Ih: 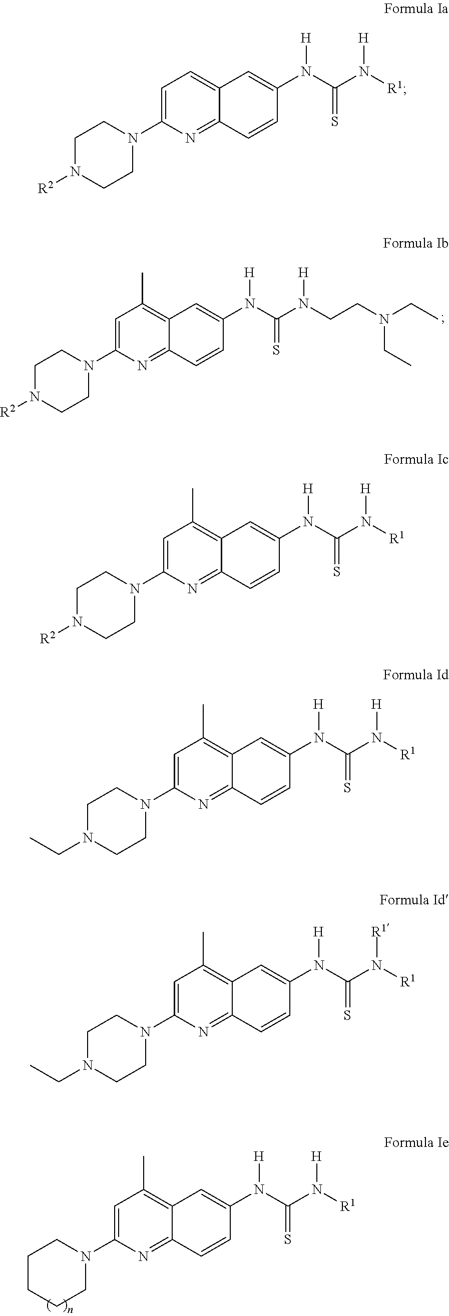 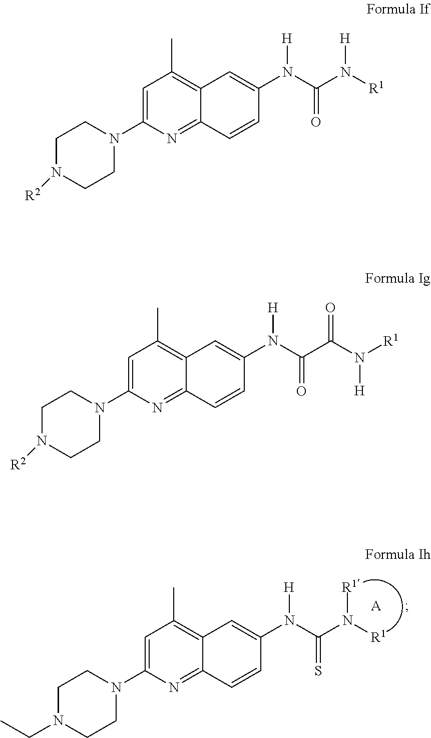
or a pharmaceutically acceptable salt thereof, wherein R¹, R^{1′}, R², and n are as described herein, and wherein ring A is a 5- to 6-membered heterocyclyl optionally substituted with one or more R⁵.

28. The compound of claim 1, which is selected from the group consisting of:

| Compound | |
|---|---|
| No. | Structure |
| 0001 | 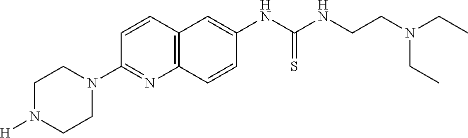 [SMILES: CCN(CC)CCNC(=S)Nc1ccc2nc(N3CCNCC3)ccc2c1] |
| 0002 | 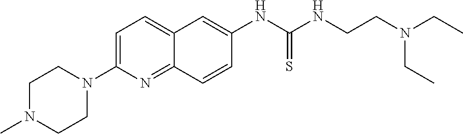 [SMILES: CCN(CC)CCNC(=S)Nc1ccc2nc(N3CCN(C)CC3)ccc2c1] |
| 0003 | 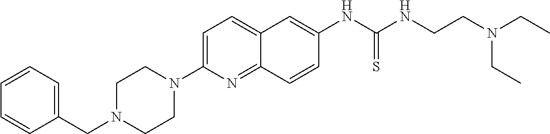 [SMILES: CCN(CC)CCNC(=S)Nc1ccc2nc(N3CCN(Cc4ccccc4)CC3)ccc2c1] |
| 0004 | 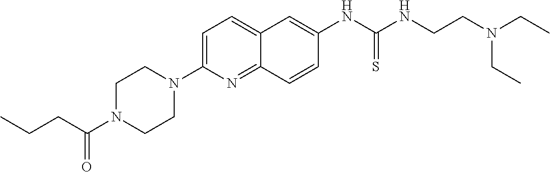 [SMILES: CCCC(=O)N1CCN(c2ccc3cc(NC(=S)NCCN(CC)CC)ccc3n2)CC1] |
| 0005 | 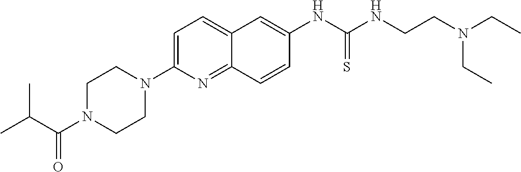 [SMILES: CCN(CC)CCNC(=S)Nc1ccc2nc(N3CCN(C(=O)C(C)C)CC3)ccc2c1] |
| 0006 | 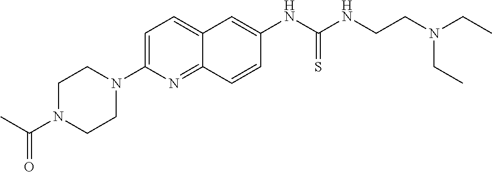 [SMILES: CCN(CC)CCNC(=S)Nc1ccc2nc(N3CCN(C(C)=O)CC3)ccc2c1] |
| 0007 | 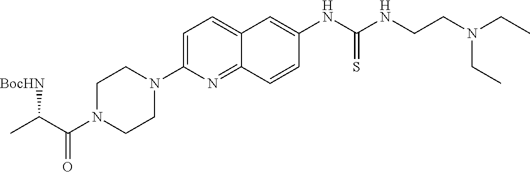 [SMILES: CCN(CC)CCNC(=S)Nc1ccc2nc(N3CCN(C(=O)[C@H](C)NC(=O)OC(C)(C)C)CC3)ccc2c1] |
| 0008 | 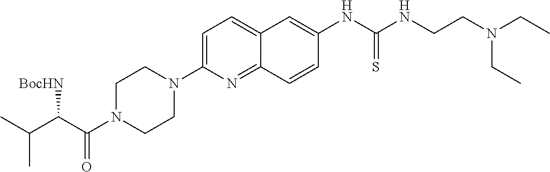 [SMILES: CCN(CC)CCNC(=S)Nc1ccc2nc(N3CCN(C(=O)[C@@H](NC(=O)OC(C)(C)C)C(C)C)CC3)ccc2c1] |
| 0009 | 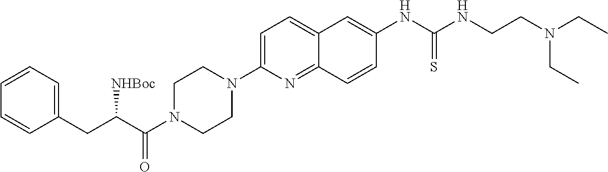 [SMILES: CCN(CC)CCNC(=S)Nc1ccc2nc(N3CCN(C(=O)[C@H](Cc4ccccc4)NC(=O)OC(C)(C)C)CC3)ccc2c1] |
| 0010 | 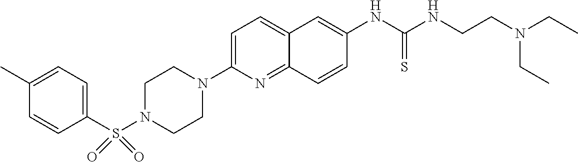 [SMILES: CCN(CC)CCNC(=S)Nc1ccc2nc(N3CCN(S(=O)(=O)c4ccc(C)cc4)CC3)ccc2c1] |
| 0011 | 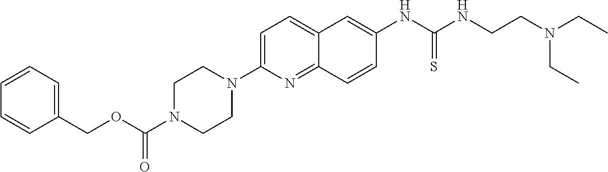 [SMILES: CCN(CC)CCNC(=S)Nc1ccc2nc(N3CCN(C(=O)OCc4ccccc4)CC3)ccc2c1] |
| 0012 | 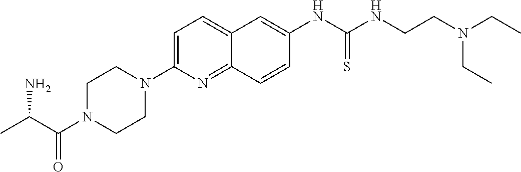 [SMILES: CCN(CC)CCNC(=S)Nc1ccc2nc(N3CCN(C(=O)[C@H](C)N)CC3)ccc2c1] |
| 0013 | 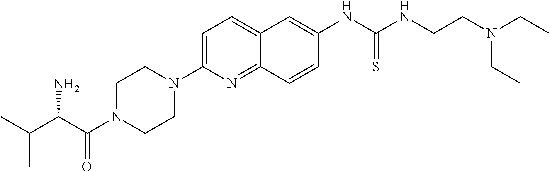 [SMILES: CCN(CC)CCNC(=S)Nc1ccc2nc(N3CCN(C(=O)[C@@H](N)C(C)C)CC3)ccc2c1] |
| 0014 | 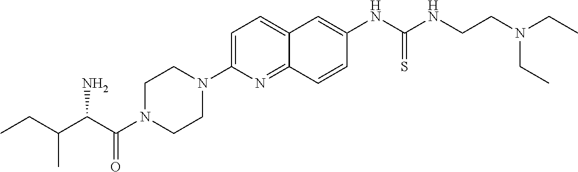 [SMILES: CCC(C)[C@H](N)C(=O)N1CCN(c2ccc3cc(NC(=S)NCCN(CC)CC)ccc3n2)CC1] |
| 0015 | 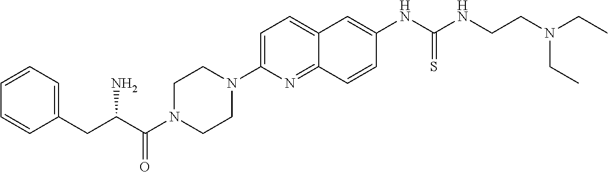 [SMILES: CCN(CC)CCNC(=S)Nc1ccc2nc(N3CCN(C(=O)[C@@H](N)Cc4ccccc4)CC3)ccc2c1] |
| 0016 | 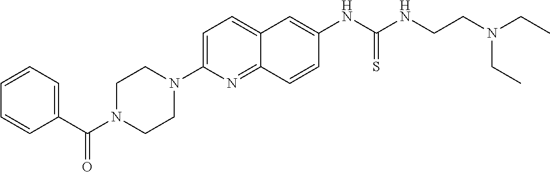 [SMILES: CCN(CC)CCNC(=S)Nc1ccc2nc(N3CCN(C(=O)c4ccccc4)CC3)ccc2c1] |
| 0017 | 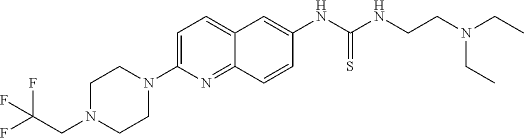 [SMILES: CCN(CC)CCNC(=S)Nc1ccc2nc(N3CCN(CC(F)(F)F)CC3)ccc2c1] |
| 0018 | 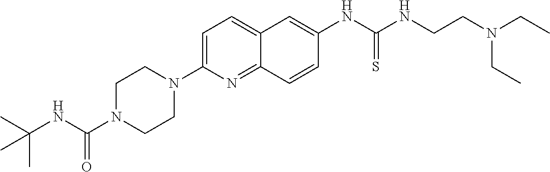 [SMILES: CCN(CC)CCNC(=S)Nc1ccc2nc(N3CCN(C(=O)NC(C)(C)C)CC3)ccc2c1] |
| 0019 | 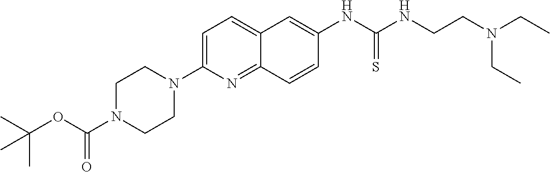 [SMILES: CCN(CC)CCNC(=S)Nc1ccc2nc(N3CCN(C(=O)OC(C)(C)C)CC3)ccc2c1] |
| 0020 | 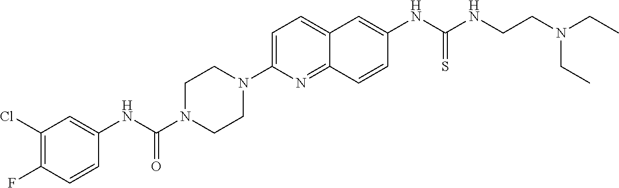 [SMILES: CCN(CC)CCNC(=S)Nc1ccc2nc(N3CCN(C(=O)Nc4ccc(F)c(Cl)c4)CC3)ccc2c1] |
| 0021 | 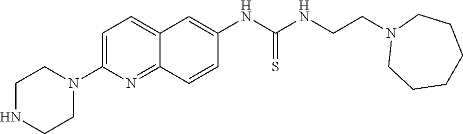 [SMILES: S=C(NCCN1CCCCCC1)Nc1ccc2nc(N3CCNCC3)ccc2c1] |
| 0022 | 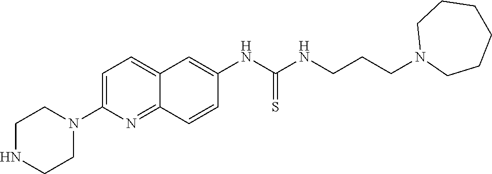 [SMILES: S=C(NCCCN1CCCCCC1)Nc1ccc2nc(N3CCNCC3)ccc2c1] |
| 0023 | 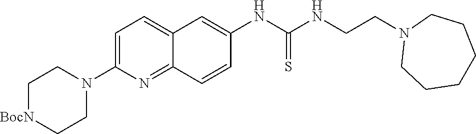 [SMILES: CC(C)(C)OC(=O)N1CCN(c2ccc3cc(NC(=S)NCCN4CCCCCC4)ccc3n2)CC1] |
| 0024 | 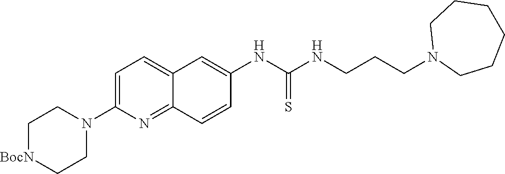 [SMILES: CC(C)(C)OC(=O)N1CCN(c2ccc3cc(NC(=S)NCCCN4CCCCCC4)ccc3n2)CC1] |
| 0025 | 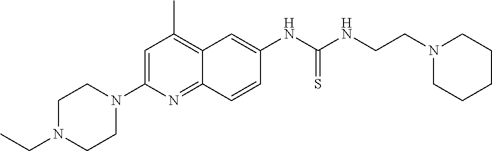 [SMILES: CCN1CCN(c2cc(C)c3cc(NC(=S)NCCN4CCCCC4)ccc3n2)CC1] |
| 0026 | 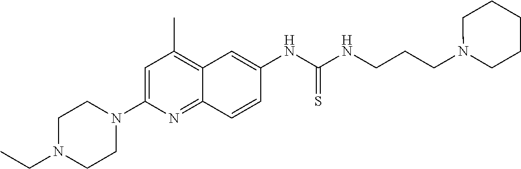 [SMILES: CCN1CCN(c2cc(C)c3cc(NC(=S)NCCCN4CCCCC4)ccc3n2)CC1] |
| 0027 | 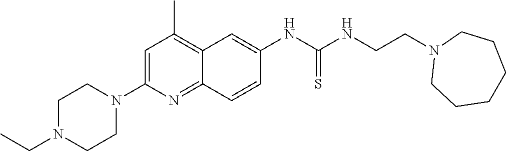 [SMILES: CCN1CCN(c2cc(C)c3cc(NC(=S)NCCN4CCCCCC4)ccc3n2)CC1] |
| 0028 | 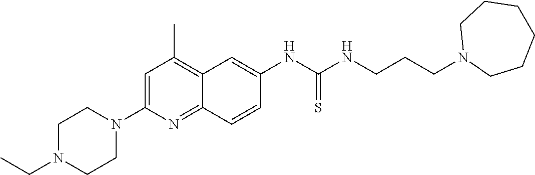 [SMILES: CCN1CCN(c2cc(C)c3cc(NC(=S)NCCCN4CCCCCC4)ccc3n2)CC1] |
| 0029 | 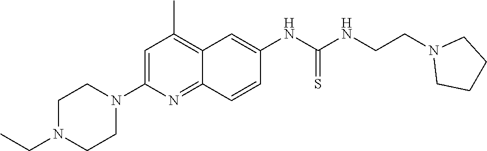 [SMILES: CCN1CCN(c2cc(C)c3cc(NC(=S)NCCN4CCCC4)ccc3n2)CC1] |
| 0030 | 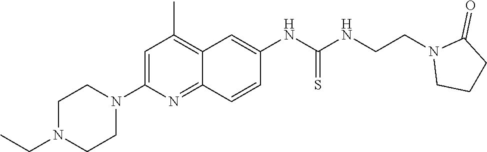 [SMILES: CCN1CCN(c2cc(C)c3cc(NC(=S)NCCN4CCCC4=O)ccc3n2)CC1] |
| 0031 | 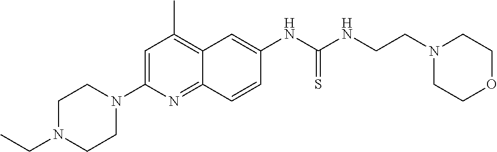 [SMILES: CCN1CCN(c2cc(C)c3cc(NC(=S)NCCN4CCOCC4)ccc3n2)CC1] |
| 0032 | 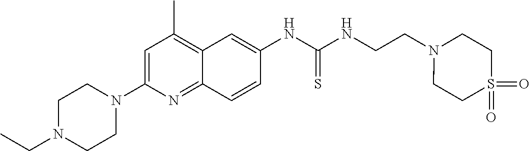 [SMILES: CCN1CCN(c2cc(C)c3cc(NC(=S)NCCN4CCS(=O)(=O)CC4)ccc3n2)CC1] |
| 0033 | 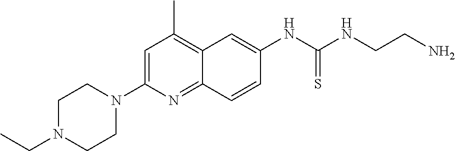 [SMILES: CCN1CCN(c2cc(C)c3cc(NC(=S)NCCN)ccc3n2)CC1] |
| 0034 | 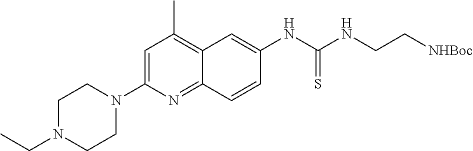 [SMILES: CCN1CCN(c2cc(C)c3cc(NC(=S)NCCNC(=O)OC(C)(C)C)ccc3n2)CC1] |
| 0035 | 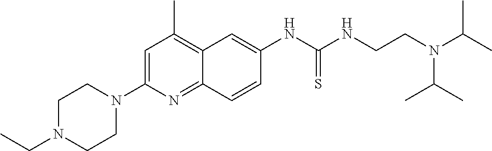 [SMILES: CCN1CCN(c2cc(C)c3cc(NC(=S)NCCN(C(C)C)C(C)C)ccc3n2)CC1] |
| 0036 | 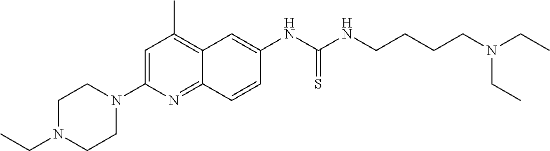 [SMILES: CCN(CC)CCCCNC(=S)Nc1ccc2nc(N3CCN(CC)CC3)cc(C)c2c1] |
| 0037 | 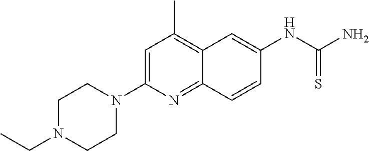 [SMILES: CCN1CCN(c2cc(C)c3cc(NC(N)=S)ccc3n2)CC1] |
| 0038 | 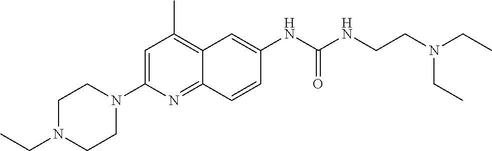 [SMILES: CCN(CC)CCNC(=O)Nc1ccc2nc(N3CCN(CC)CC3)cc(C)c2c1] |
| 0039 | 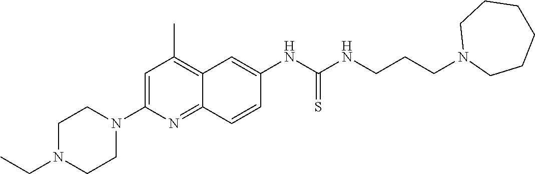 [SMILES: CCN1CCN(c2cc(C)c3cc(NC(=S)NCCCN4CCCCCC4)ccc3n2)CC1] |
| 0040 | 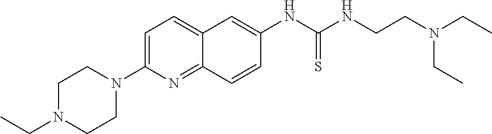 [SMILES: CCN(CC)CCNC(=S)Nc1ccc2nc(N3CCN(CC)CC3)ccc2c1] |
| 0041 | 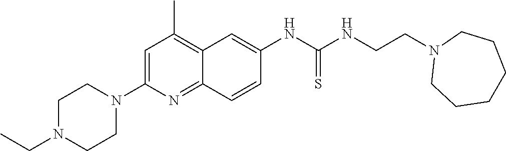 [SMILES: CCN1CCN(c2cc(C)c3cc(NC(=S)NCCN4CCCCCC4)ccc3n2)CC1] |
| 0042 | 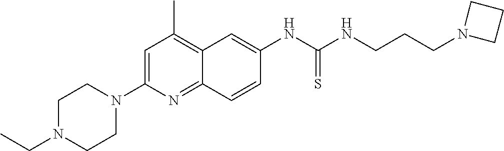 [SMILES: CCN1CCN(c2cc(C)c3cc(NC(=S)NCCCN4CCC4)ccc3n2)CC1] |
| 0043 | 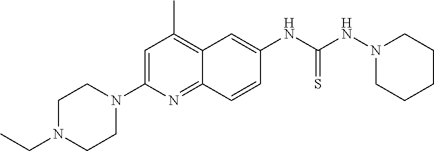 [SMILES: CCN1CCN(c2cc(C)c3cc(NC(=S)NN4CCCCC4)ccc3n2)CC1] |
| 0044 | 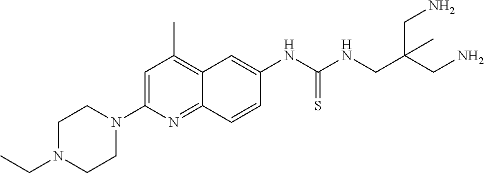 [SMILES: CCN1CCN(c2cc(C)c3cc(NC(=S)NCC(C)(CN)CN)ccc3n2)CC1] |
| 0045 | 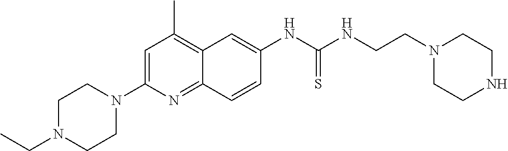 [SMILES: CCN1CCN(c2cc(C)c3cc(NC(=S)NCCN4CCNCC4)ccc3n2)CC1] |
| 0046 | 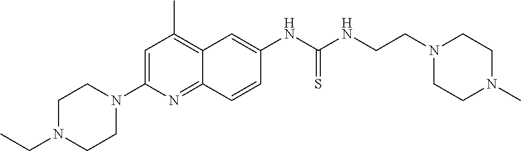 [SMILES: CCN1CCN(c2cc(C)c3cc(NC(=S)NCCN4CCN(C)CC4)ccc3n2)CC1] |
| 0047 | 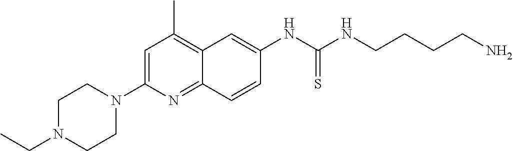 [SMILES: CCN1CCN(c2cc(C)c3cc(NC(=S)NCCCCN)ccc3n2)CC1] |
| 0048 | 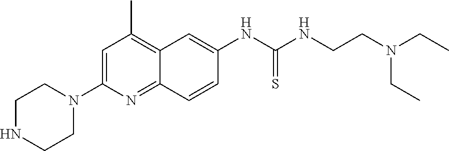 [SMILES: CCN(CC)CCNC(=S)Nc1ccc2nc(N3CCNCC3)cc(C)c2c1] |
| 0049 | 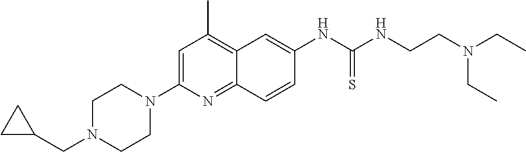 [SMILES: CCN(CC)CCNC(=S)Nc1ccc2nc(N3CCN(CC4CC4)CC3)cc(C)c2c1] |
| 0050 | 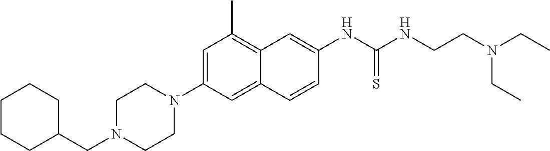 [SMILES: CCN(CC)CCNC(=S)Nc1ccc2cc(N3CCN(CC4CCCCC4)CC3)cc(C)c2c1] |
| 0051 | 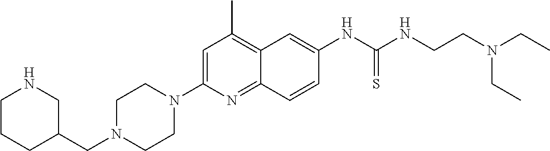 [SMILES: CCN(CC)CCNC(=S)Nc1ccc2nc(N3CCN(CC4CCCNC4)CC3)cc(C)c2c1] |
| 0052 | 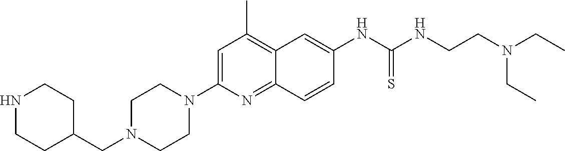 [SMILES: CCN(CC)CCNC(=S)Nc1ccc2nc(N3CCN(CC4CCNCC4)CC3)cc(C)c2c1] |
| 0053 | 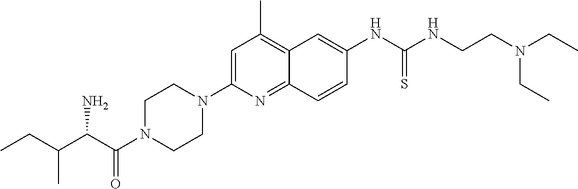 [SMILES: CCC(C)[C@H](N)C(=O)N1CCN(c2cc(C)c3cc(NC(=S)NCCN(CC)CC)ccc3n2)CC1] |
| 0054 | 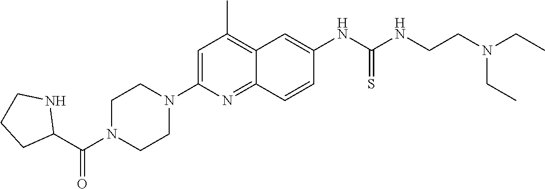 [SMILES: CCN(CC)CCNC(=S)Nc1ccc2nc(N3CCN(C(=O)C4CCCN4)CC3)cc(C)c2c1] |
| 0055 | 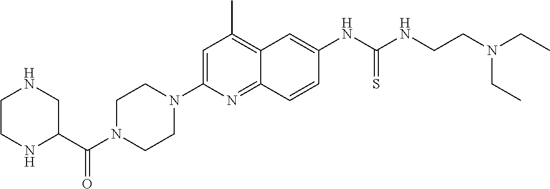 [SMILES: CCN(CC)CCNC(=S)Nc1ccc2nc(N3CCN(C(=O)C4CNCCN4)CC3)cc(C)c2c1] |
| 0056 | 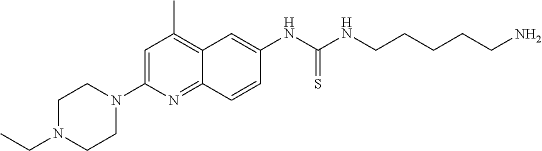 [SMILES: CCN1CCN(c2cc(C)c3cc(NC(=S)NCCCCCN)ccc3n2)CC1] |
| 0057 | 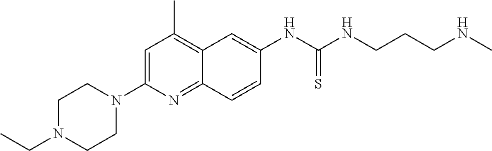 [SMILES: CCN1CCN(c2cc(C)c3cc(NC(=S)NCCCNC)ccc3n2)CC1] |
| 0058 | 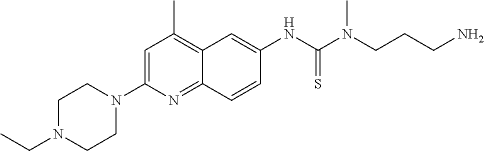 [SMILES: CCN1CCN(c2cc(C)c3cc(NC(=S)N(C)CCCN)ccc3n2)CC1] |
| 0059 | 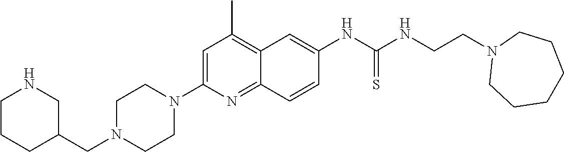 [SMILES: Cc1cc(N2CCN(CC3CCCNC3)CC2)nc2ccc(NC(=S)NCCN3CCCCCC3)cc12] |
| 0060 | 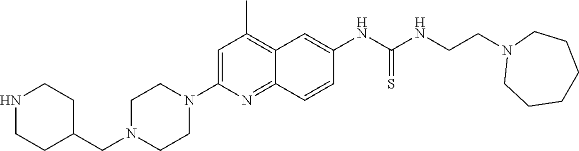 [SMILES: Cc1cc(N2CCN(CC3CCNCC3)CC2)nc2ccc(NC(=S)NCCN3CCCCCC3)cc12] |
| 0061 | 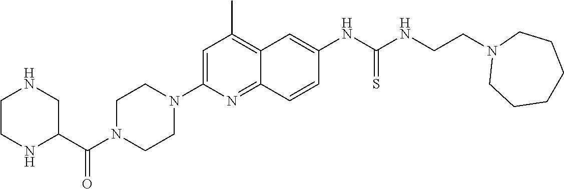 [SMILES: Cc1cc(N2CCN(C(=O)C3CNCCN3)CC2)nc2ccc(NC(=S)NCCN3CCCCCC3)cc12] |
| 0062 | 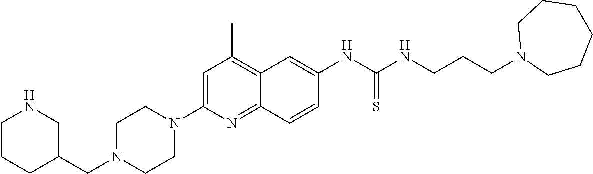 [SMILES: Cc1cc(N2CCN(CC3CCCNC3)CC2)nc2ccc(NC(=S)NCCCN3CCCCCC3)cc12] |
| 0063 | 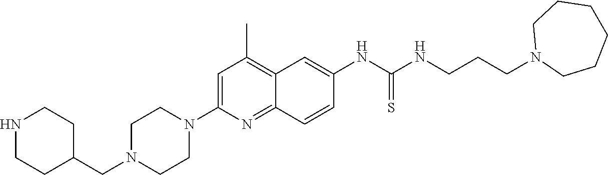 [SMILES: Cc1cc(N2CCN(CC3CCNCC3)CC2)nc2ccc(NC(=S)NCCCN3CCCCCC3)cc12] |
| 0064 | 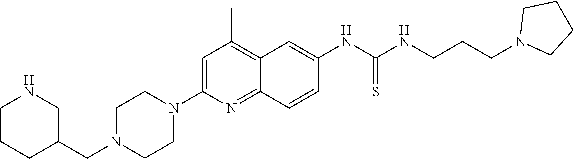 [SMILES: Cc1cc(N2CCN(CC3CCCNC3)CC2)nc2ccc(NC(=S)NCCCN3CCCC3)cc12] |
| 0065 | 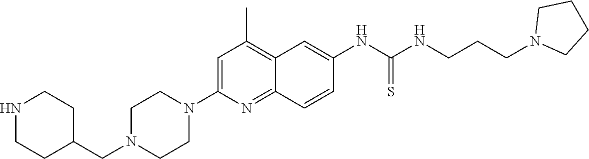 [SMILES: Cc1cc(N2CCN(CC3CCNCC3)CC2)nc2ccc(NC(=S)NCCCN3CCCC3)cc12] |
| 0066 | 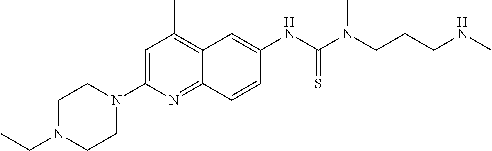 [SMILES: CCN1CCN(c2cc(C)c3cc(NC(=S)N(C)CCCNC)ccc3n2)CC1] |
| 0067 | 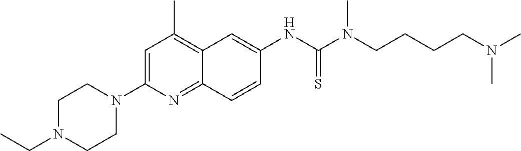 [SMILES: CCN1CCN(c2cc(C)c3cc(NC(=S)N(C)CCCCN(C)C)ccc3n2)CC1] |
| 0068 | 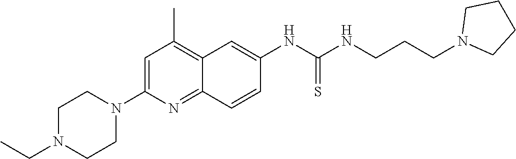 [SMILES: CCN1CCN(c2cc(C)c3cc(NC(=S)NCCCN4CCCC4)ccc3n2)CC1] |
| 0069 | 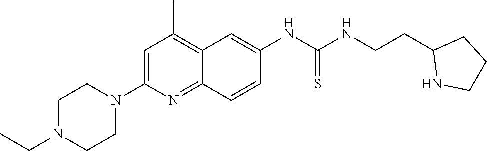 [SMILES: CCN1CCN(c2cc(C)c3cc(NC(=S)NCCC4CCCN4)ccc3n2)CC1] |
| 0070 | 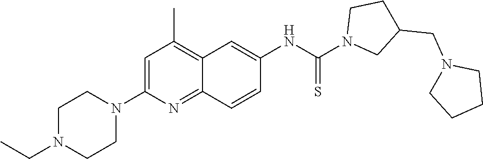 [SMILES: CCN1CCN(c2cc(C)c3cc(NC(=S)N4CCC(CN5CCCC5)C4)ccc3n2)CC1] |
| 0071 | 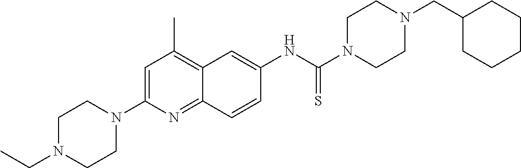 [SMILES: CCN1CCN(c2cc(C)c3cc(NC(=S)N4CCN(CC5CCCCC5)CC4)ccc3n2)CC1] |
| 0072 | 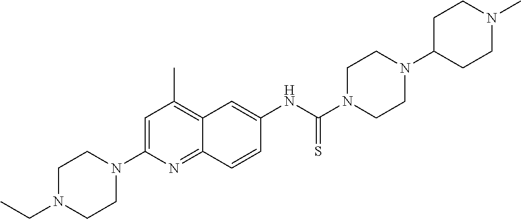 [SMILES: CCN1CCN(c2cc(C)c3cc(NC(=S)N4CCN(C5CCN(C)CC5)CC4)ccc3n2)CC1] |
| 0073 | 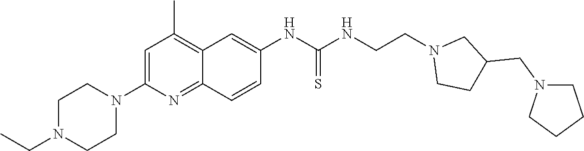 [SMILES: CCN1CCN(c2cc(C)c3cc(NC(=S)NCCN4CCC(CN5CCCC5)C4)ccc3n2)CC1] |
| 0074 | 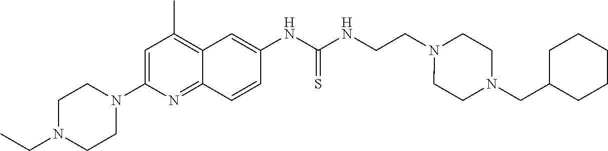 [SMILES: CCN1CCN(c2cc(C)c3cc(NC(=S)NCCN4CCN(CC5CCCCC5)CC4)ccc3n2)CC1] |
| 0075 | 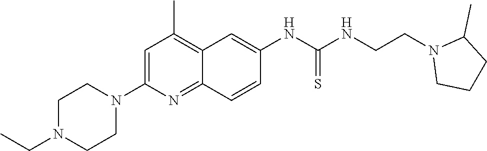 [SMILES: CCN1CCN(c2cc(C)c3cc(NC(=S)NCCN4CCCC4C)ccc3n2)CC1] |
| 0076 | 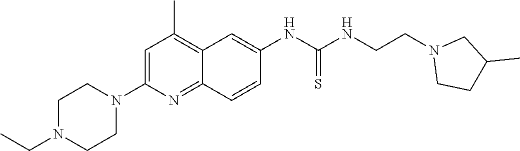 [SMILES: CCN1CCN(c2cc(C)c3cc(NC(=S)NCCN4CCC(C)C4)ccc3n2)CC1] |
| 0077 | 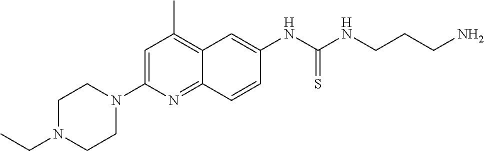 [SMILES: CCN1CCN(c2cc(C)c3cc(NC(=S)NCCCN)ccc3n2)CC1] |
| 0078 | 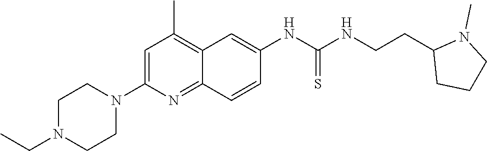 [SMILES: CCN1CCN(c2cc(C)c3cc(NC(=S)NCCC4CCCN4C)ccc3n2)CC1] |
| 0079 | 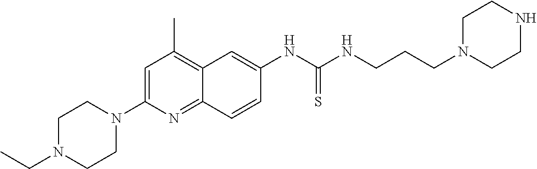 [SMILES: CCN1CCN(c2cc(C)c3cc(NC(=S)NCCCN4CCNCC4)ccc3n2)CC1] |
| 0080 | 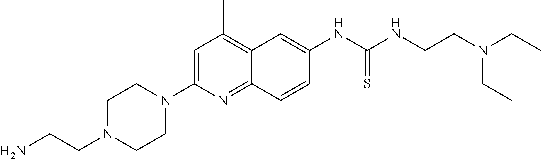 [SMILES: CCN(CC)CCNC(=S)Nc1ccc2nc(N3CCN(CCN)CC3)cc(C)c2c1] |
| 0082 | 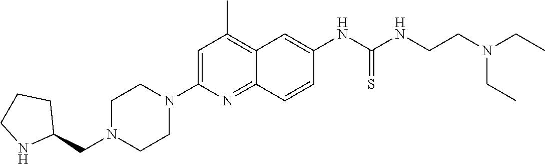 [SMILES: CCN(CC)CCNC(=S)Nc1ccc2nc(N3CCN(C[C@@H]4CCCN4)CC3)cc(C)c2c1] |
| 0083 | 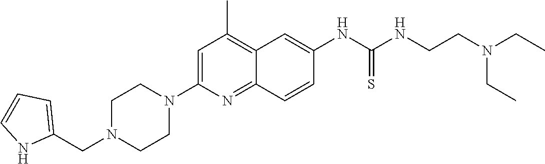 [SMILES: CCN(CC)CCNC(=S)Nc1ccc2nc(N3CCN(Cc4ccc[nH]4)CC3)cc(C)c2c1] |
| 0084 | 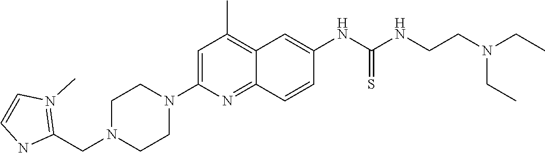 |
| 0085 | 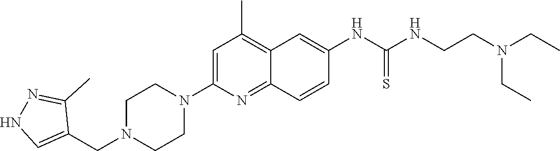 [SMILES: CCN(CC)CCNC(=S)Nc1ccc2nc(N3CCN(Cc4c[nH]nc4C)CC3)cc(C)c2c1] |
| 0086 | 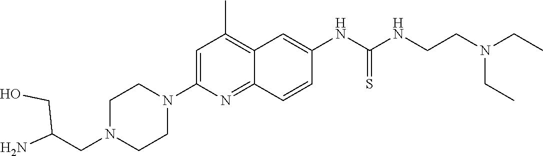 [SMILES: CCN(CC)CCNC(=S)Nc1ccc2nc(N3CCN(CC(N)CO)CC3)cc(C)c2c1] |
| 0087 | 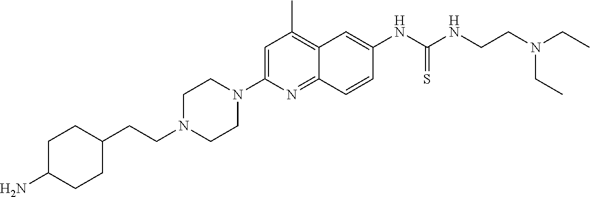 [SMILES: CCN(CC)CCNC(=S)Nc1ccc2nc(N3CCN(CCC4CCC(N)CC4)CC3)cc(C)c2c1] |
| 0088 | 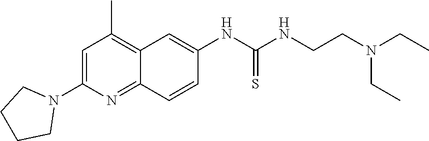 [SMILES: CCN(CC)CCNC(=S)Nc1ccc2nc(N3CCCC3)cc(C)c2c1] |
| 0089 | 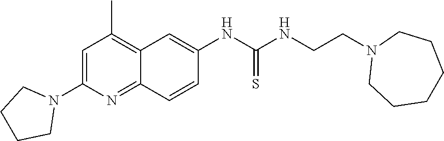 [SMILES: Cc1cc(N2CCCC2)nc2ccc(NC(=S)NCCN3CCCCCC3)cc12] |
| 0090 | 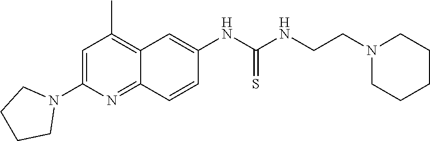 [SMILES: Cc1cc(N2CCCC2)nc2ccc(NC(=S)NCCN3CCCCC3)cc12] |
| 0091 | 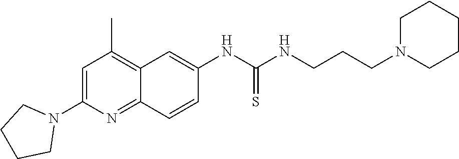 [SMILES: Cc1cc(N2CCCC2)nc2ccc(NC(=S)NCCCN3CCCCC3)cc12] |
| 0092 | 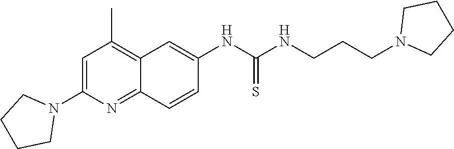 [SMILES: Cc1cc(N2CCCC2)nc2ccc(NC(=S)NCCCN3CCCC3)cc12] |
| 0093 | 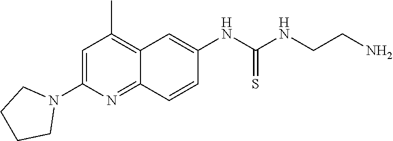 [SMILES: Cc1cc(N2CCCC2)nc2ccc(NC(=S)NCCN)cc12] |
| 0094 | 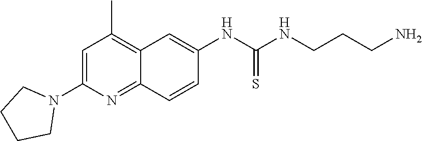 [SMILES: Cc1cc(N2CCCC2)nc2ccc(NC(=S)NCCCN)cc12] |
| 0095 | 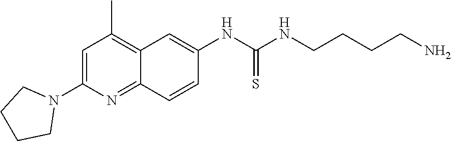 [SMILES: Cc1cc(N2CCCC2)nc2ccc(NC(=S)NCCCCN)cc12] |
| 0096 | 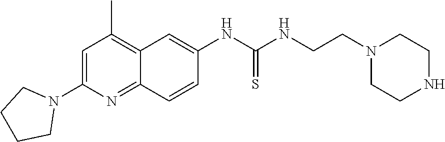 [SMILES: Cc1cc(N2CCCC2)nc2ccc(NC(=S)NCCN3CCNCC3)cc12] |
| 0097 | 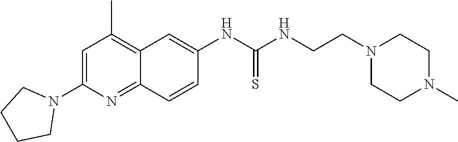 [SMILES: Cc1cc(N2CCCC2)nc2ccc(NC(=S)NCCN3CCN(C)CC3)cc12] |
| 0098 | 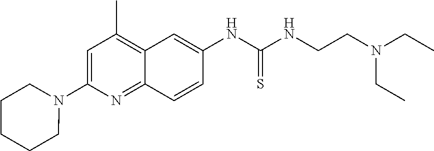 [SMILES: CCN(CC)CCNC(=S)Nc1ccc2nc(N3CCCCC3)cc(C)c2c1] |
| 0099 | 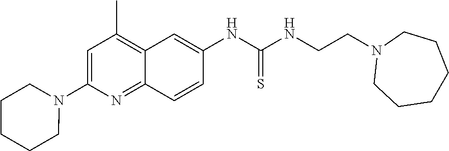 [SMILES: Cc1cc(N2CCCCC2)nc2ccc(NC(=S)NCCN3CCCCCC3)cc12] |
| 0100 | 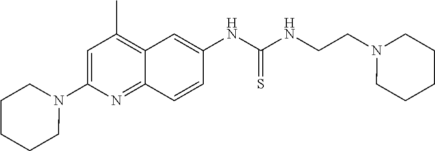 [SMILES: Cc1cc(N2CCCCC2)nc2ccc(NC(=S)NCCN3CCCCC3)cc12] |
| 0101 | 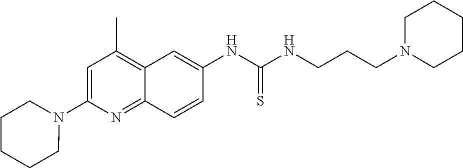 [SMILES: Cc1cc(N2CCCCC2)nc2ccc(NC(=S)NCCCN3CCCCC3)cc12] |
| 0102 | 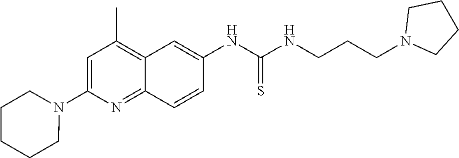 [SMILES: Cc1cc(N2CCCCC2)nc2ccc(NC(=S)NCCCN3CCCC3)cc12] |
| 0103 | 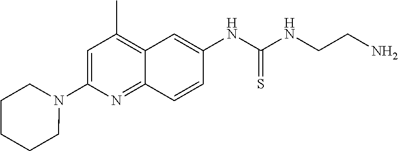 [SMILES: Cc1cc(N2CCCCC2)nc2ccc(NC(=S)NCCN)cc12] |
| 0104 | 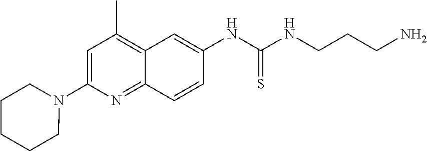 [SMILES: Cc1cc(N2CCCCC2)nc2ccc(NC(=S)NCCCN)cc12] |
| 0105 | 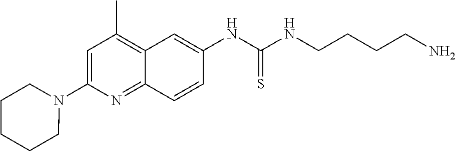 [SMILES: Cc1cc(N2CCCCC2)nc2ccc(NC(=S)NCCCCN)cc12] |
| 0106 | 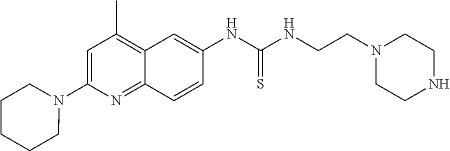 [SMILES: Cc1cc(N2CCCCC2)nc2ccc(NC(=S)NCCN3CCNCC3)cc12] |
| 0107 | 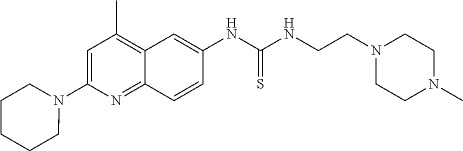 [SMILES: Cc1cc(N2CCCCC2)nc2ccc(NC(=S)NCCN3CCN(C)CC3)cc12] |
| 0108 | 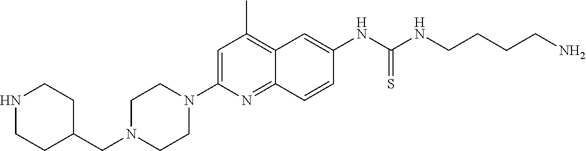 [SMILES: Cc1cc(N2CCN(CC3CCNCC3)CC2)nc2ccc(NC(=S)NCCCCN)cc12] |
| 0109 | 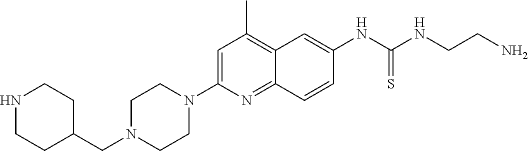 [SMILES: Cc1cc(N2CCN(CC3CCNCC3)CC2)nc2ccc(NC(=S)NCCN)cc12] |
| 0110 | 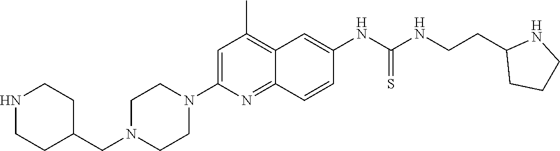 [SMILES: Cc1cc(N2CCN(CC3CCNCC3)CC2)nc2ccc(NC(=S)NCCC3CCCN3)cc12] |
| 0112 | 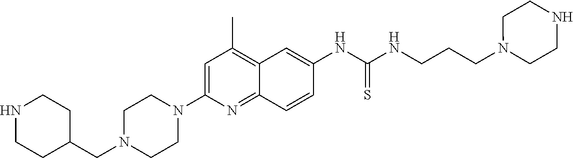 [SMILES: Cc1cc(N2CCN(CC3CCNCC3)CC2)nc2ccc(NC(=S)NCCCN3CCNCC3)cc12] |
| 0113 | 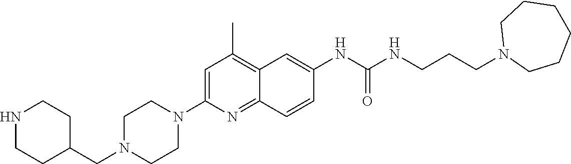 [SMILES: Cc1cc(N2CCN(CC3CCNCC3)CC2)nc2ccc(NC(=O)NCCCN3CCCCCC3)cc12] |
| 0114 | 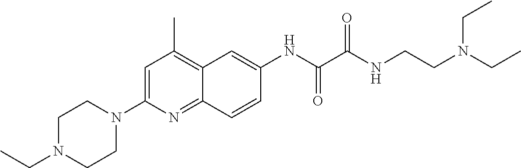 [SMILES: CCN(CC)CCNC(=O)C(=O)Nc1ccc2nc(N3CCN(CC)CC3)cc(C)c2c1] |

optionally further comprising a prodrug thereof,
optionally further comprising a salt thereof.

29-30. (canceled)

31. A pharmaceutical composition comprising the compound of claim 1 or a pharmaceutically acceptable salt thereof, and a pharmaceutically acceptable diluent or carrier.

32. A pharmaceutical composition comprising the compound of claim 28, or a pharmaceutically acceptable salt thereof, and a pharmaceutically acceptable diluent or carrier.

33. A method of modulating RAD52 activity, the method comprising contacting a cell with an effective amount of a compound of claim 1.

34. A method of treating or preventing a disease or disorder in a subject in need thereof, the method comprising administering to the subject a therapeutically effective amount of a compound of claim 1.

35-38. (canceled)

39. The method of claim 34, wherein the disease or disorder is associated with an implicated RAD52 activity.

40. The method of claim 34, wherein the disease or disorder is a cancer.

41. The method of claim 40, wherein the cancer has a dysfunctional BRCA1, BRCA2, PALB2, or RAD51 paralog activity.

42. The method of claim 40, wherein the cancer is squamous cell cancer, lung cancer, vulval cancer, thyroid cancer, adenocarcinoma of the lung, squamous carcinoma of the lung, cancer of the peritoneum, hepatocellular cancer, gastric or stomach cancer, gastroesophageal, pancreatic cancer, brain cancer, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, biliary tract, anal carcinoma, penile carcinoma, leukemia, lymphoma, melanoma, or head and neck cancer.

43. The method of claim 42, wherein the cancer is ovarian cancer.

44. The method of claim 43, wherein the ovarian cancer has at least one of a BRCA1 mutation and a BRCA2 mutation.

45. The method of claim 42, wherein the cancer is breast cancer.

46. The method of claim 45, wherein the breast cancer has at least one of a BRCA1 mutation and a BRCA2 mutation.


SEQUENCE LISTING (4 sequences)

SEQ ID NO: 1 - DNA, 60 bp, Artificial Sequence
  Chemically synthesized
cactgtgatgcacgatgatcgacgacagtagtcagtgctgggtcaacatctgtatgcagg

SEQ ID NO: 2 - DNA, 39 bp, Artificial Sequence
  Chemically synthesized
agcactgactactgtcgtcgatcatcgtgcatcacagtg

SEQ ID NO: 3 - DNA, 55 bp, Artificial Sequence
  Chemically synthesized
atacagatgttgacccagcactgactactgtcgtcaatcatcgtgcatcacagtg

SEQ ID NO: 4 - DNA, 90 bp, Artificial Sequence
  Chemicaly synthesized
cgggtgtcggggctggcttaactatgcggcatcagagcagattgtactgagagtgcaccatatgcggtgtgaaataccgcacagatgcgt